(19) United States
(12) Patent Application Publication
(10) Pub. No.: US 2012/0104941 A1
(43) Pub. Date: May 3, 2012

(54) COMPOUND FOR AN ORGANIC PHOTOELECTRIC DEVICE AND ORGANIC PHOTOELECTRIC DEVICE INCLUDING THE SAME

(76) Inventors: Sung-Hyun JUNG, Uiwang-si (KR); Myeong-Soon KANG, Uiwang-si (KR); Ho-Kuk JUNG, Uiwang-si (KR); Nam-Soo KIM, Uiwang-si (KR); Dong-Min KANG, Uiwang-si (KR); Nam-Heon LEE, Uiwang-si (KR); Mi-Young CHAE, Uiwang-si (KR)

(21) Appl. No.: 13/346,080

(22) Filed: Jan. 9, 2012

Related U.S. Application Data
(63) Continuation of application No. PCT/KR2010/004495, filed on Jul. 9, 2010.

(30) Foreign Application Priority Data
Jul. 10, 2009 (KR) 10-2009-0063234

(51) Int. Cl. H05B 33/14; C07D 401/14; C07D 401/10; C07D 403/10

(52) U.S. Cl. 313/504; 544/296; 546/167; 544/294; 544/180

(57) ABSTRACT

A compound for an organic photoelectric device, an organic photoelectric device, and a display device, the compound being represented by the following Chemical Formula 3a, 3b, or 3c:

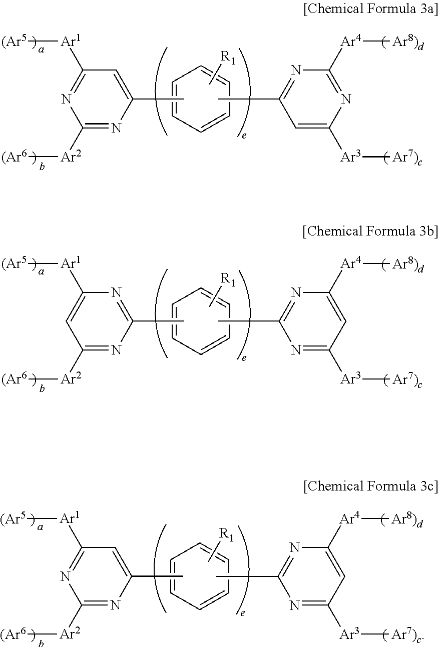

### CROSS-REFERENCE TO RELATED APPLICATION

This application is a continuation of pending International Application No. PCT/KR2010/004495, entitled “Compound for Organic Photoelectric Device and Organic Photoelectric Device,” which was filed on Jul. 9, 2010, the entire contents of which are hereby incorporated by reference.

### BACKGROUND

1. Field

Embodiments relate to a compound for an organic photoelectric device and an organic photoelectric device including the same.

2. Description of the Related Art

A photoelectric device is, in a broad sense, a device for transforming photo-energy to electrical energy and conversely, for transforming electrical energy to photo-energy. The photoelectric device may include an organic light emitting diode (OLED), a solar cell, a transistor, and the like. Particularly, an organic light emitting diode has recently drawn attention due to the increasing demand for a flat panel display.

When current is applied to an organic light emitting diode, holes and electrons may be respectively injected from the anode and the cathode, and the injected holes and electrons may go through the hole transport layer (HTL) and the electron transport layer (ETL) and then, may be recombined together in the emission layer to provide light emitting excitons. The light emitting excitons transit into the ground state and emit light. The emitted light may be classified as fluorescence (using singlet excitons) and phosphorescence (using triplet excitons). The fluorescence and phosphorescence may be used as a light emitting source of organic light emitting diodes.

When electrons are transited from a ground state to an exited state, singlet excitons may be transited to triplet excitons through intersystem crossing without emitting light. Then, the triplet excitons may be transited to a ground state to emit light. Such a light emitting is referred to as phosphorescence. The triplet excitons may not directly transit to the ground state. Herein, the electron spin may be flipped, since the electron spin may be forbidden. Accordingly, phosphorescence may have longer lifetime (emission duration) than fluorescence.

### SUMMARY

Embodiments are directed to a compound for an organic photoelectric device and an organic photoelectric device including the same.

The embodiments may be realized by providing a compound for an organic photoelectric device, the compound being represented by the following Chemical Formula 3a, 3b, or 3c:

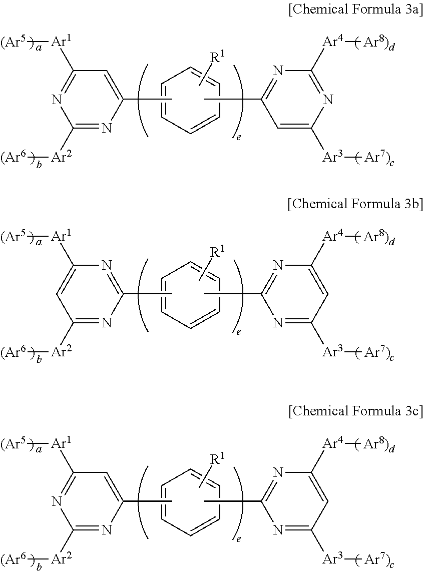

wherein, in Chemical Formulae 3a to 3c R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof, Ar⁵ to Ar⁸ are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, a, b, c, and d are each independently integers ranging from 0 to 2, provided that when a, b, c, and d are 0, at least one selected from Ar¹ to Ar⁴ has 7 to 30 carbon atoms, and e is 1 or 2, provided that when e is 2, two phenylene groups having the R1 substituents are in a para position relative to each other.

When any of R¹ and Ar¹ to Ar⁸ in the above Chemical Formulae 3a to 3c is an aryl group, the aryl group may be a phenyl group, a biphenyl group, a terphenyl group, a stilbenzyl group, a naphthyl group, an anthracenyl group, a phenanthrenyl group, a pyrenyl group, a perylenyl group, or a combination thereof, and when any of Ar¹ to Ar⁴ in the above Chemical Formulae 3a to 3c is an arylene group, the arylene group may be a phenylene group, a biphenylene group, a terphenylene group, a stilbenzylene group, a naphthylene group, an anthracenylene group, a phenanthrenylene group, a pyrenylene group, a perylenylene group, or a combination thereof.

When any of R¹ and Ar¹ to Ar⁸ in the above Chemical Formulae 3a to 3c is a heteroaryl group, the heteroaryl group may be a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and when any of Ar¹ to Ar⁴ in the above Chemical Formulas 3a to 3c is a heteroarylene group, the heteroarylene group may be a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

The compound for an organic photoelectric device may be a charge transporting material or a host material.

The embodiments may also be realized by providing a compound for an organic photoelectric device, the compound being represented by the following Chemical Formula 5a, 5b, 5c, or 5d:

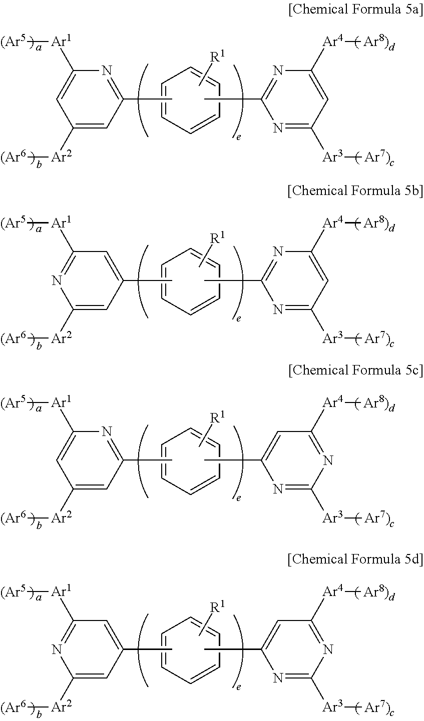

wherein, in Chemical Formulae 5a to 5d R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof, Ar⁵ to Ar⁸ are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, a, b, c, and d are each independently integers ranging from 0 to 2, and e is 1 or 2, provided that when e is 2, two phenylene groups having the R1 substituents are in a para position relative to each other.

When any of R¹ and Ar¹ to Ar⁸ in the above Chemical Formulae 5a to 5d is an aryl group, the aryl group may be a phenyl group, a biphenyl group, a terphenyl group, a stilbenzyl group, a naphthyl group, an anthracenyl group, a phenanthrenyl group, a pyrenyl group, a perylenyl group, or a combination thereof, and when any of Ar¹ to Ar⁴ in the above Chemical Formulae 5a to 5d is an arylene group, the arylene group may be a phenylene group, a biphenylene group, a terphenylene group, a stilbenzylene group, a naphthylene group, an anthracenylene group, a phenanthrenylene group, a pyrenylene group, a perylenylene group, or a combination thereof.

When any of R¹ and Ar¹ to Ar⁸ in the above Chemical Formulae 5a to 5d is a heteroaryl group, the heteroaryl group may be a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and when any of Ar¹ to Ar⁴ in the above Chemical Formulae 5a to 5d is a heteroarylene group, the heteroarylene group may be a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

The compound for an organic photoelectric device may be a charge transporting material or a host material.

The embodiments may also be realized by providing a compound for an organic photoelectric device, the compound being represented by the following Chemical Formula 6a or 6b:

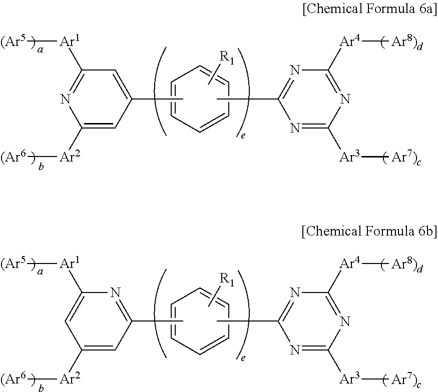

wherein, in Chemical Formulae 6a and 6b R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof, Ar⁵ to Ar⁸ are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, a, b, c, and d are each independently integers ranging from 0 to 2, and e is 1 or 2, provided that when e is 2, two phenylene groups having the R1 substituents are in a para position relative to each other.

When any of R¹ and Ar¹ to Ar⁸ in the above Chemical Formulae 6a and 6b is an aryl group, the aryl group may be a phenyl group, a biphenyl group, a terphenyl group, a stilbenzyl group, a naphthyl group, an anthracenyl group, a phenanthrenyl group, a pyrenyl group, a perylenyl group, or a combination thereof, and when any of Ar¹ to Ar⁴ in the above Chemical Formulae 6a and 6b is an arylene group, the arylene group may be a phenylene group, a biphenylene group, a terphenylene group, a stilbenzylene group, a naphthylene group, an anthracenylene group, a phenanthrenylene group, a pyrenylene group, a perylenylene group, or a combination thereof.

When any of R¹ and Ar¹ to Ar⁸ in the above Chemical Formulae 6a and 6b is a heteroaryl group, the heteroaryl group may be a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and when any of Ar¹ to Ar⁴ in the above Chemical Formulae 6a and 6b is a heteroarylene group, the heteroarylene group may be a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

The compound for an organic photoelectric device may be a charge transporting material or a host material.

The embodiments may also be realized by providing a compound for an organic photoelectric device, the compound being represented by the following Chemical Formula 7a or 7b:

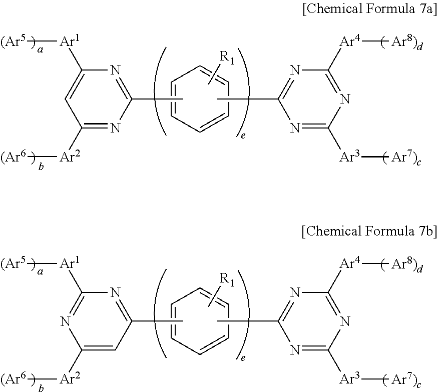

wherein, in Chemical Formulae 7a and 7b R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof, Ar⁵ to Ar⁸ are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof, a, b, c, and d are each independently integers ranging from 0 to 2, and e is 1 or 2, provided that when e is 2, two phenylene groups having the R1 substituents are in a para position relative to each other.

When any of R¹ and Ar¹ to Ar⁸ in the above Chemical Formulae 7a and 7b is an aryl group, the aryl group may be a phenyl group, a biphenyl group, a terphenyl group, a stilbenzyl group, a naphthyl group, an anthracenyl group, a phenanthrenyl group, a pyrenyl group, a perylenyl group, or a combination thereof, and when any of Ar¹ to Ar⁴ in the above Chemical Formulae 7a and 7b is an arylene group, the arylene group may be a phenylene group, a biphenylene group, a terphenylene group, a stilbenzylene group, a naphthylene group, an anthracenylene group, a phenanthrenylene group, a pyrenylene group, a perylenylene group, or a combination thereof.

When any of R¹ and Ar¹ to Ar⁸ in the above Chemical Formulae 7a and 7b is a heteroaryl group, the heteroaryl group may be a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and when any of Ar¹ to Ar⁴ in the above Chemical Formulae 7a and 7b is a heteroarylene group, the heteroarylene group may be a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

The compound for an organic photoelectric device may be a charge transporting material or a host material.

The embodiments may also be realized by providing a compound for an organic photoelectric device, the compound being represented by one of the following Compounds 8-1 to 8-258:

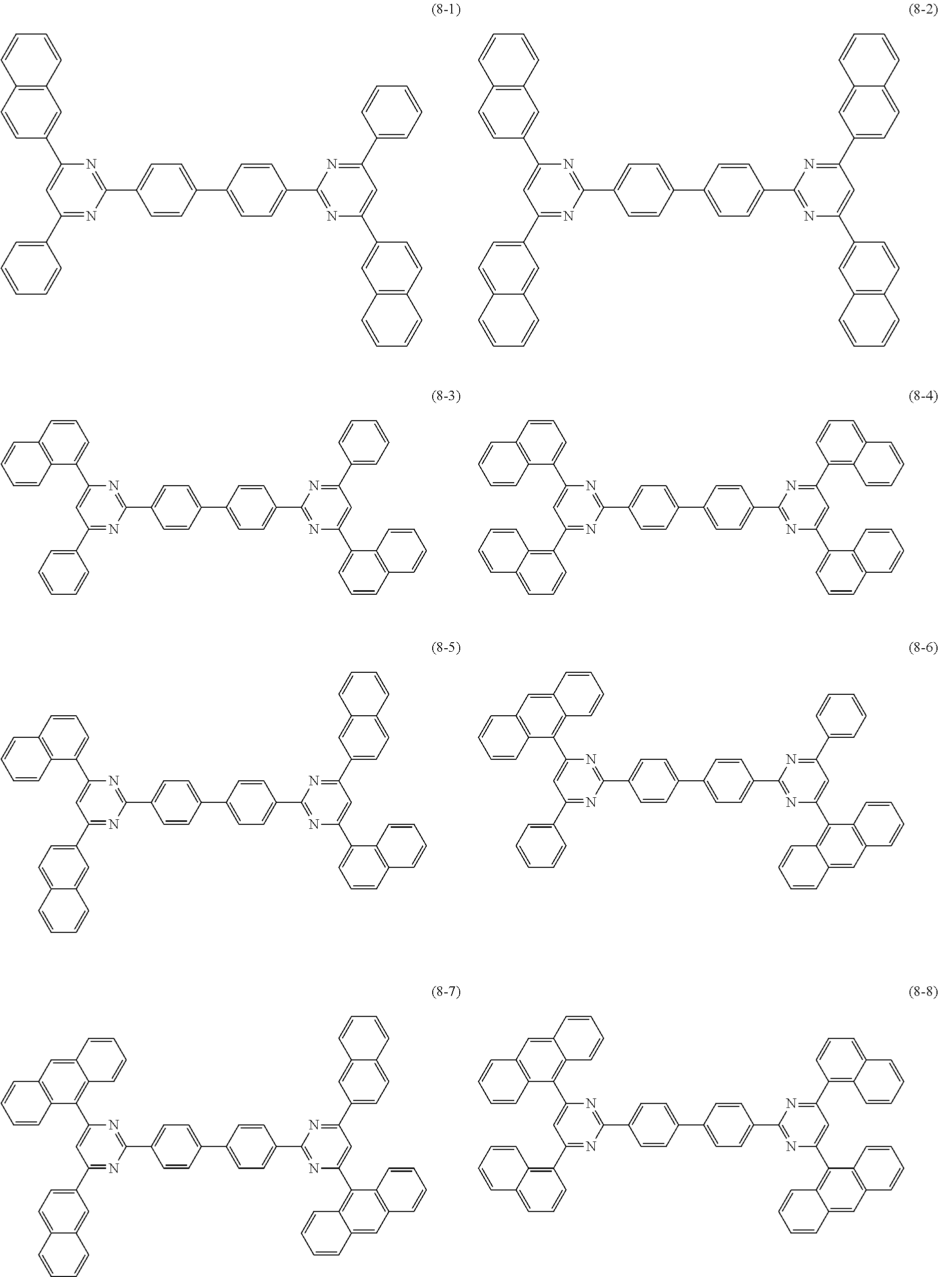

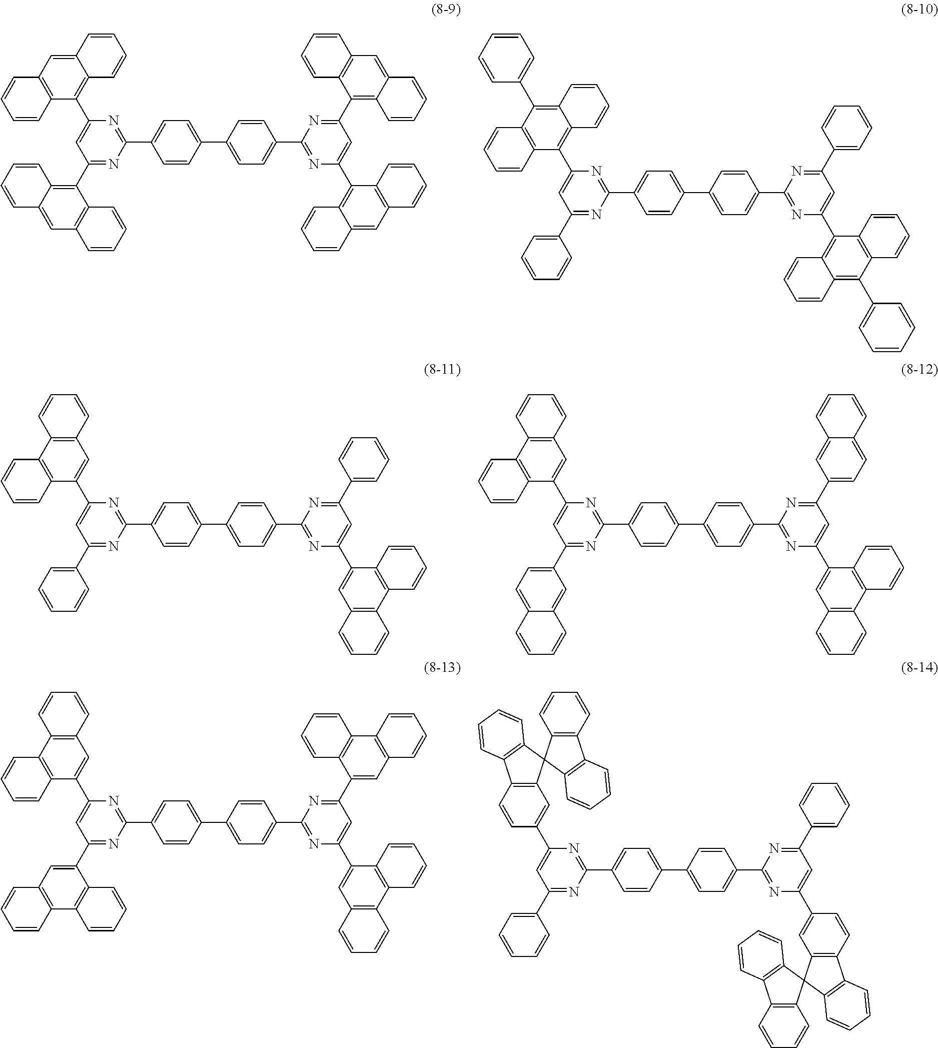

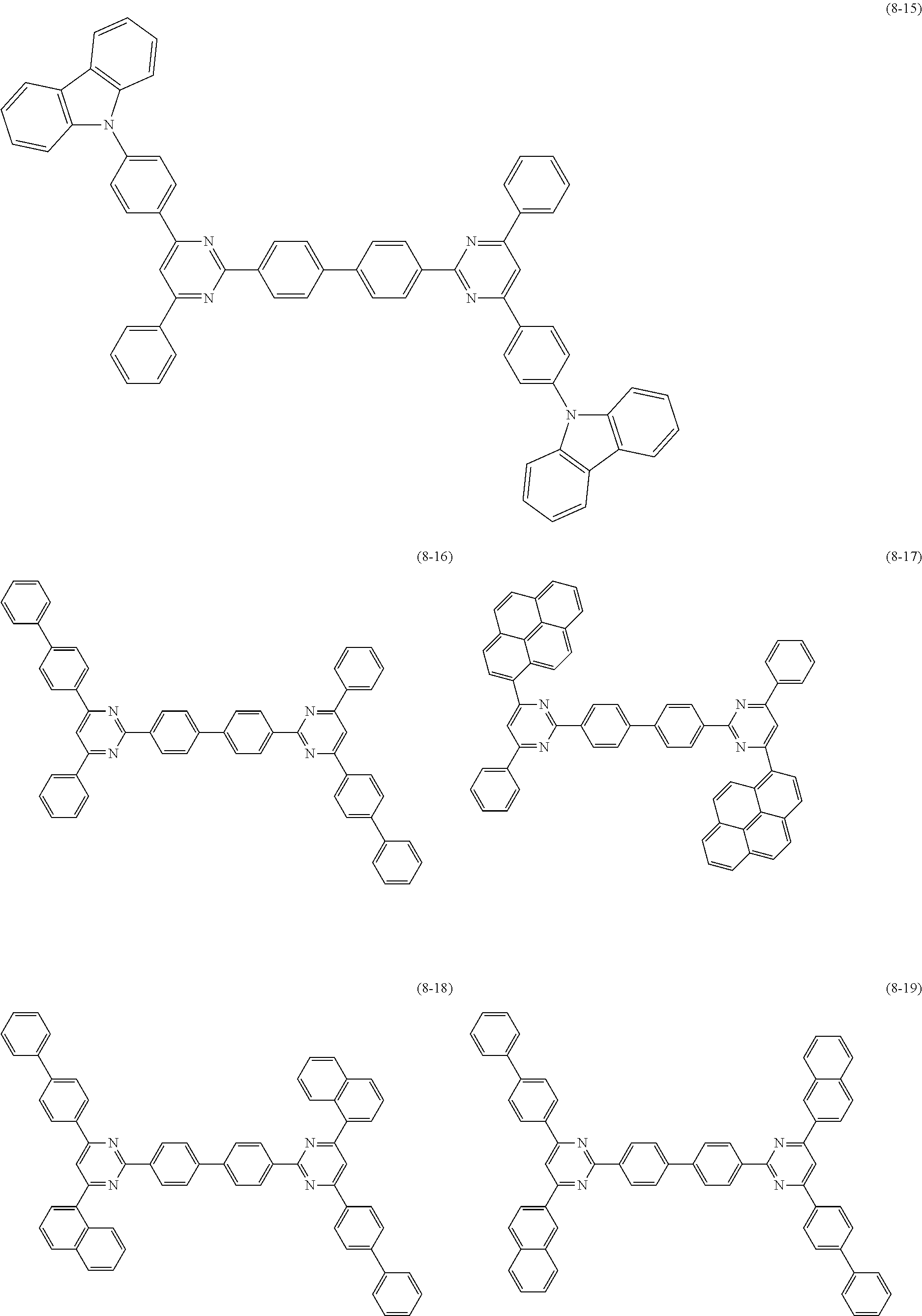

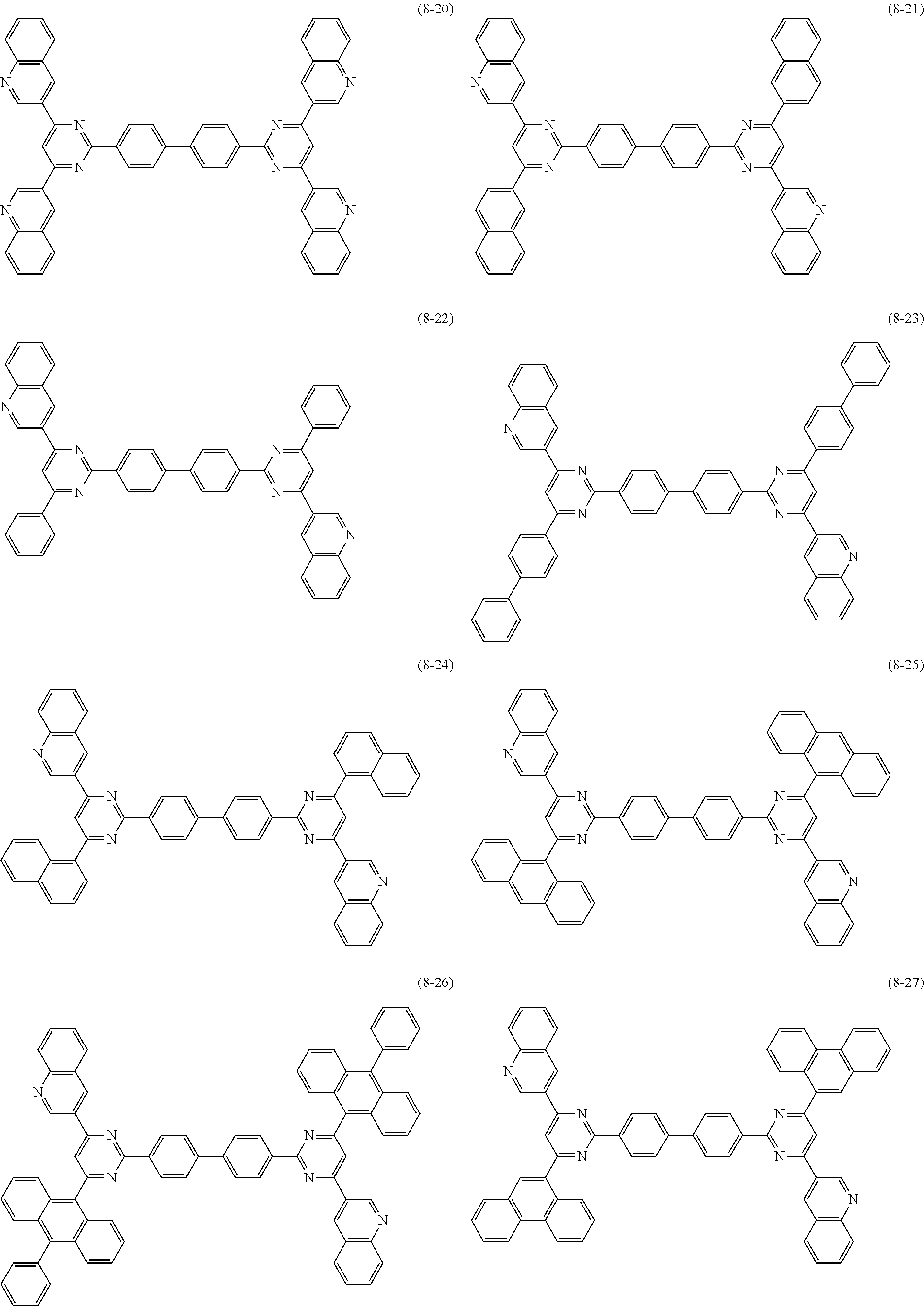

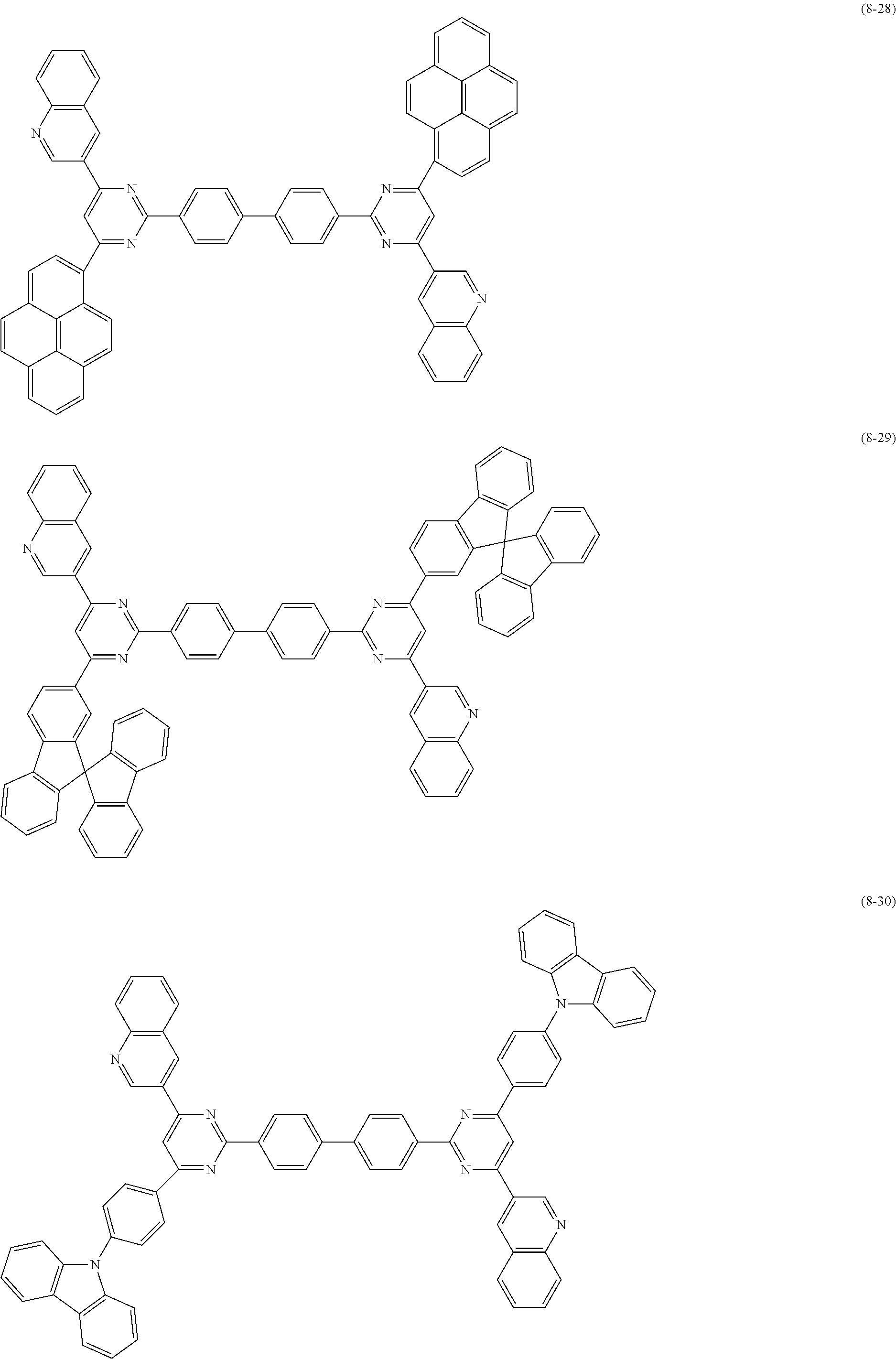

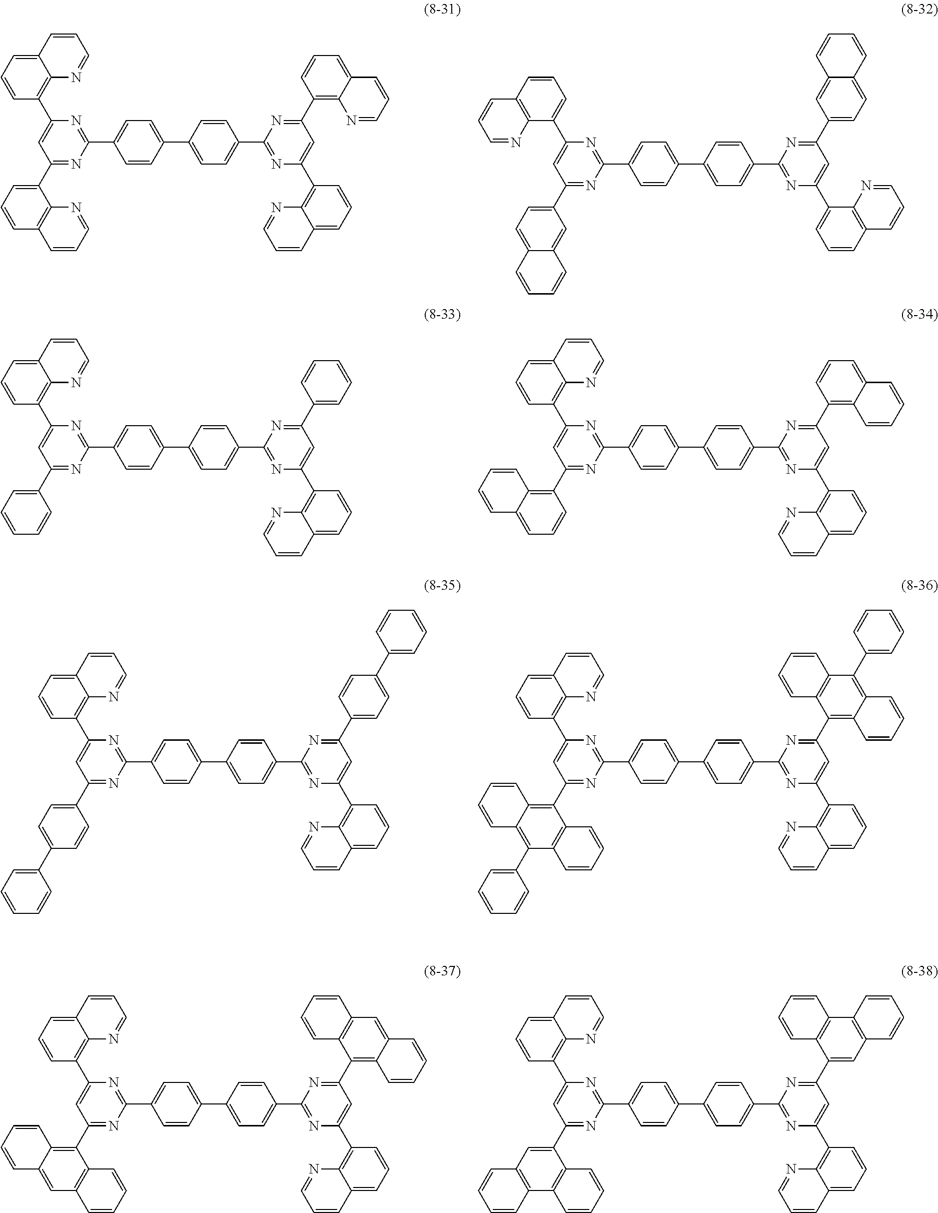

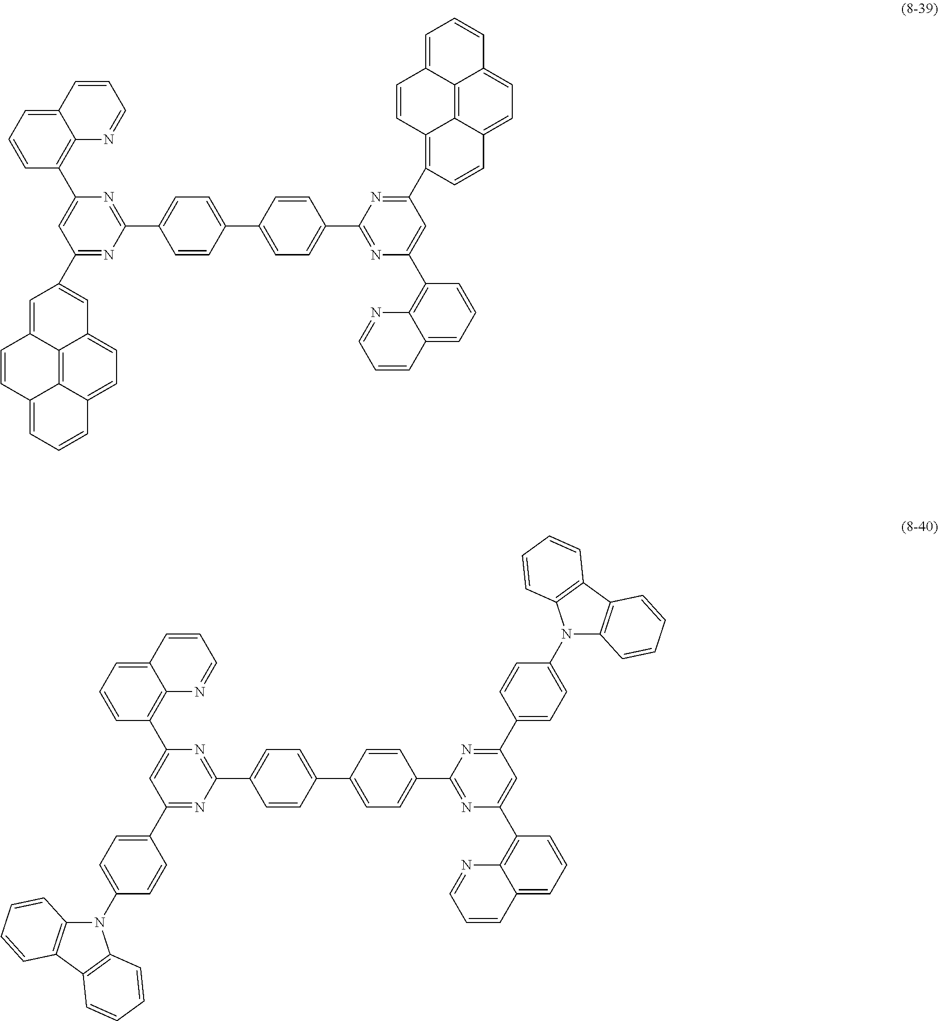

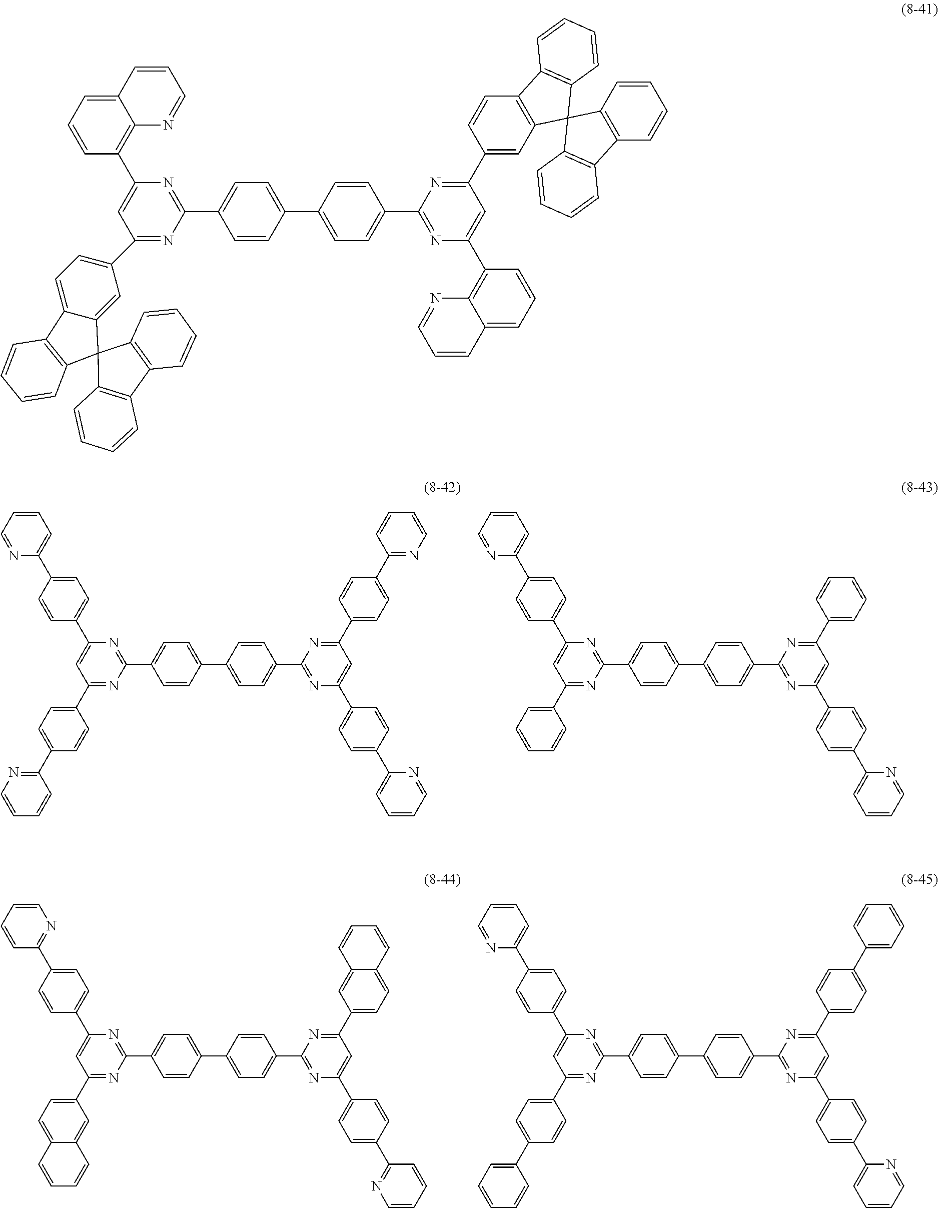

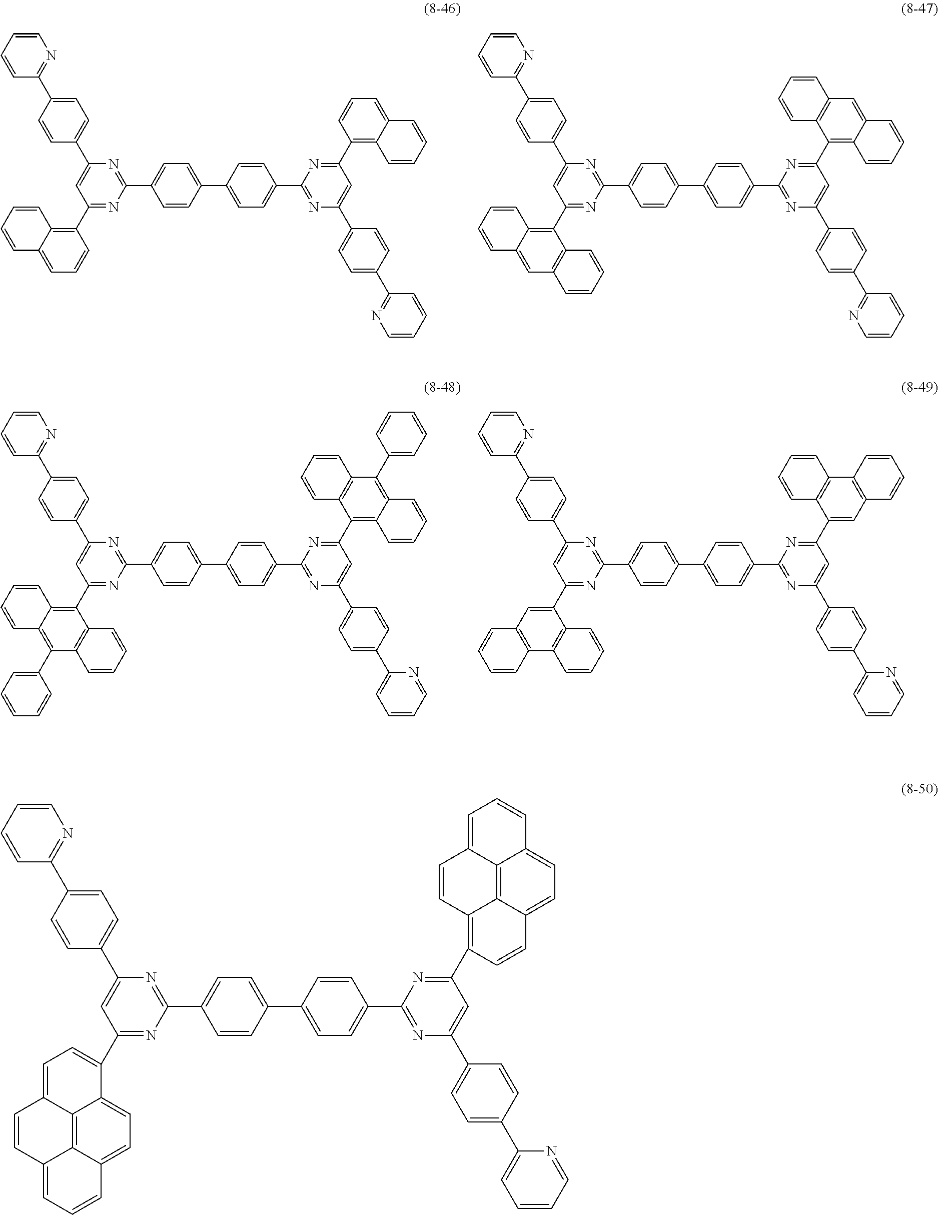

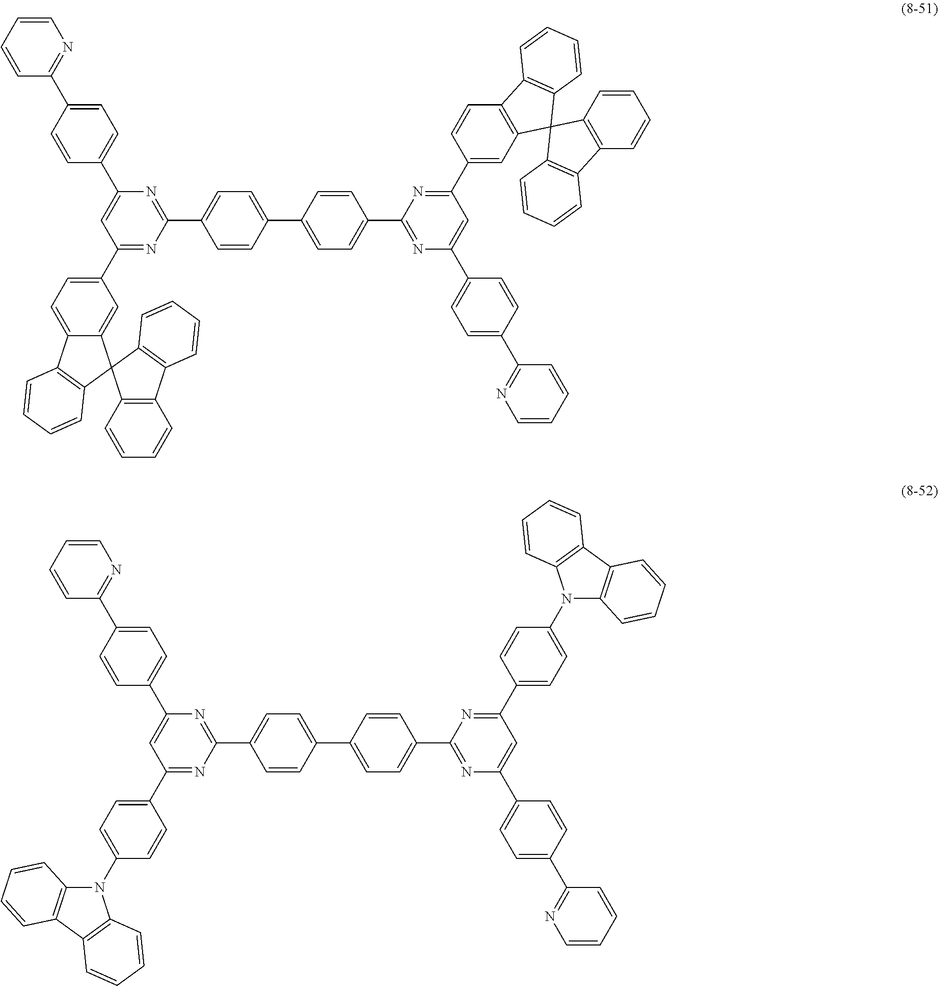

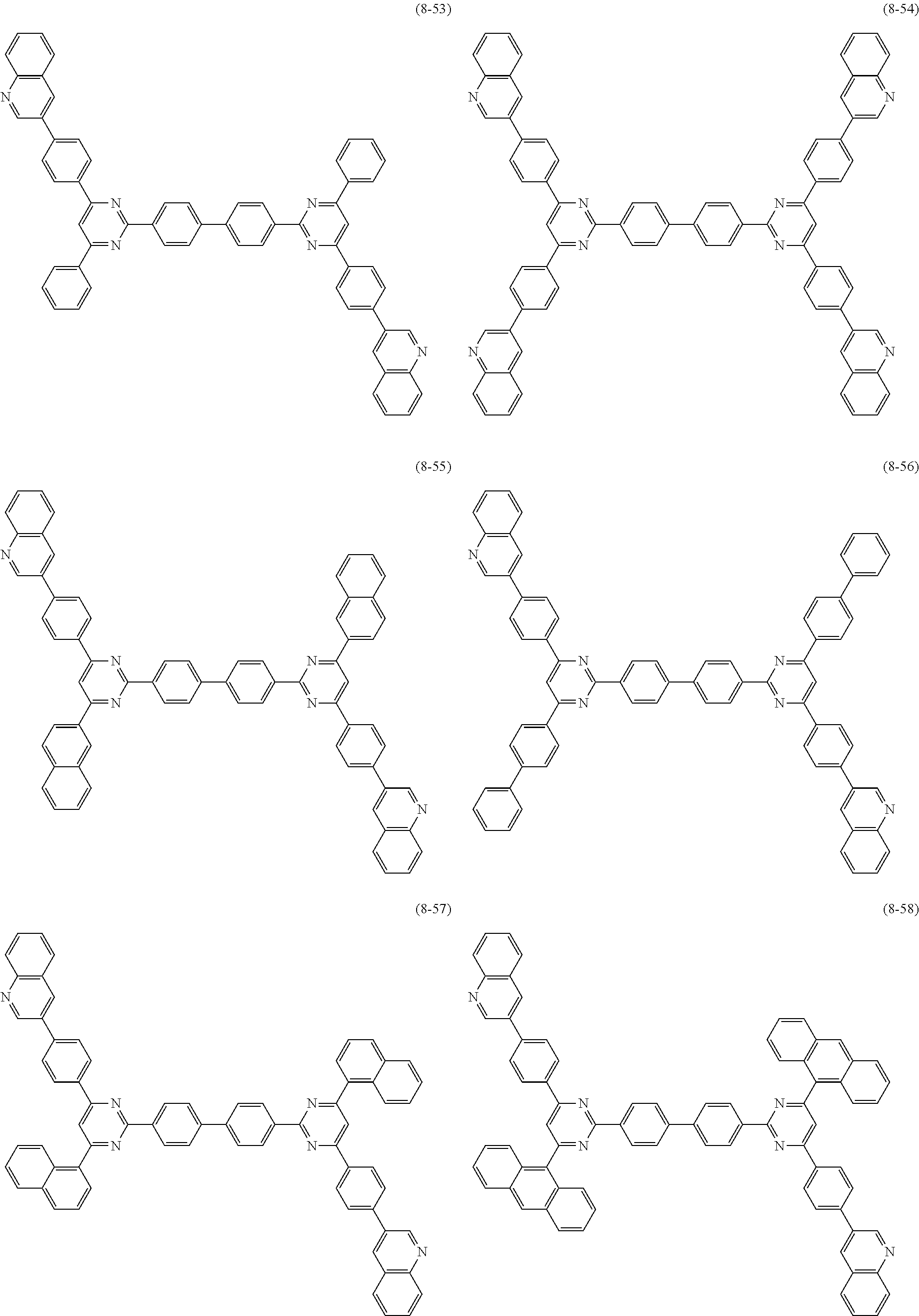

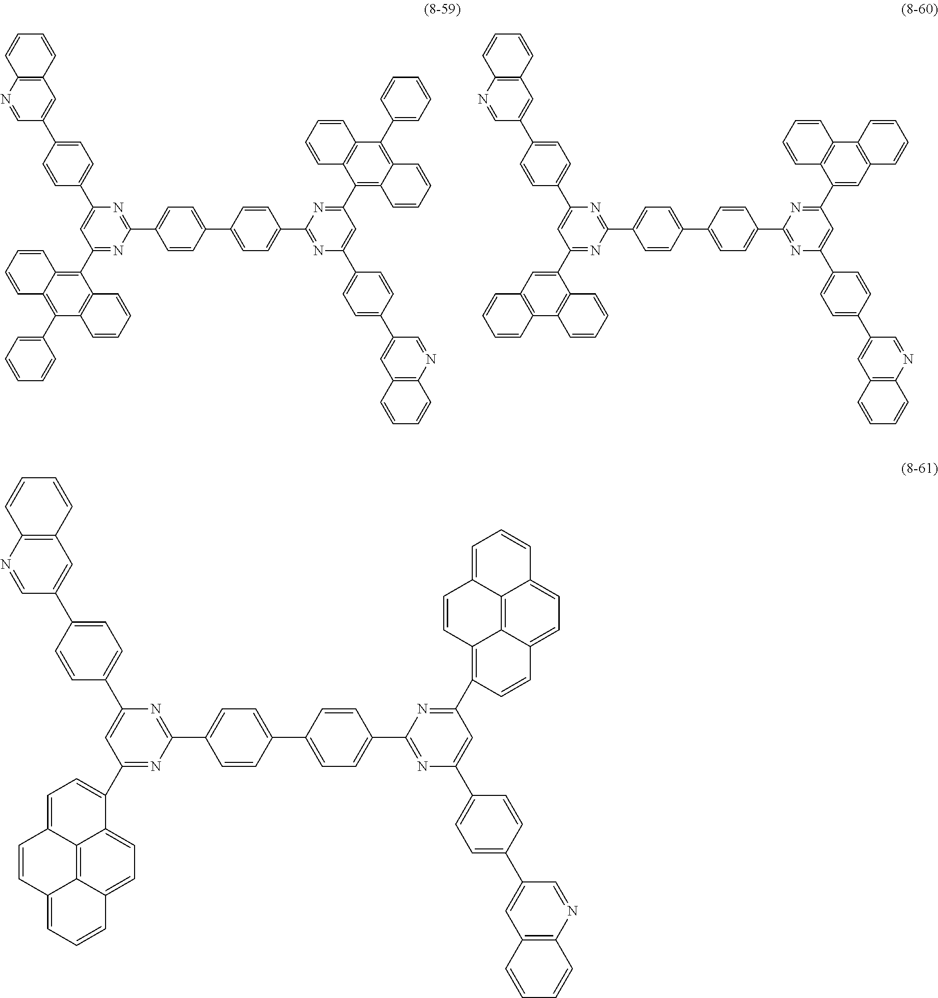

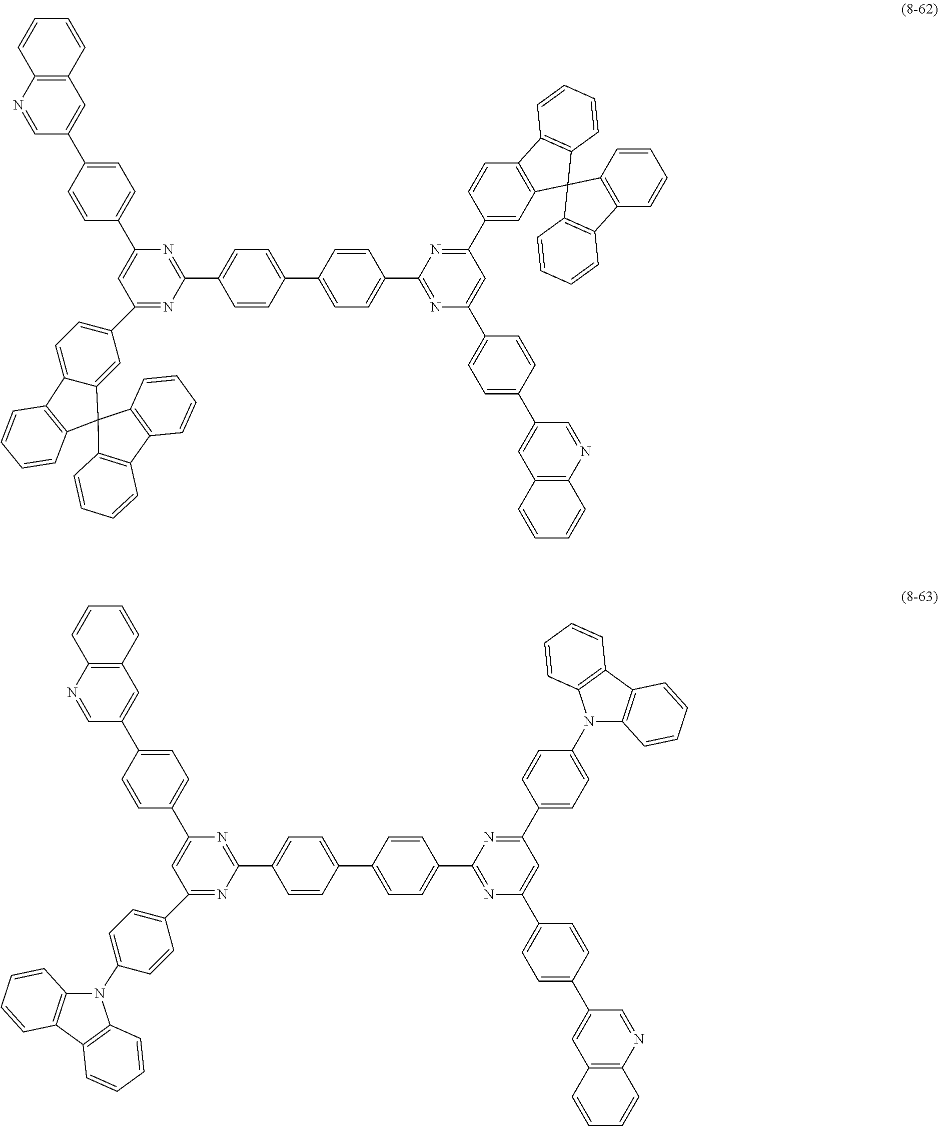

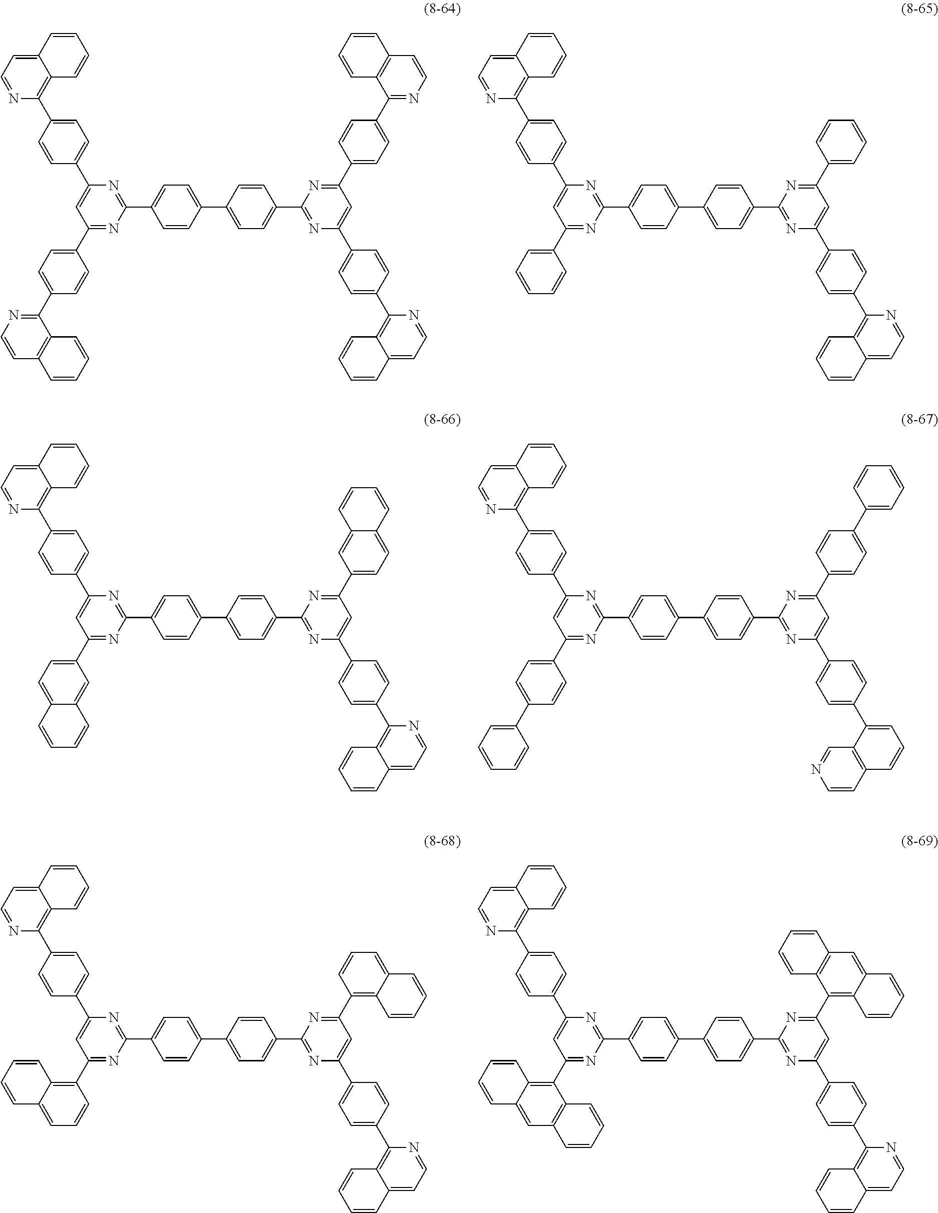

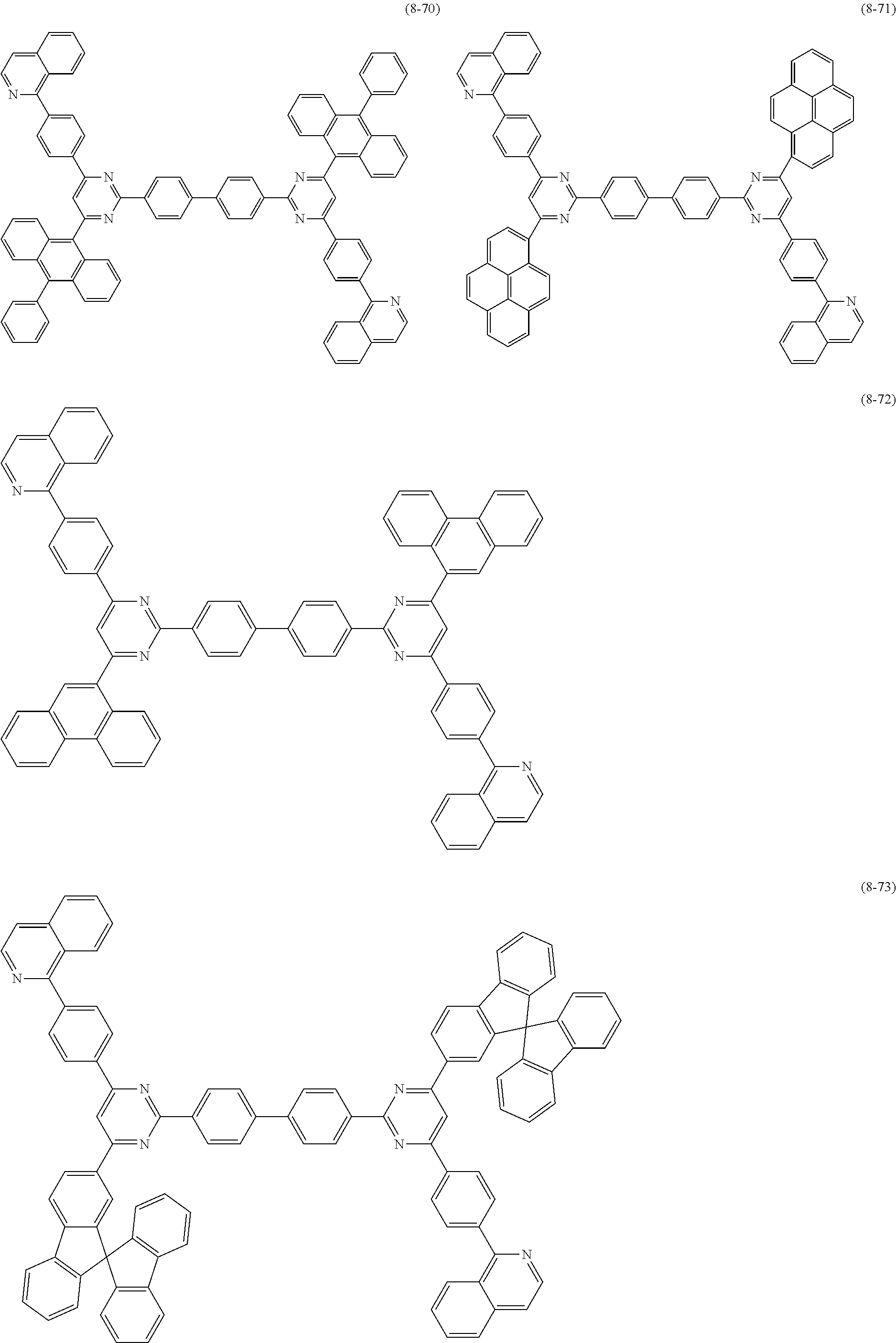

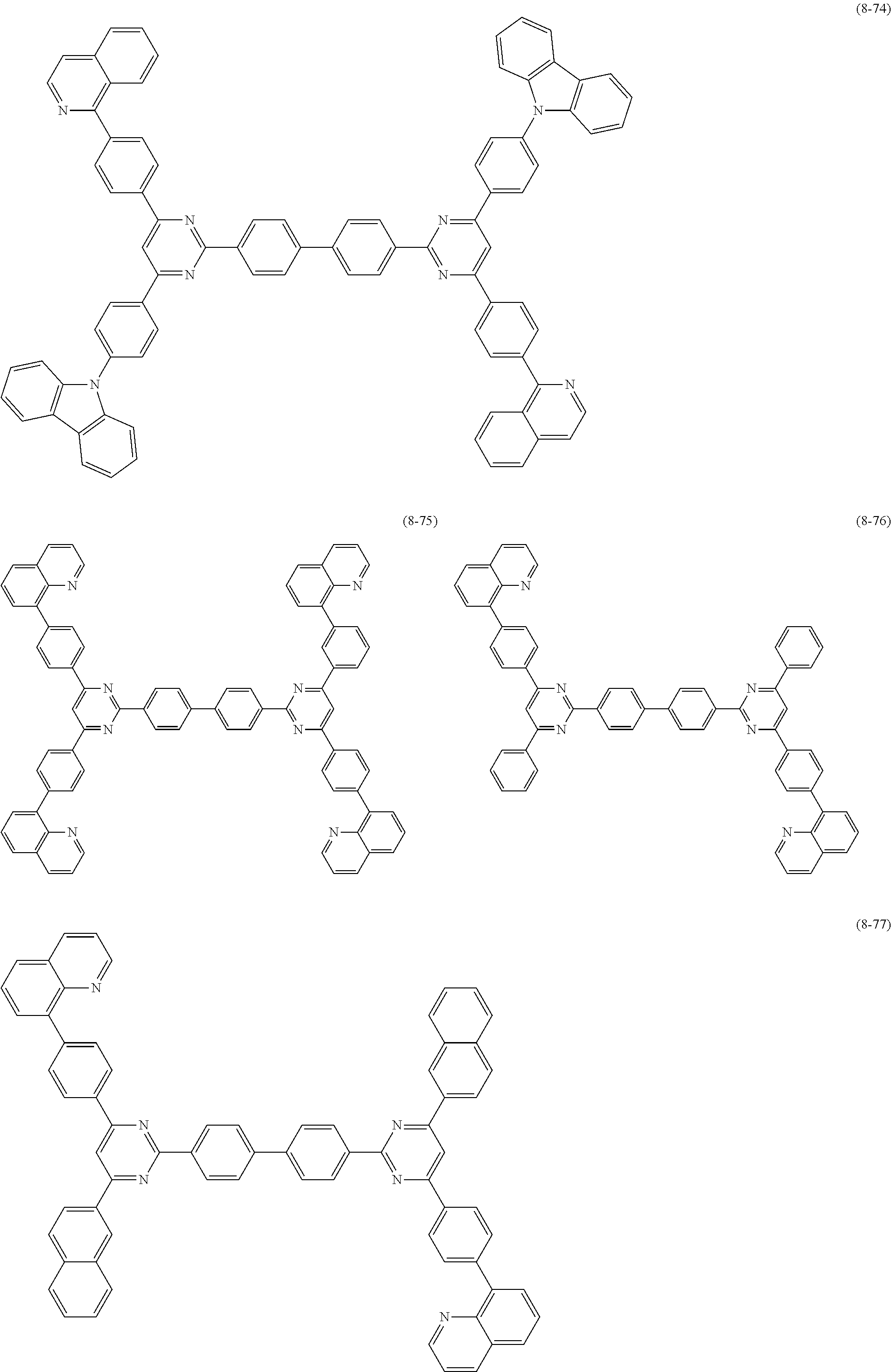

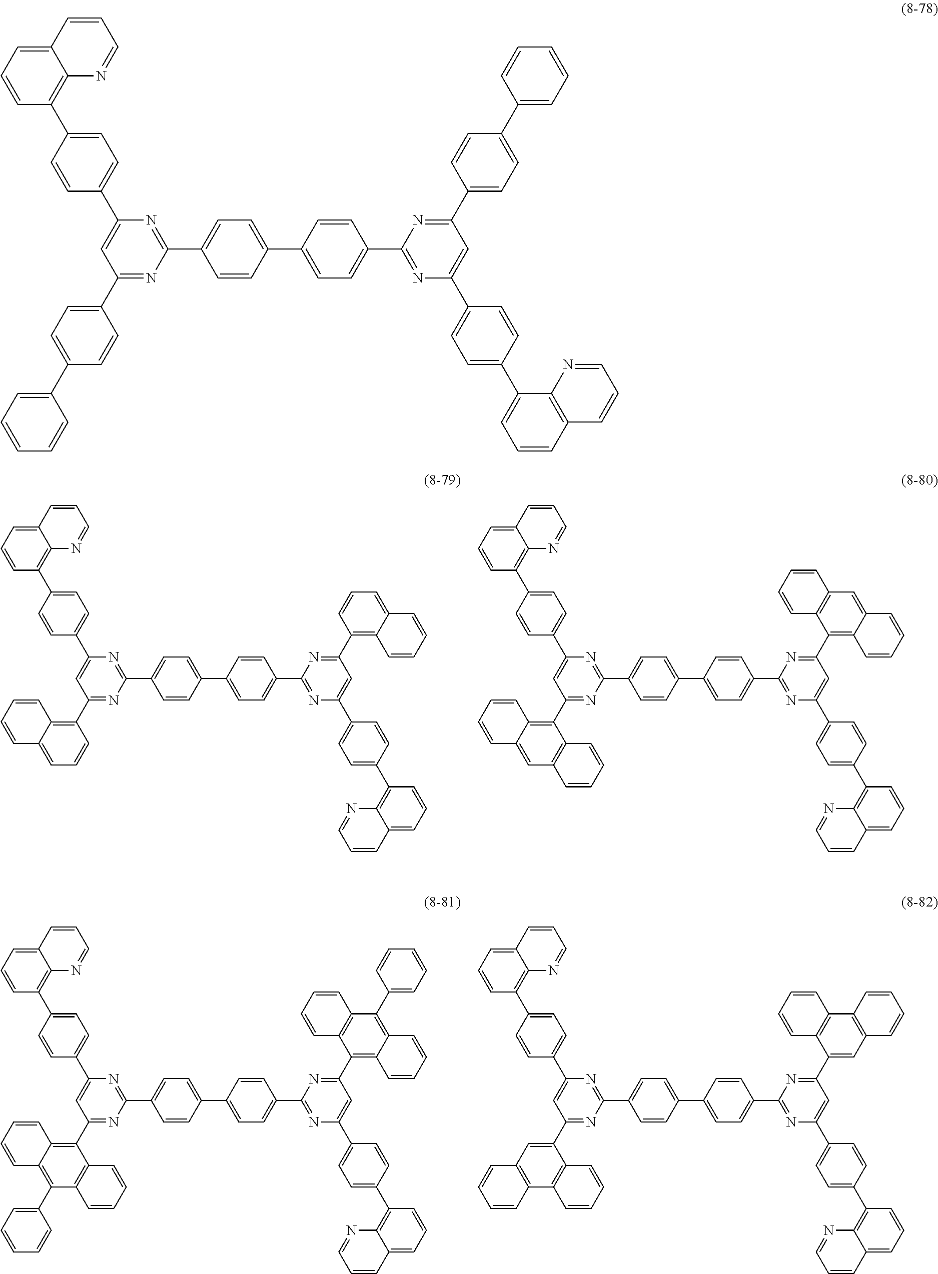

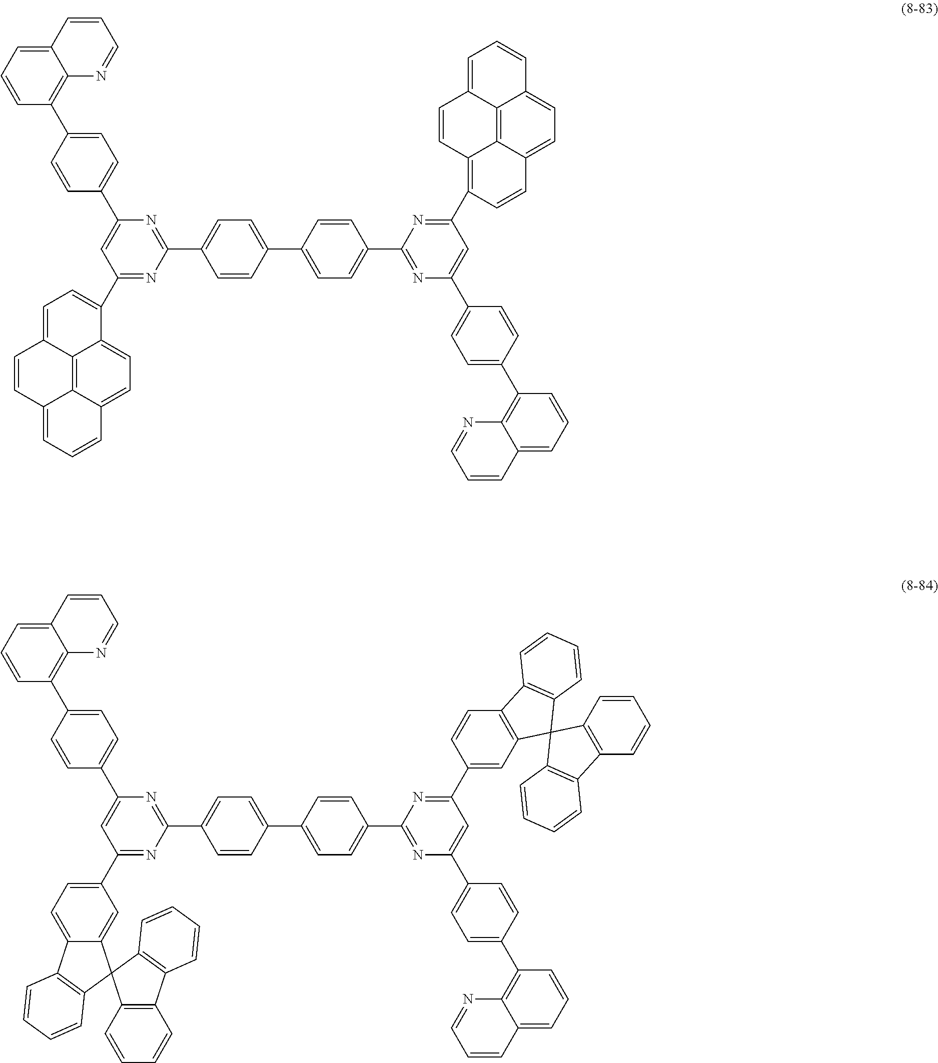

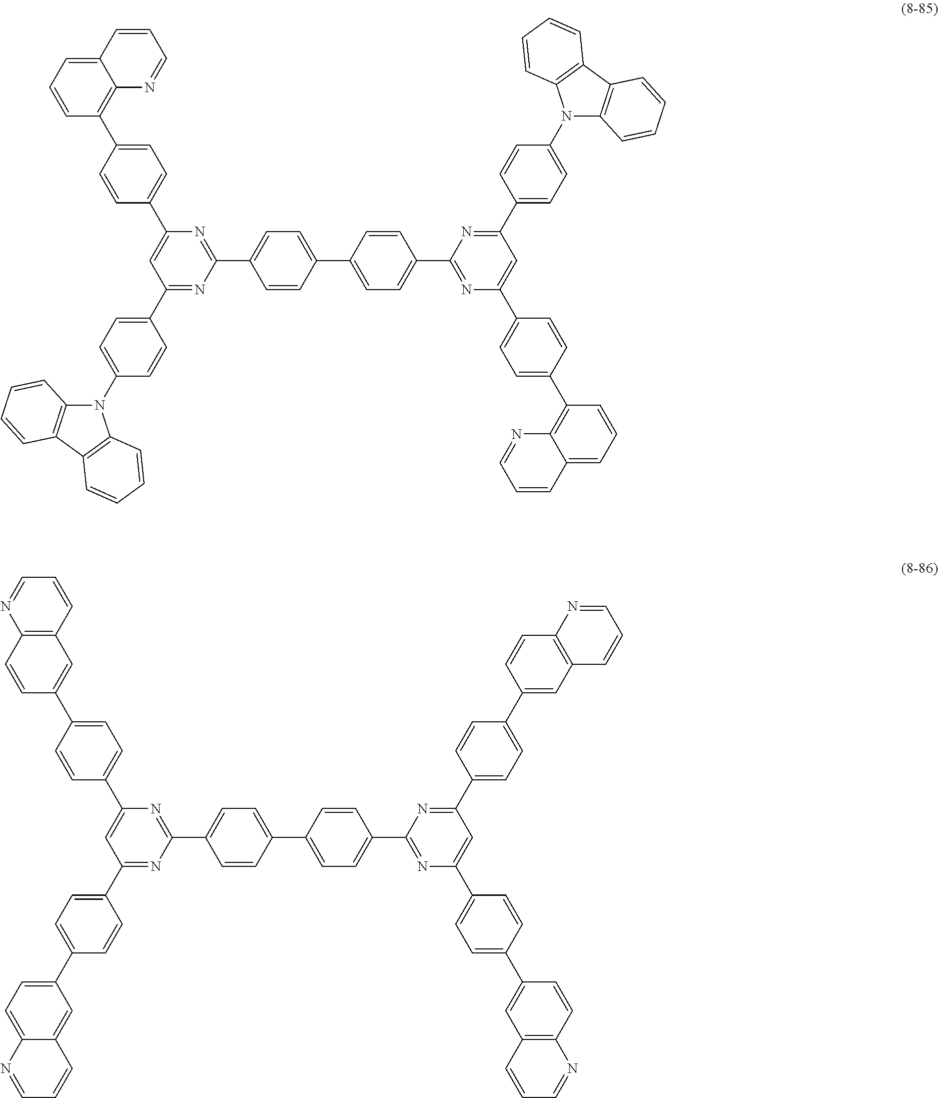

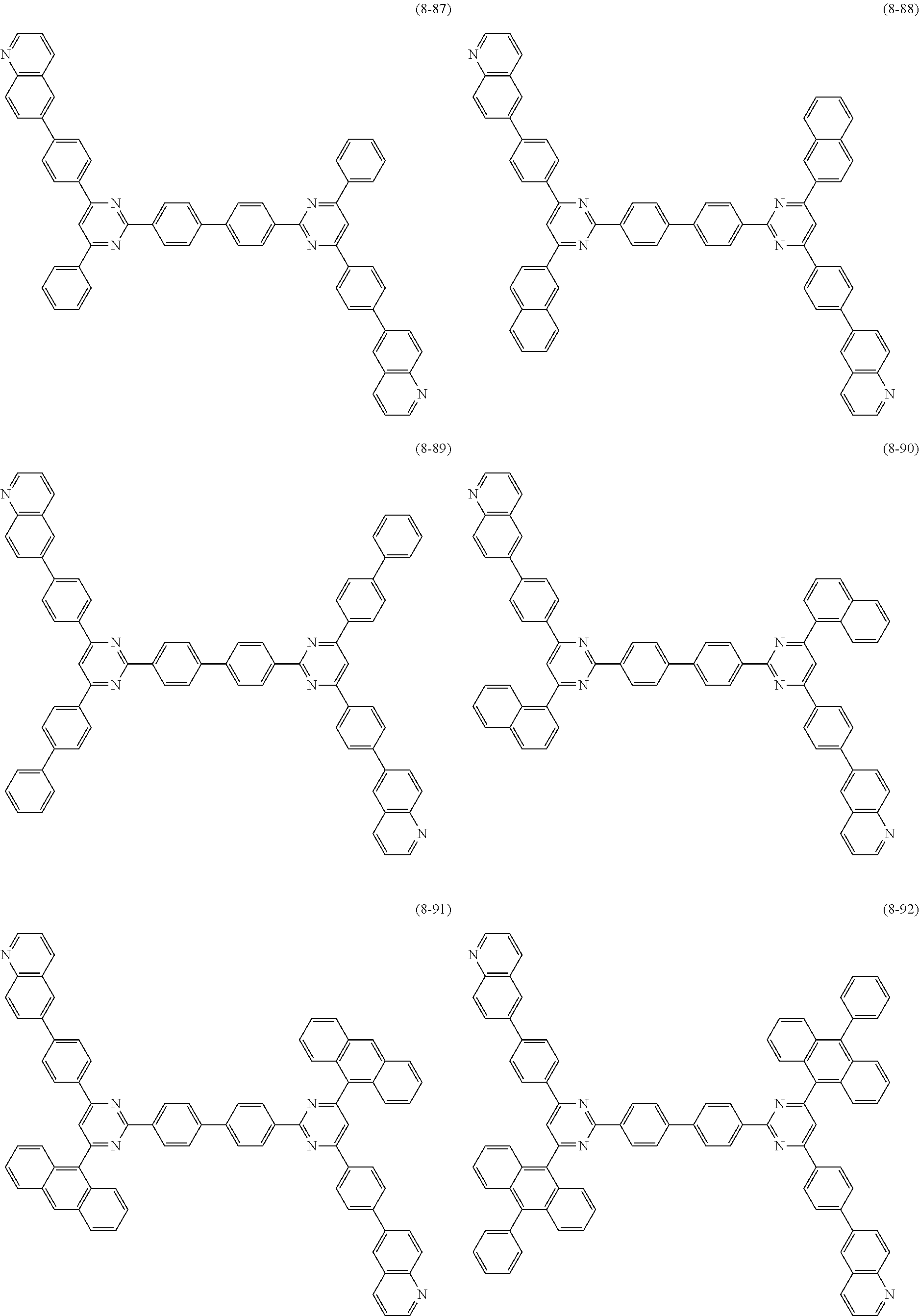

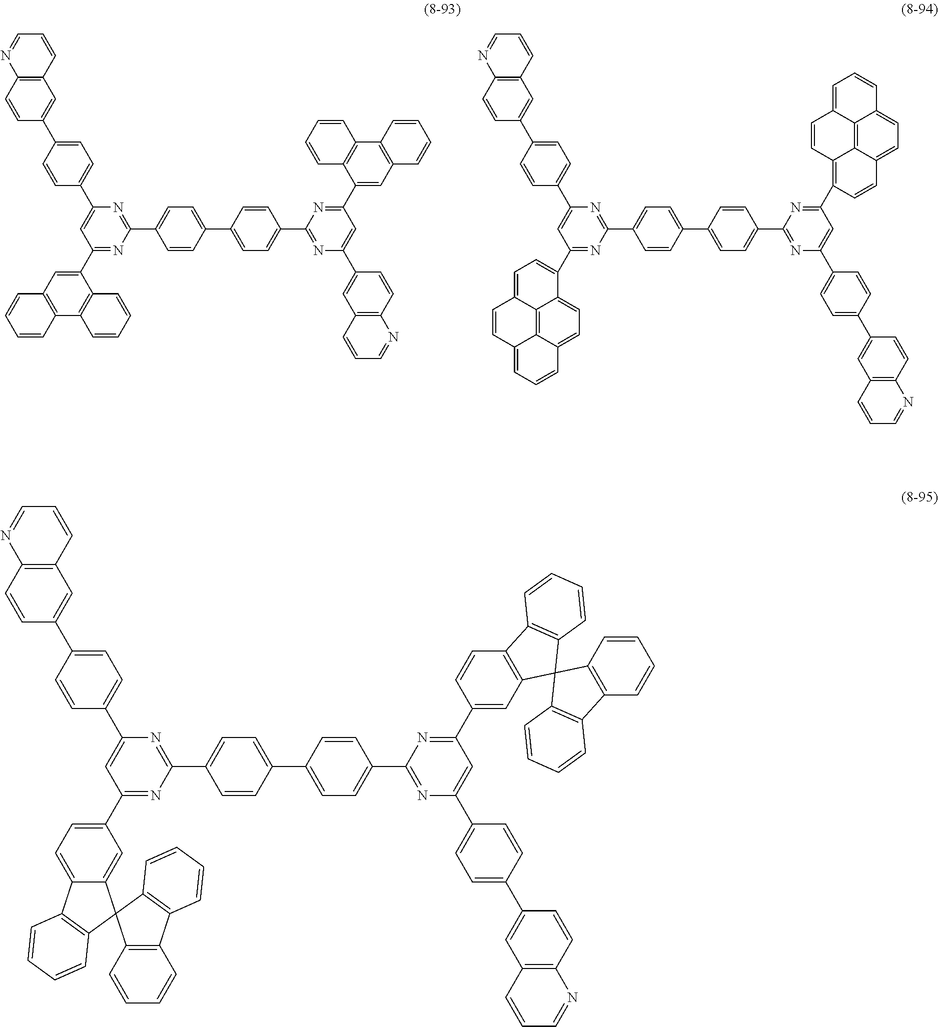

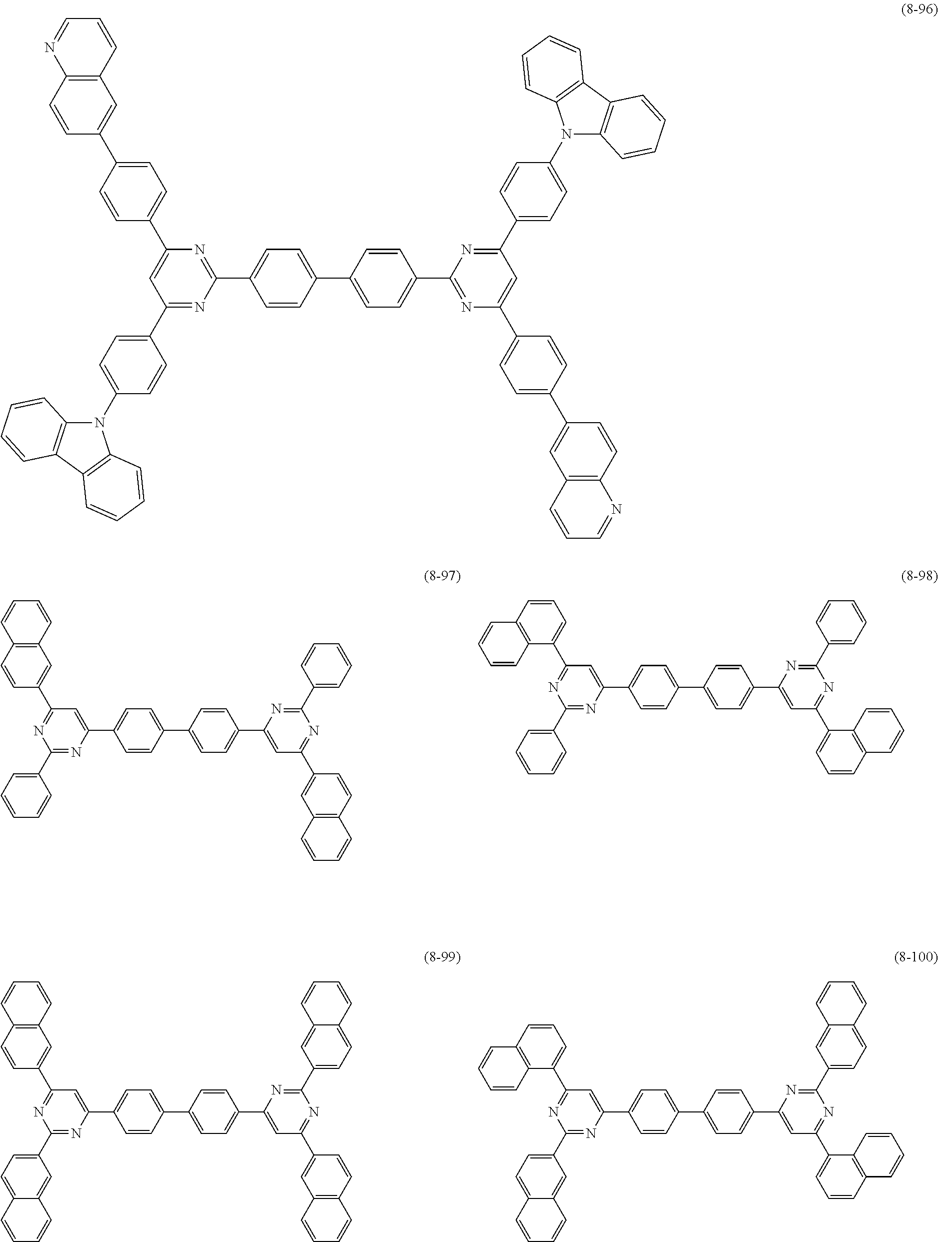

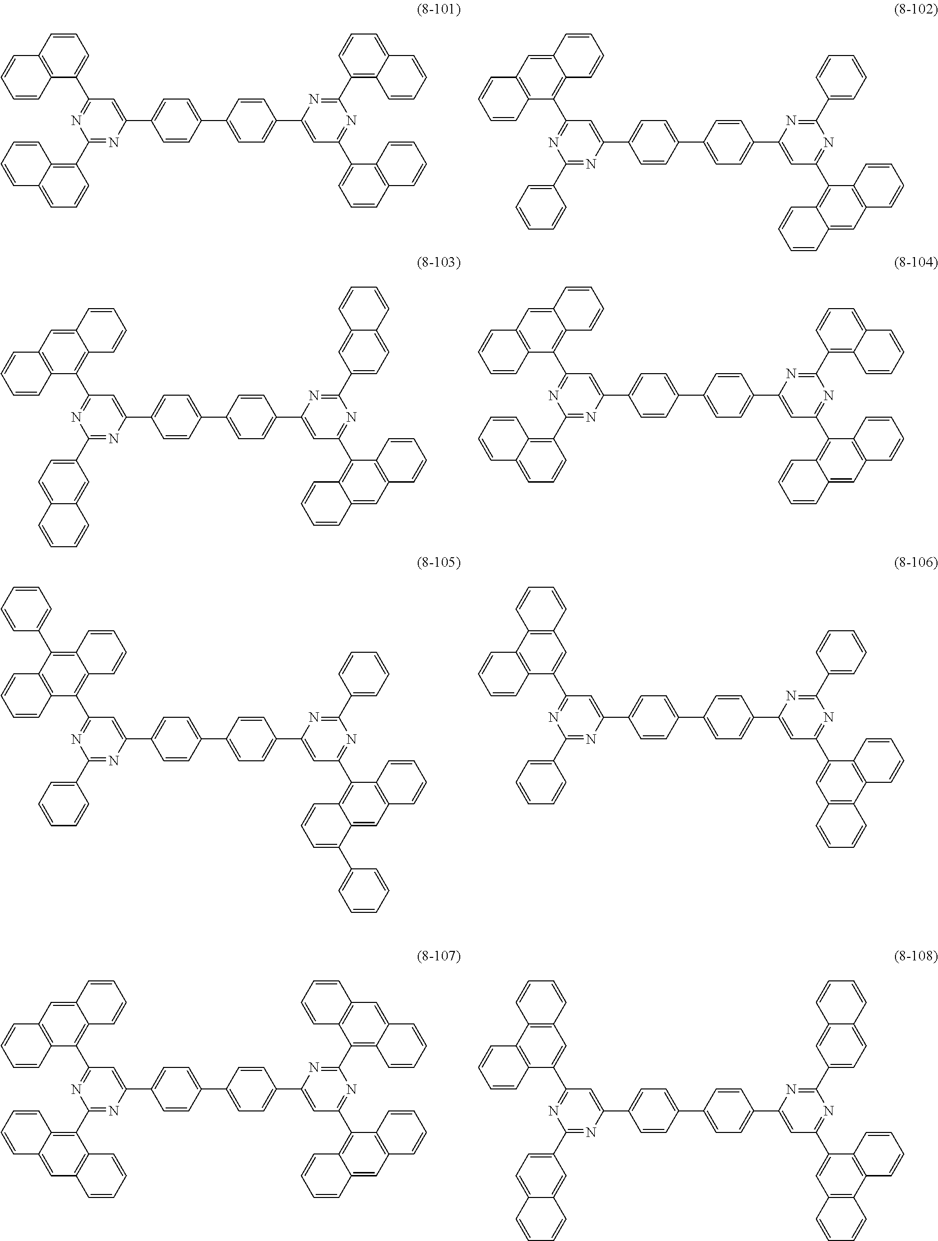

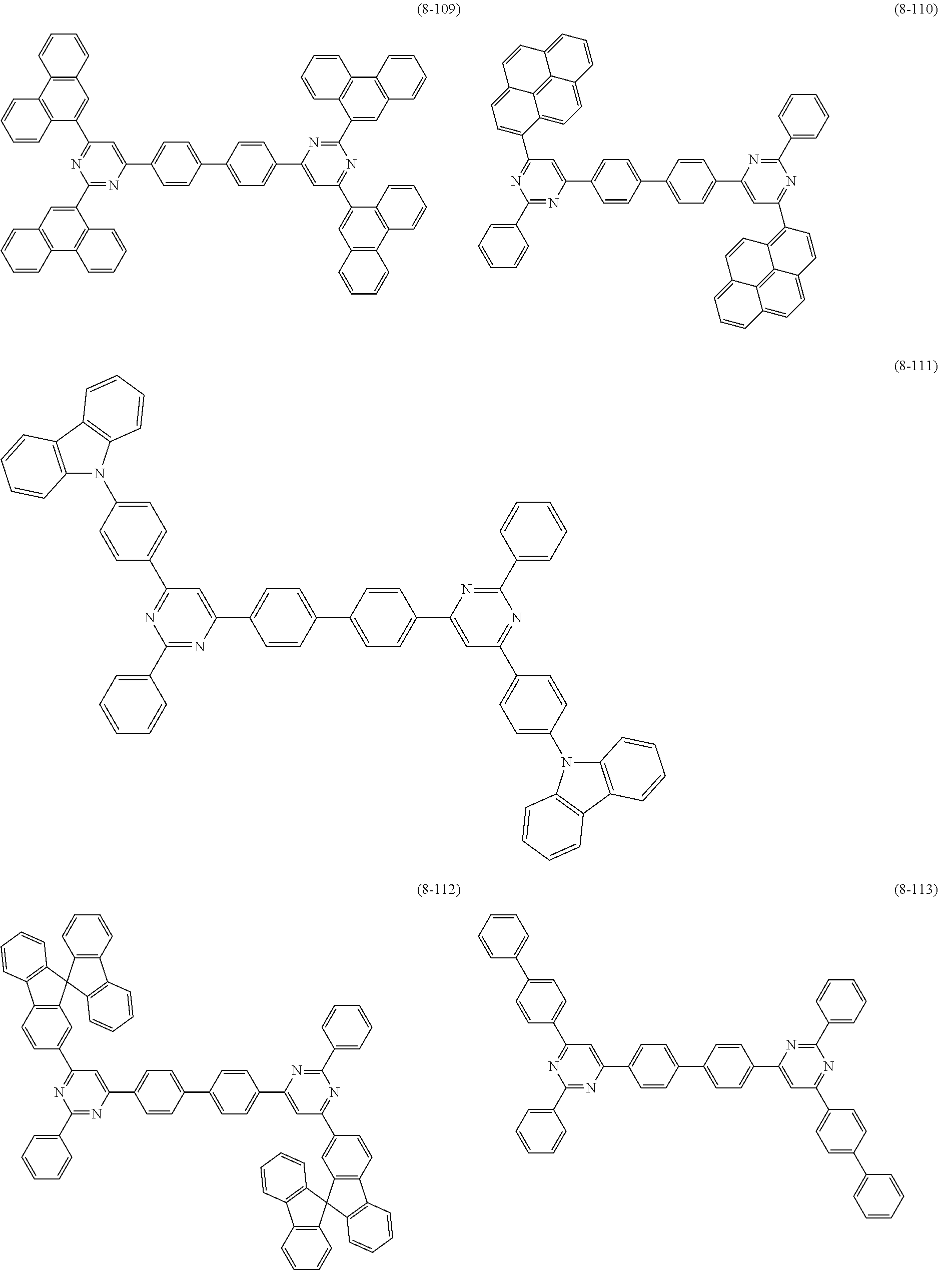

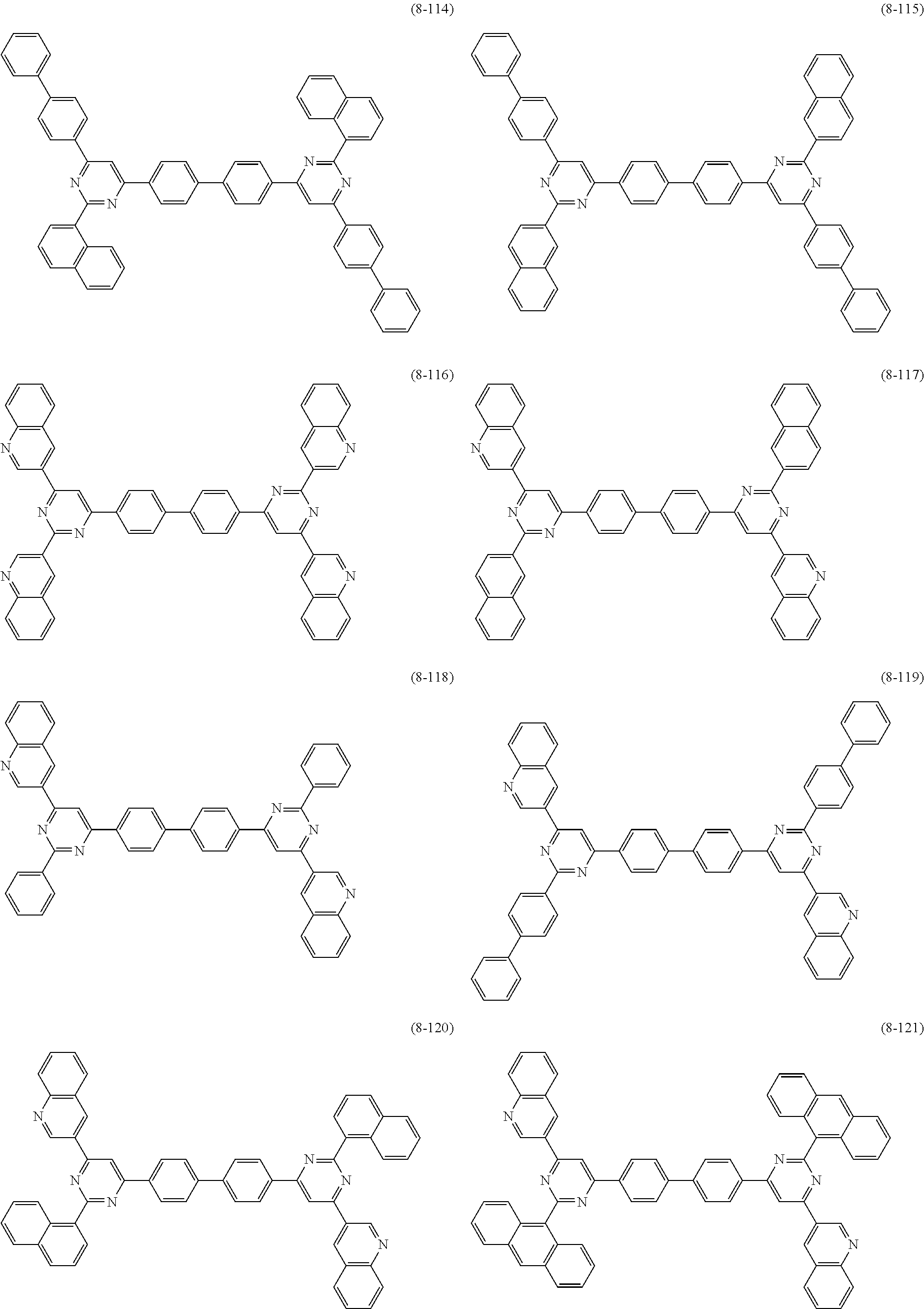

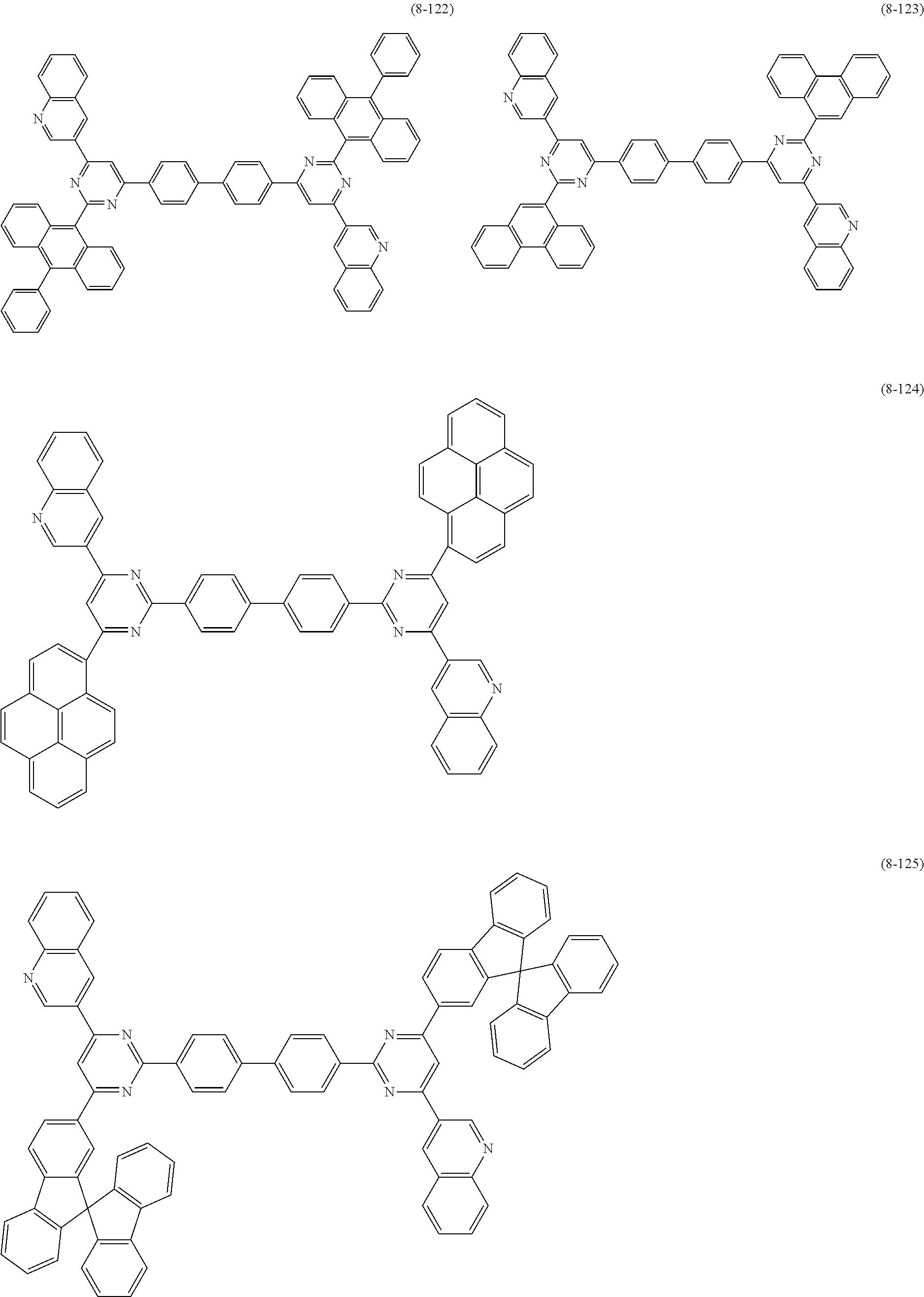

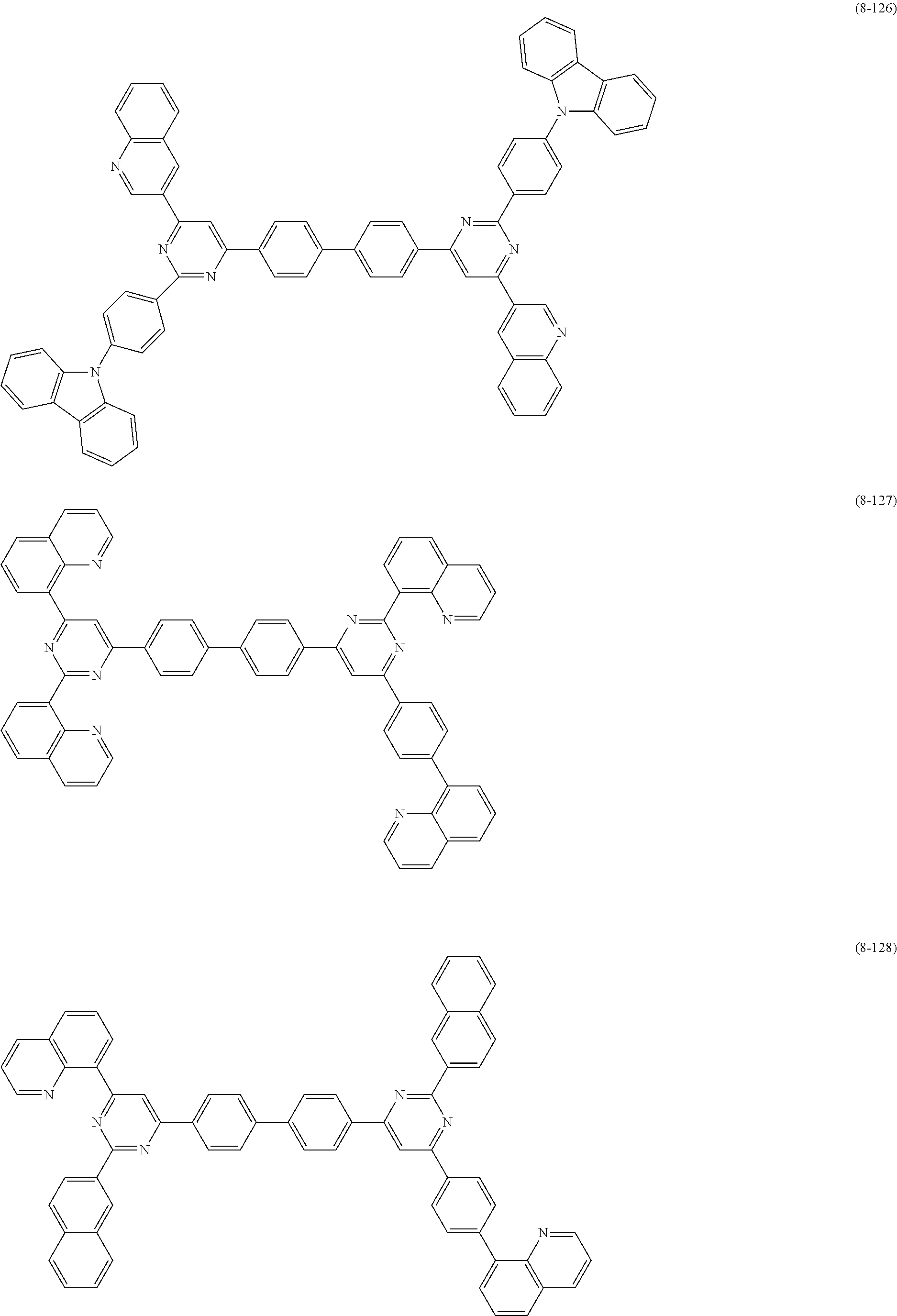

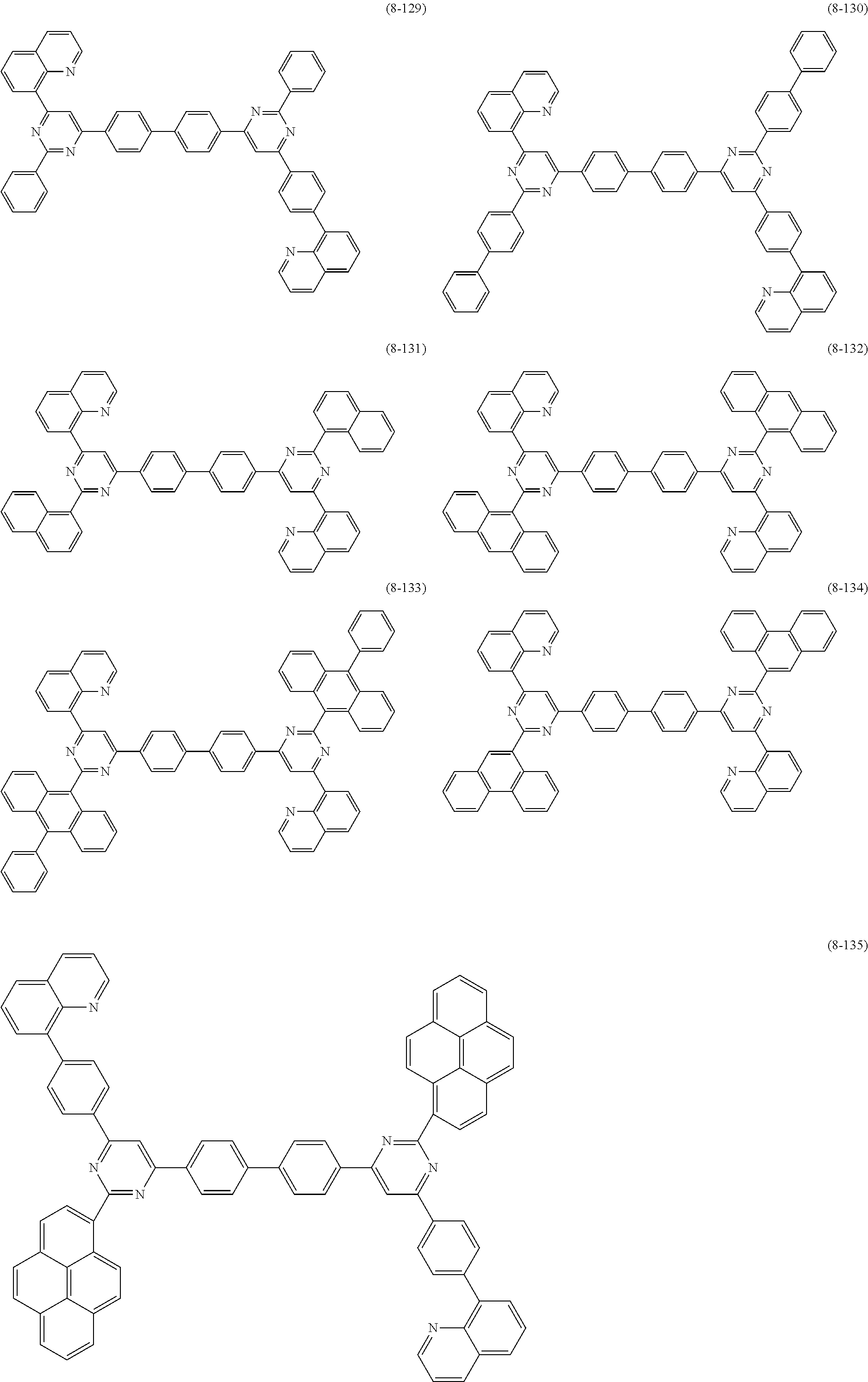

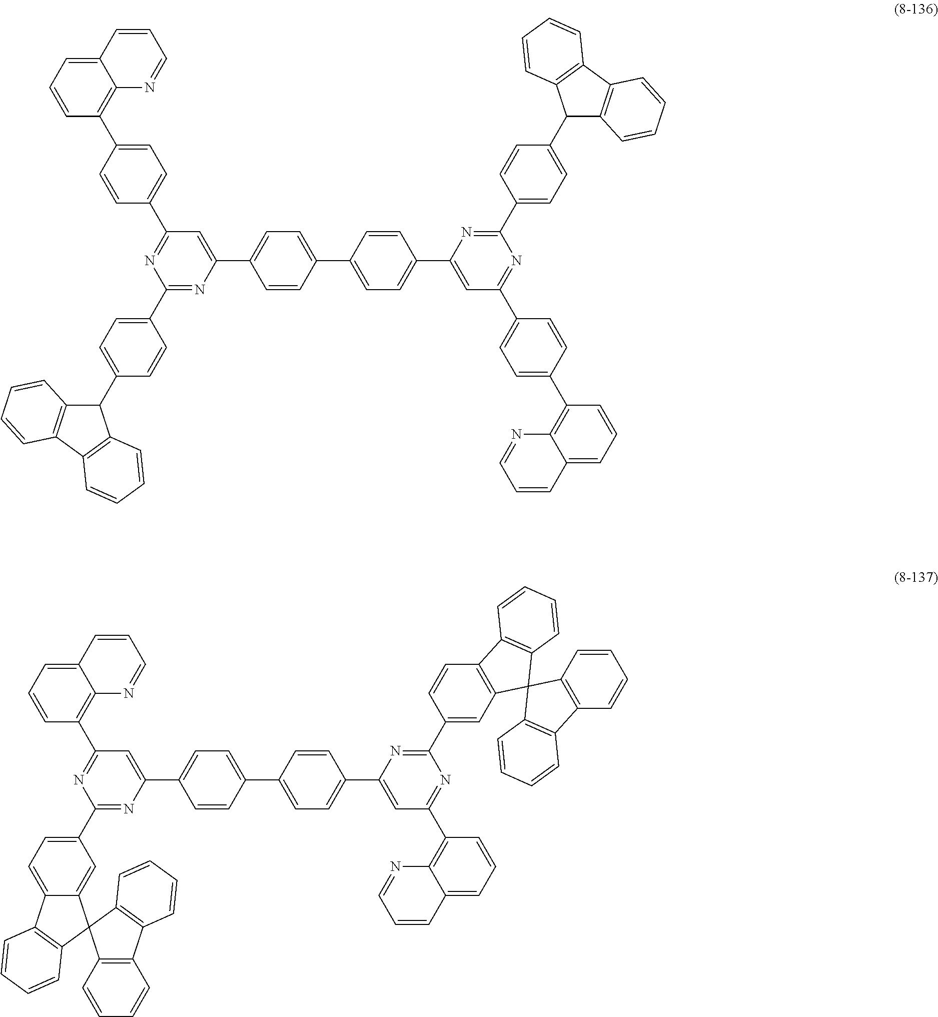

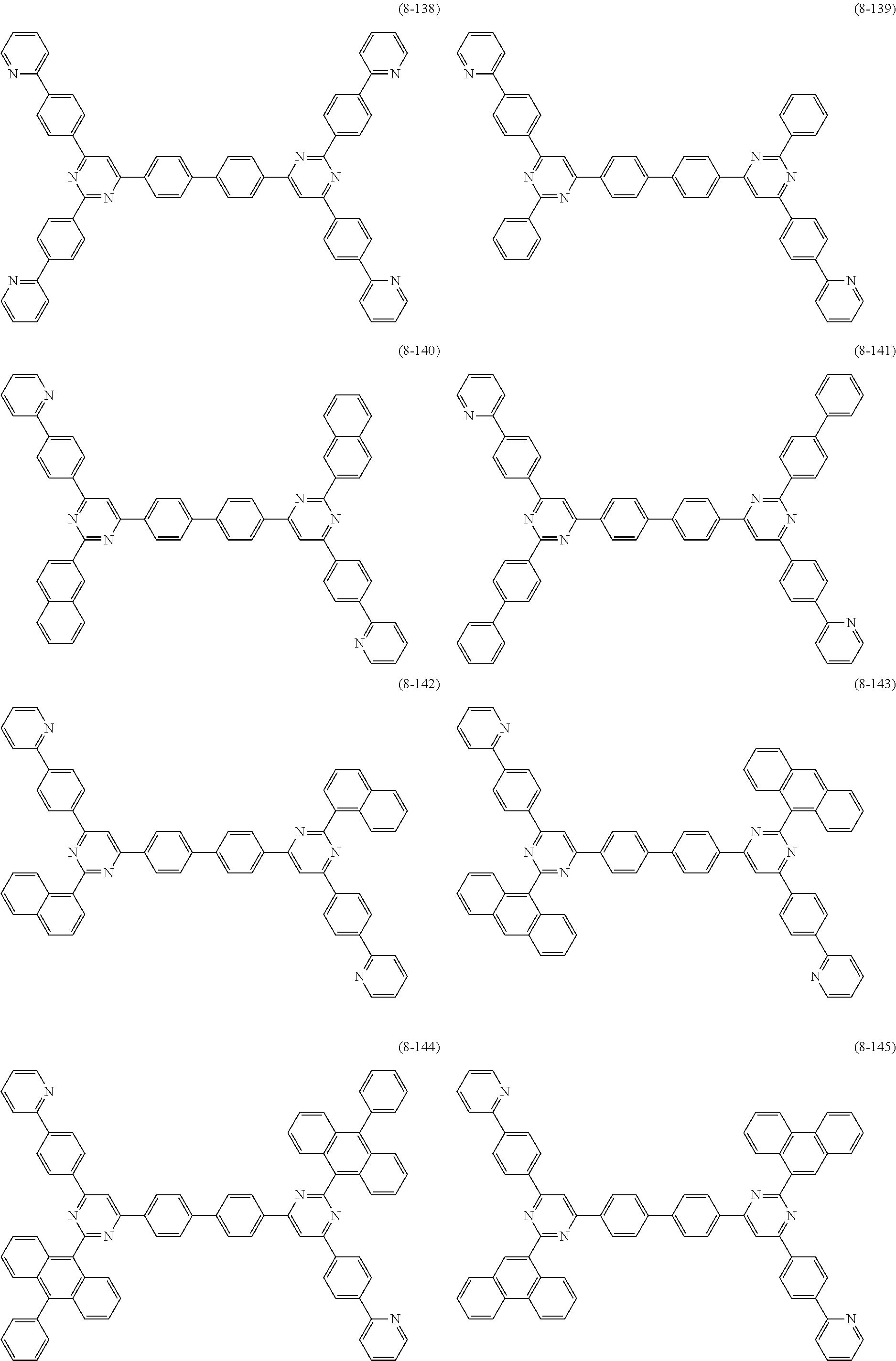

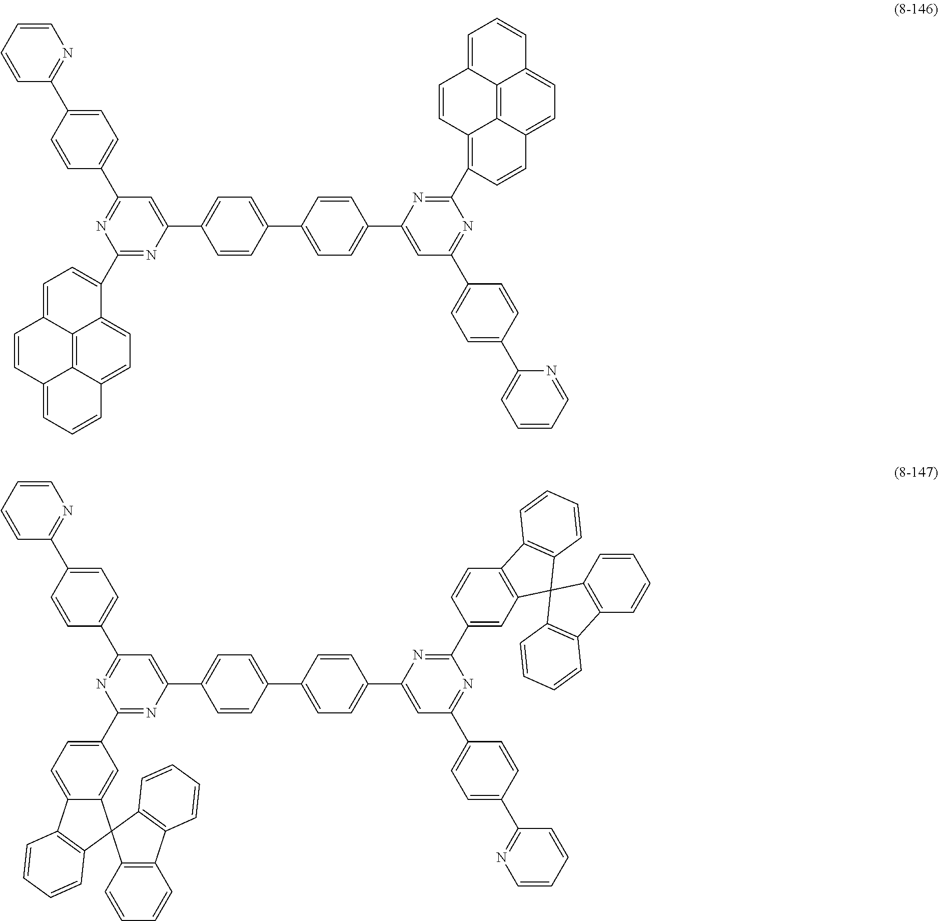

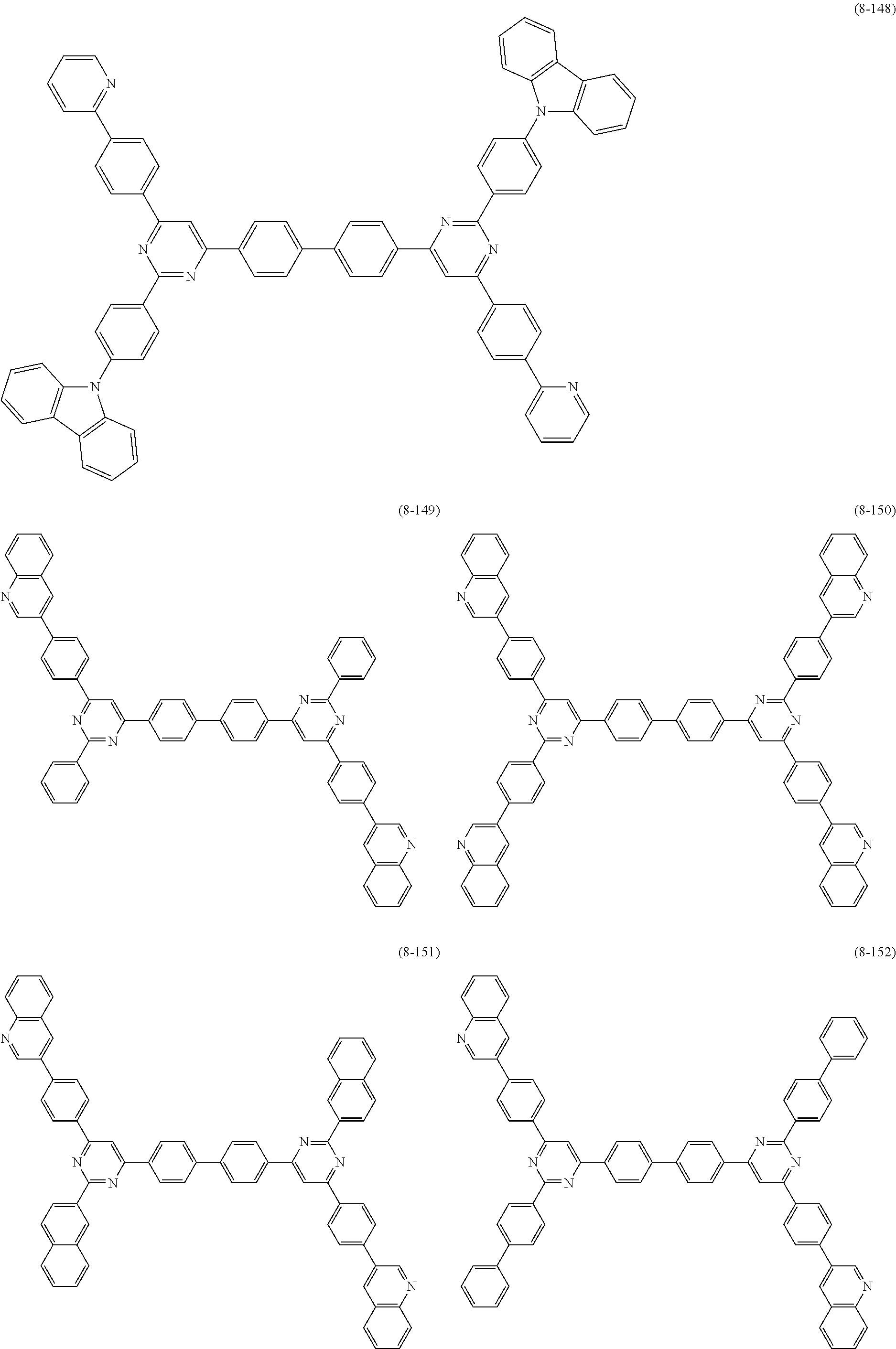

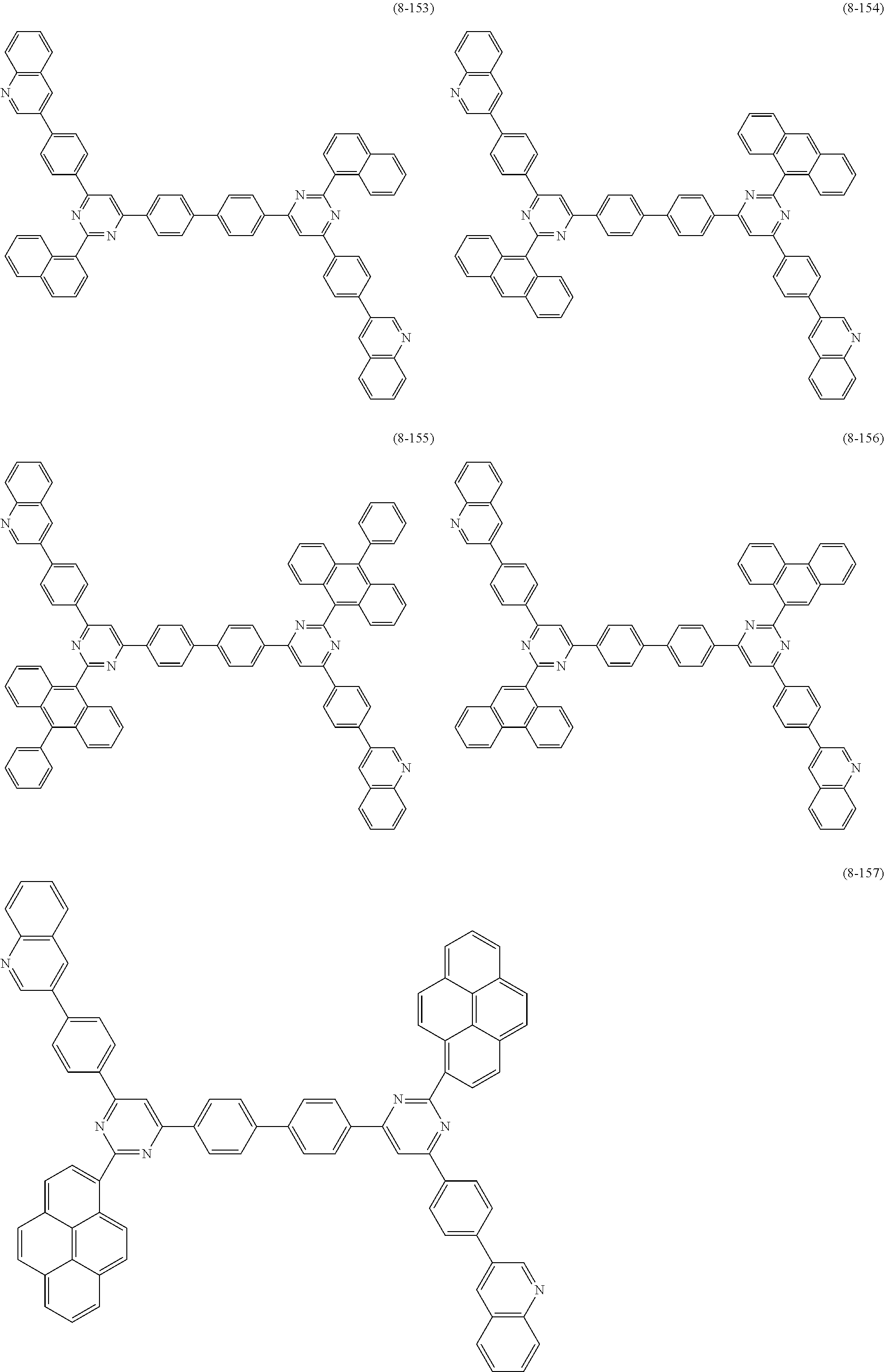

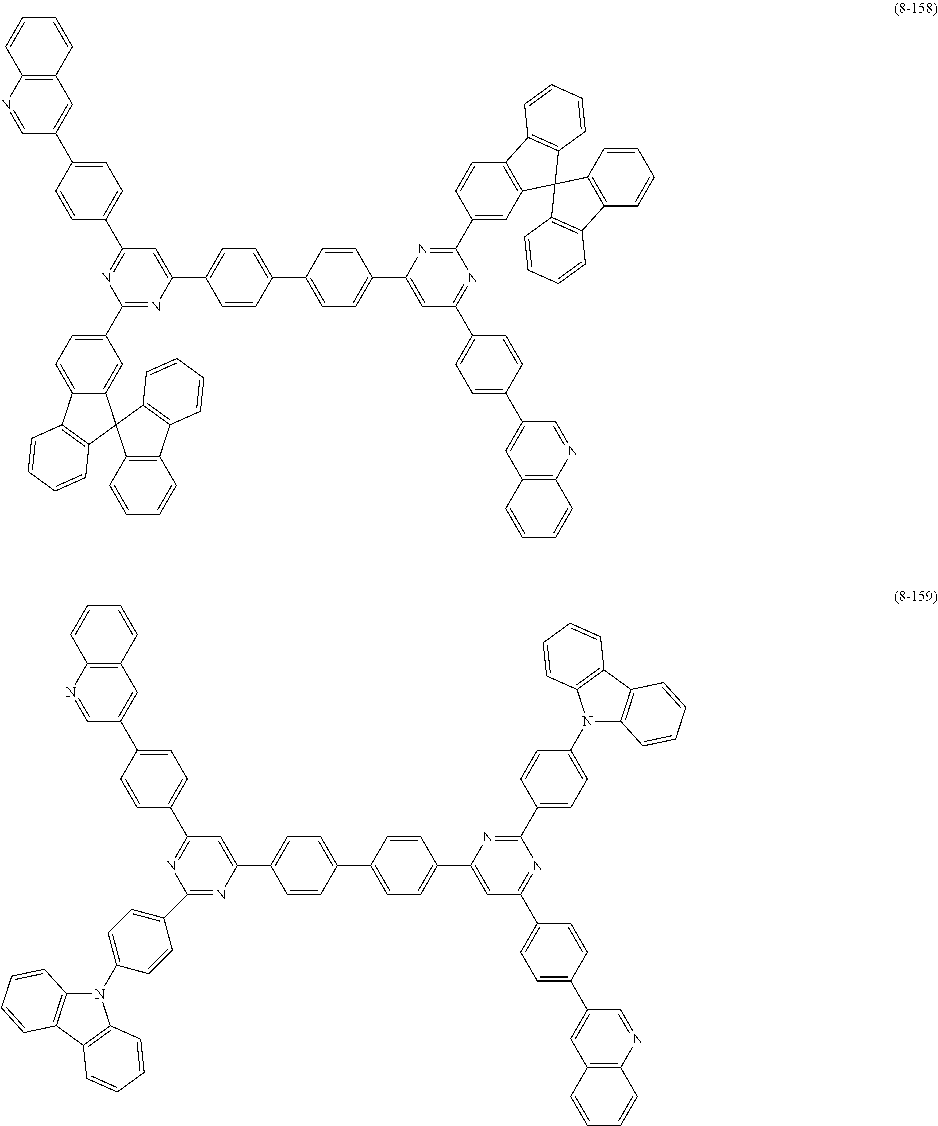

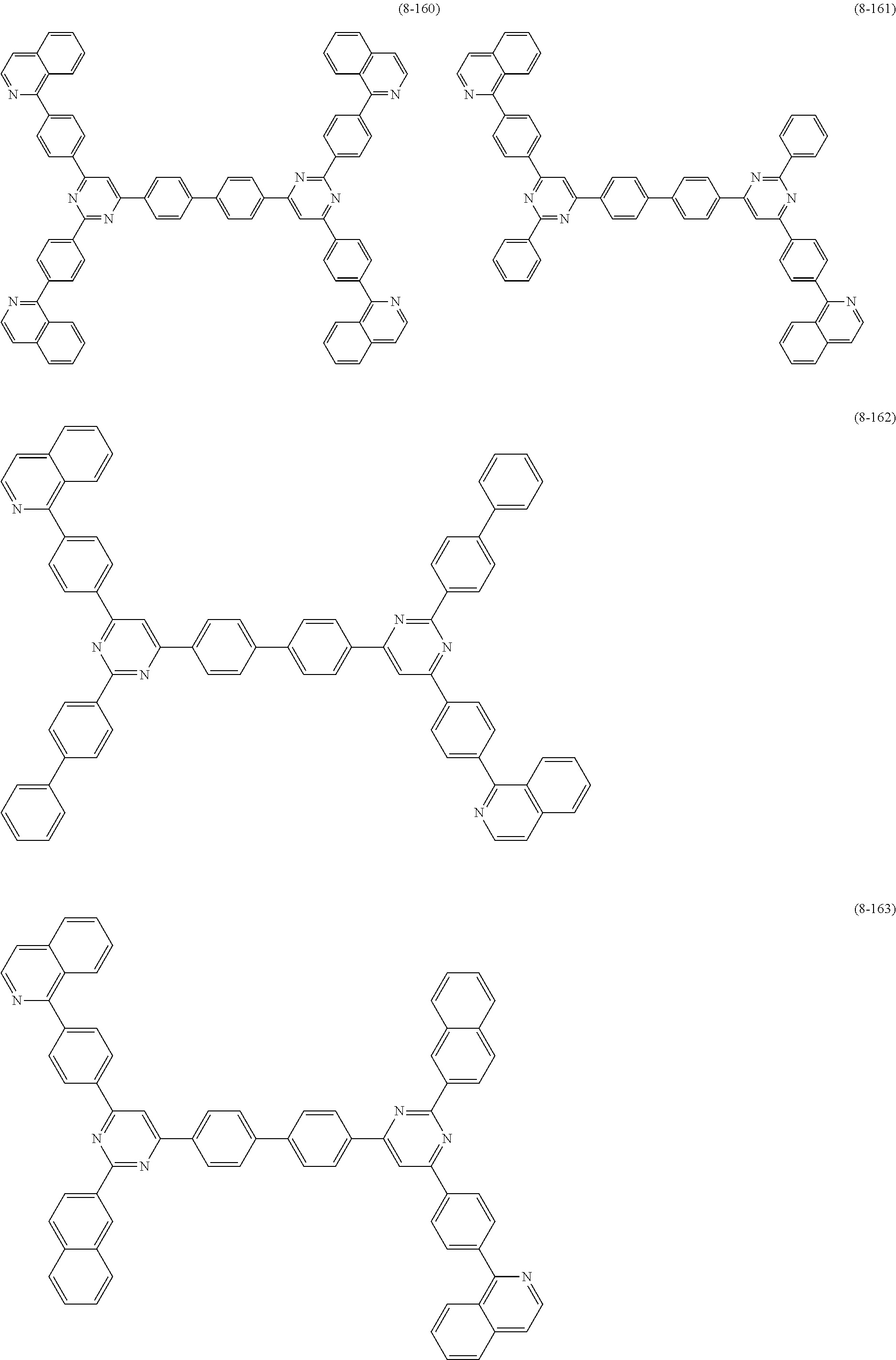

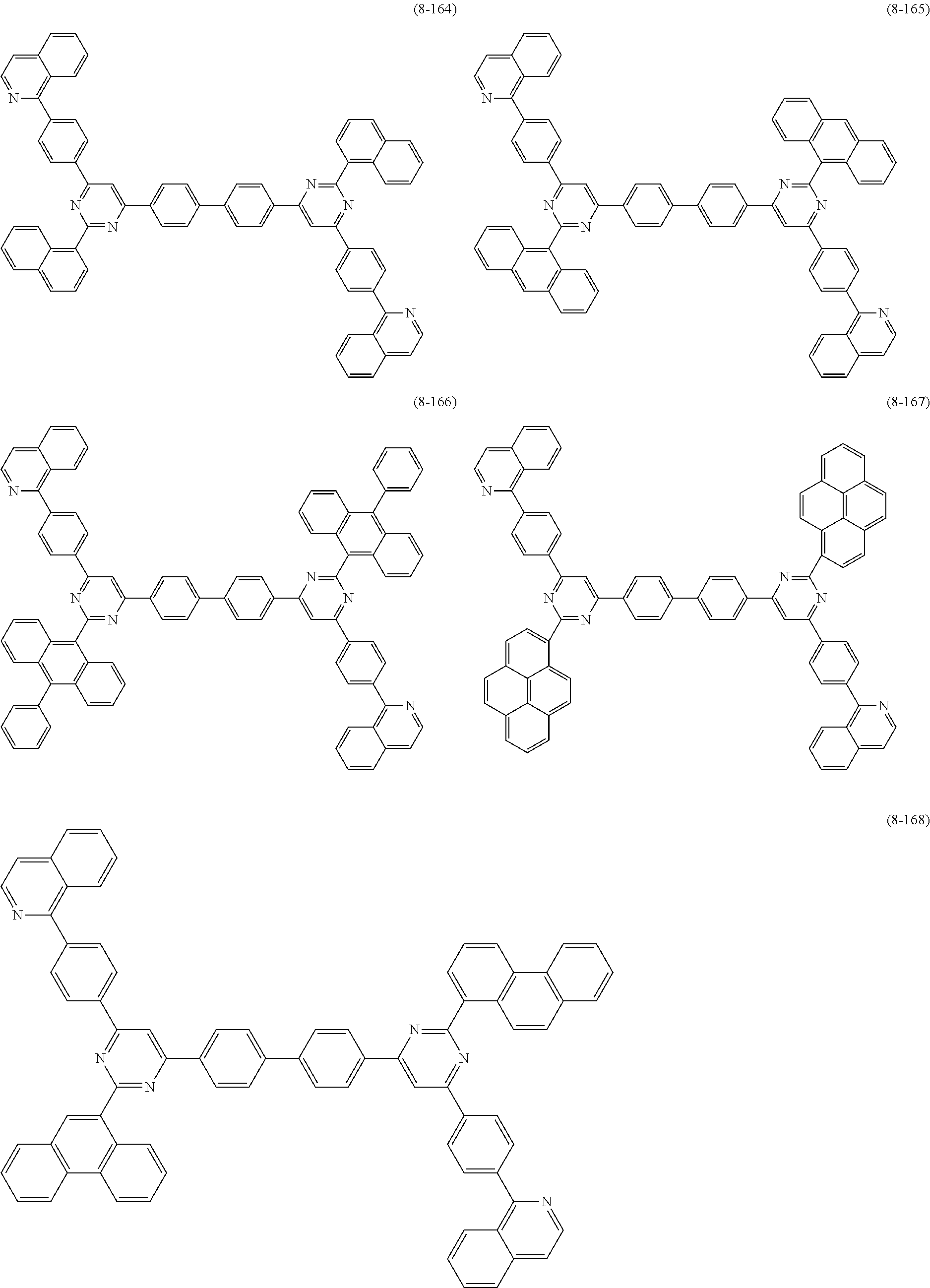

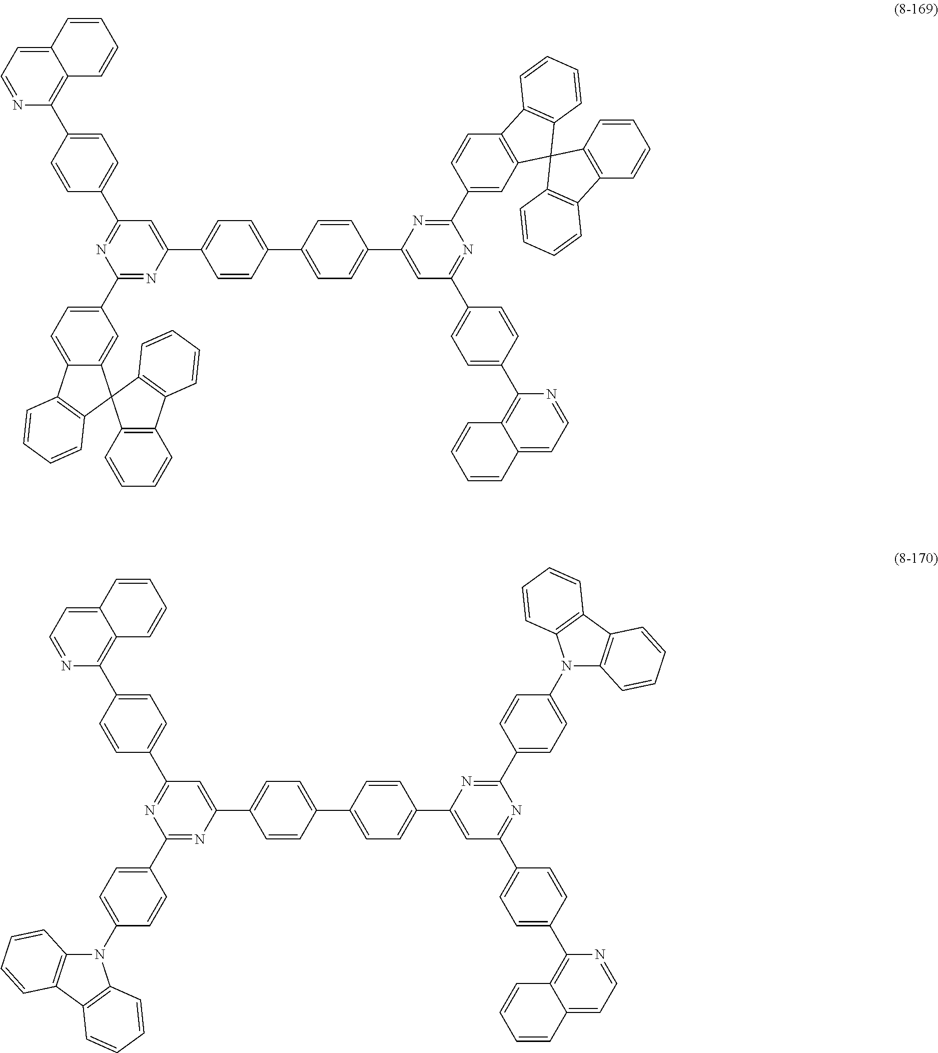

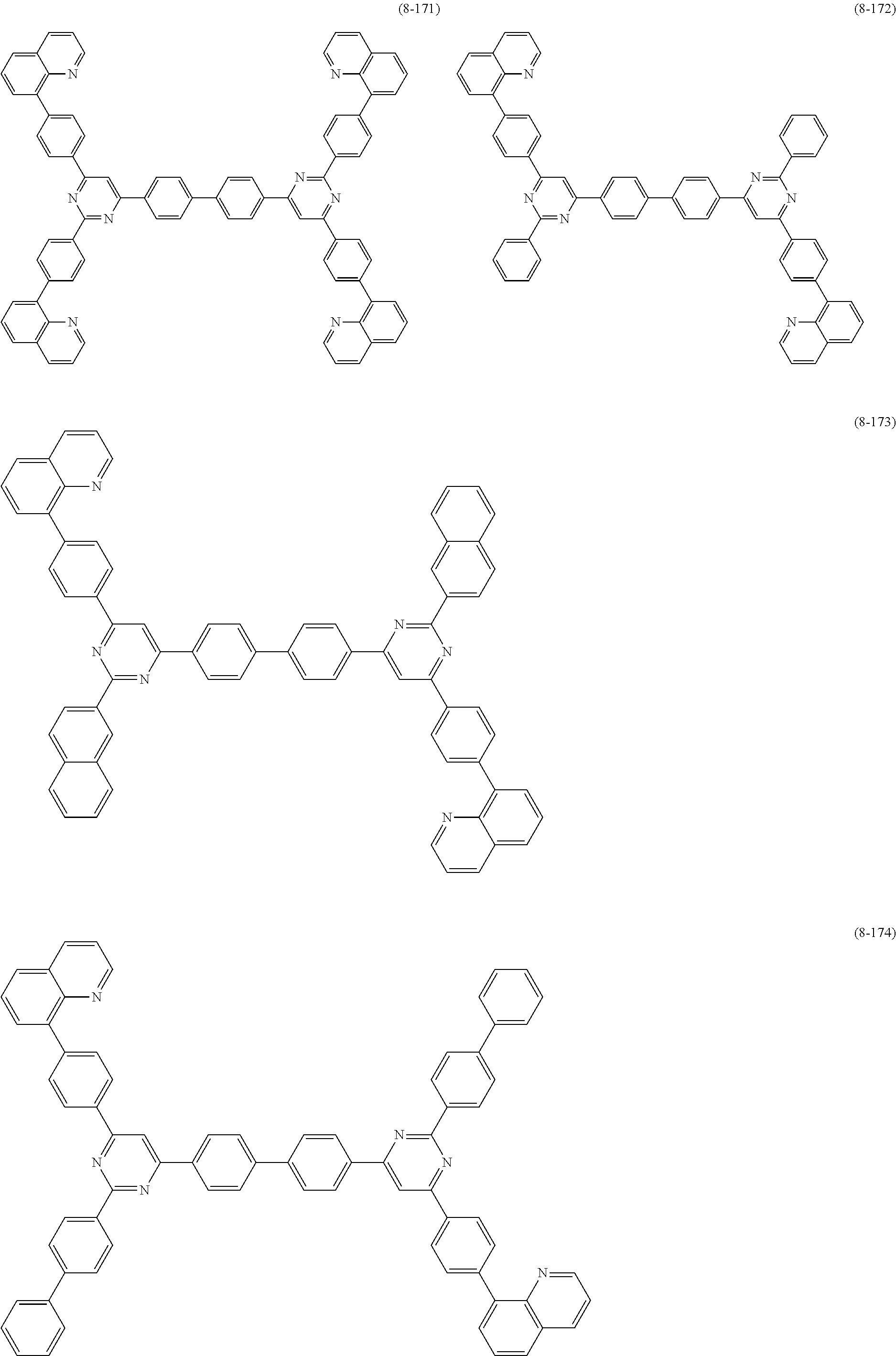

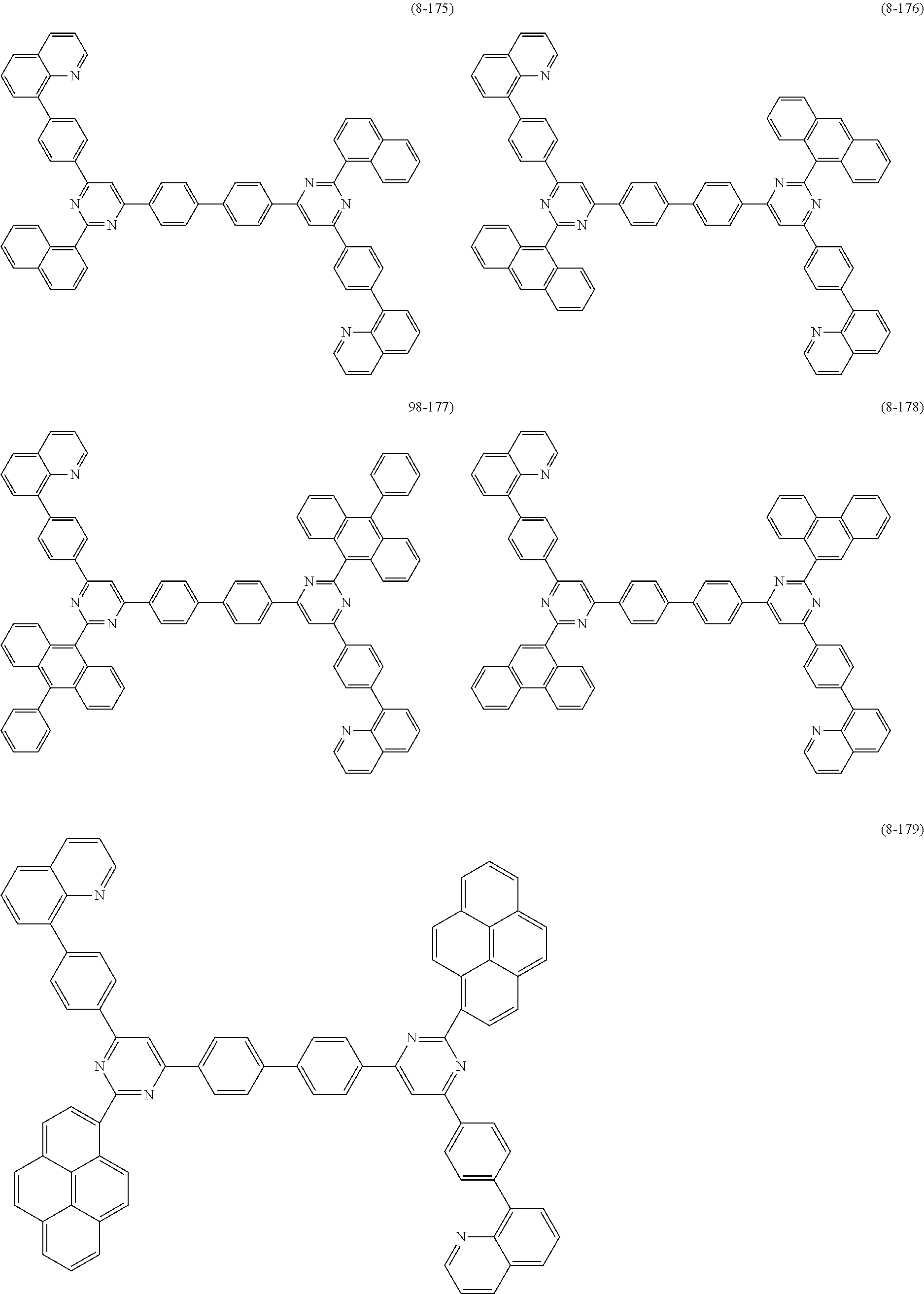

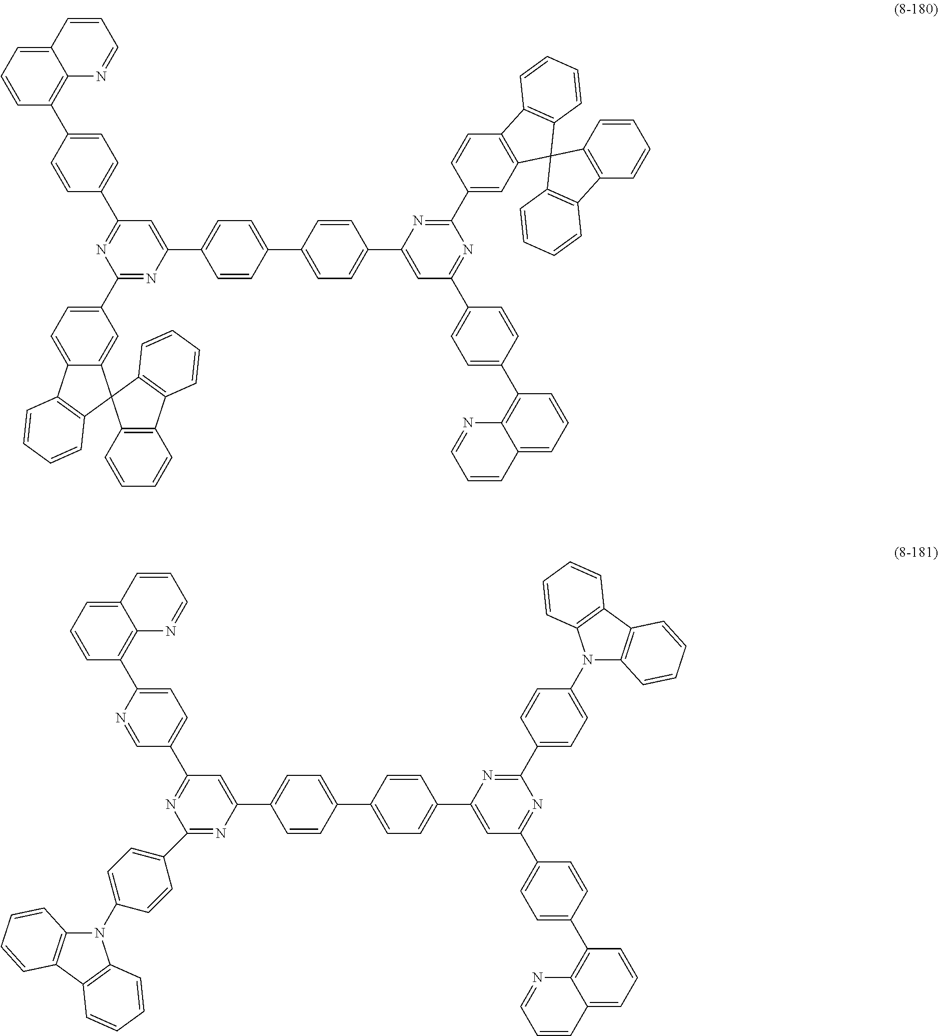

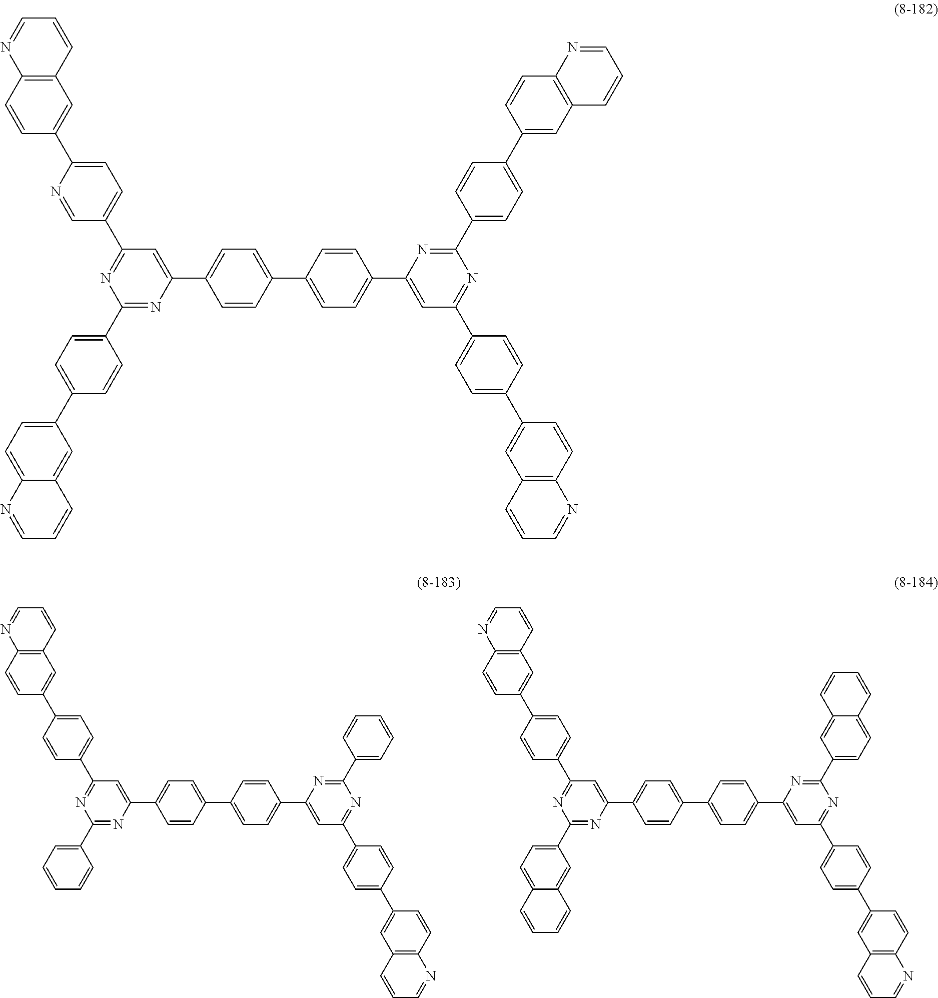

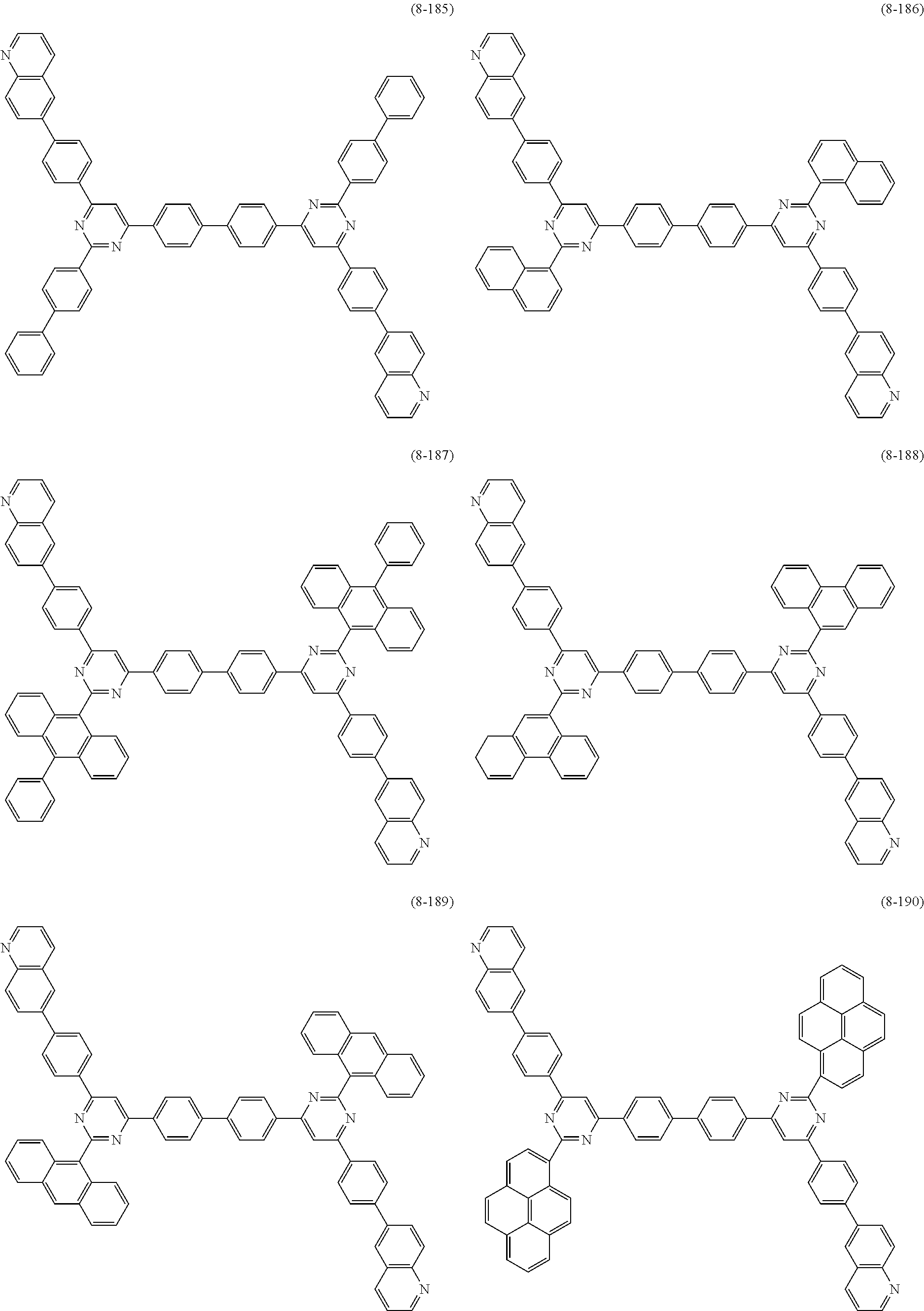

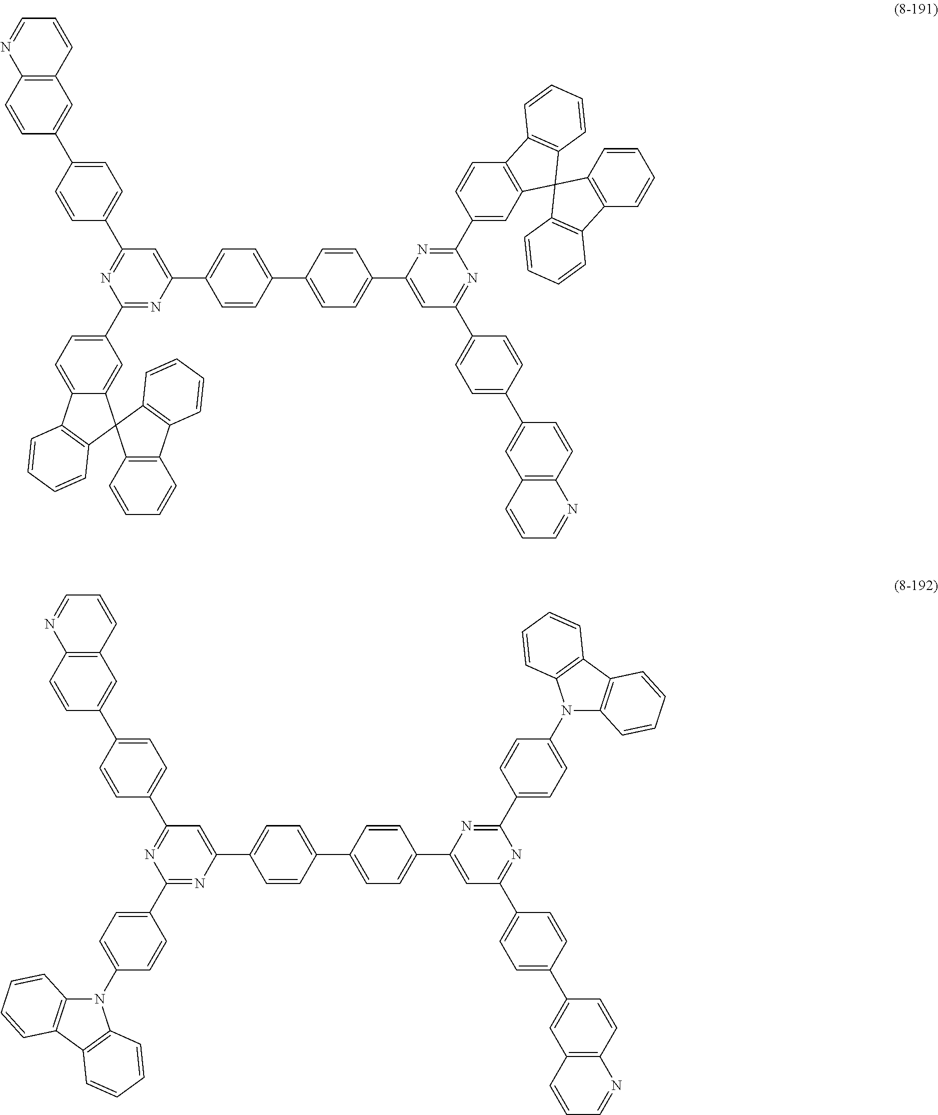

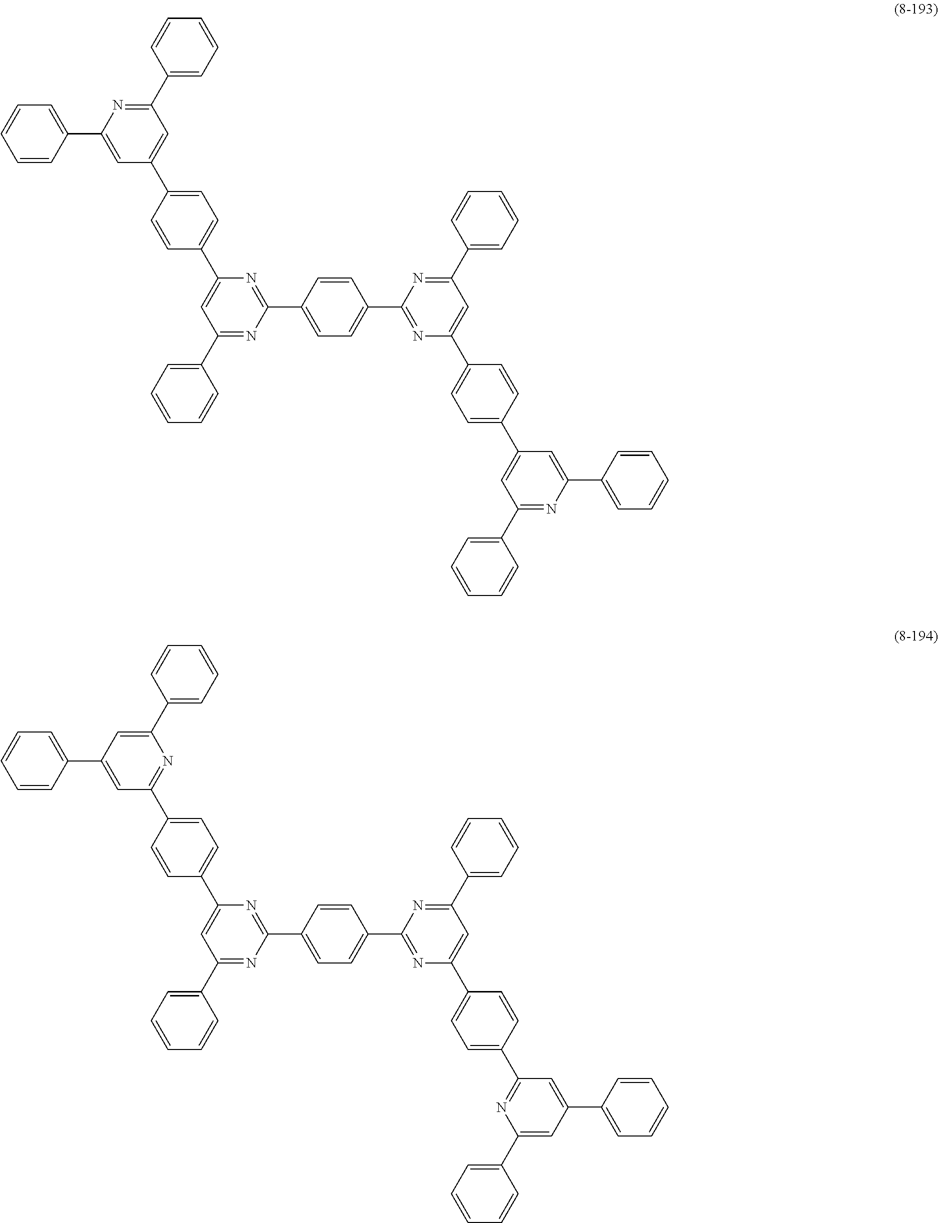

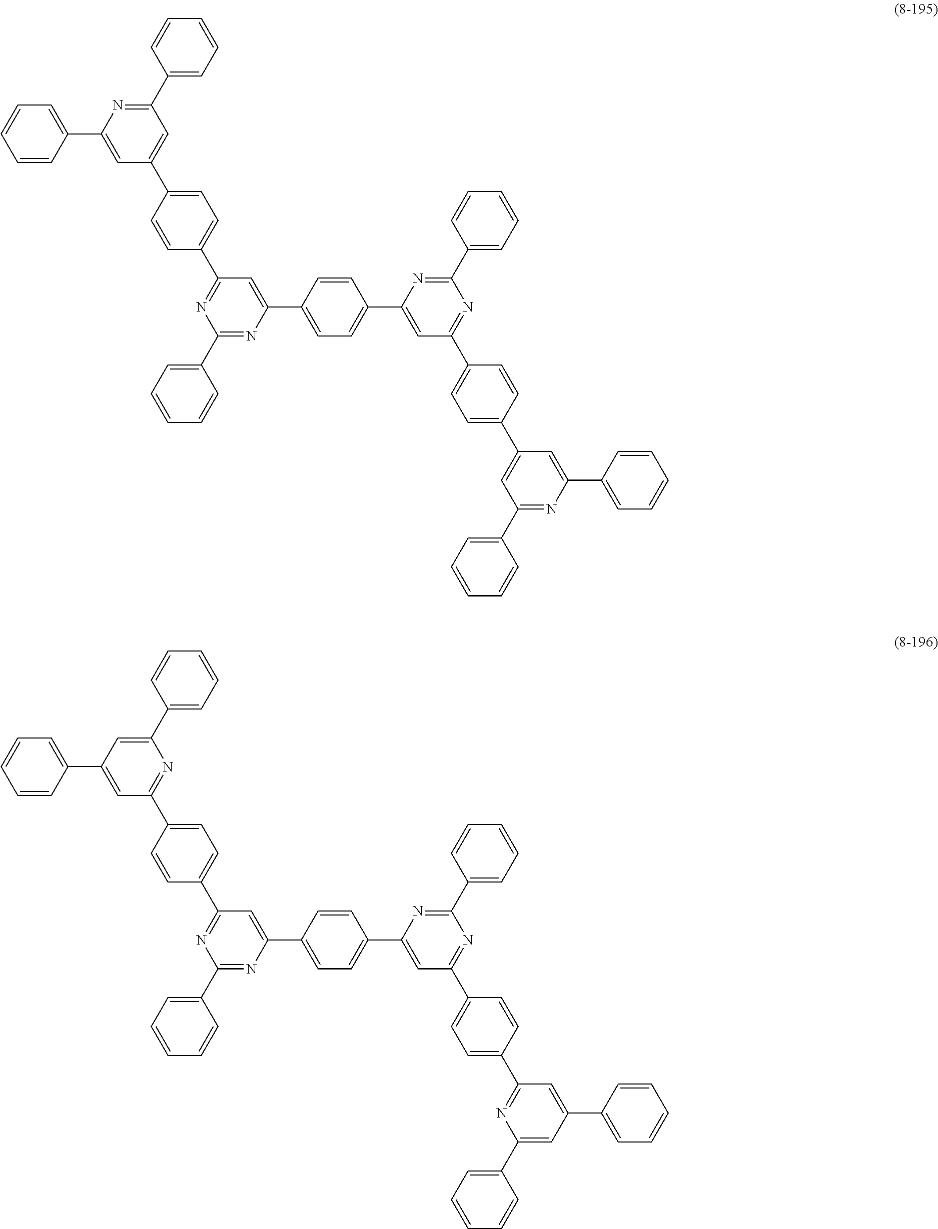

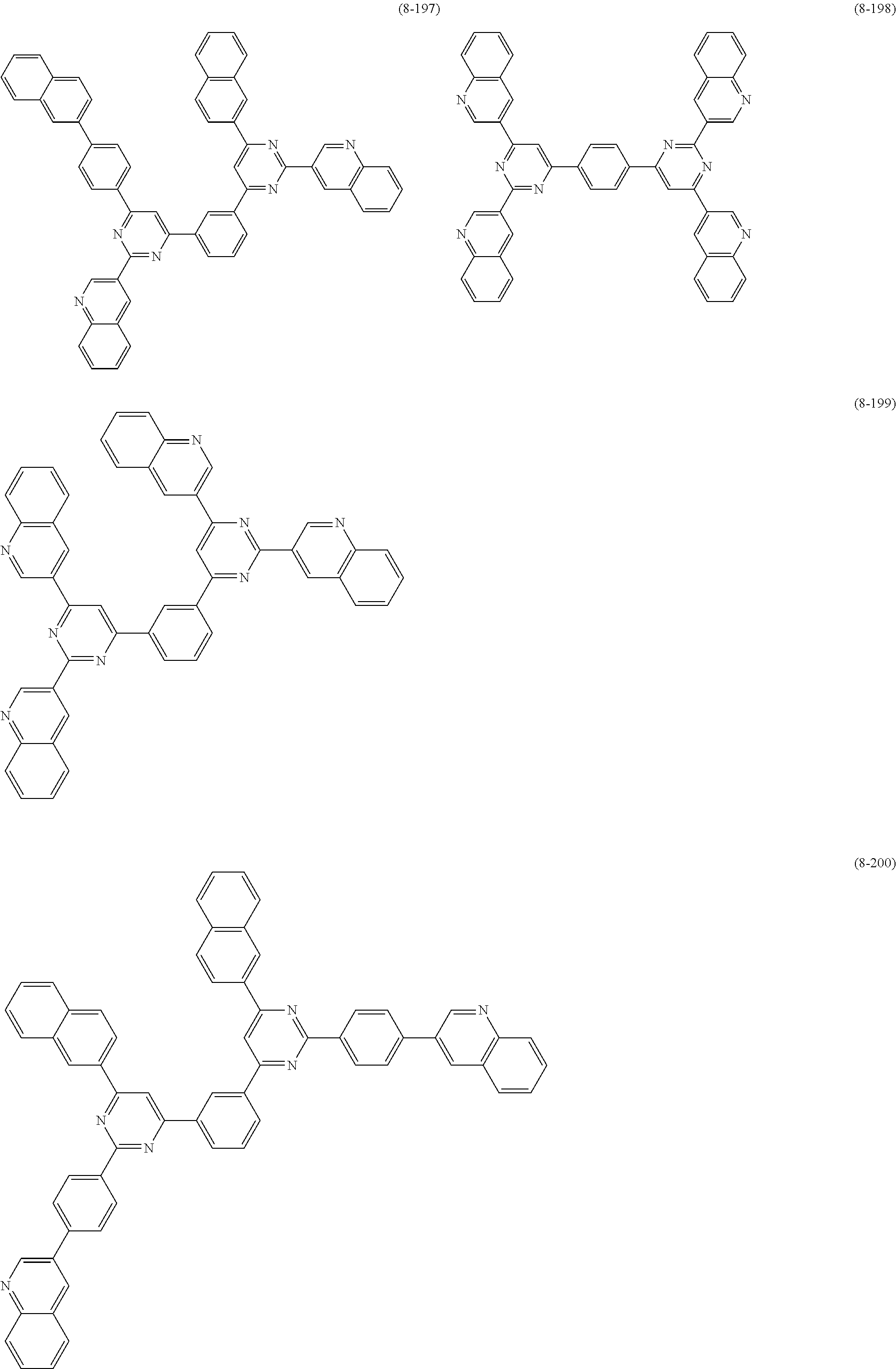

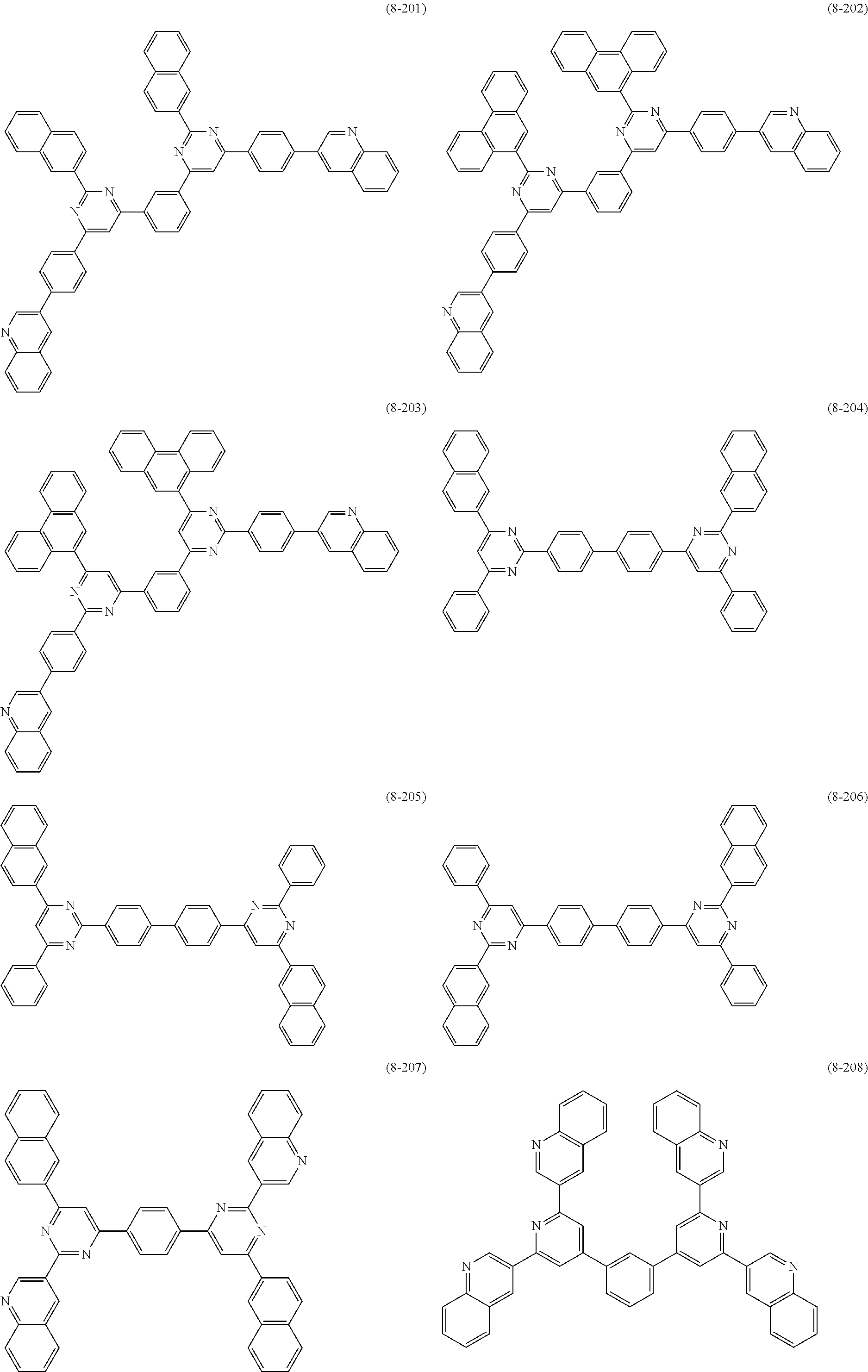

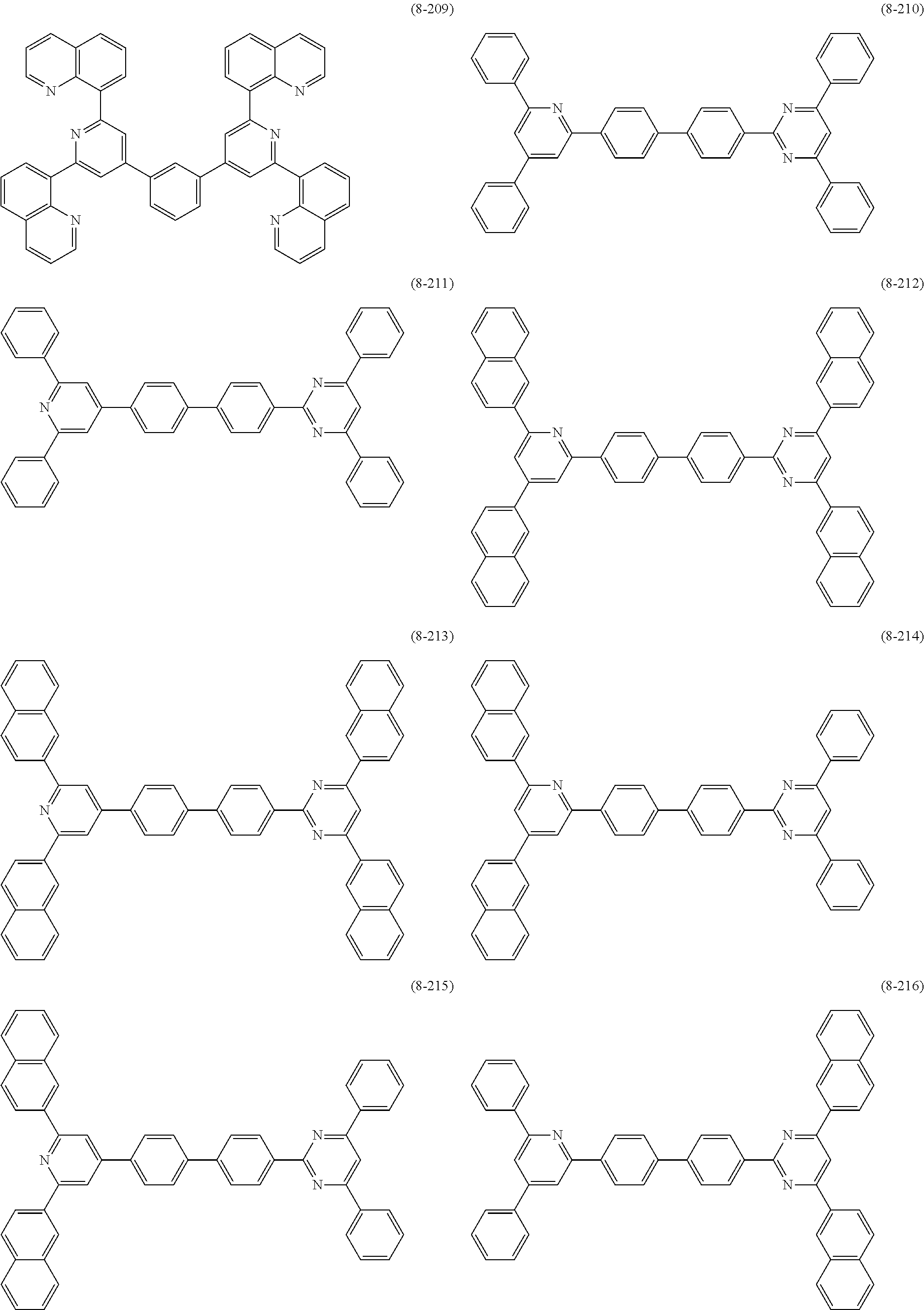

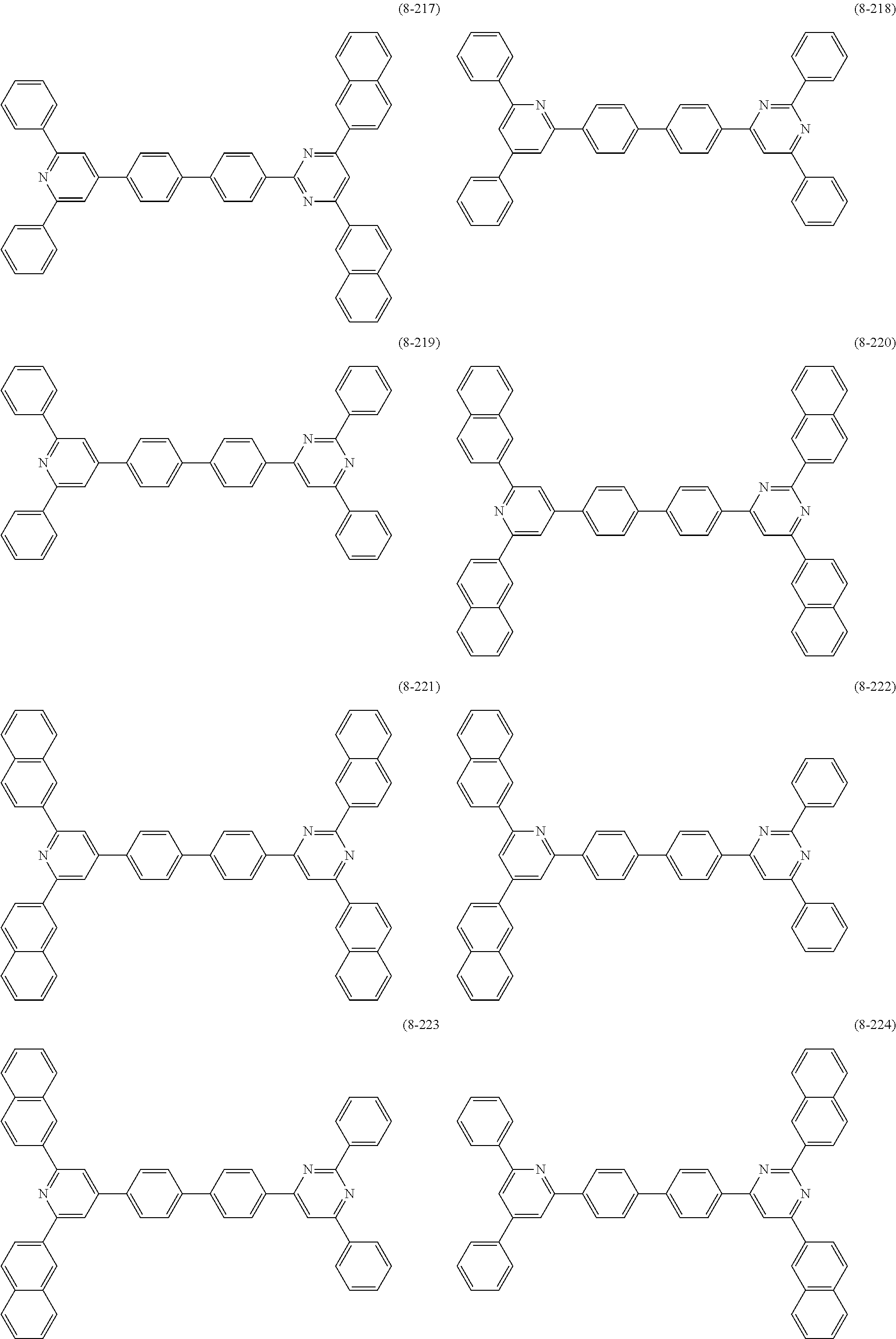

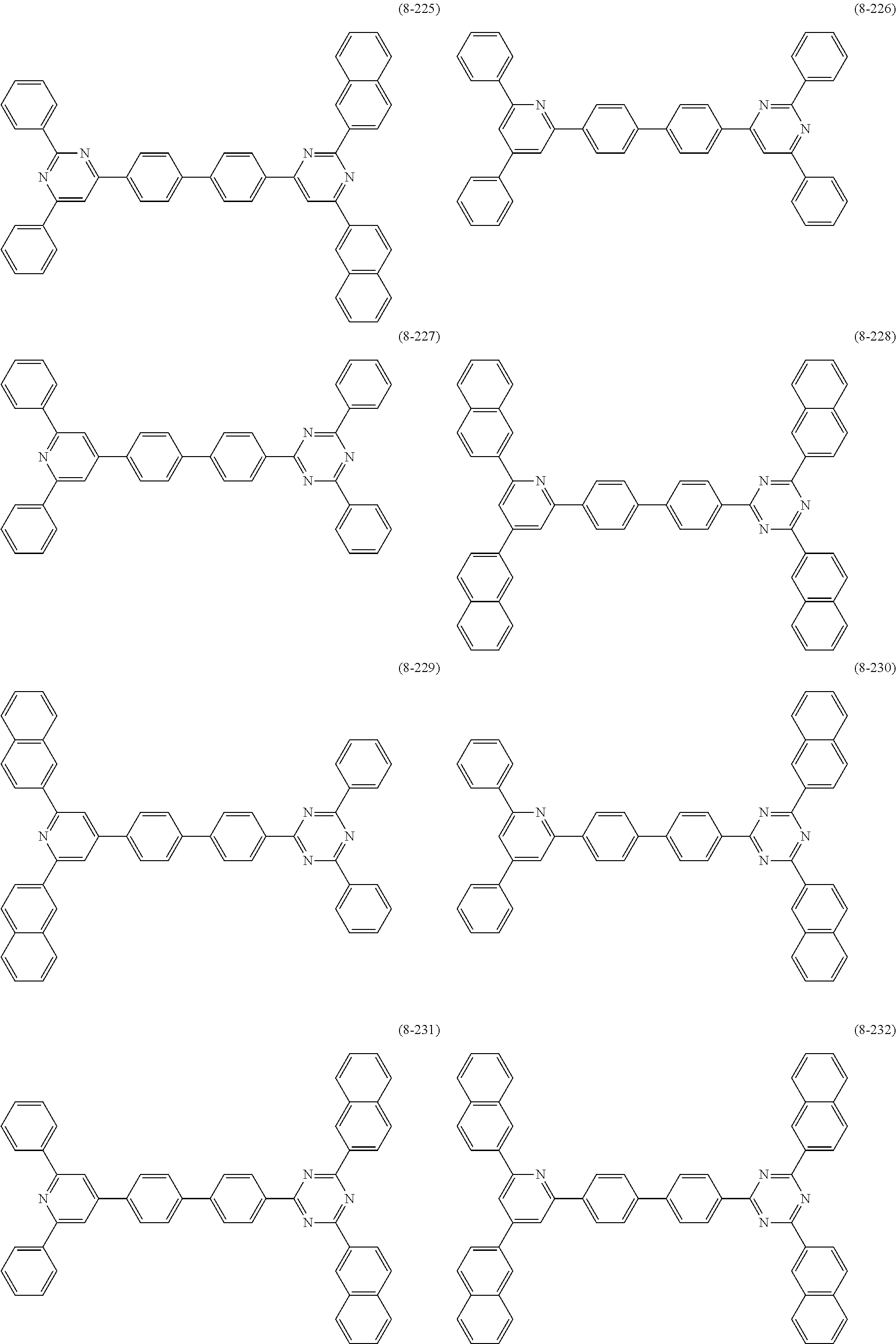

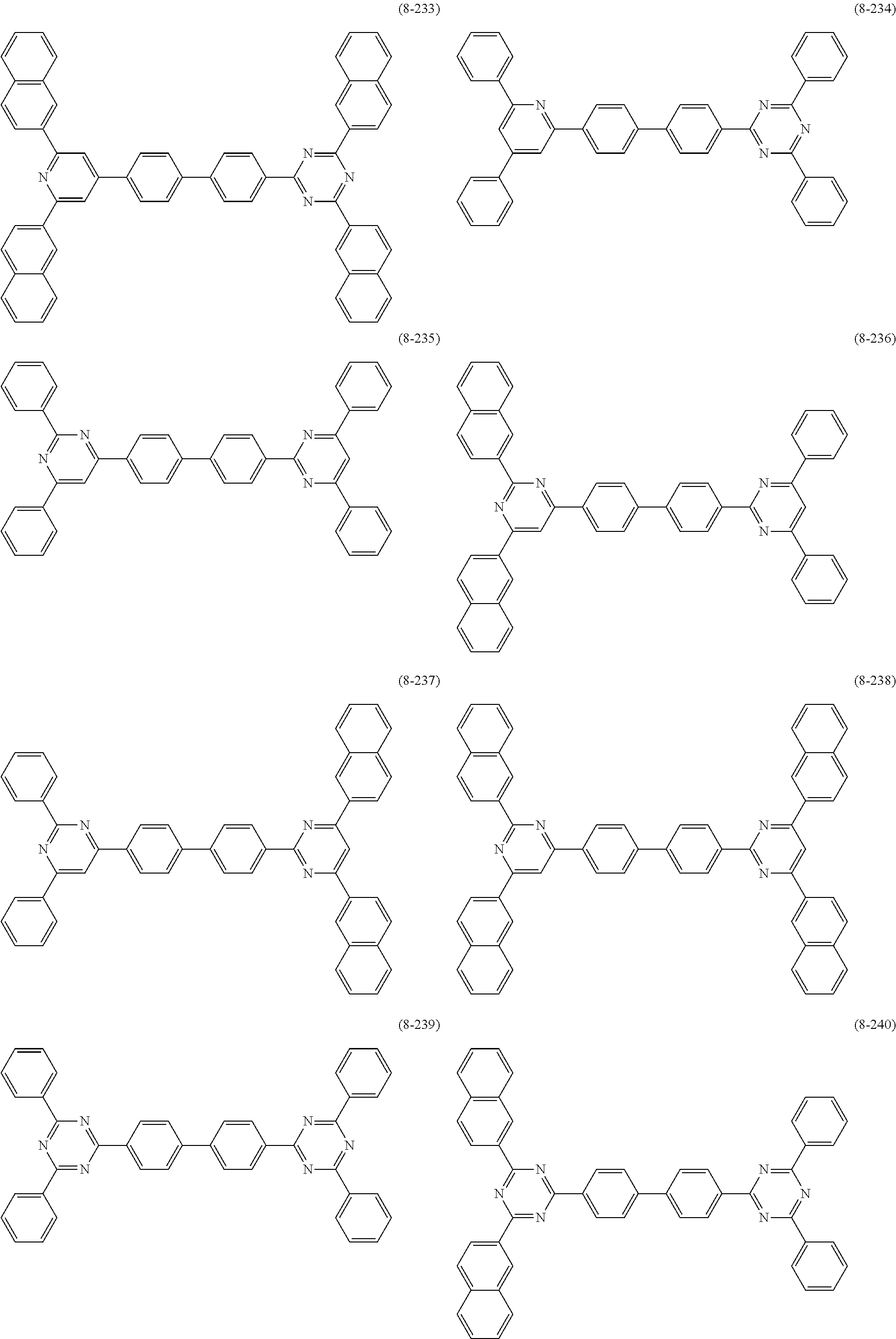

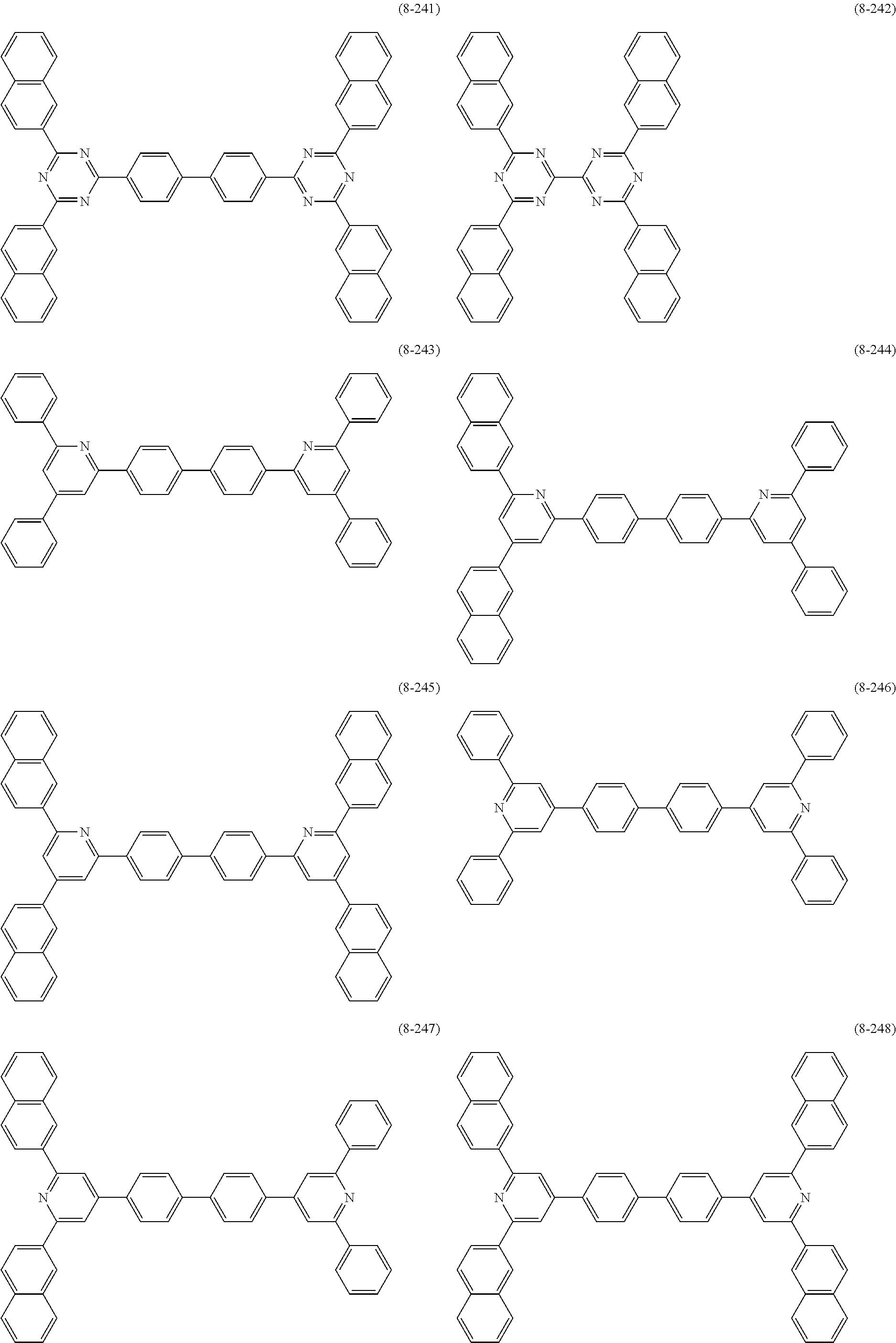

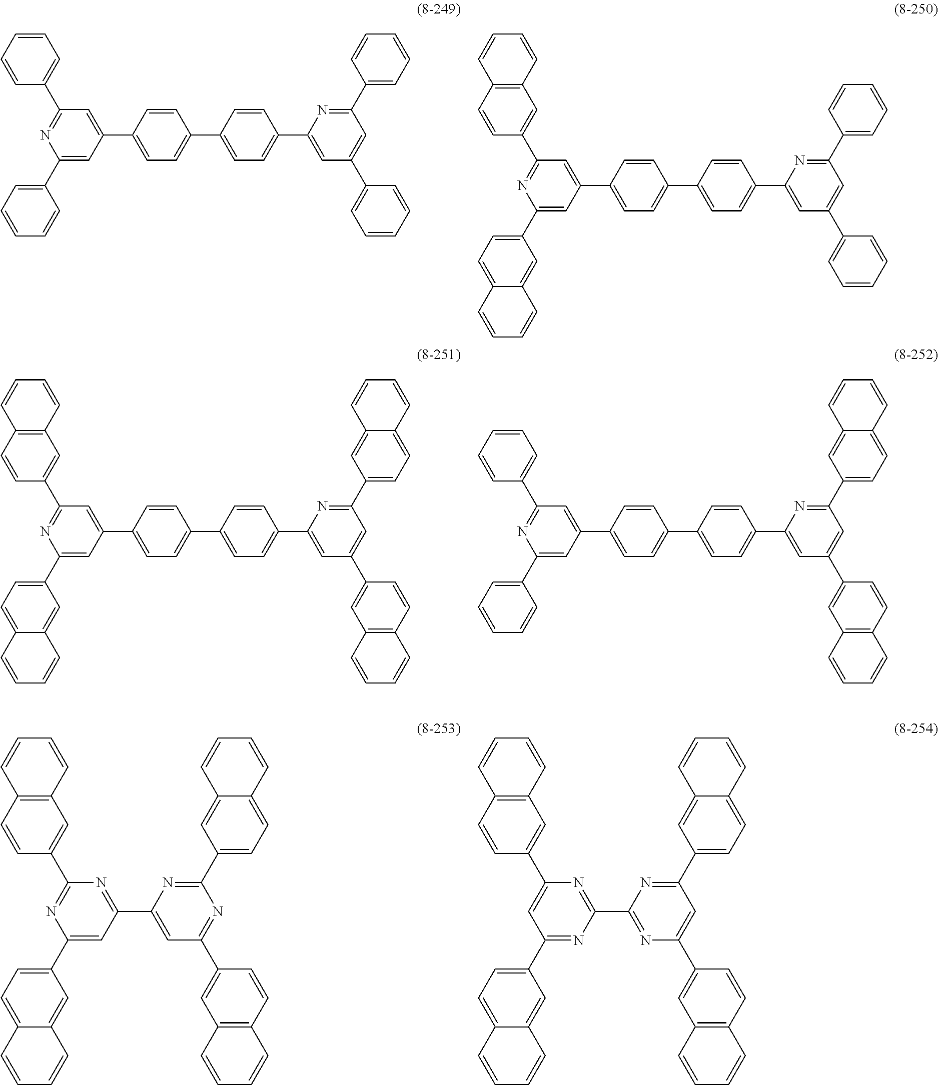

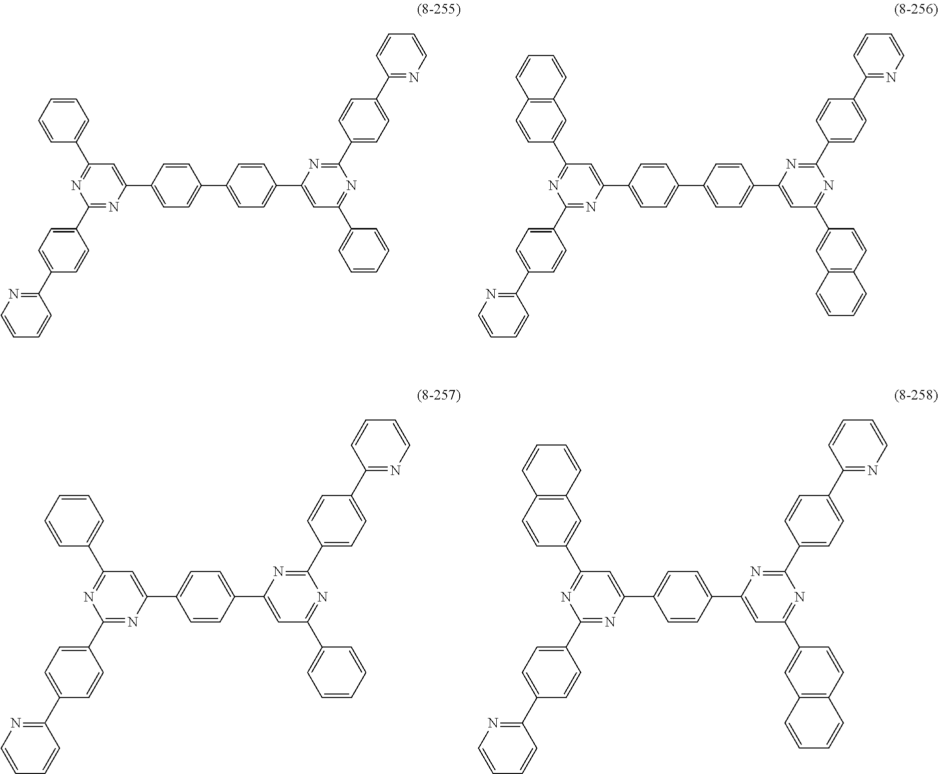

The embodiments may also be realized by providing an organic photoelectric device including an anode; a cathode; and at least one organic thin layer between the anode and the cathode, wherein the organic thin layer includes the compound for an organic photoelectric device according to an embodiment.

The organic thin layer including the compound for an organic photoelectric device may include an emission layer, a hole transport layer (HTL), a hole injection layer (HIL), a hole blocking layer, an electron transport layer (ETL), an electron injection layer (EIL), an electron blocking layer, or a combination thereof.

The organic thin layer including the compound for an organic photoelectric device may be an emission layer, and the compound for an organic photoelectric device may be a phosphorescent host or a fluorescent host in the emission layer.

The organic thin layer including the compound for an organic photoelectric device may be an emission layer, and the compound for an organic photoelectric device may be a fluorescent blue dopant in the emission layer.

The embodiments may also be realized by providing a display device comprising the organic photoelectric device according to an embodiment.

### BRIEF DESCRIPTION OF THE DRAWINGS

The embodiments will become apparent to those of ordinary skill in the art by describing in detail exemplary embodiments with reference to the attached drawings in which:

FIGS. 1 to 5 illustrate cross-sectional views of organic photoelectric devices including compounds for an organic photoelectric device according to various embodiments.

### DETAILED DESCRIPTION

Korean Patent Application No. 10-2009-0063234, filed on Jul. 10, 2009, in the Korean Intellectual Property Office, and entitled: “Compound for Organic Photoelectric Device and Organic Photoelectric Device,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. In addition, it will also be understood that when a layer is referred to as being “between” two layers, it can be the only layer between the two layers, or one or more intervening layers may also be present. Like reference numerals refer to like elements throughout.

Throughout the specification, the term “substituted” may refer to one substituted with a C1 to C30 alkyl group, a C6 to C30 aryl group, or a combination thereof.

Throughout the specification, the term “hetero” may refer to one including 1 to 3 of N, O, S, P, or a combination thereof and carbons in a rest thereof in one substituent. For example, a compound including N may better function as an electron transport group.

Throughout the specification, the term “a combination thereof” may refer to at least two substituents bound to each other by a linker or at least two substituents fused to each other.

According to an embodiment, a compound for an organic photoelectric device represented by the following Chemical Formula 1 is provided.

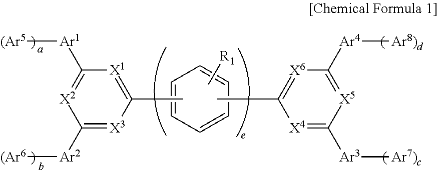

In Chemical Formula 1, X¹ to X⁶ may each independently be N or CR′. R′ may be hydrogen, a C1 to C30 alkyl group, a C6 to C30 aryl group, or a combination thereof. In an implementation, at least one selected from X¹ to X³ may be N, and at least one selected from X⁴ to X⁶ may be N.

R¹ may be hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof.

Ar¹ to Ar⁴ may each independently be a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof.

Ar⁵ to Ar⁸ may each independently be a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof.

a, b, c, and d may each independently be integers ranging from 0 to 2, and e may be an integer ranging from 0 to 3. In an implementation, when a, b, c, d, and e are integers of greater than or equal to 2, each unit thereof may be the same or different from each other. In an implementation, e may be 1 or 2. In an implementation, when e is 2, two phenylene groups having the R1 substituent are in a para position relative to each other.

The compound for an organic photoelectric device may be represented by the following Chemical Formula 2a, 2b, or 2c.

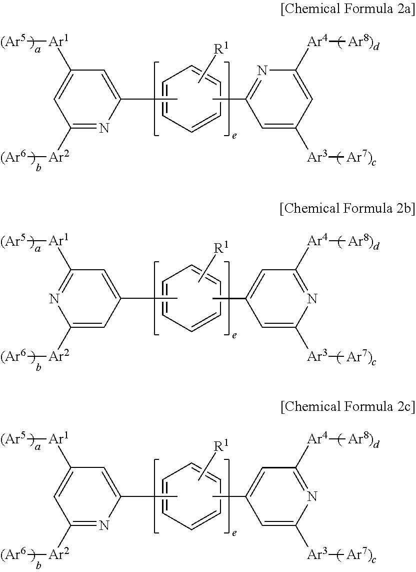

In Chemical Formulae 2a to 2c, Ar⁵ to Ar⁸ may each independently be a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof. In an implementation, at least one selected from Ar⁵ to Ar⁸ may be a C6 to C30 aryl group.

R¹, Ar¹ to Ar⁴, a, b, c, d, and e may be the same as defined in the above Chemical Formula 1.

The compound for an organic photoelectric device may be represented by the following Chemical Formula 3a, 3b, or 3c.

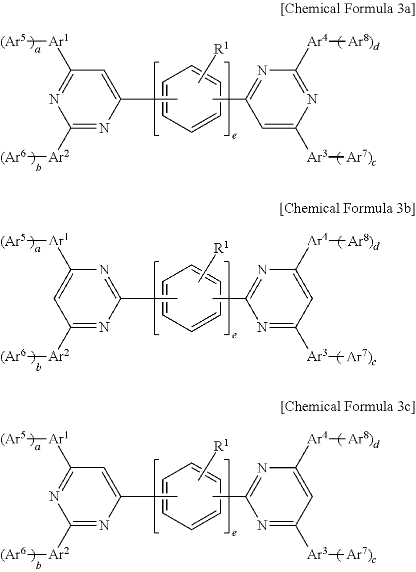

In Chemical Formulae 3a to 3c, R¹, Ar¹ to Ar⁸, a, b, c, d, and e may be the same as defined in the above Chemical Formula 1. In an implementation, when a, b, c, and d are 0, at least one selected from Ar¹ to Ar⁴ may have C7 to C30 carbon numbers, e.g., at least one selected from Ar¹ to Ar⁴ may include about 7 to about 30 carbon atoms.

The compound for an organic photoelectric device may be represented by the following Chemical Formula 4a.

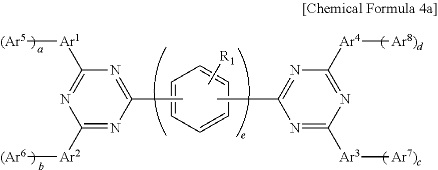

In Chemical Formula 4a, R¹, Ar¹ to Ar⁸, a, b, c, d, and e may be the same as defined in the above Chemical Formula 1. It an implementation, at least one selected from Ar¹ to Ar⁴ may be an aryl group or an arylene group.

The compound for an organic photoelectric device may be represented by the following Chemical Formula 5a, 5b, 5c, or 5d.

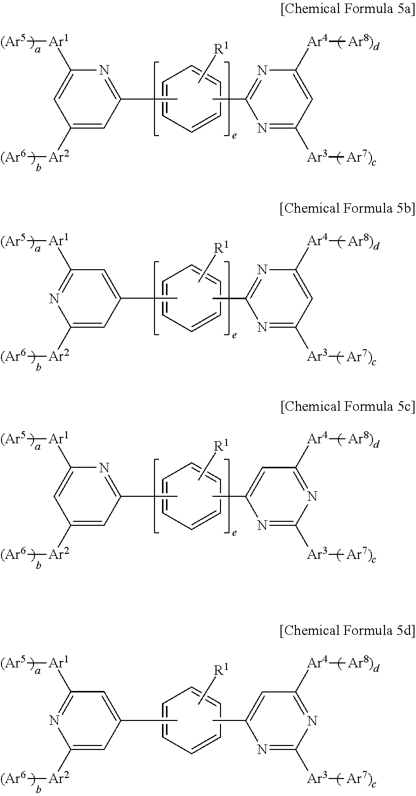

In Chemical Formulae 5a to 5d, R¹, Ar¹ to Ar⁸, a, b, c, d, and e may be the same as defined in the above Chemical Formula 1.

The compound for an organic photoelectric device may be represented by the following Chemical Formula 6a or 6b.

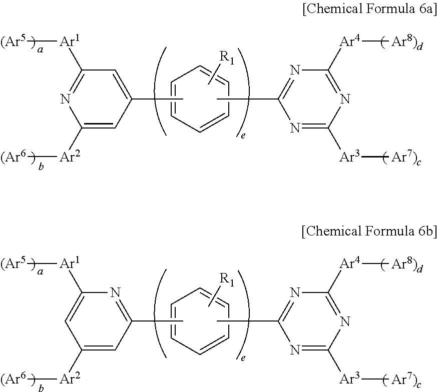

In Chemical Formulae 6a and 6b, R¹, Ar¹ to Ar⁸, a, b, c, d and e may be the same as defined in the above Chemical Formula 1.

The compound for an organic photoelectric device may be represented by the following Chemical Formula 7a or 7b.

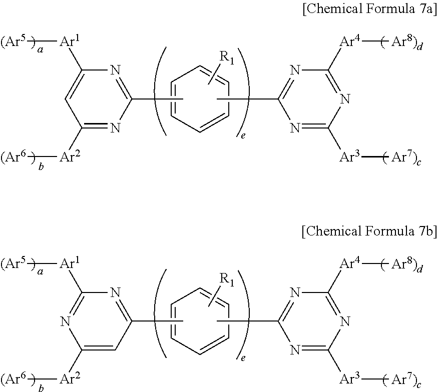

In Chemical Formulae 7a and 7b, R¹, Ar¹ to Ar⁸, a, b, c, d and e may be the same as defined in the above Chemical Formula 1.

In an implementation, when any of R¹ and Ar¹ to Ar⁸ in the above Chemical Formulae 7a and 7b is an aryl group, the aryl group may be a phenyl group, a biphenyl group, a terphenyl group, a stilbenzyl group, a naphthyl group, an anthracenyl group, a phenanthrenyl group, a pyrenyl group, a perylenyl group, or a combination thereof. In an implementation, when any of Ar¹ to Ar⁴ in the above Chemical Formulae 7a and 7b is an arylene group, the arylene group may be a phenylene group, a biphenylene group, a terphenylene group, a stilbenzylene group, a naphthylene group, an anthracenylene group, a phenanthrenylene group, a pyrenylene group, a perylenylene group, or a combination thereof. However, the aryl group and the arylene group are not limited to the aforementioned examples.

In an implementation, when any of R¹ and Ar¹ to Ar⁸ in the above Chemical Formulae 7a and 7b is a heteroaryl group, and/or any of Ar¹ to Ar⁴ is a heteroarylene group, a LUMO (Lowest Unoccupied Molecular Orbital) energy level may be lowered and injection and transportation characteristics of an electron may be improved. Accordingly, an organic photoelectric device may be operated with a lower voltage and thus, may have improved electric power efficiency.

The heteroaryl group may include, e.g., a thiophenyl group, a furanyl group, a pyrrolyl group, an imidazolyl group, a thiazolyl group, an oxazolyl group, an oxadiazoly group, a triazolyl group, a pyridinyl group, a pyradazinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof. The heteroarylene group may include, e.g., a thiophenylene group, a furanylene group, pyrrolene group, an imidazolylene group, a thiazolylene group, an oxazolylene group, an oxadiazoly group, a triazolyl group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof. However, the heteroaryl group and the heteroarylene group are not limited to the aforementioned examples.

In an implementation, when any of R¹ and Ar¹ to Ar⁸ in the above Chemical Formulae 7a and 7b includes an arylamine group, a carbazolyl group, or a fluorenyl group, the compound may exhibit conductive characteristics depending on a HOMO (highest occupied molecular orbital) energy level and thus, may be prepared as a p-type having cation characteristics due to formation of holes.

In addition, when e in the above Chemical Formulae 7a and 7b is an integer ranging from 0 to 2, the compound may be used to prepare a device having a lower driving voltage and high photo efficiency.

The compound for an organic photoelectric device may be represented by Chemical Formula 8, e.g., one of the following Compounds 8-1 to 8-258. However, the compound for organic photoelectric device according to an embodiment is not limited to the compounds illustrated below.

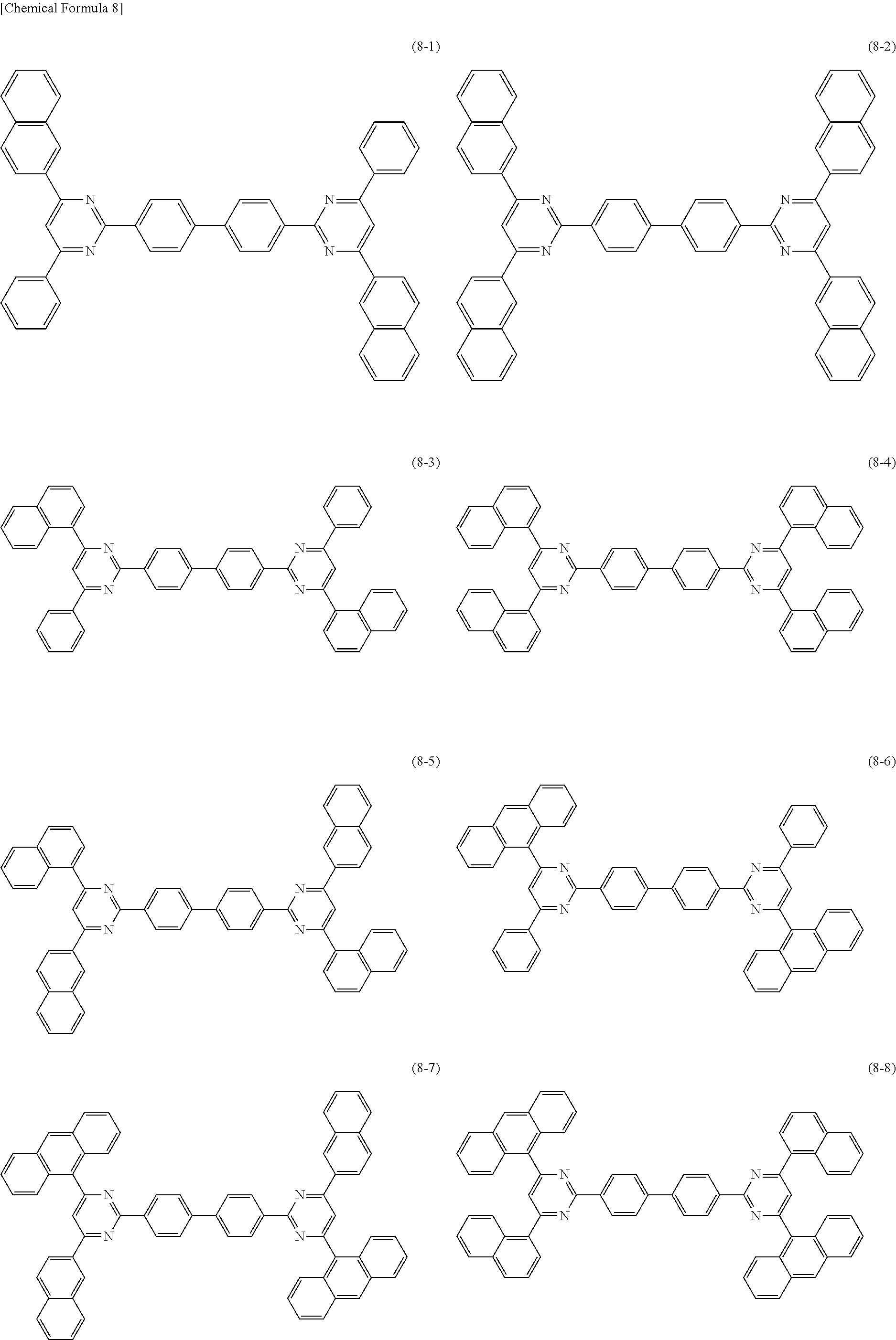

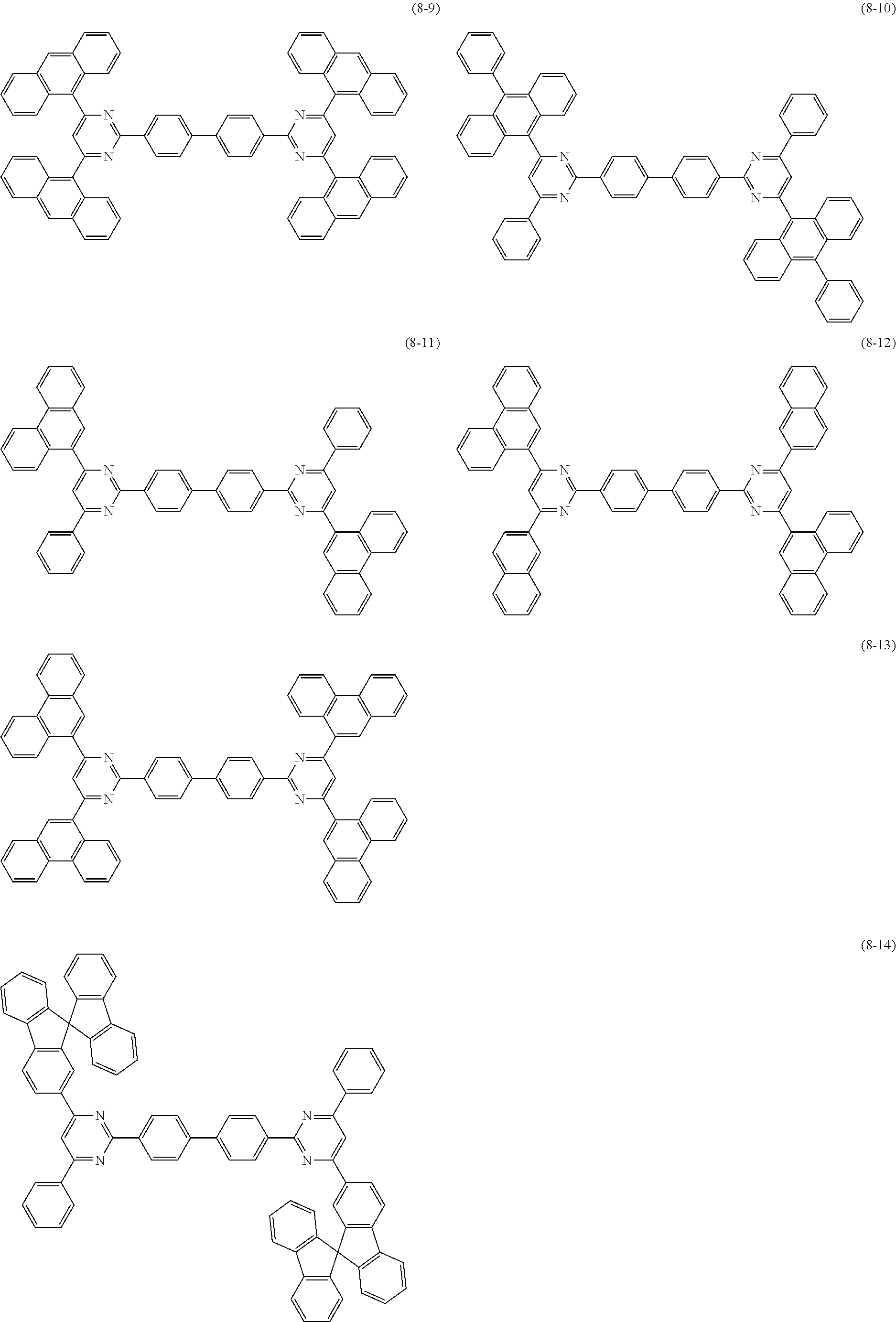

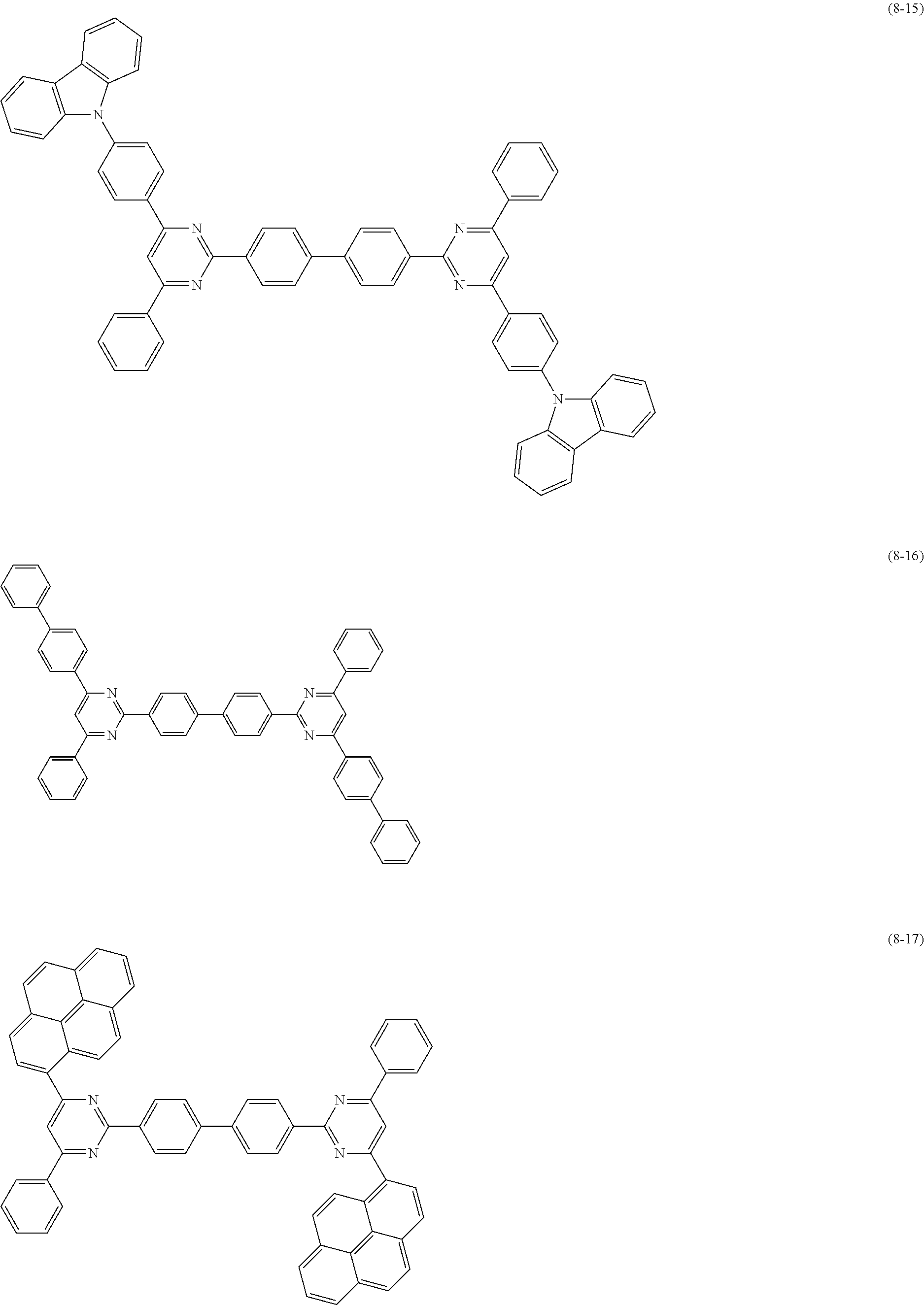

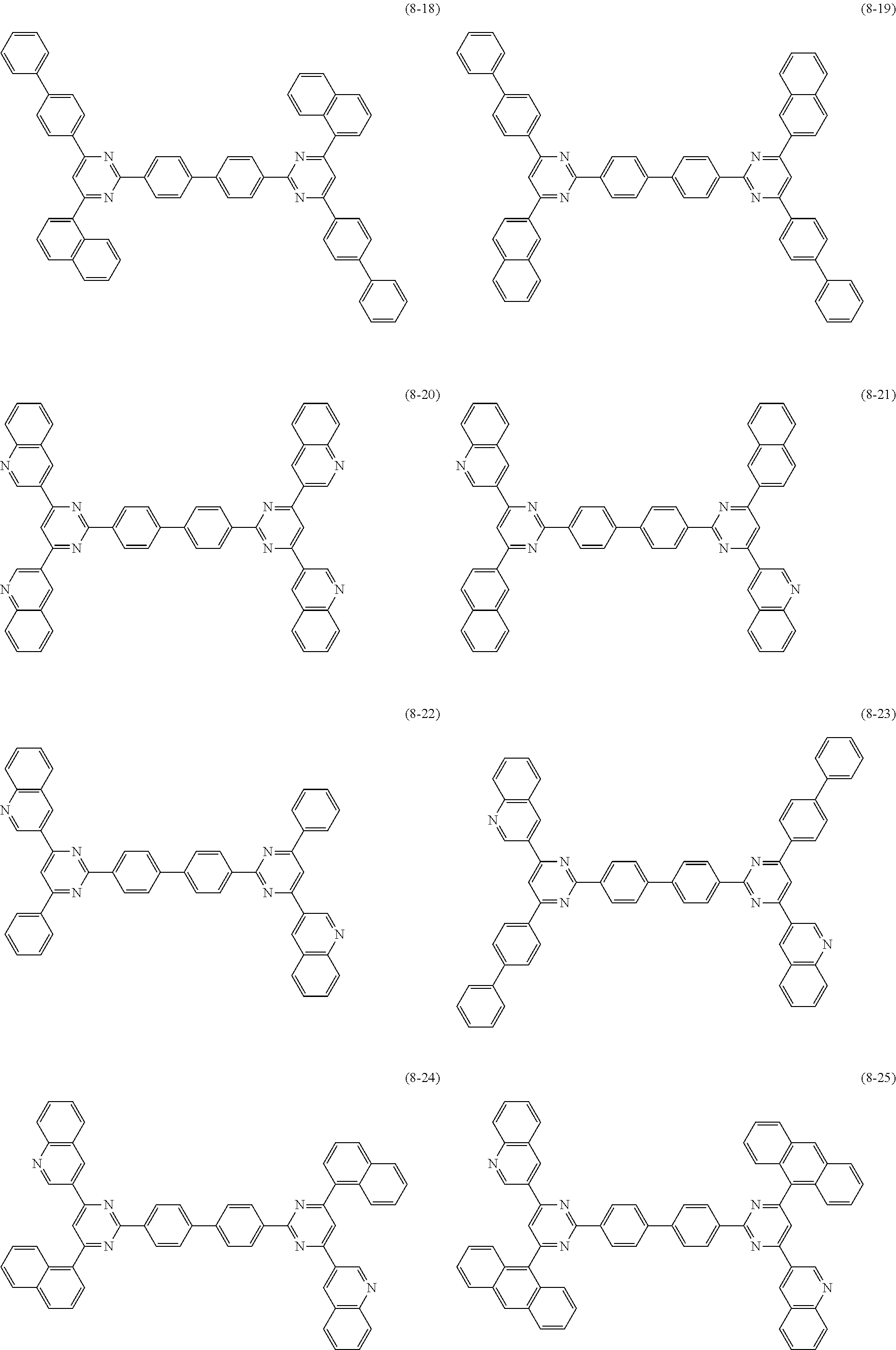

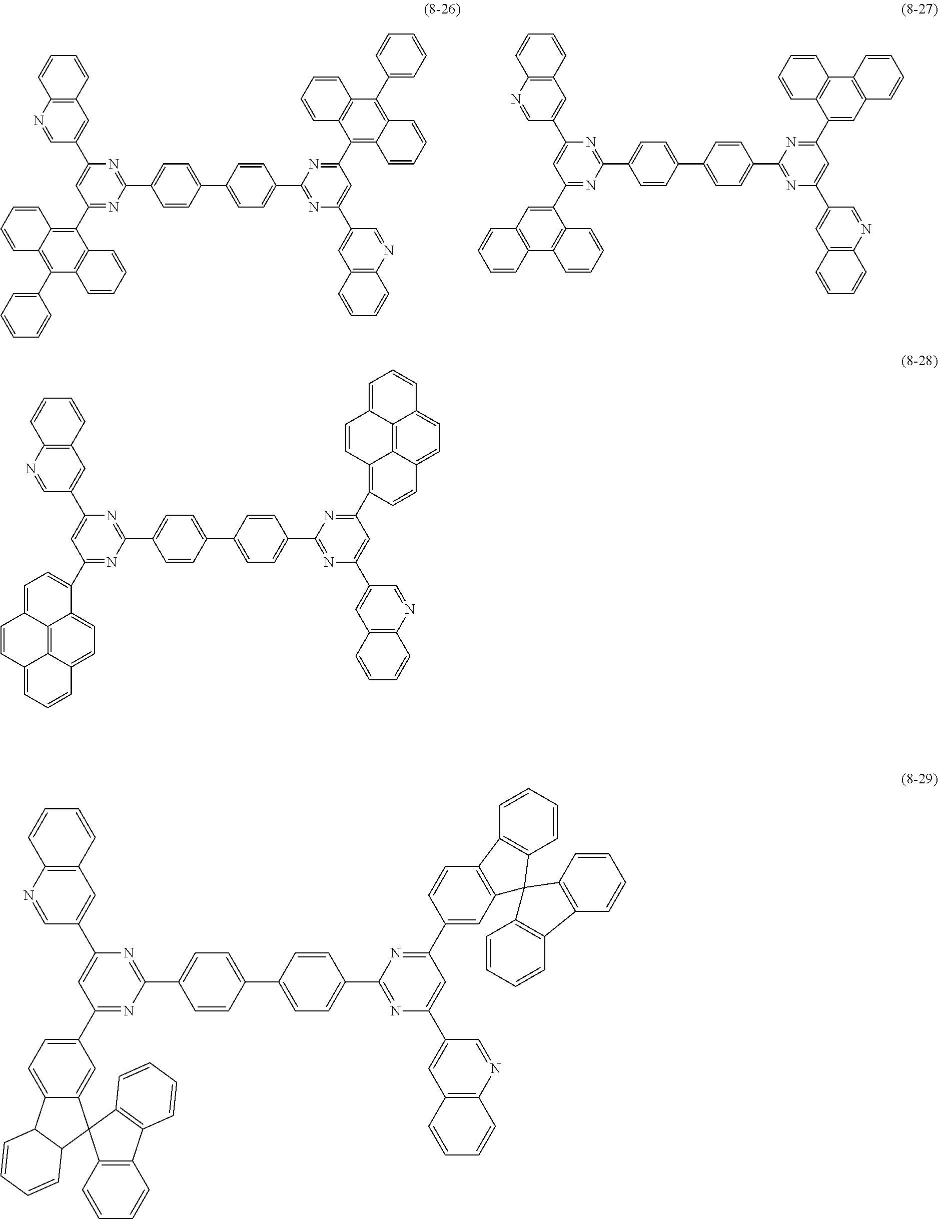

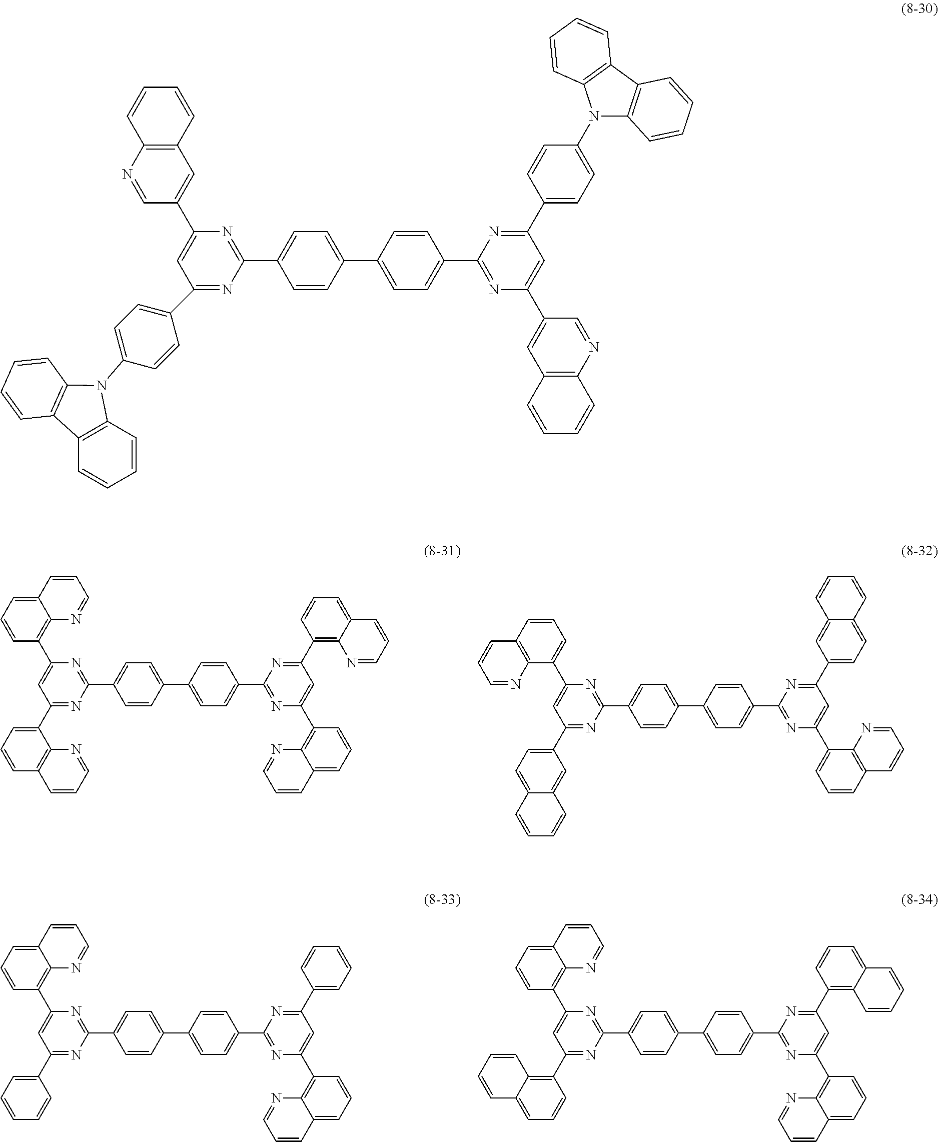

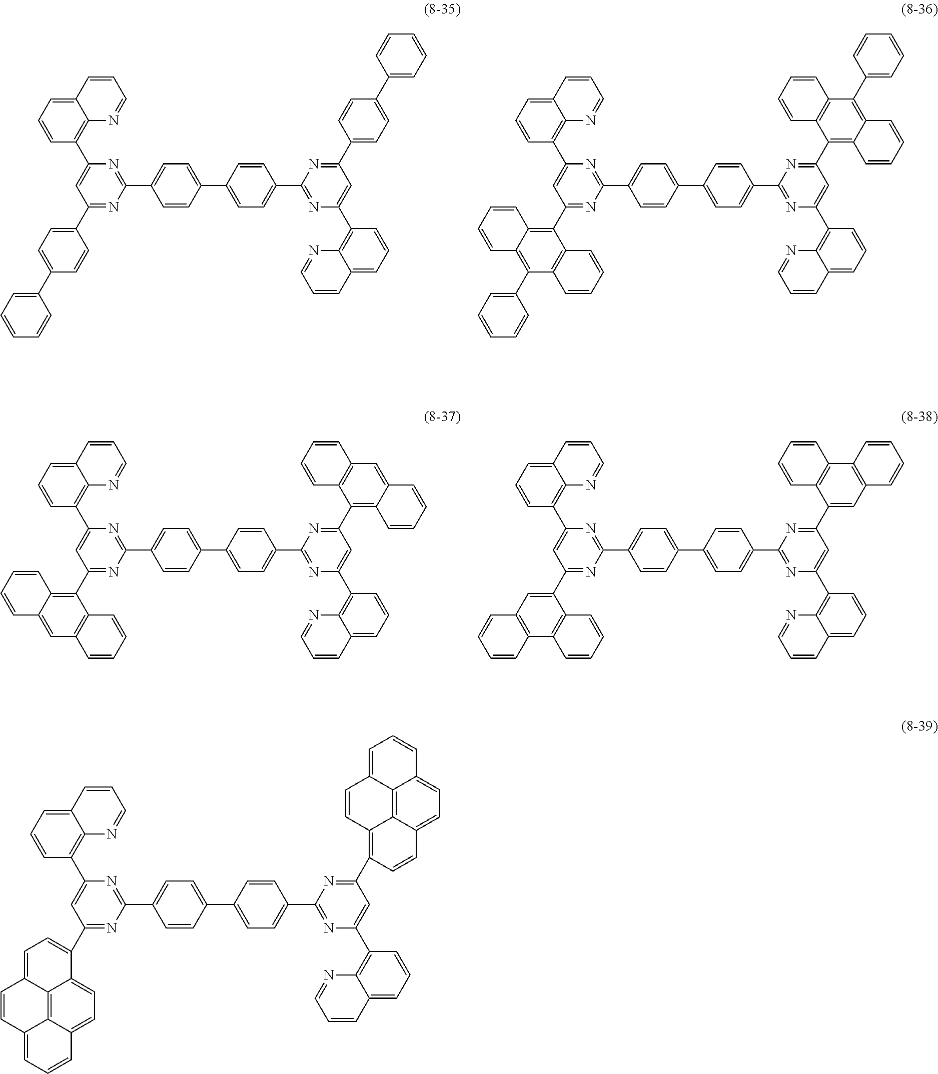

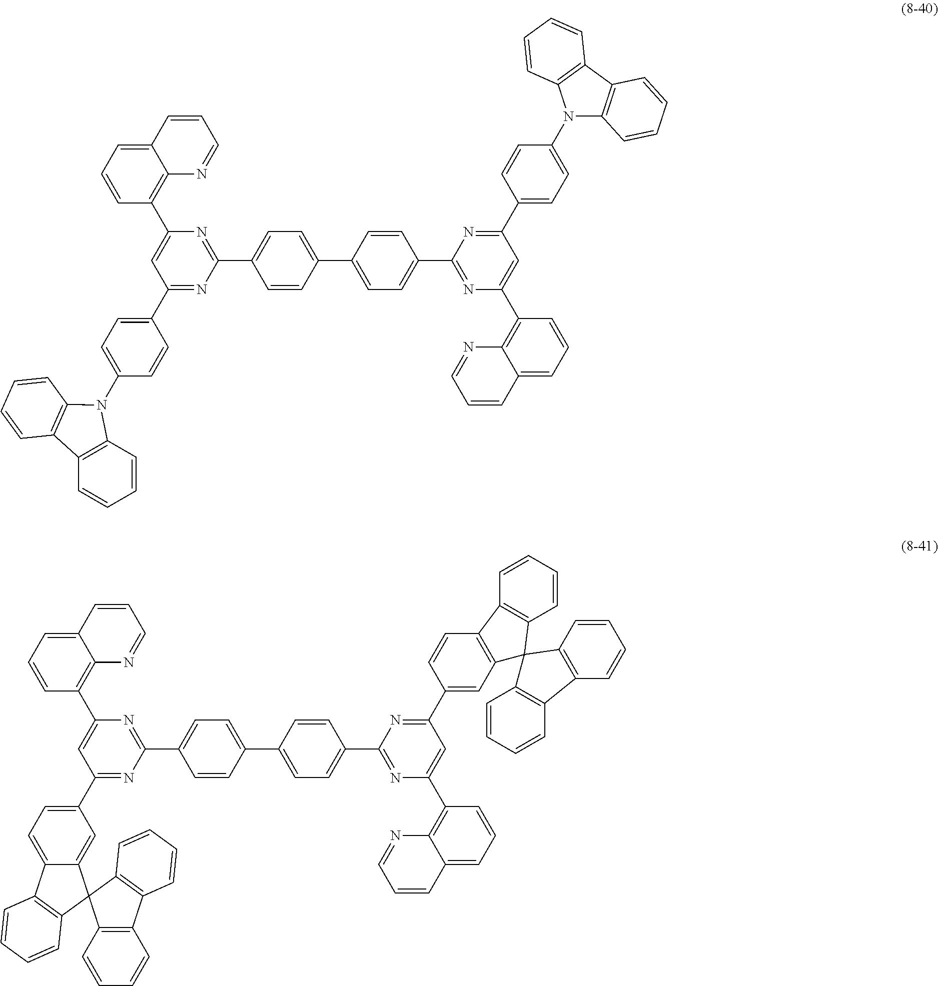

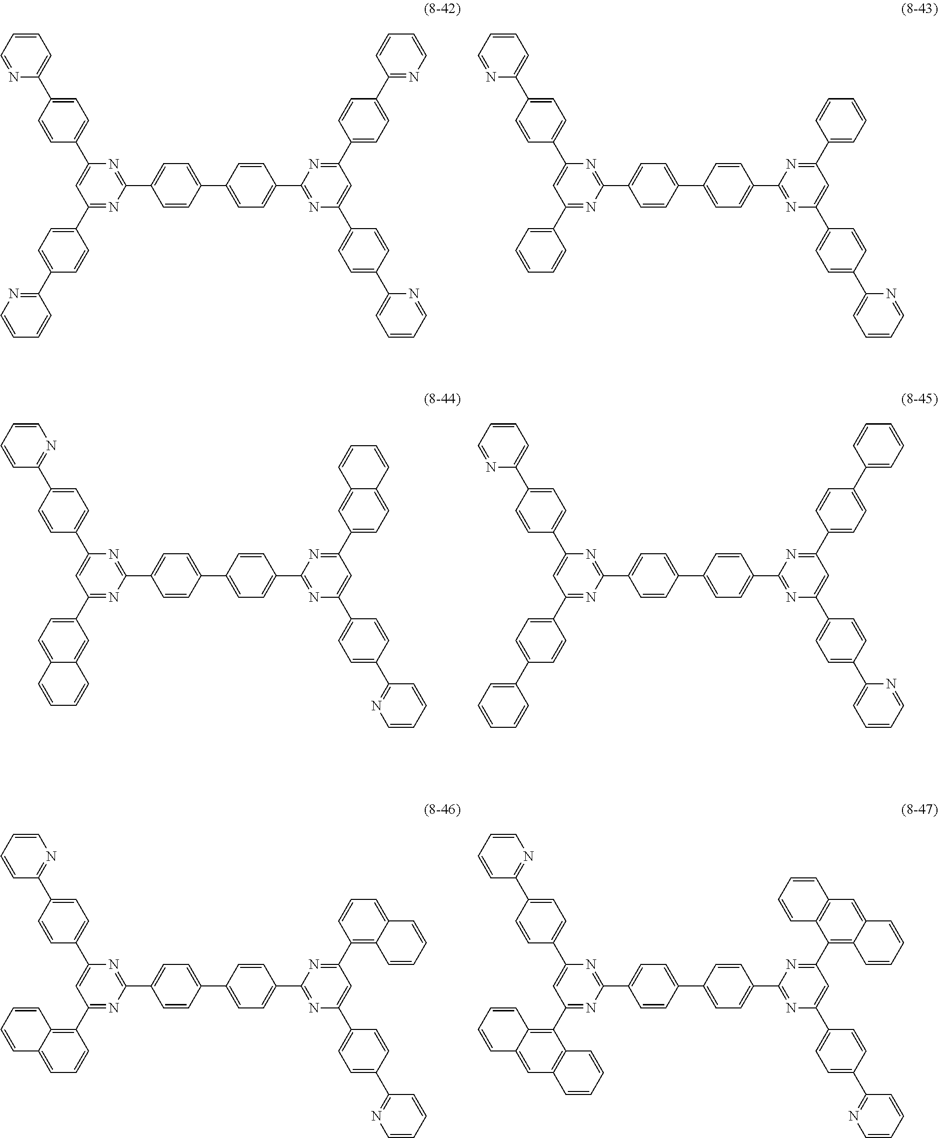

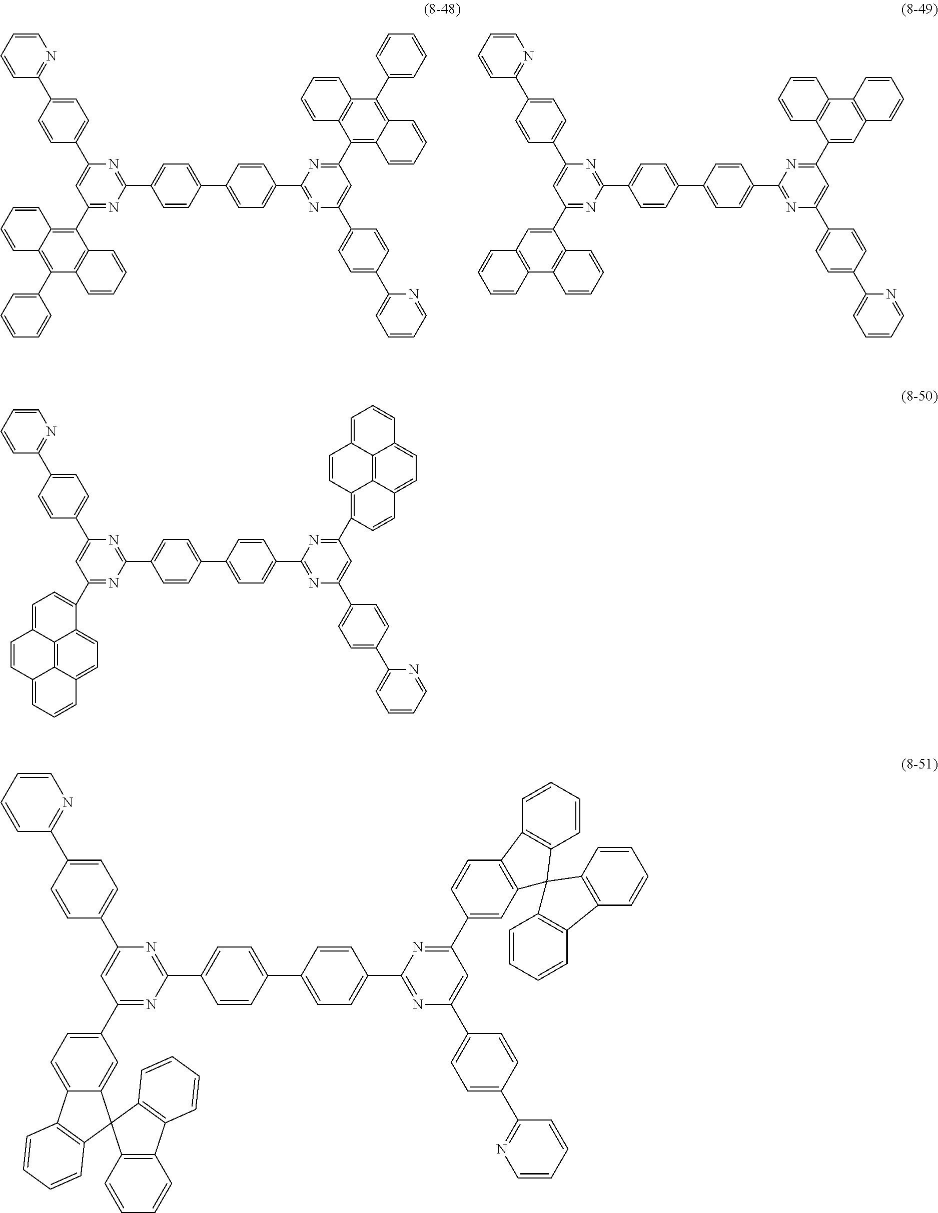

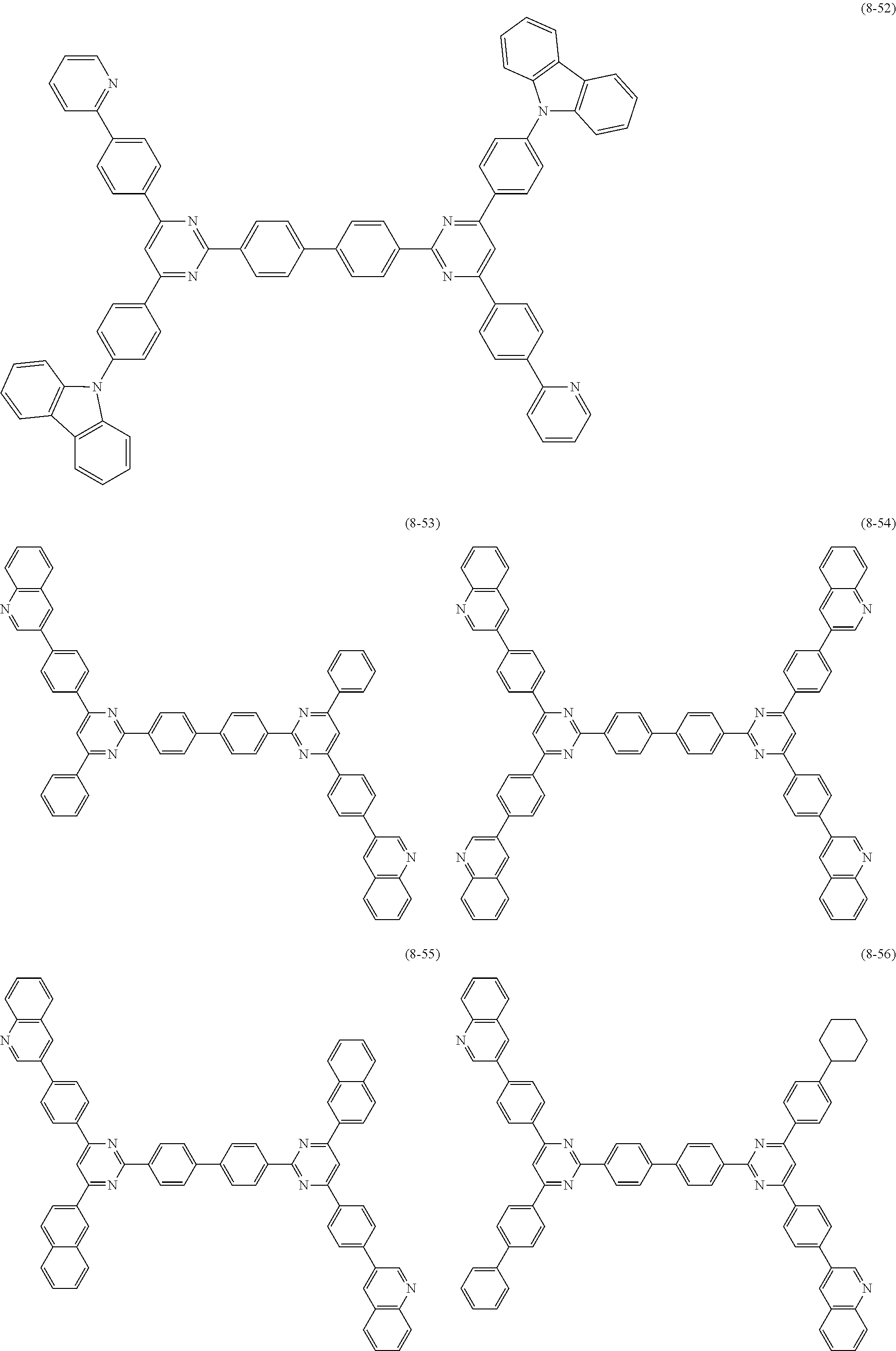

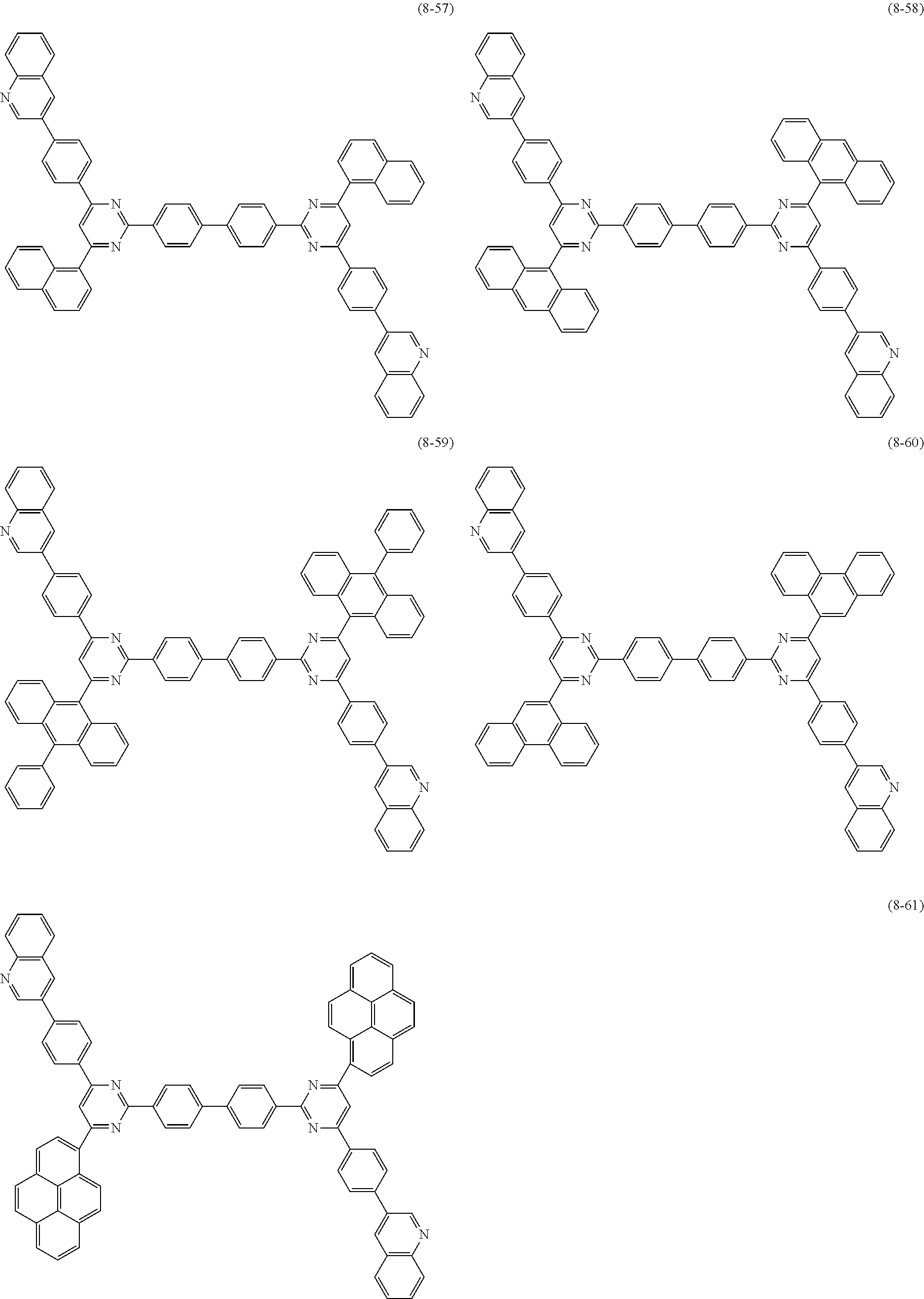

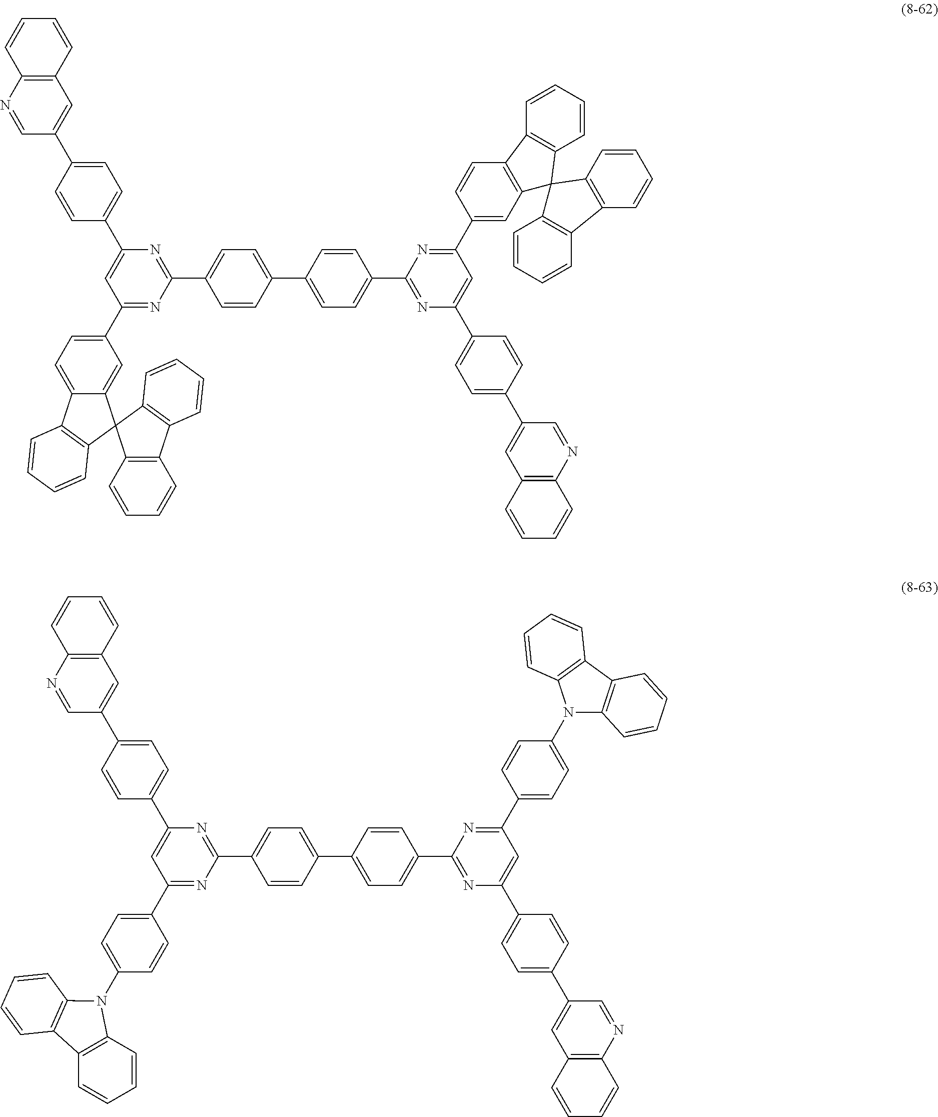

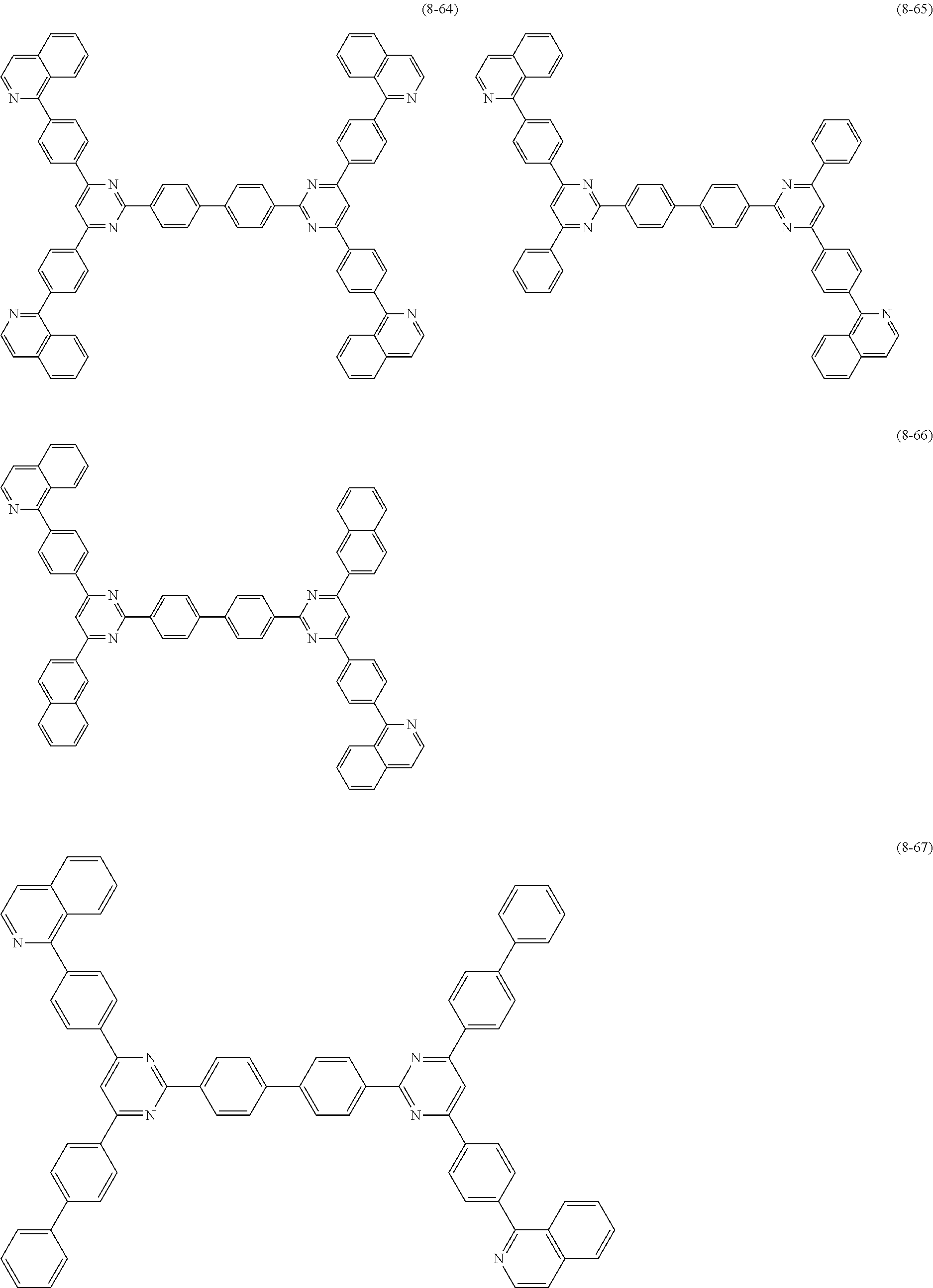

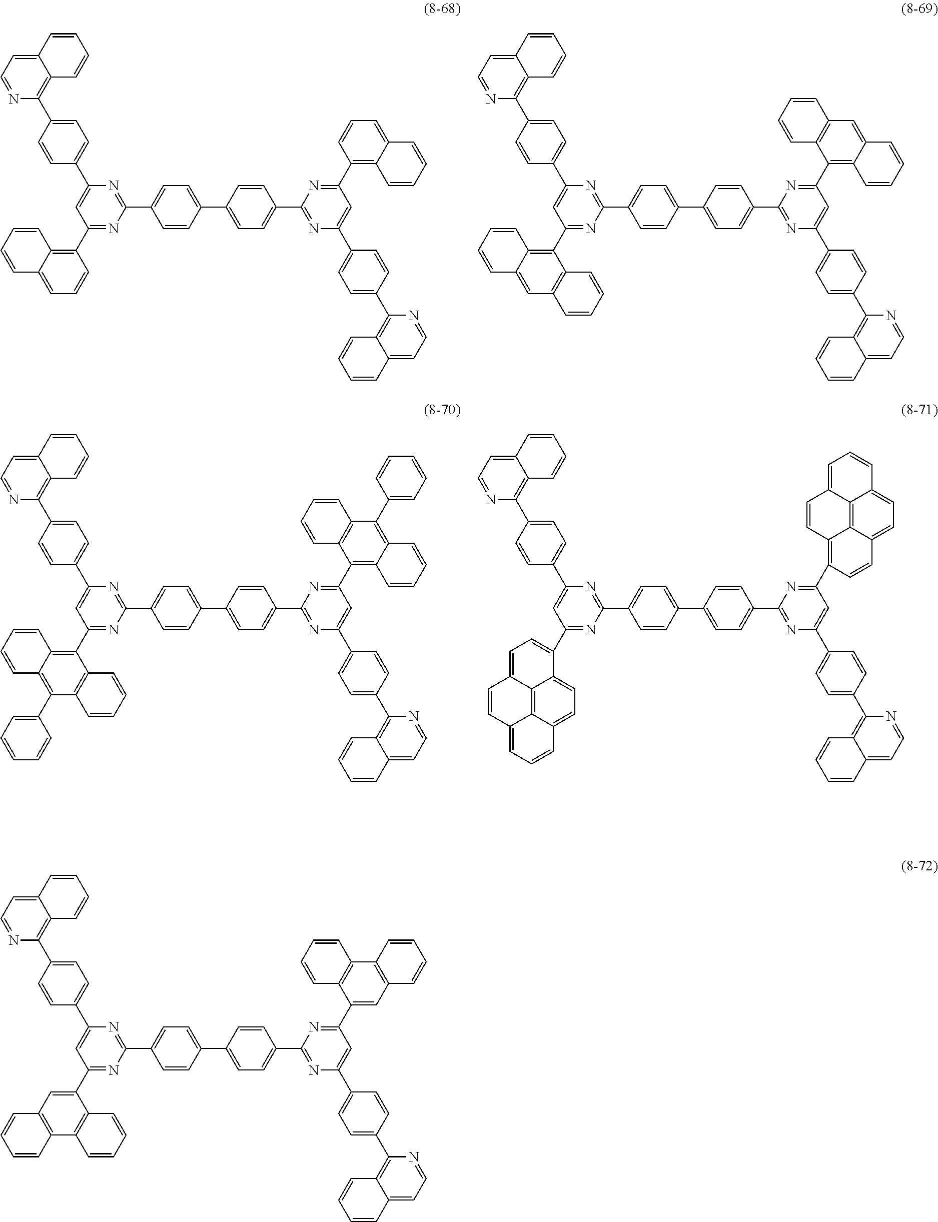

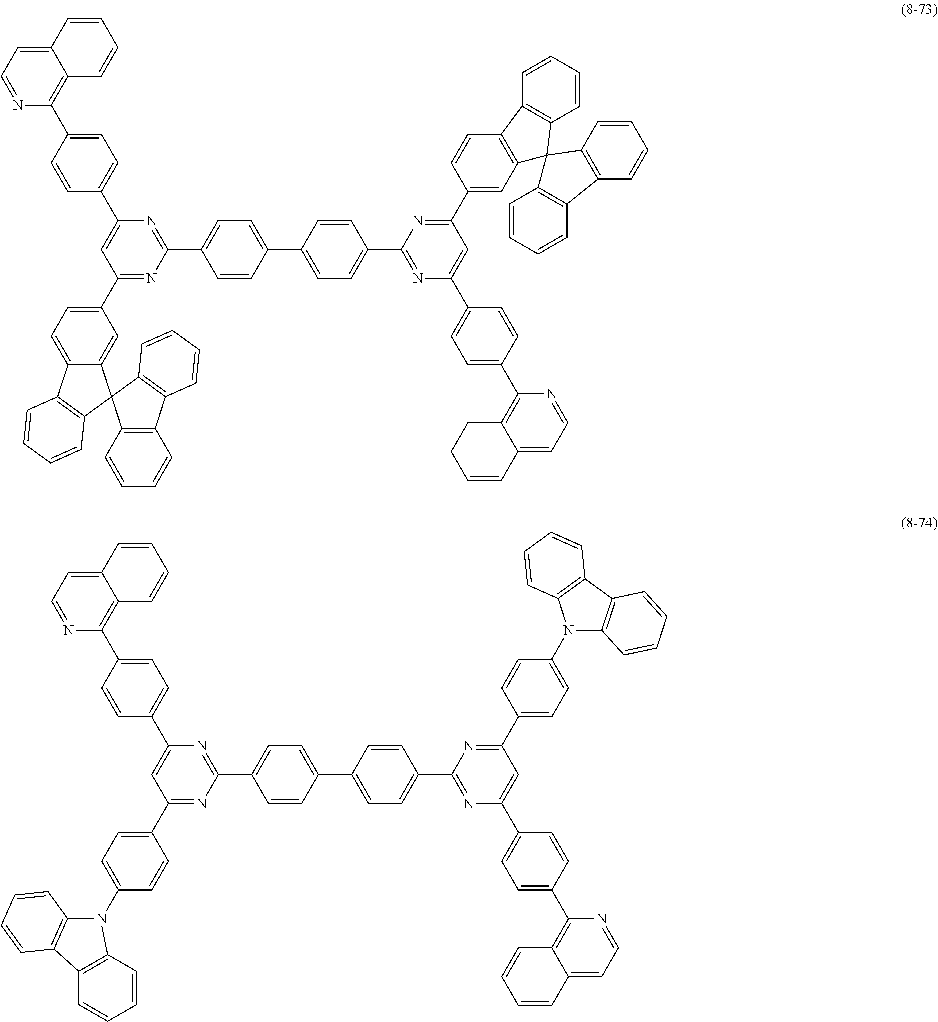

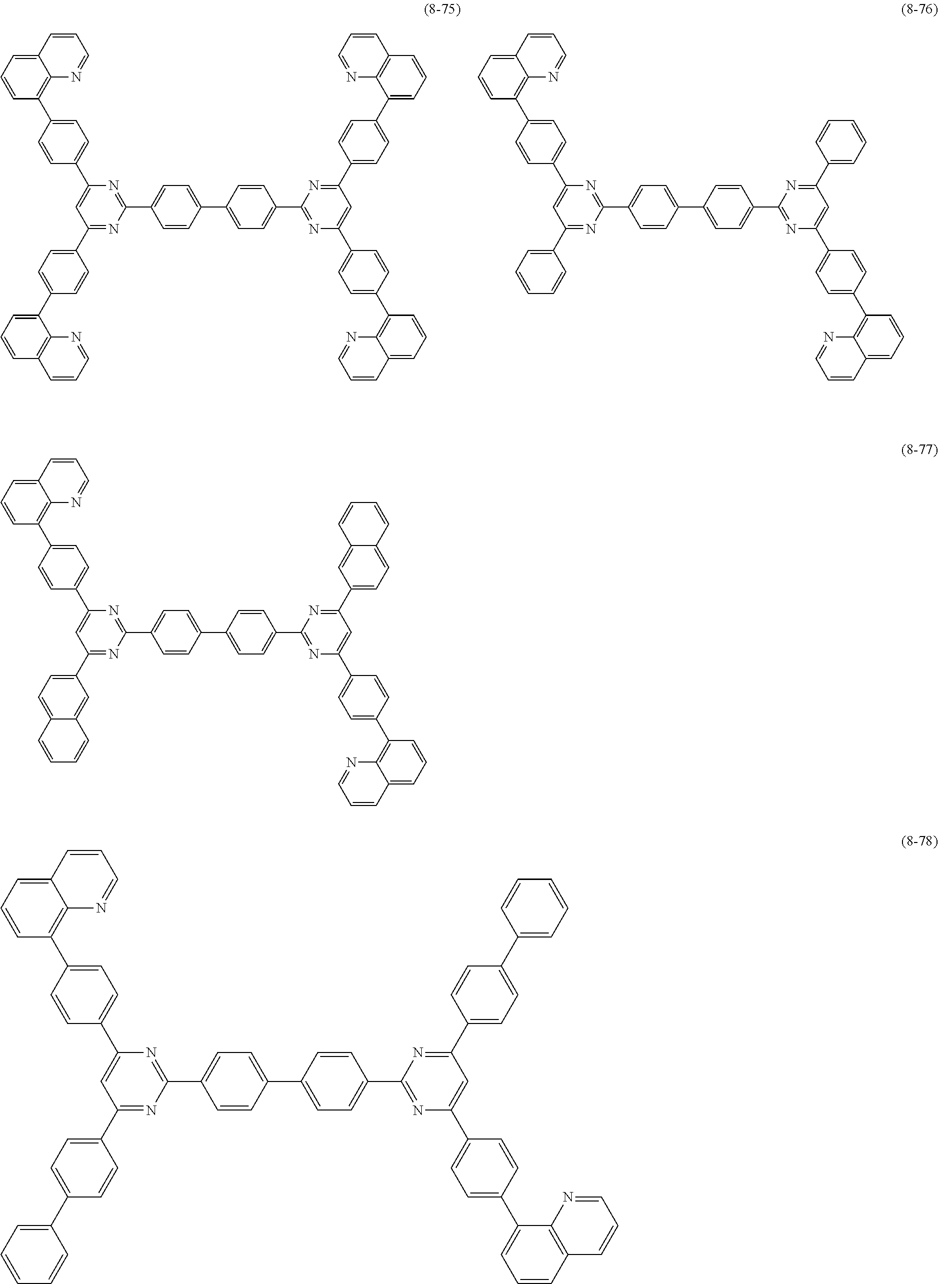

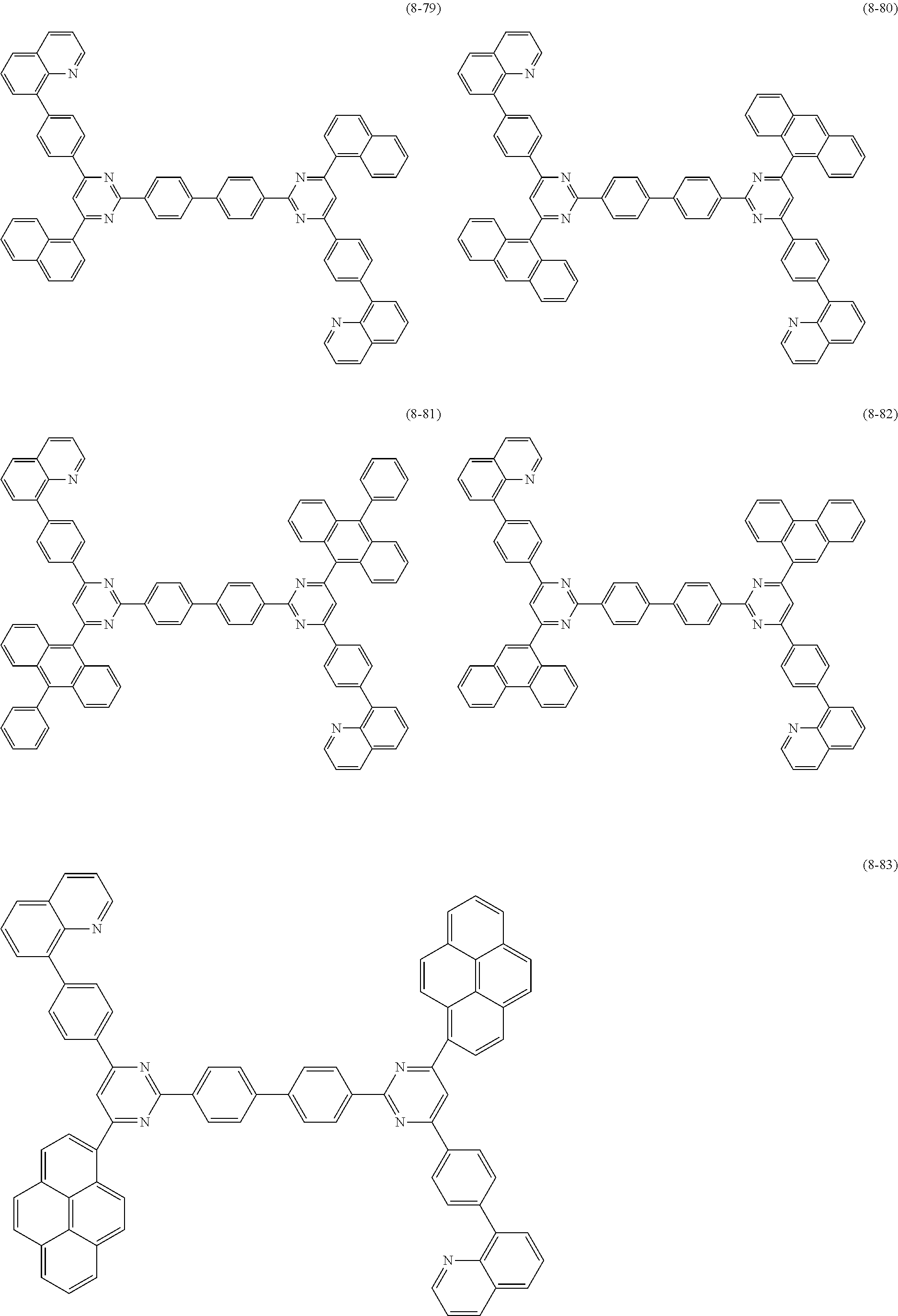

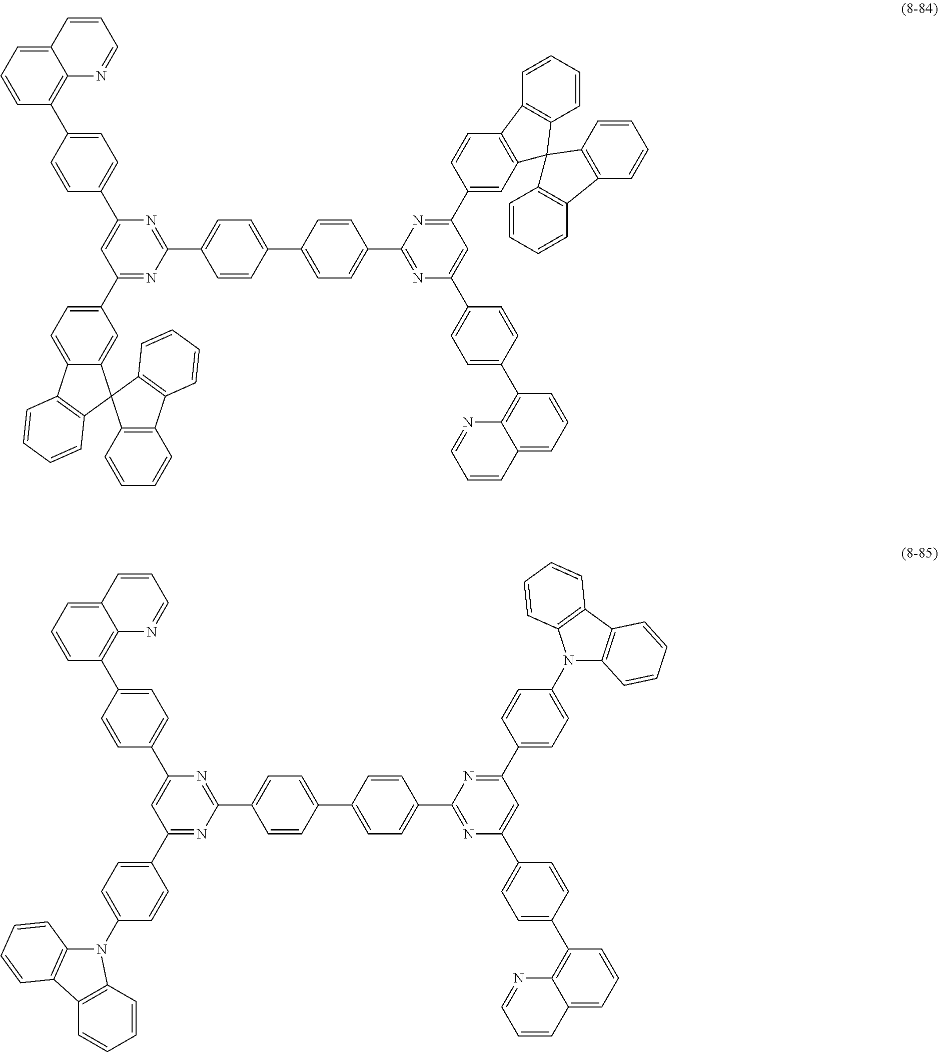

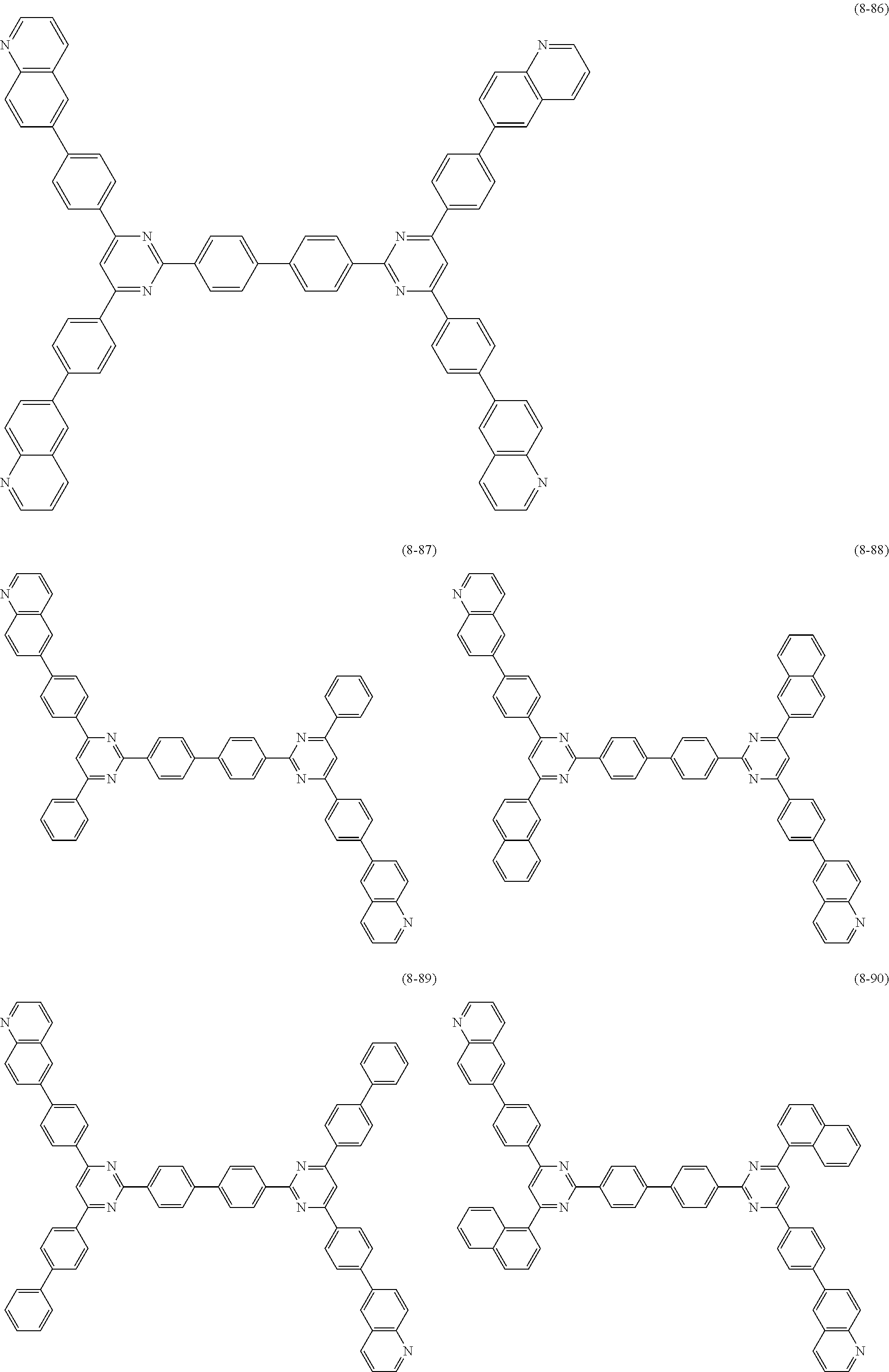

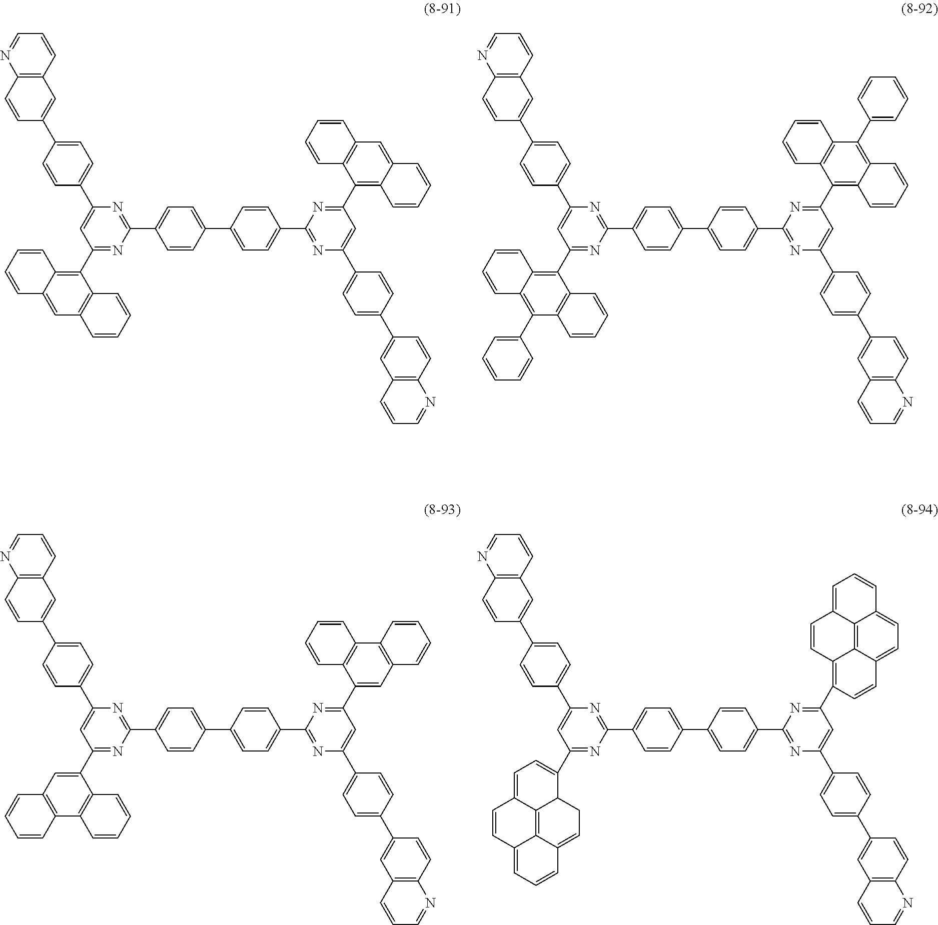

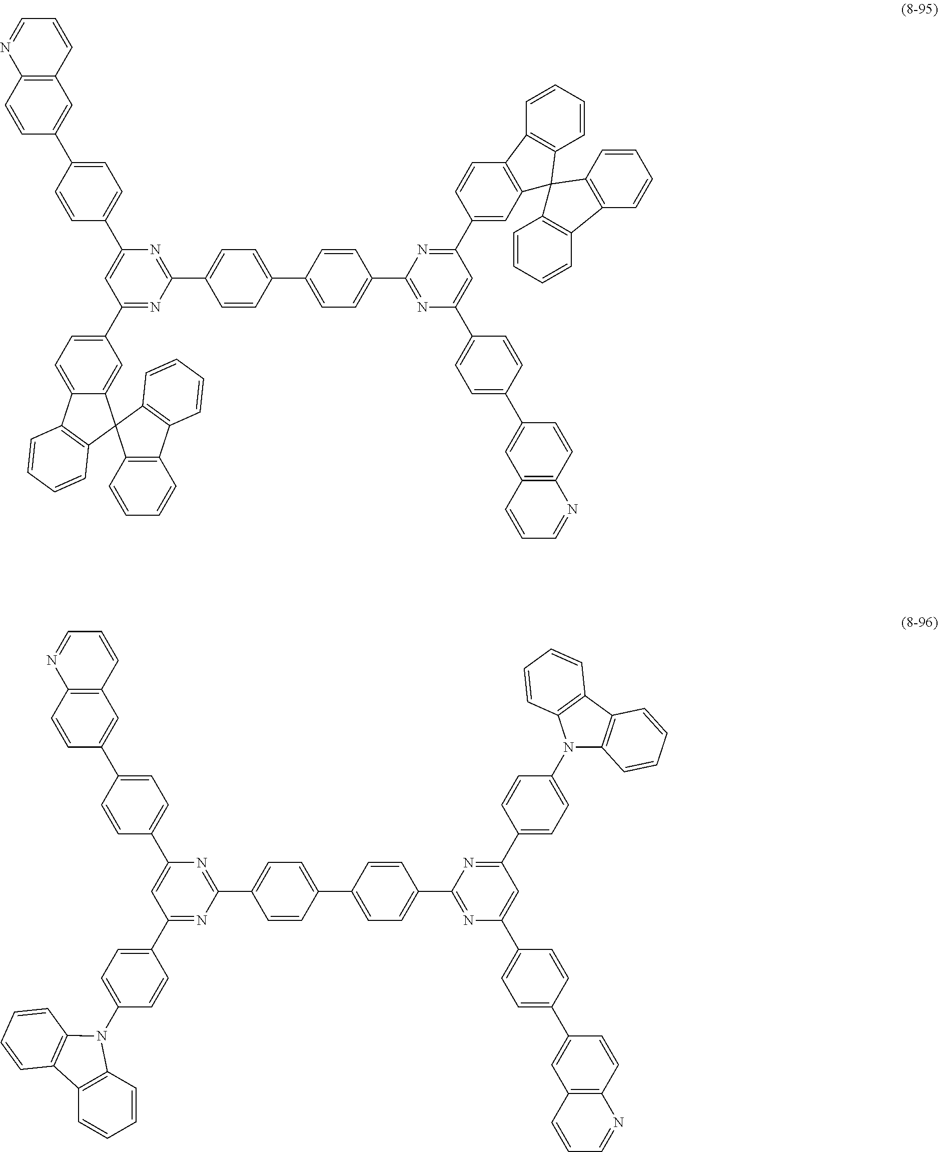

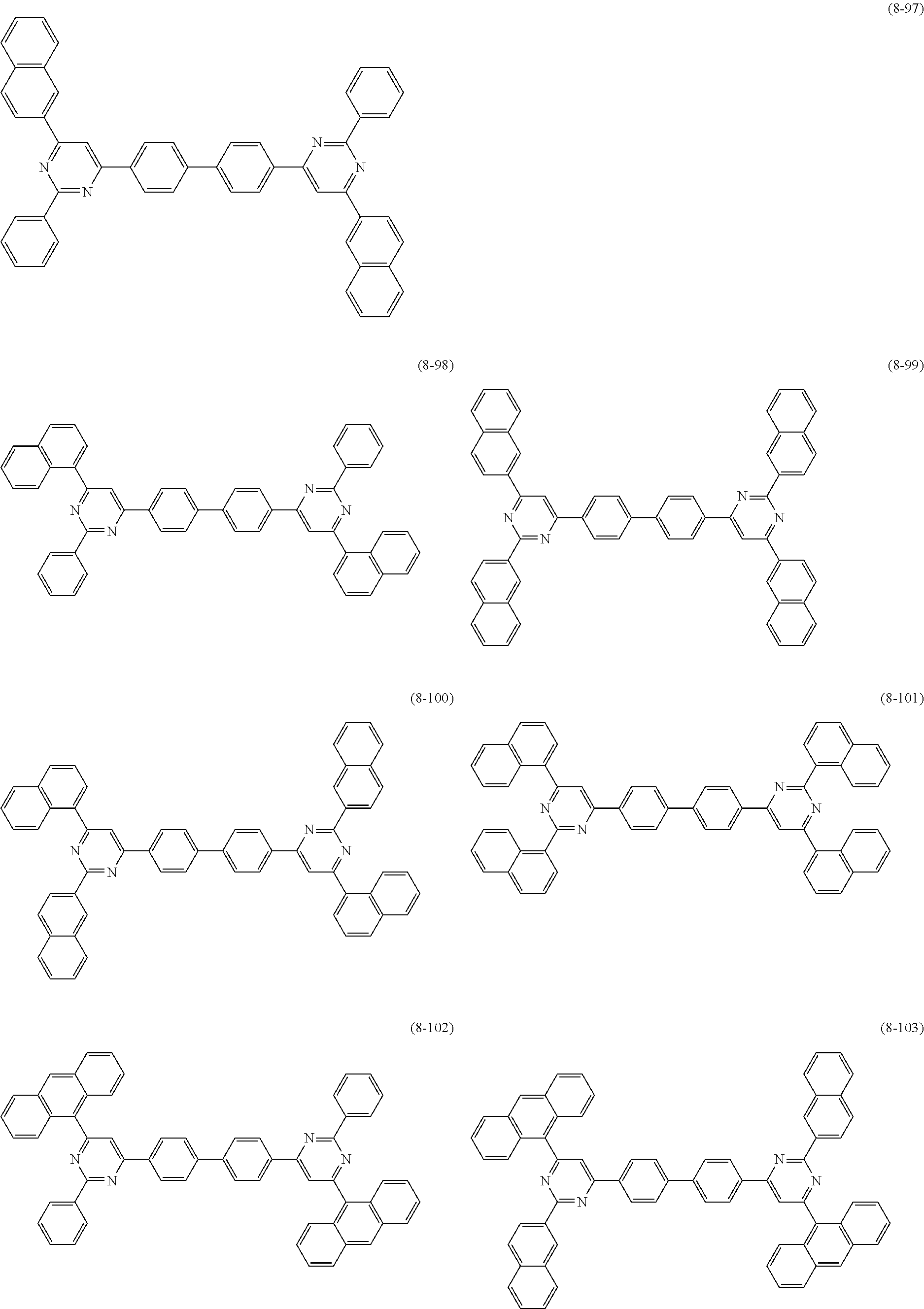

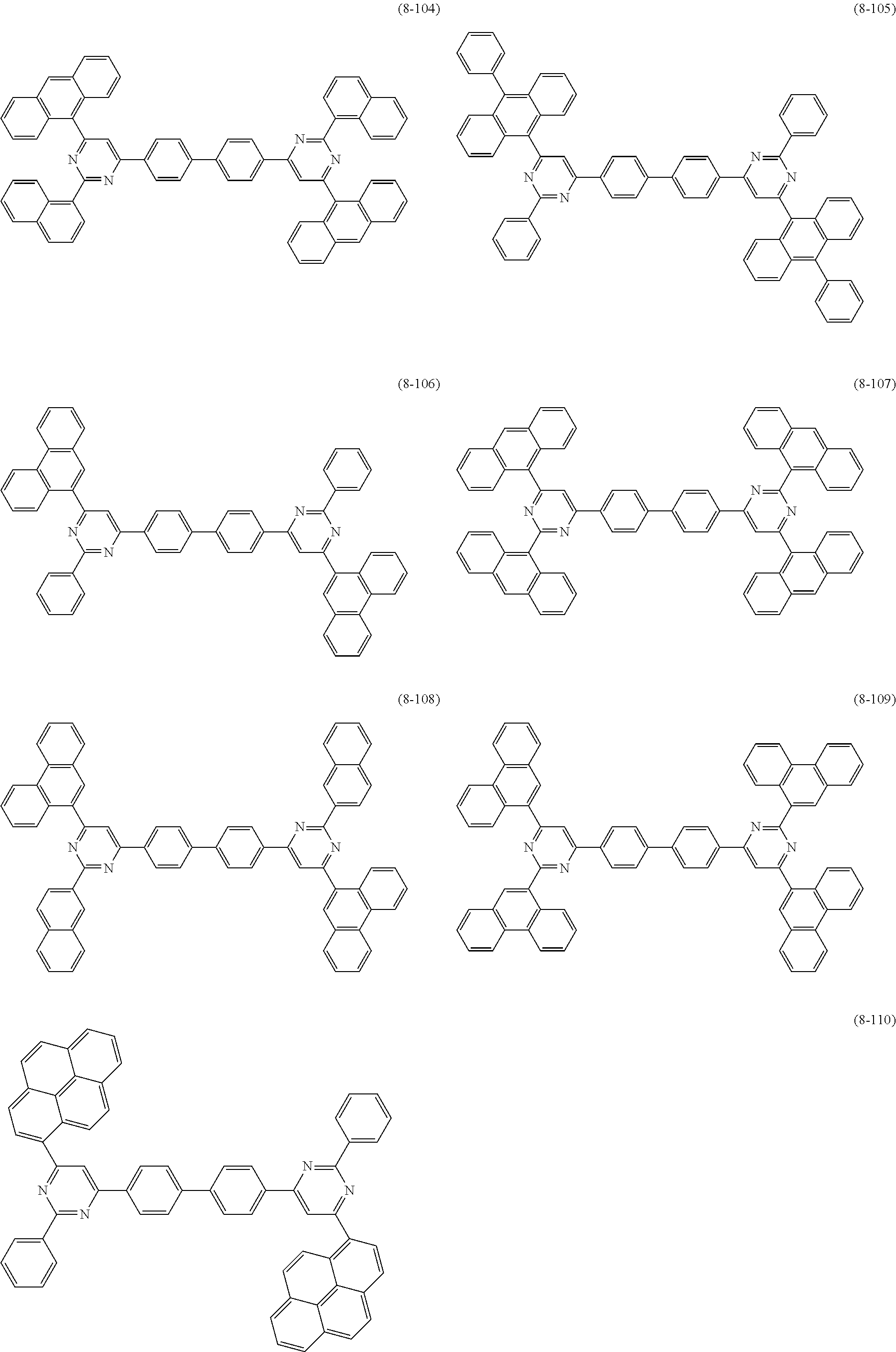

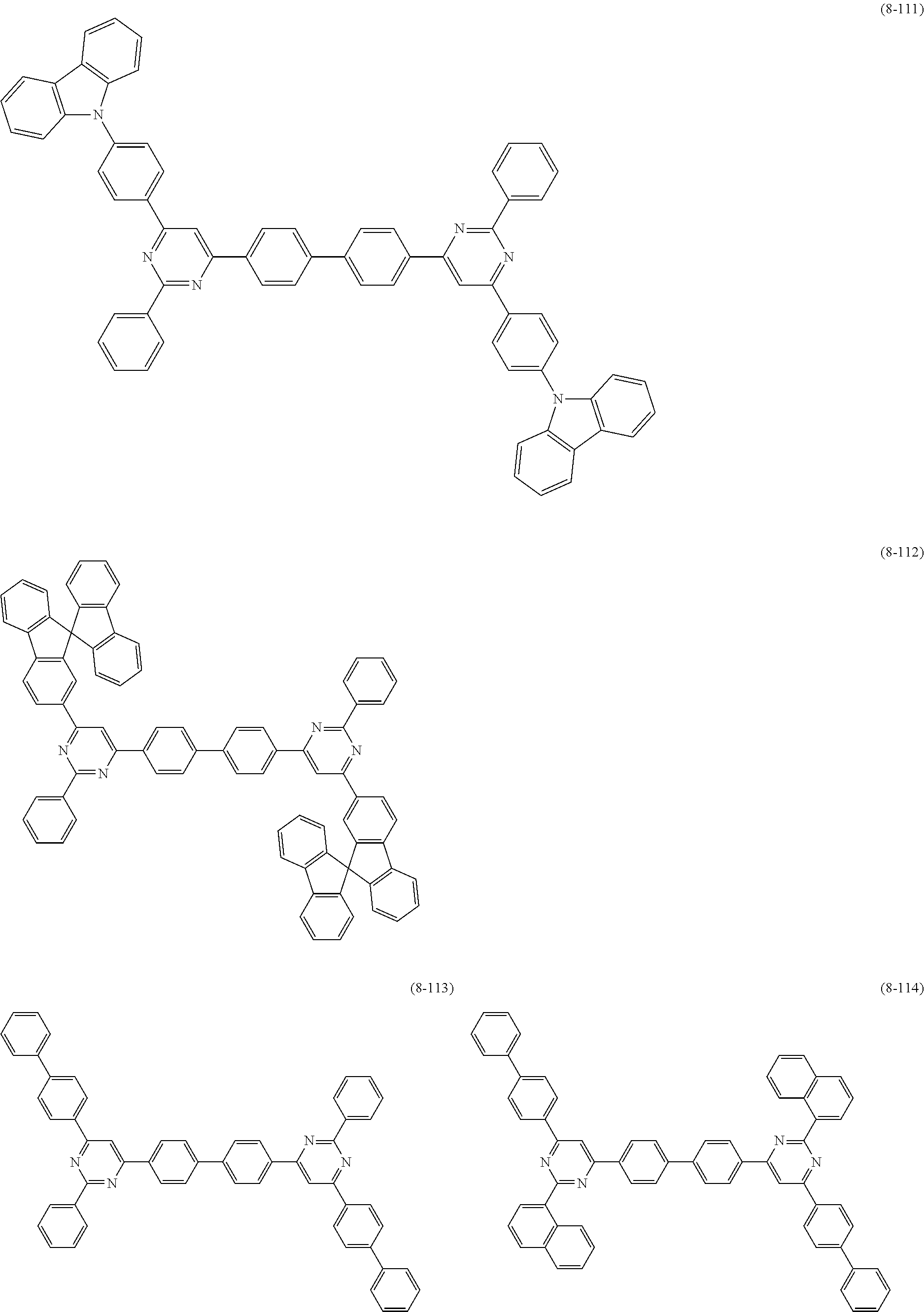

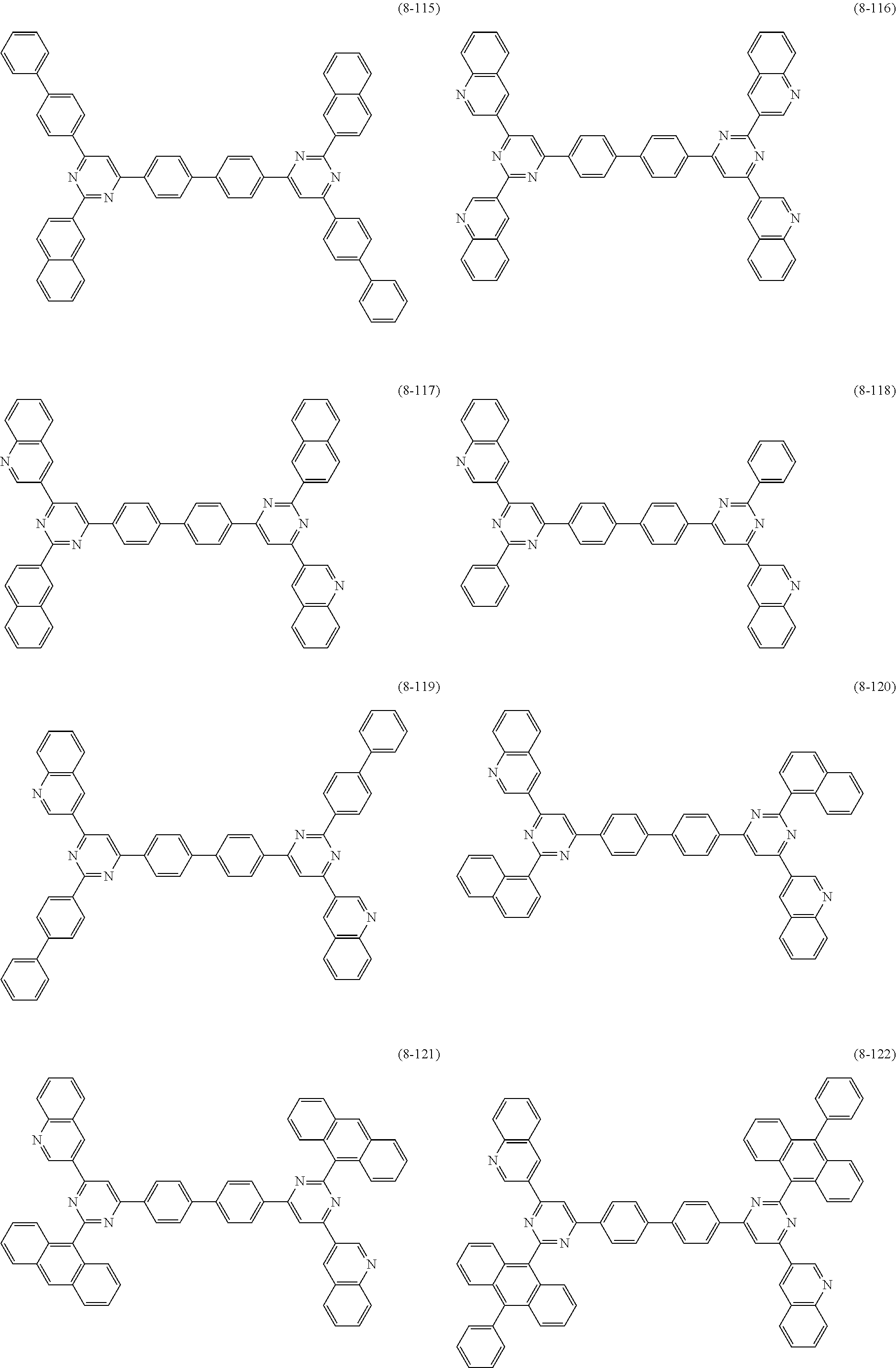

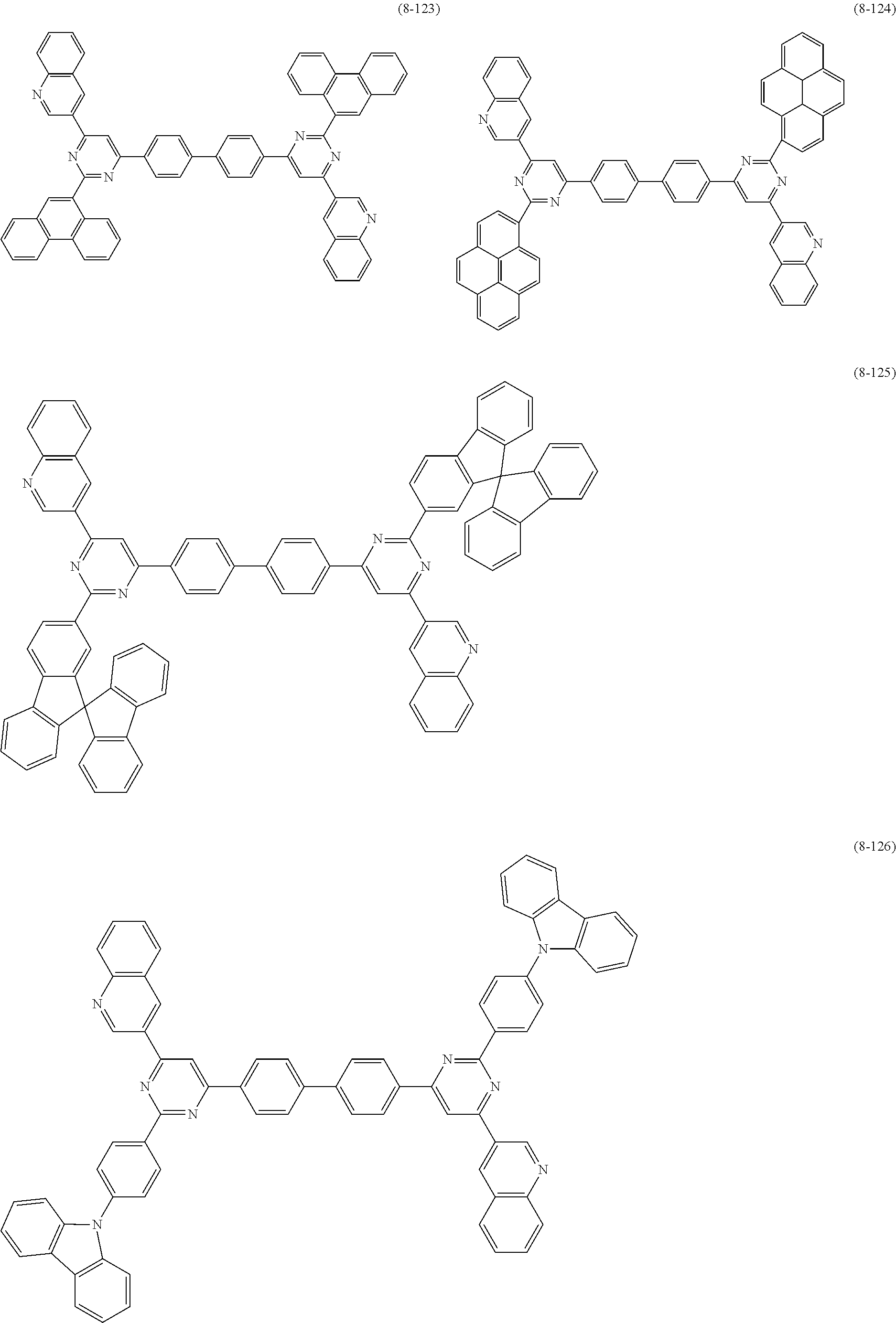

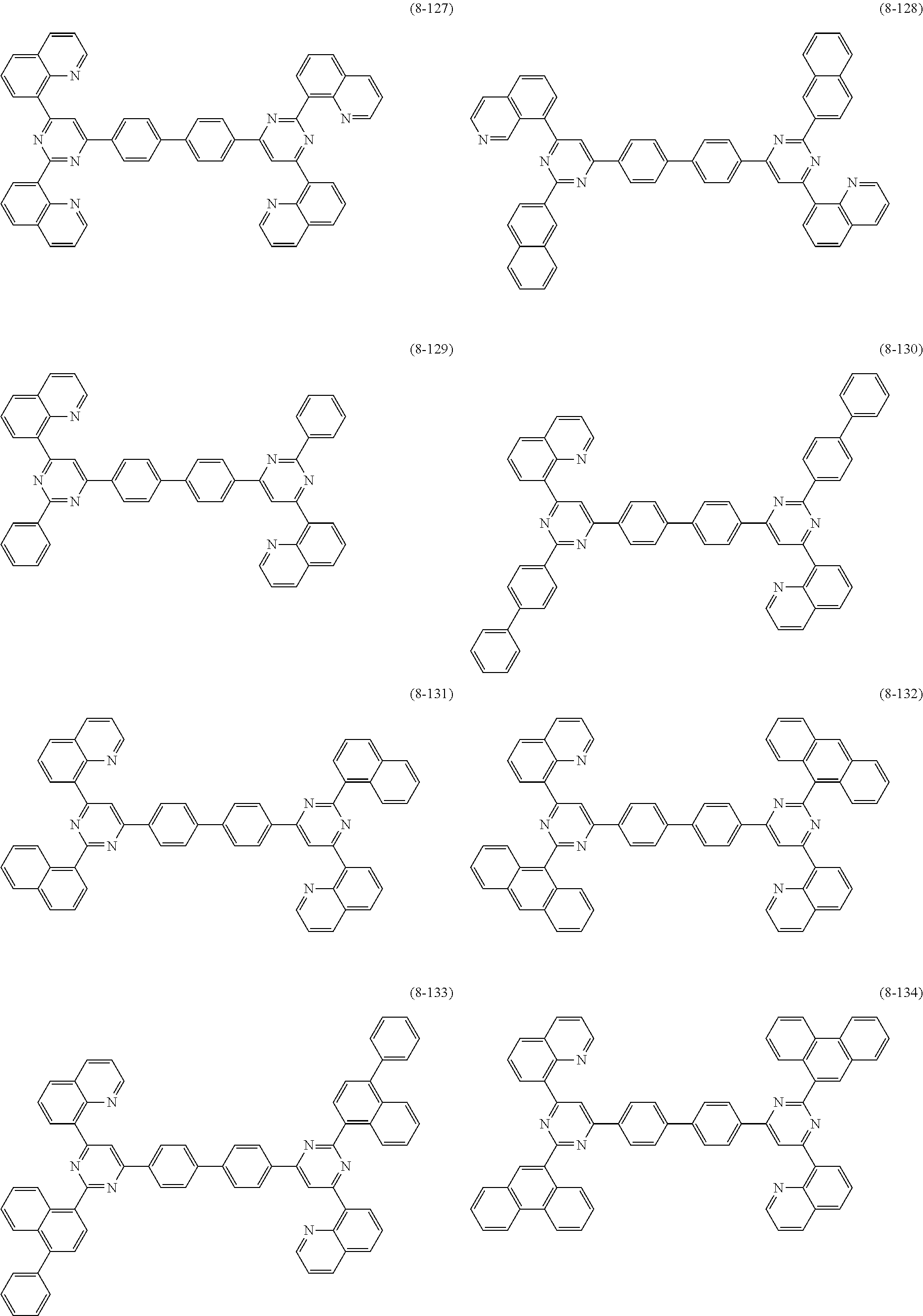

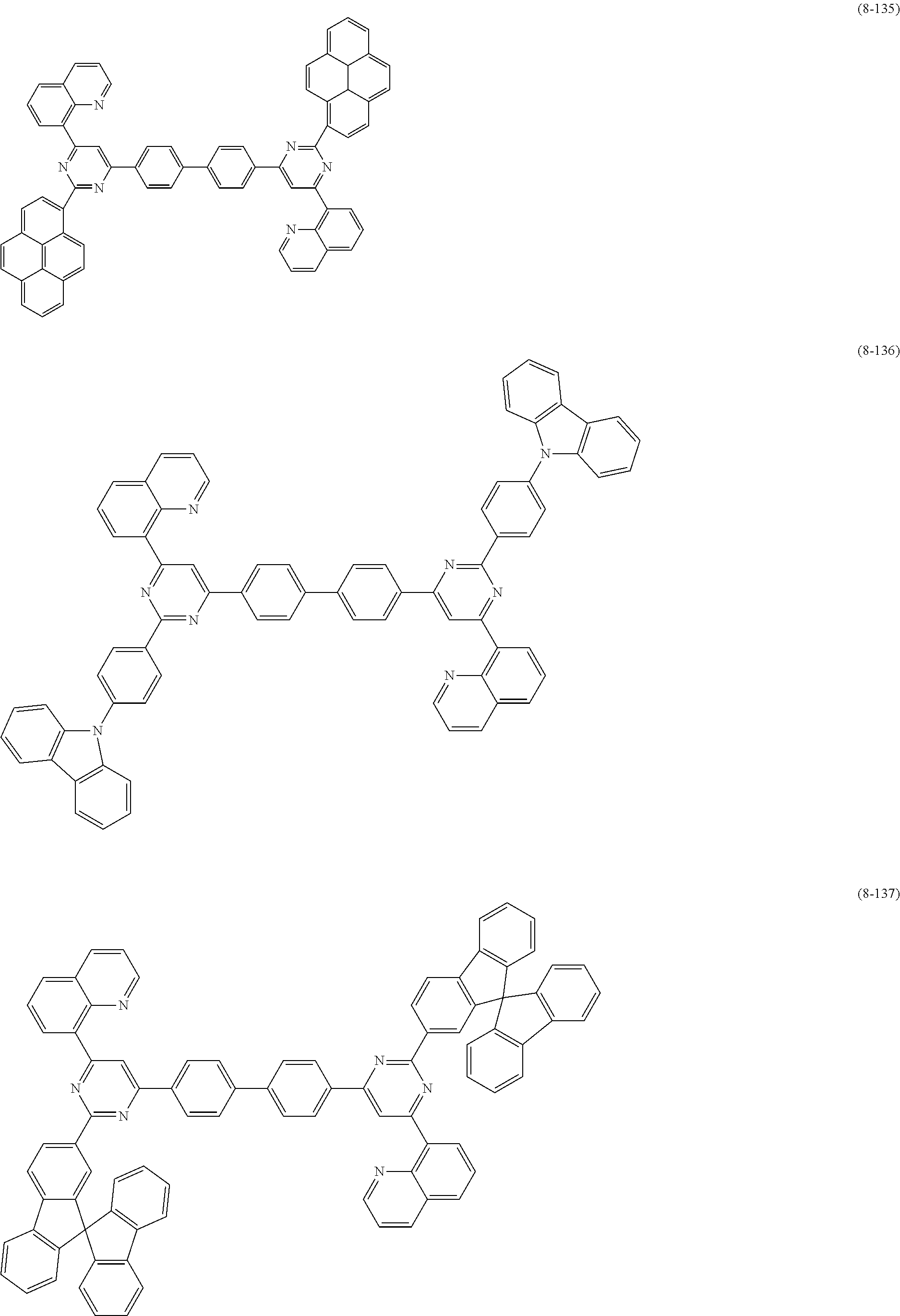

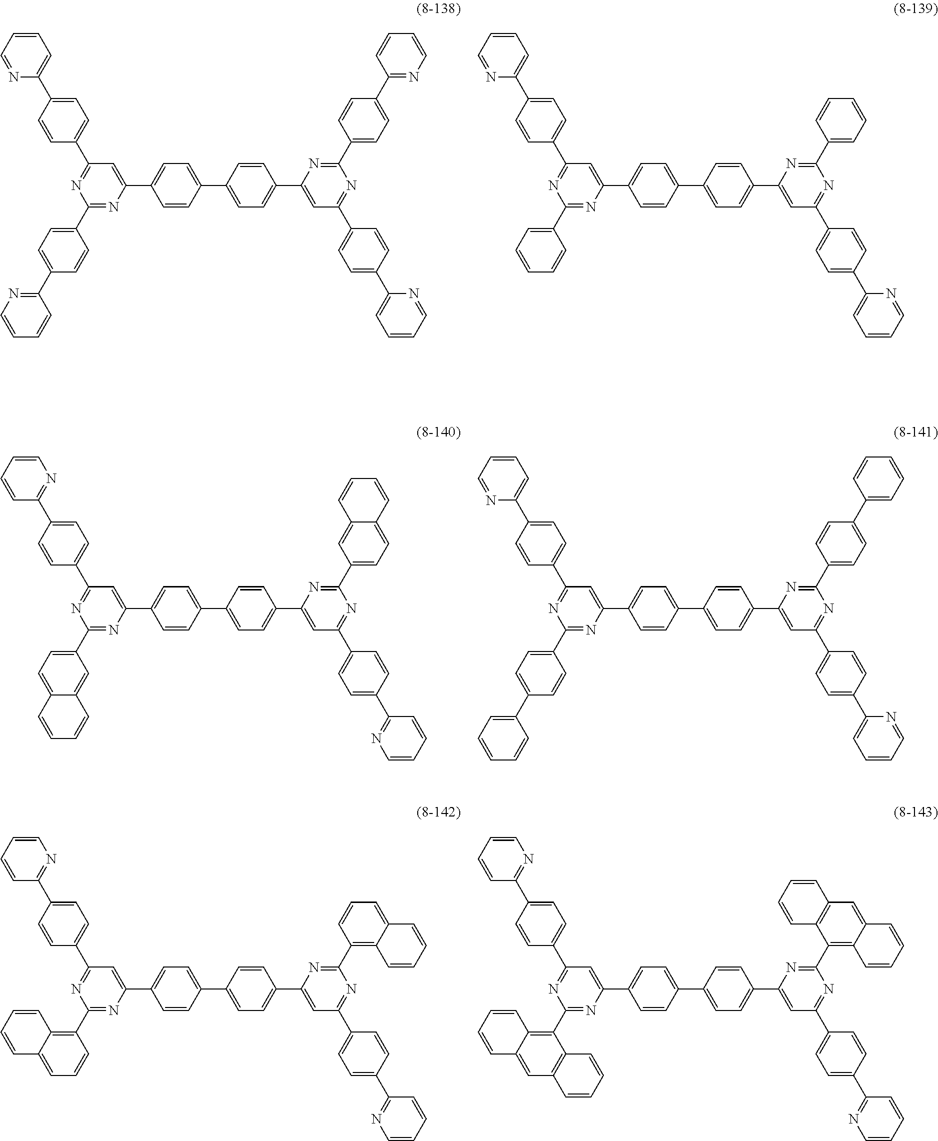

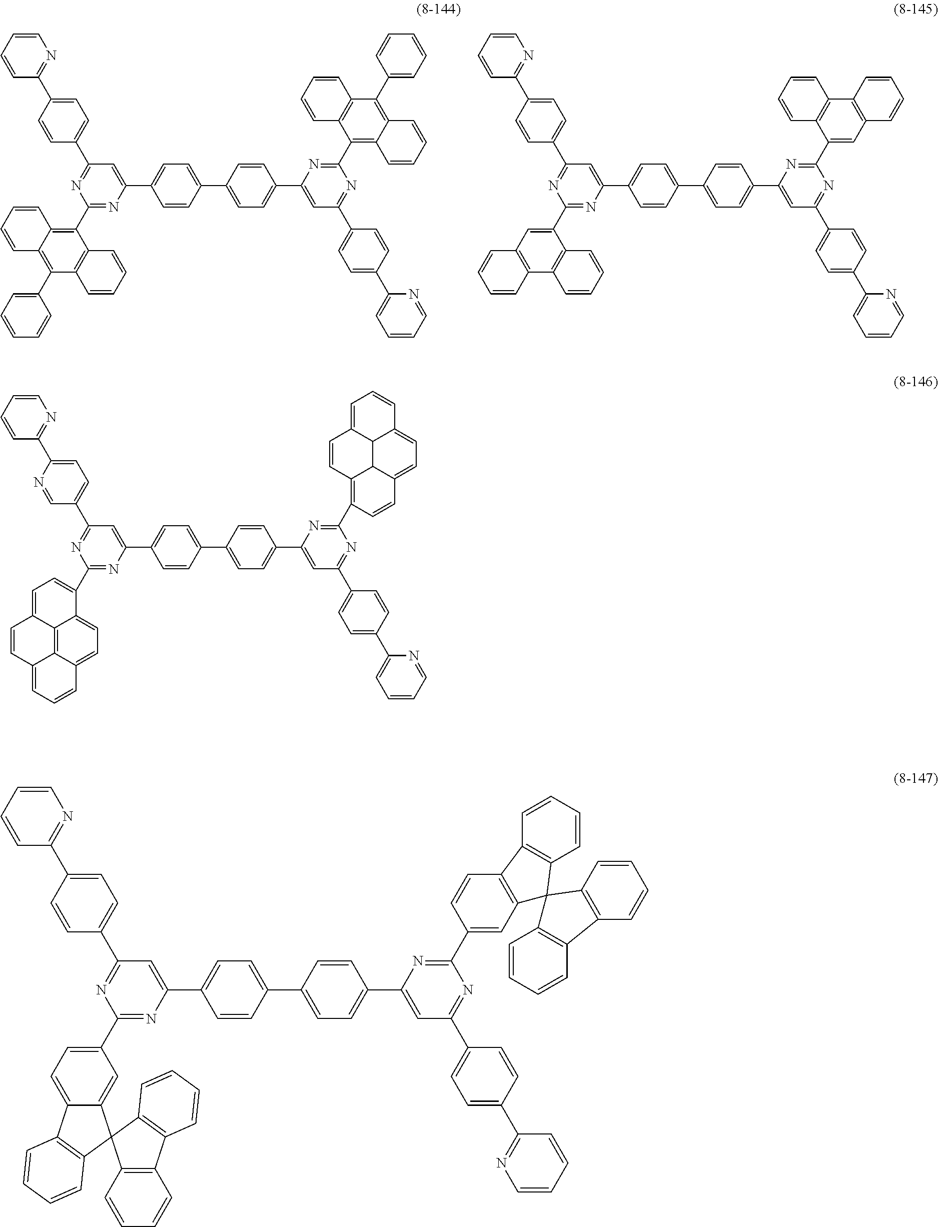

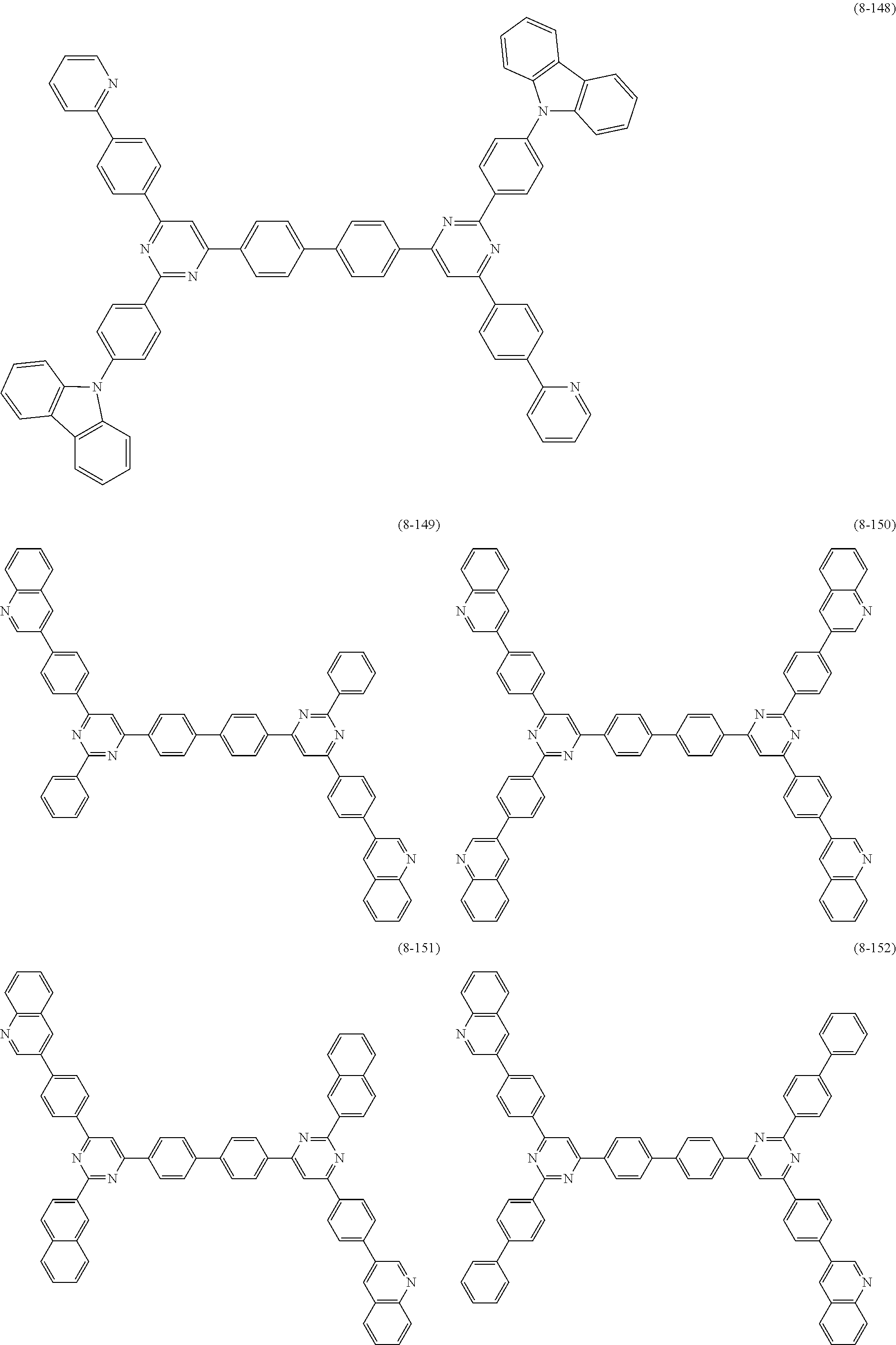

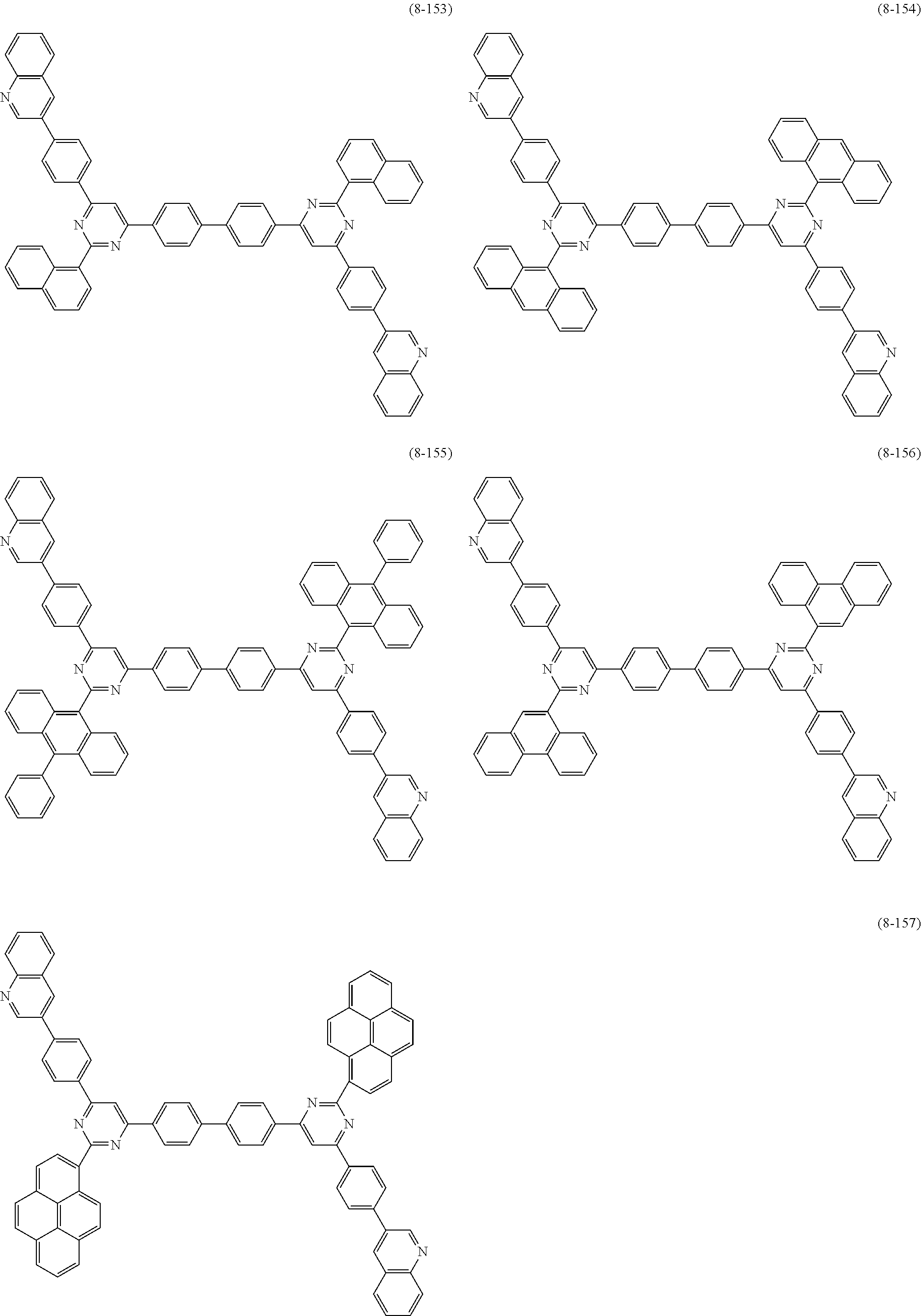

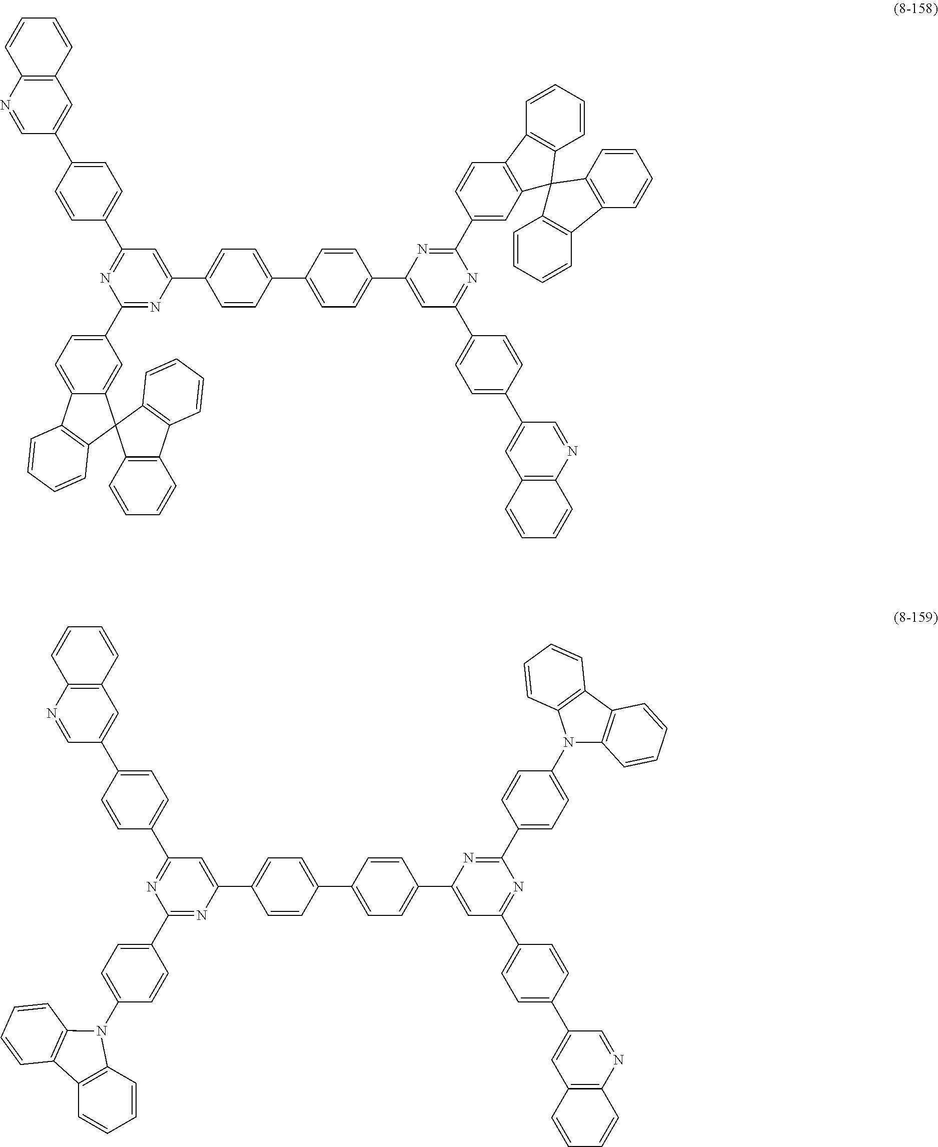

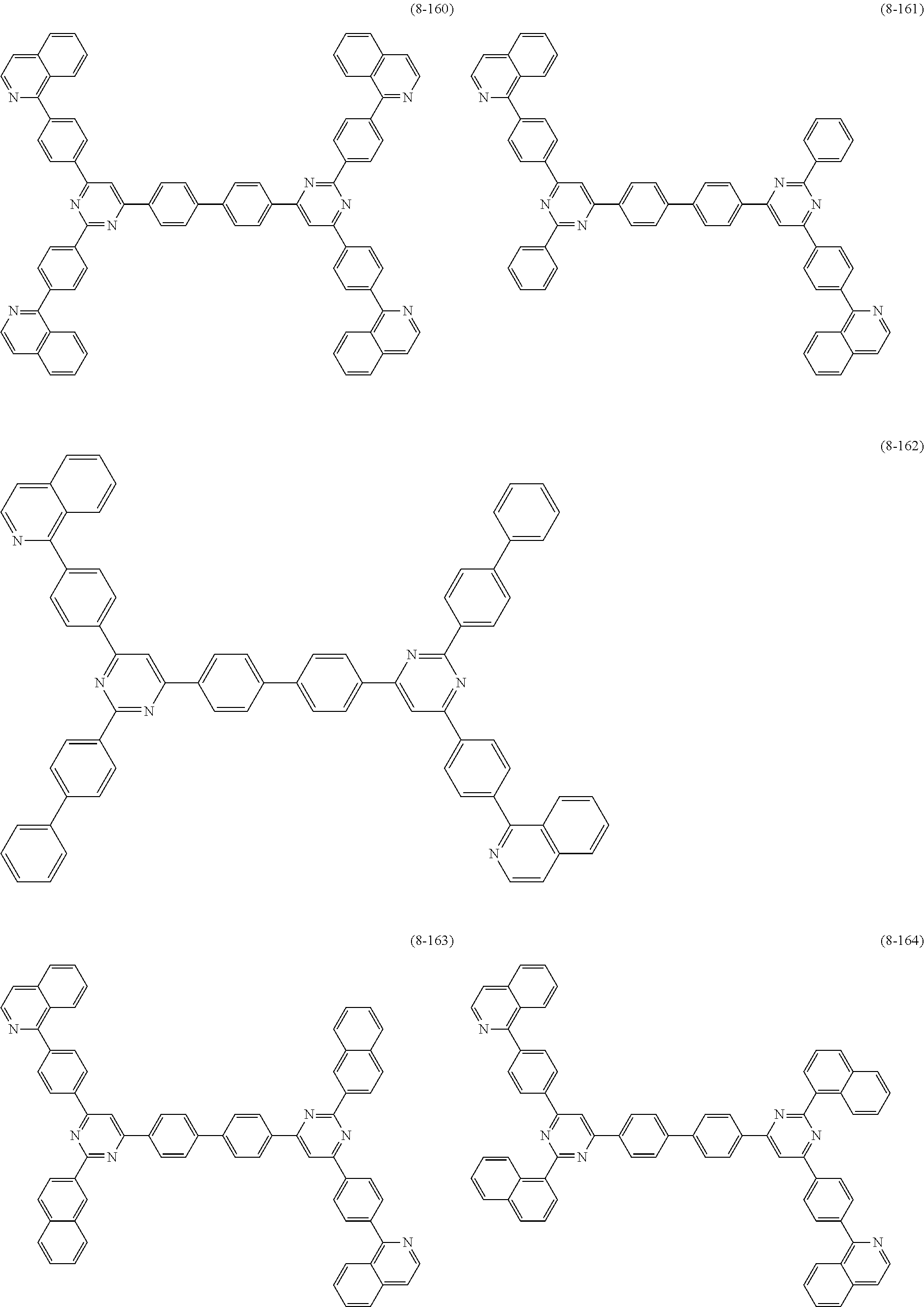

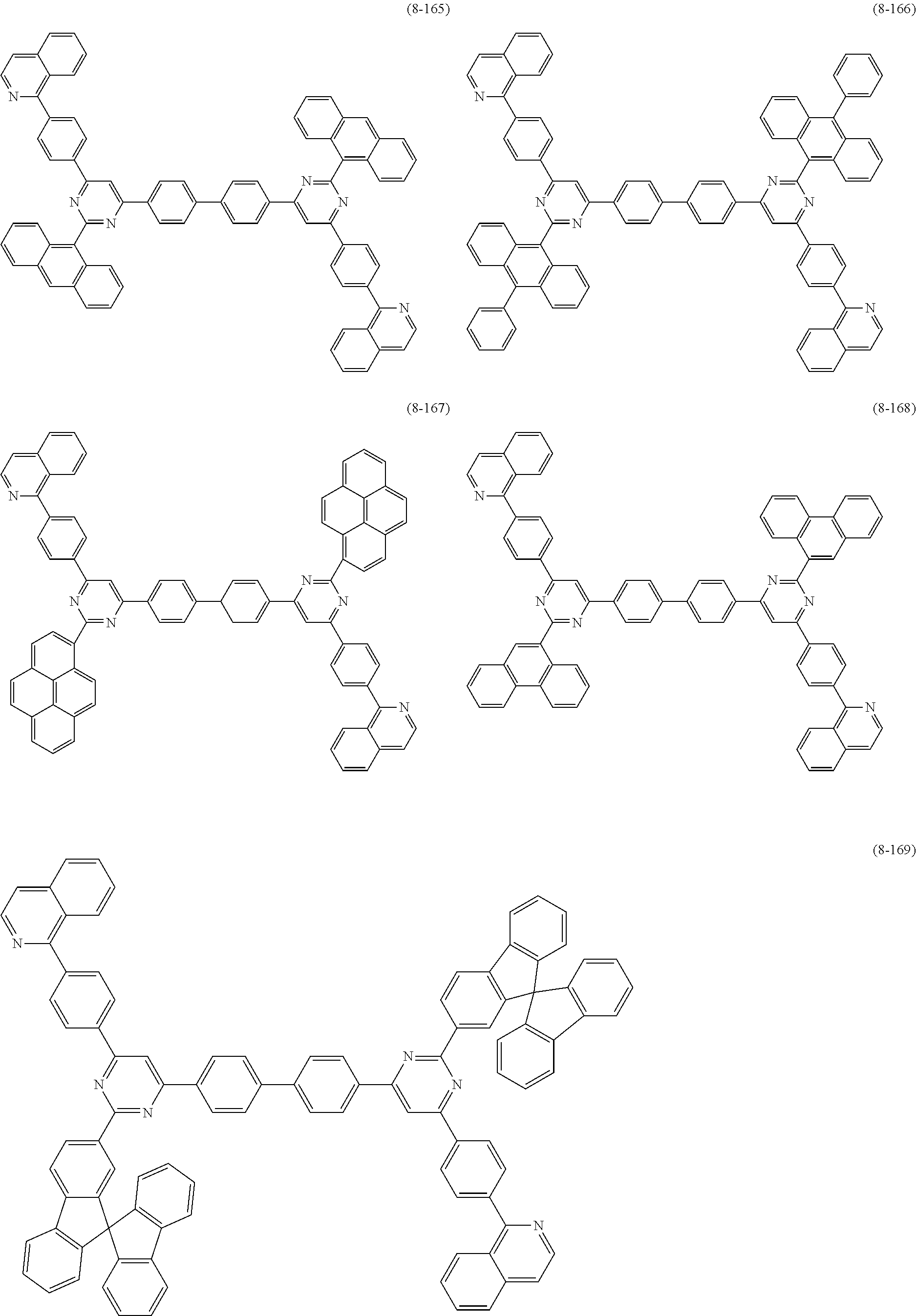

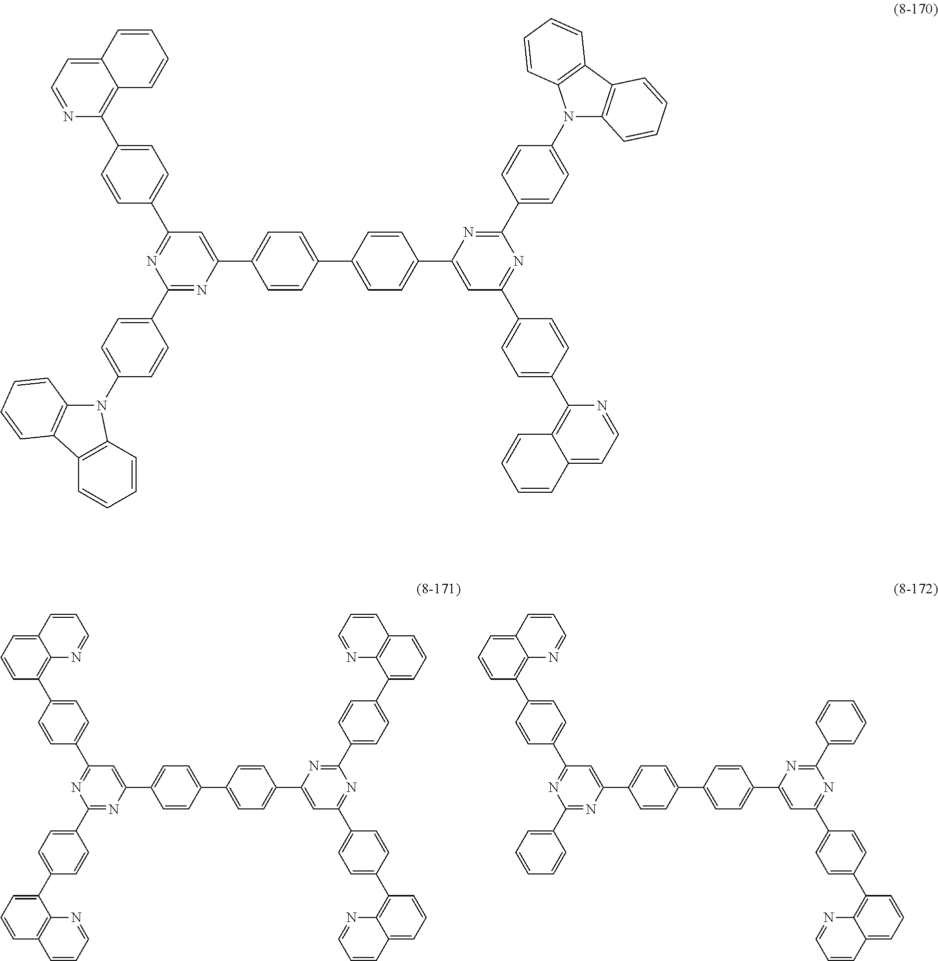

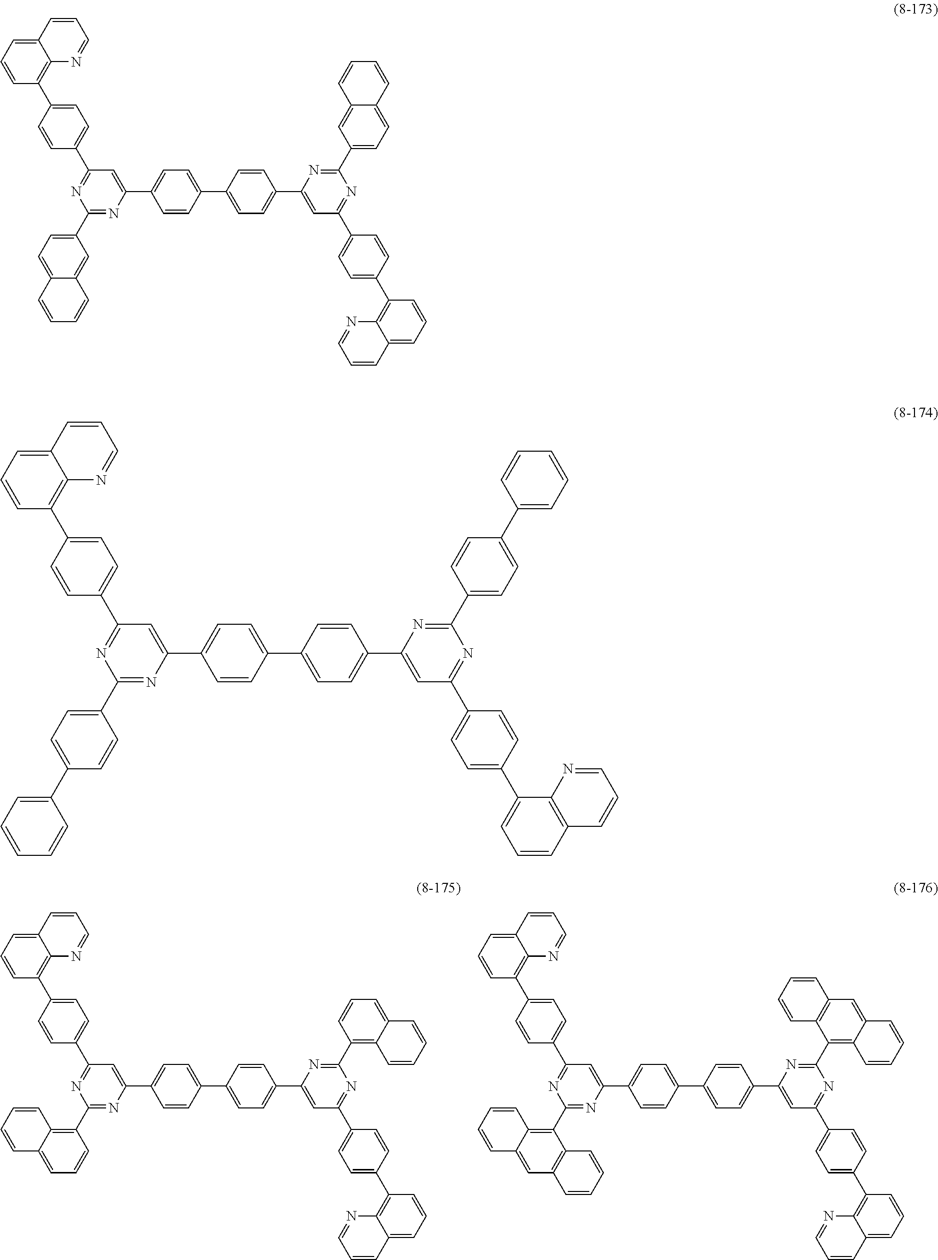

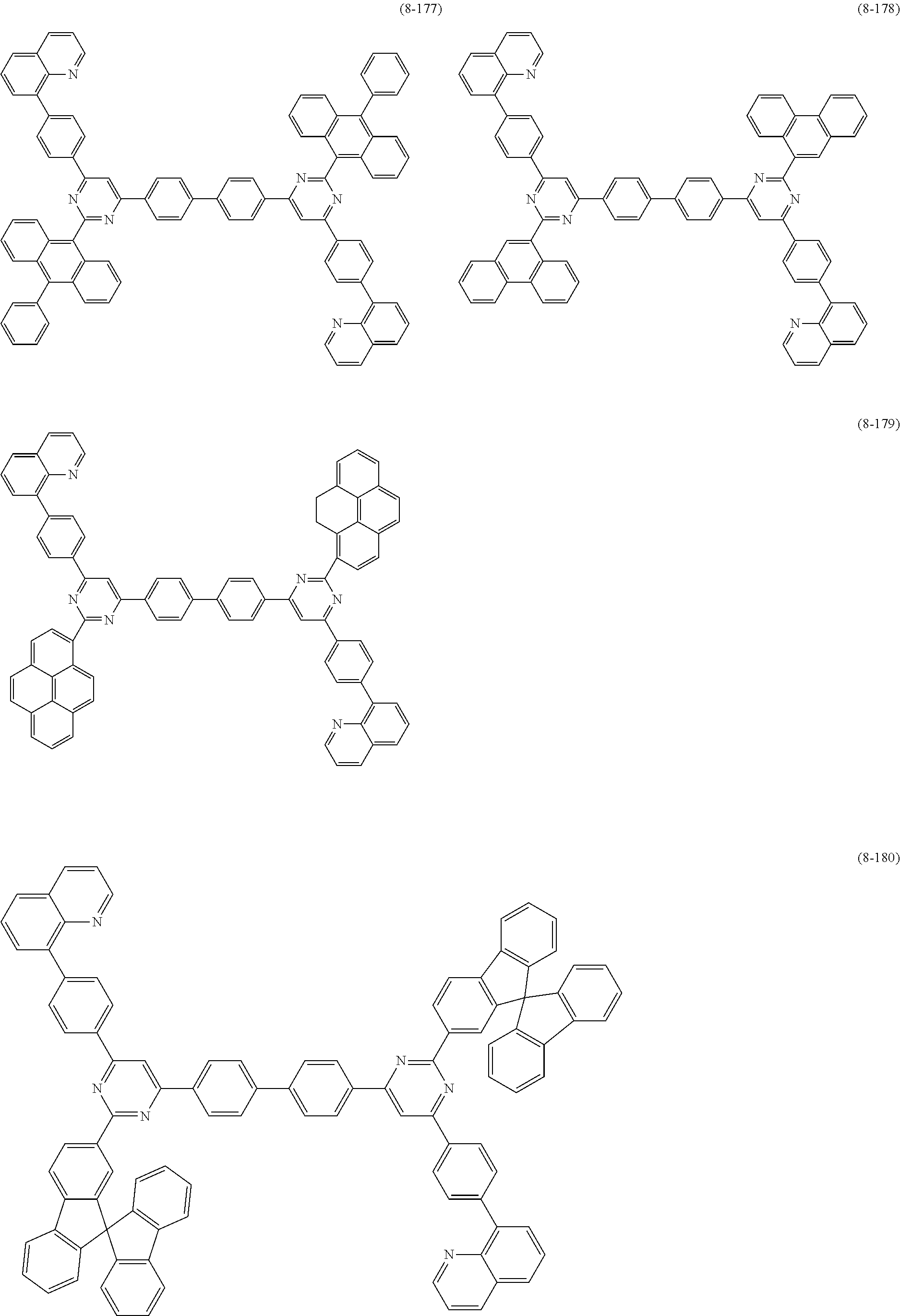

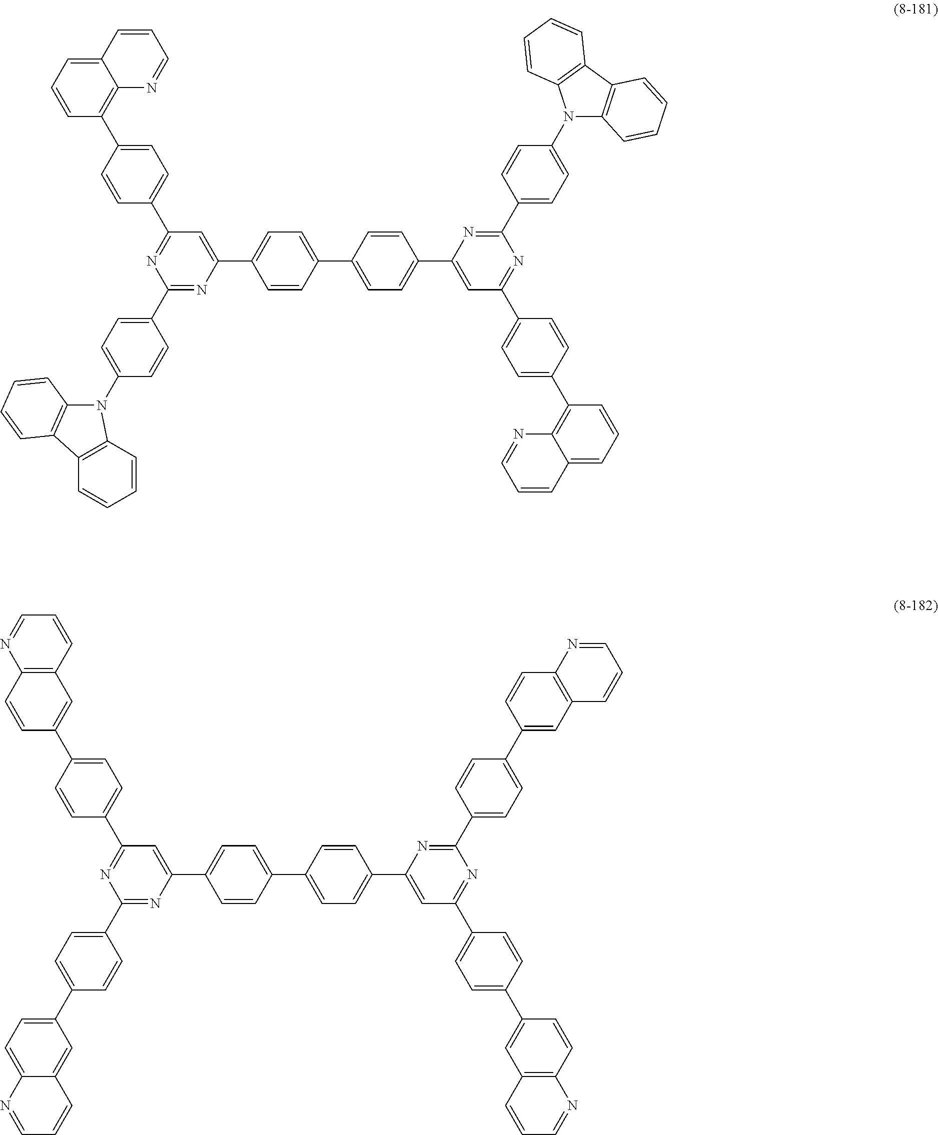

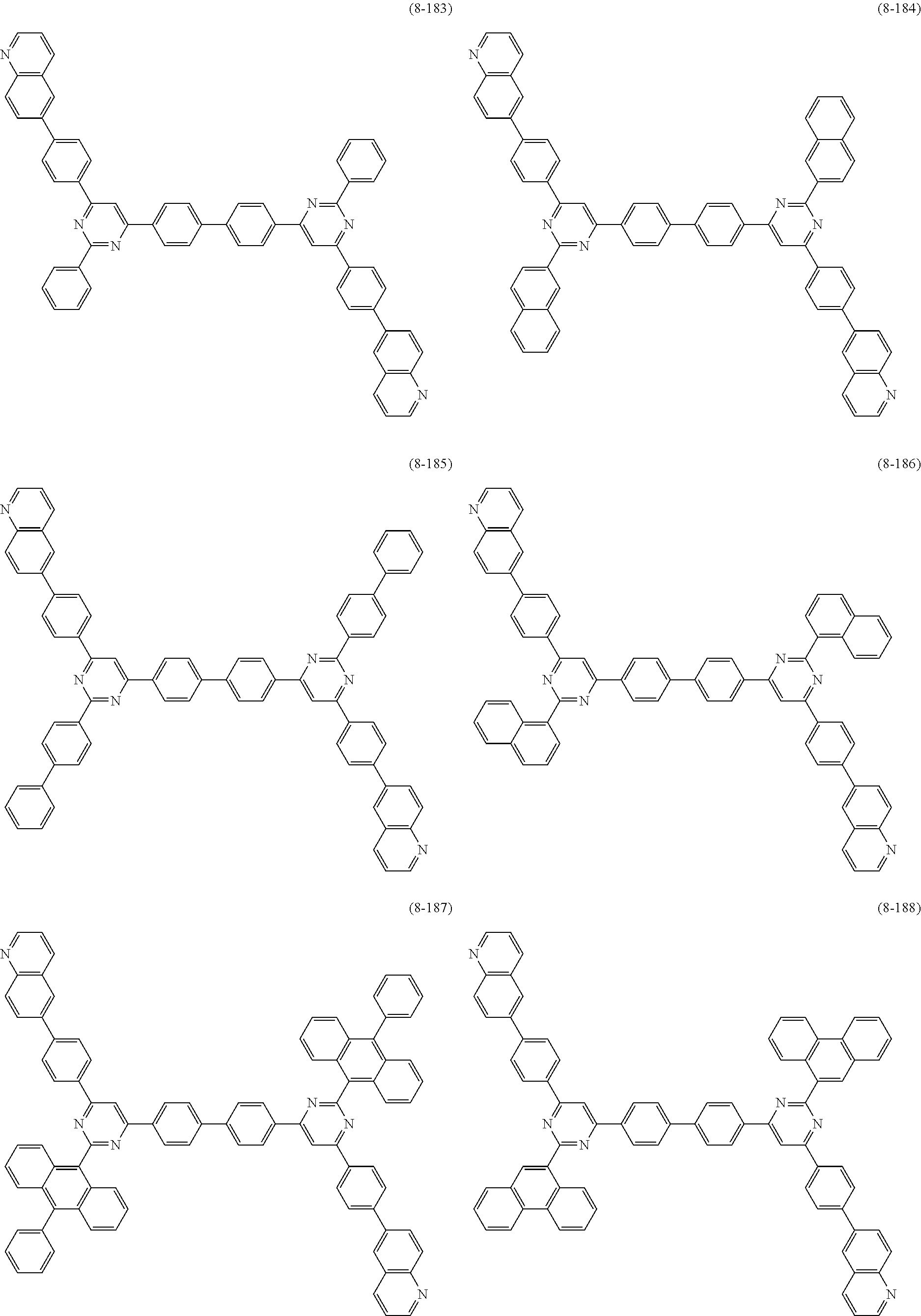

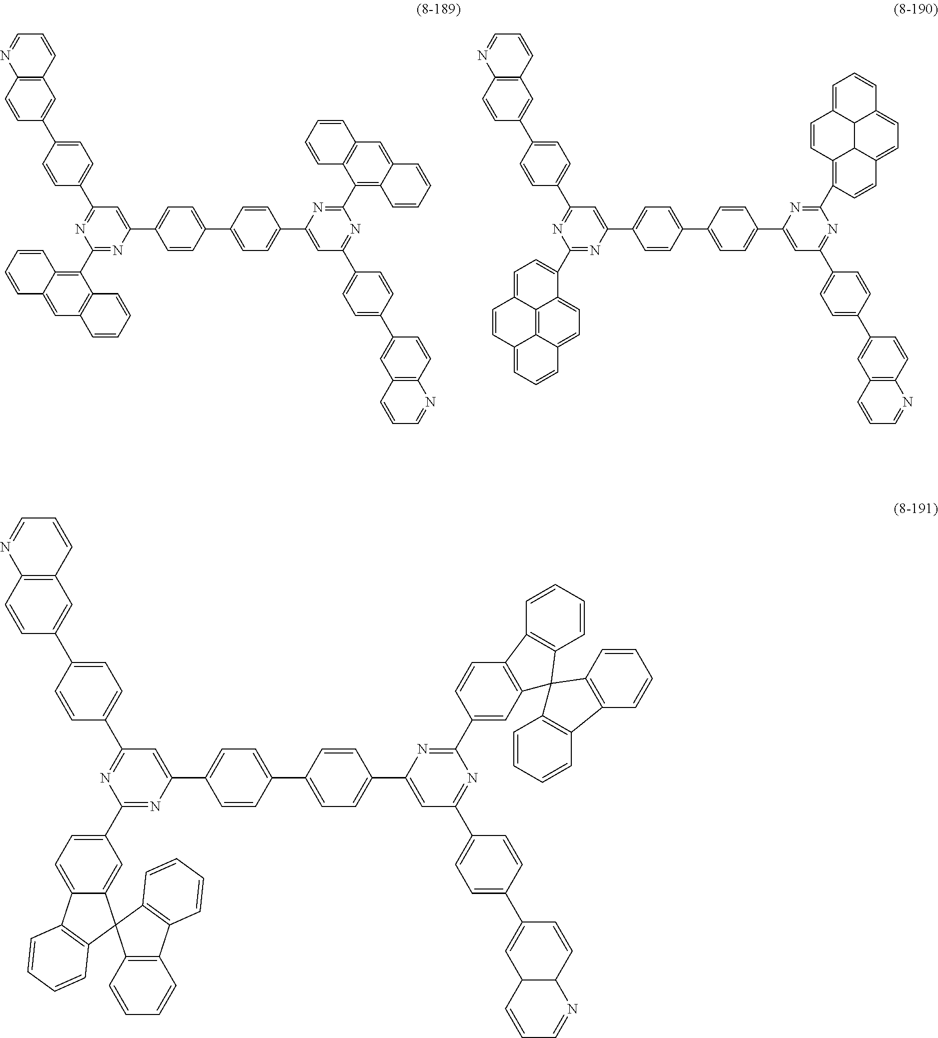

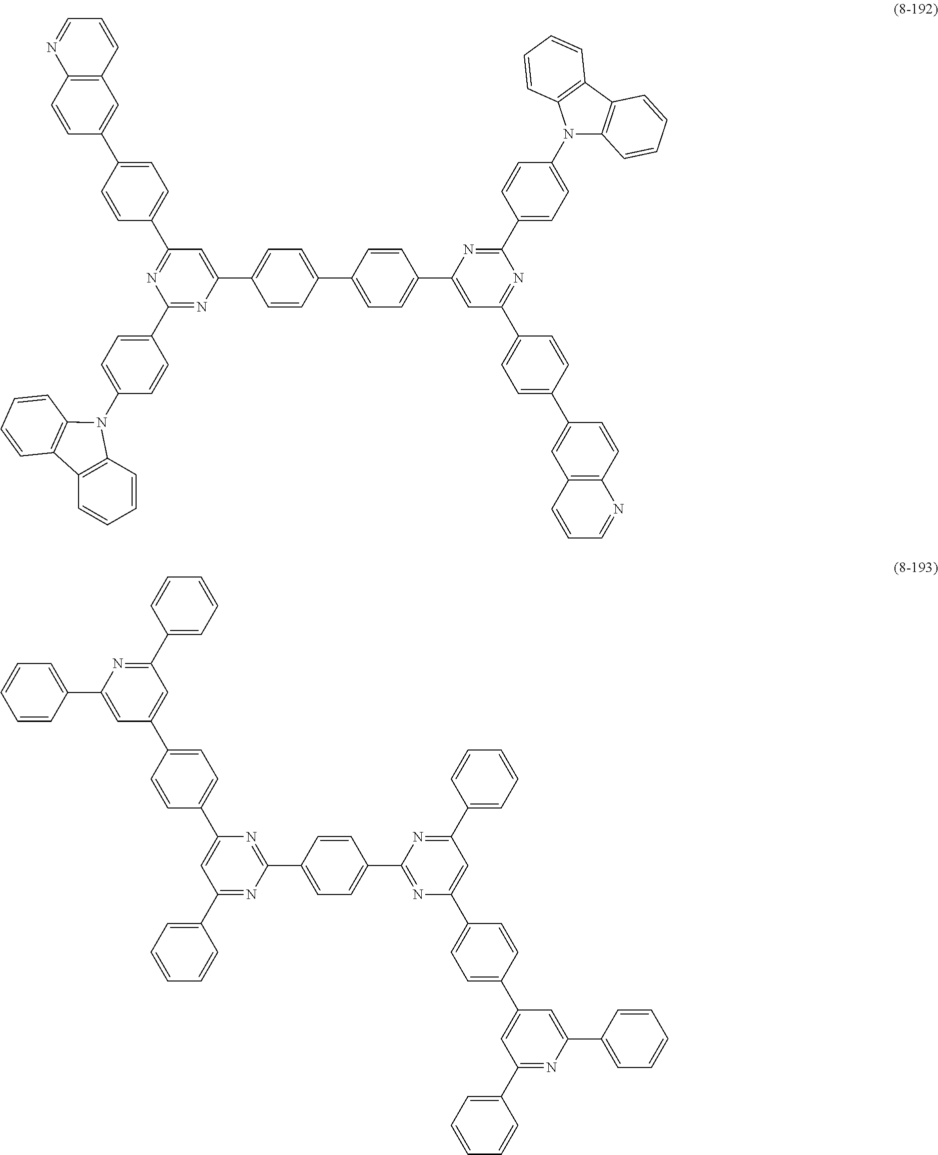

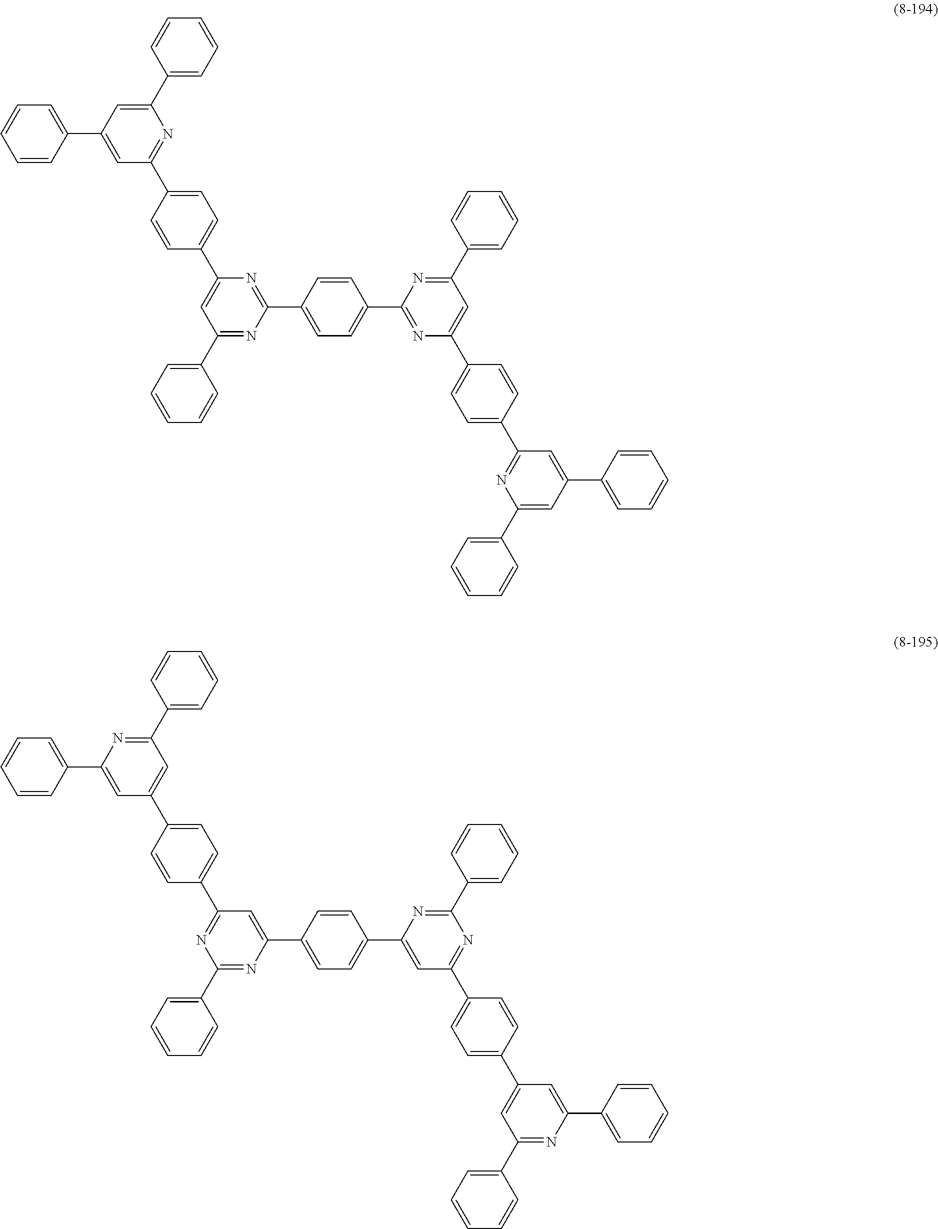

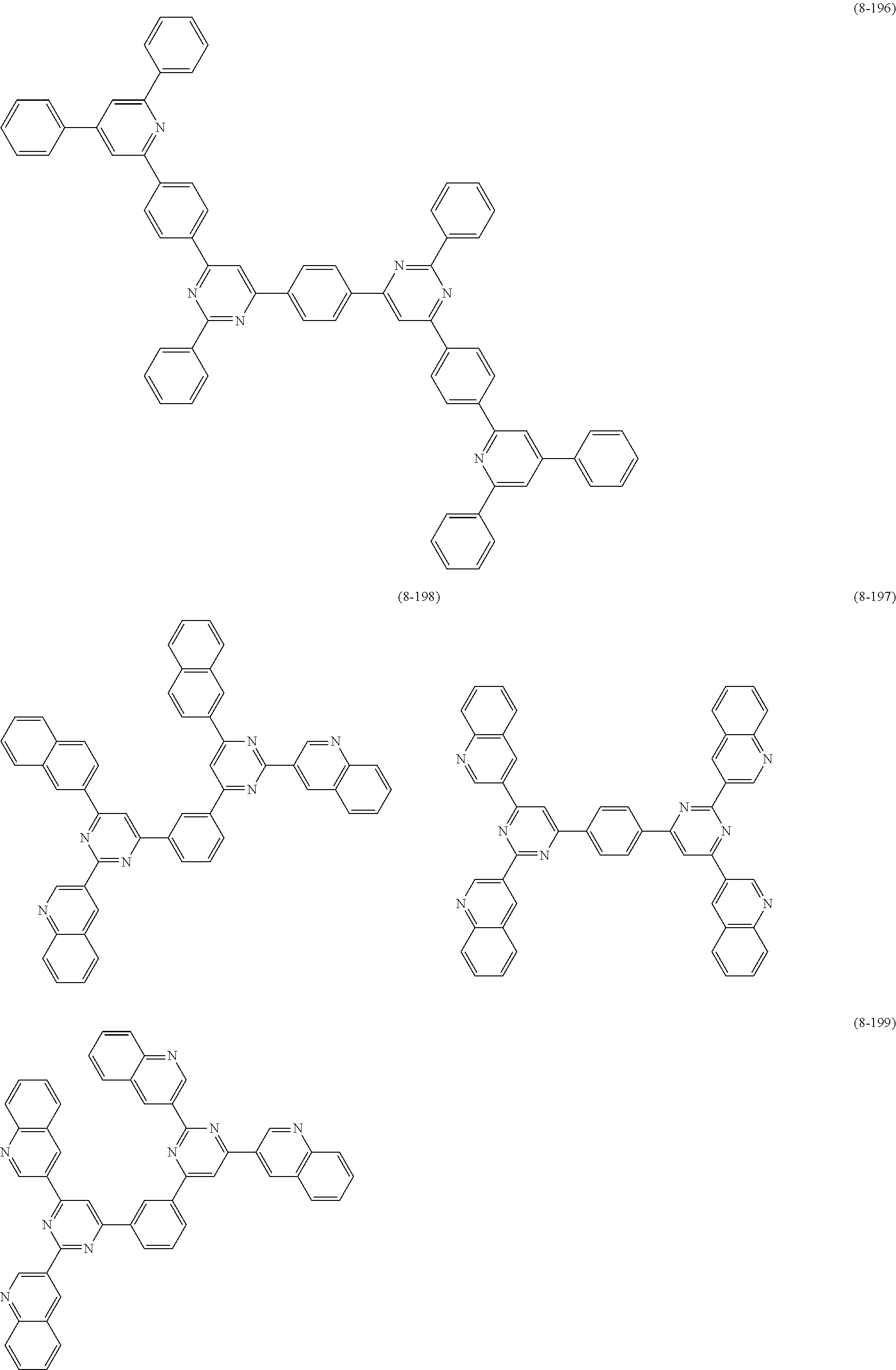

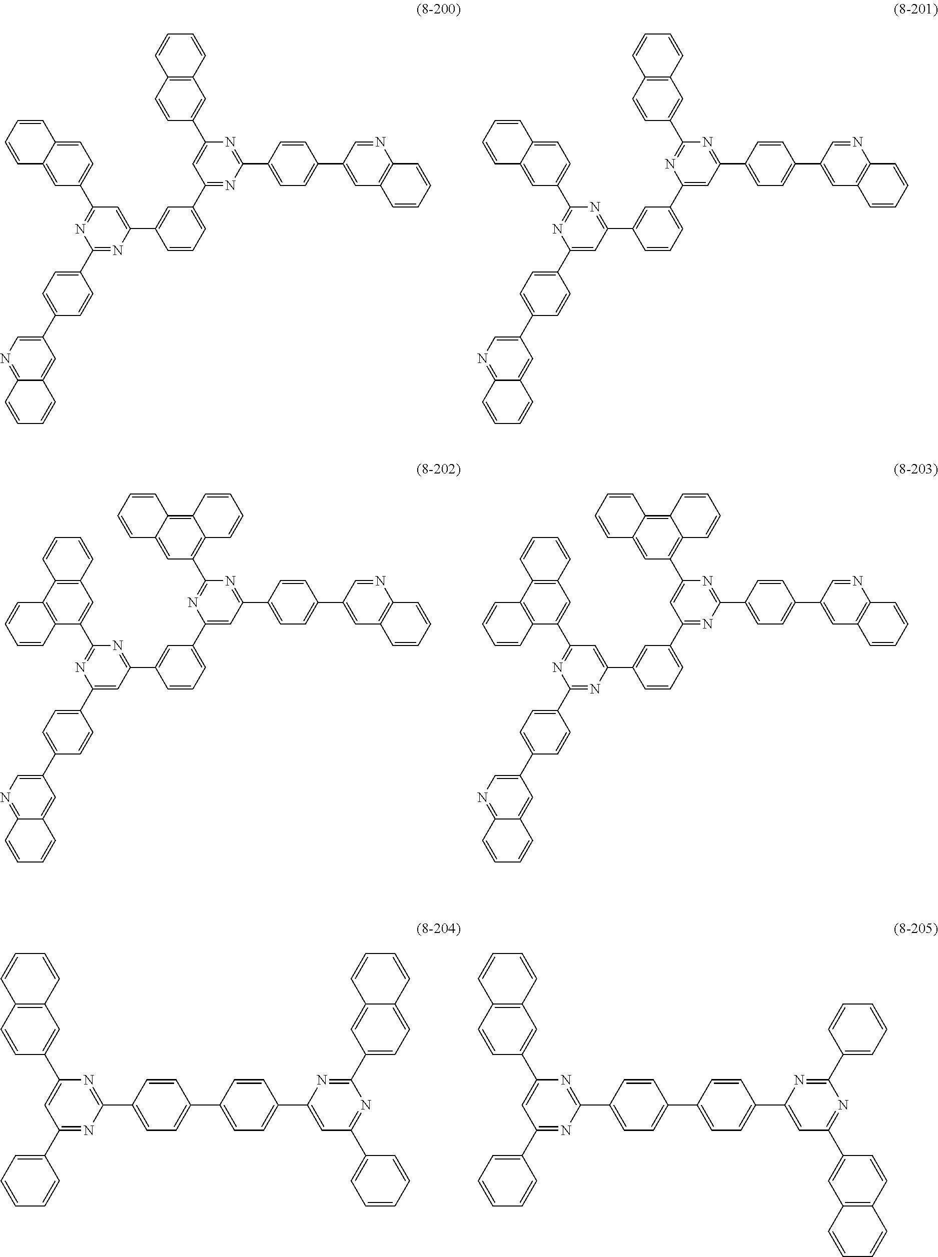

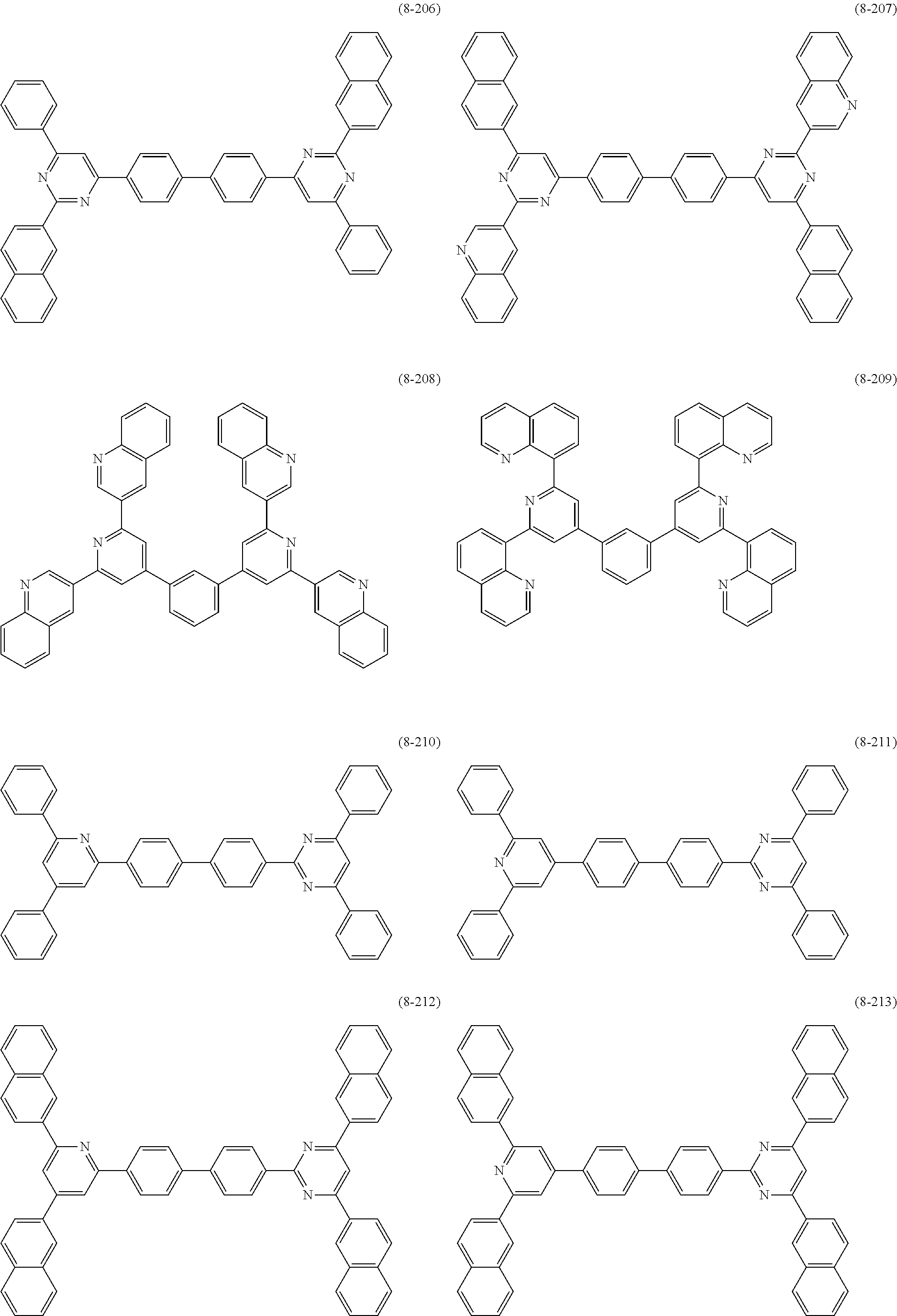

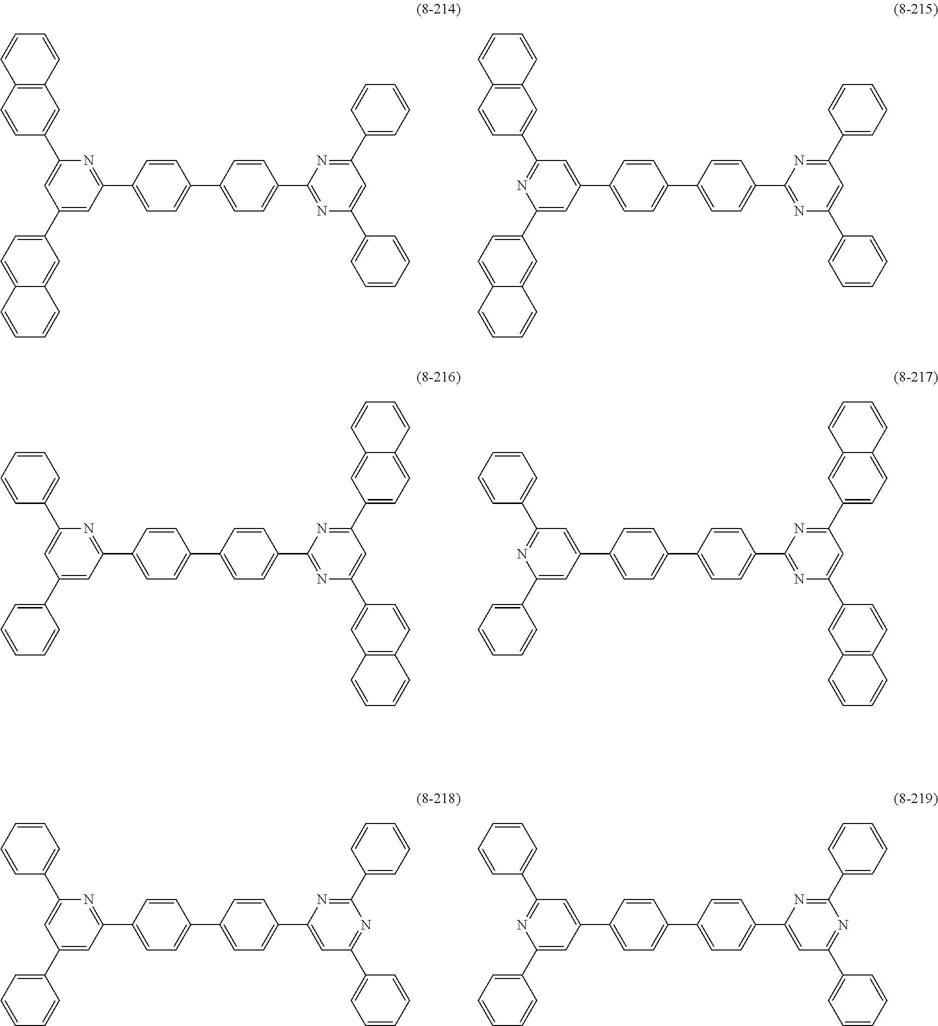

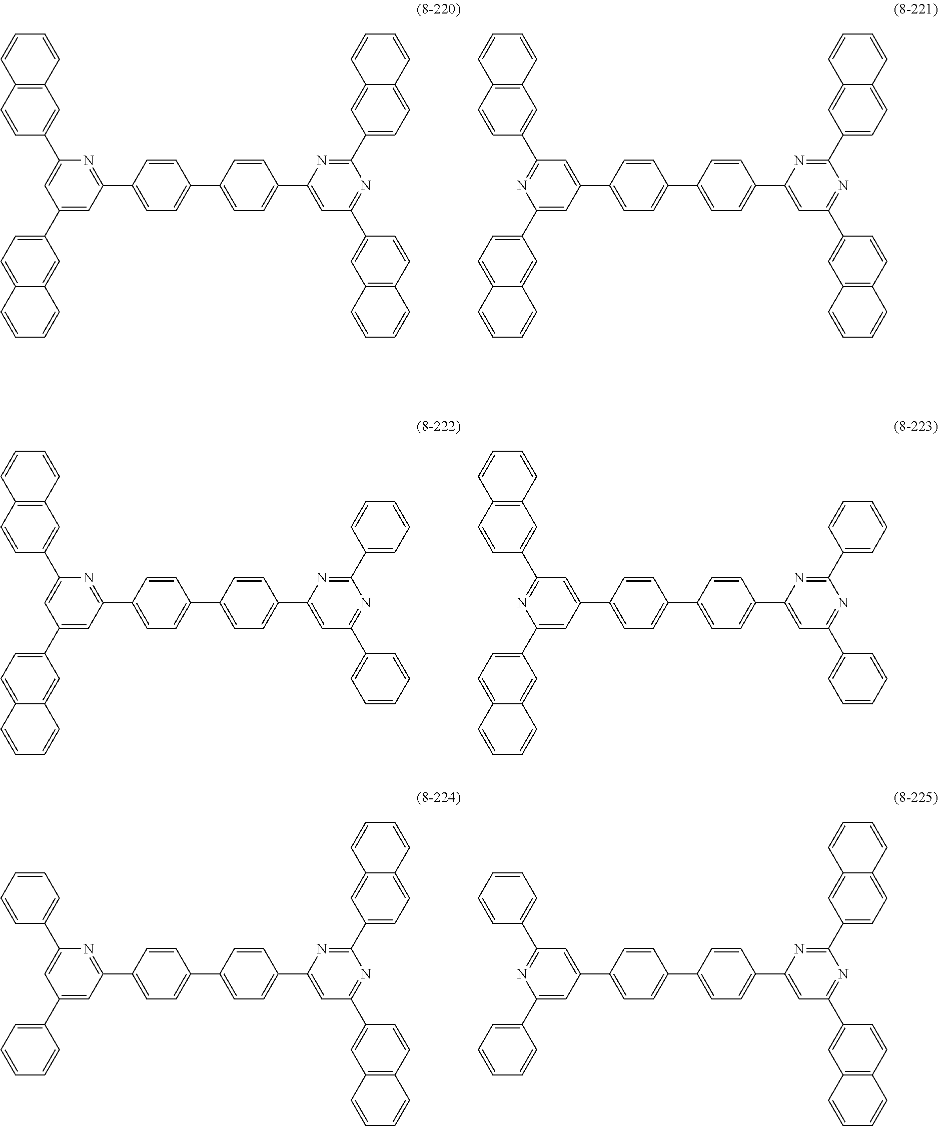

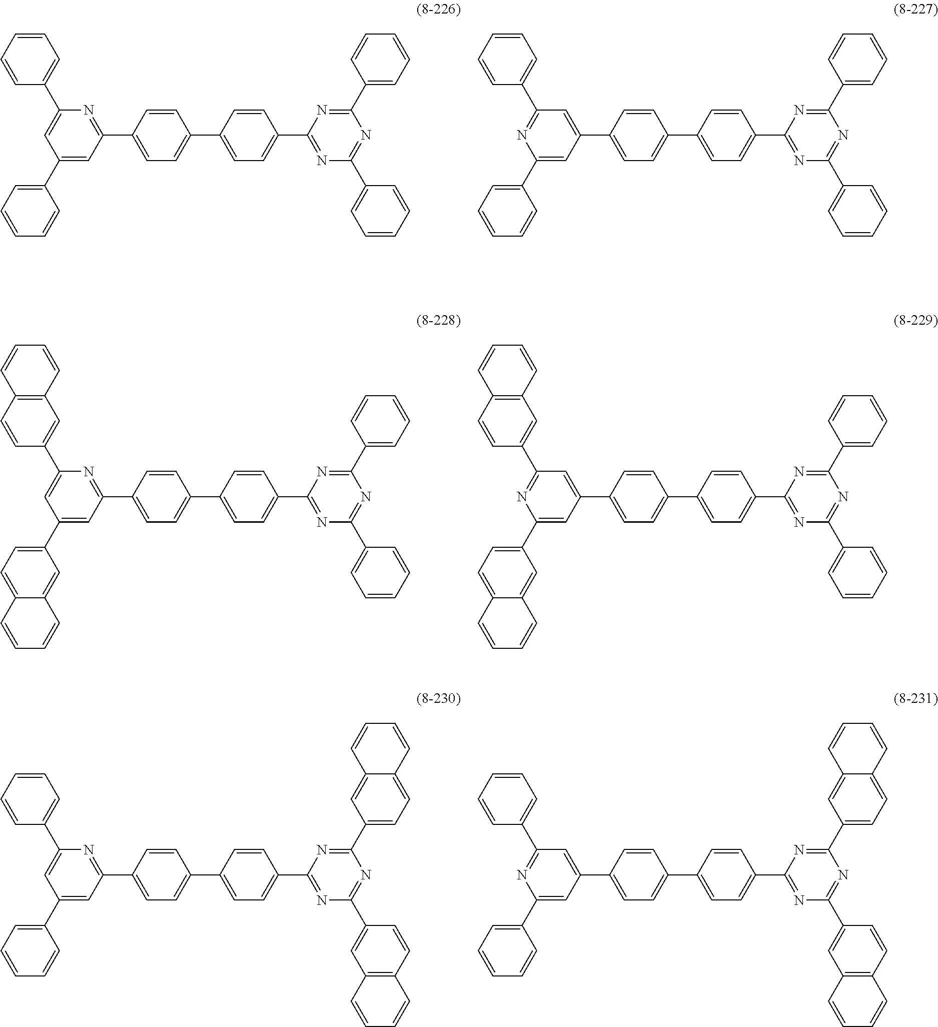

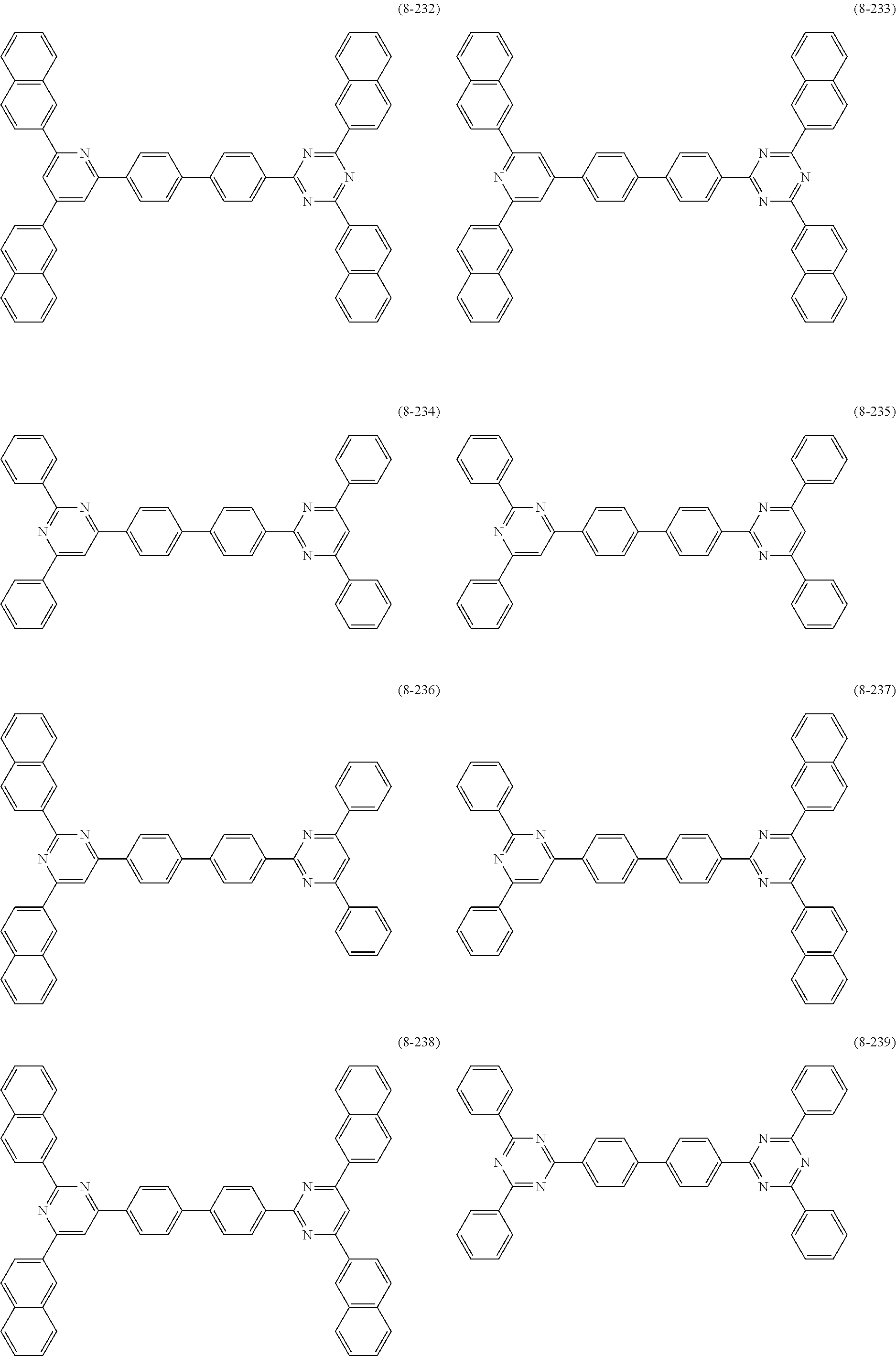

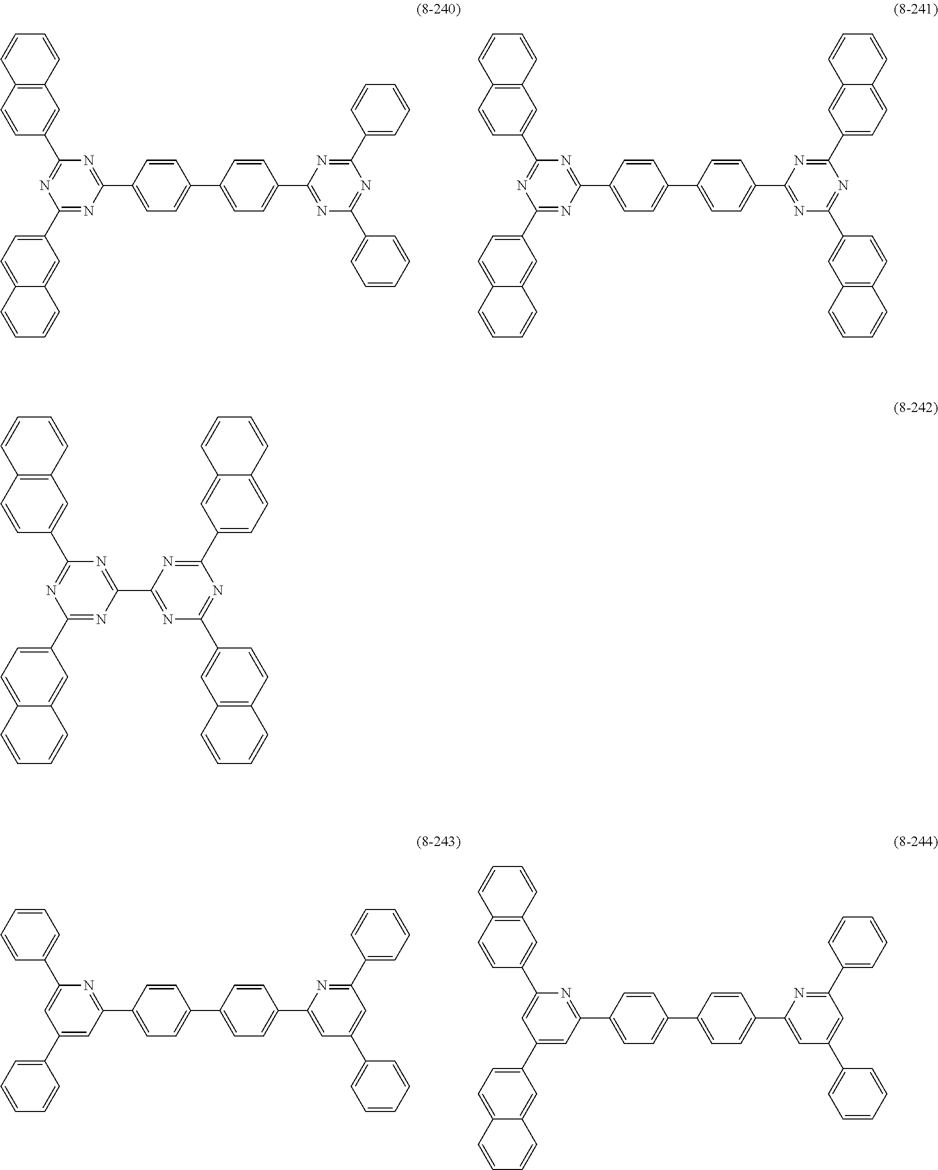

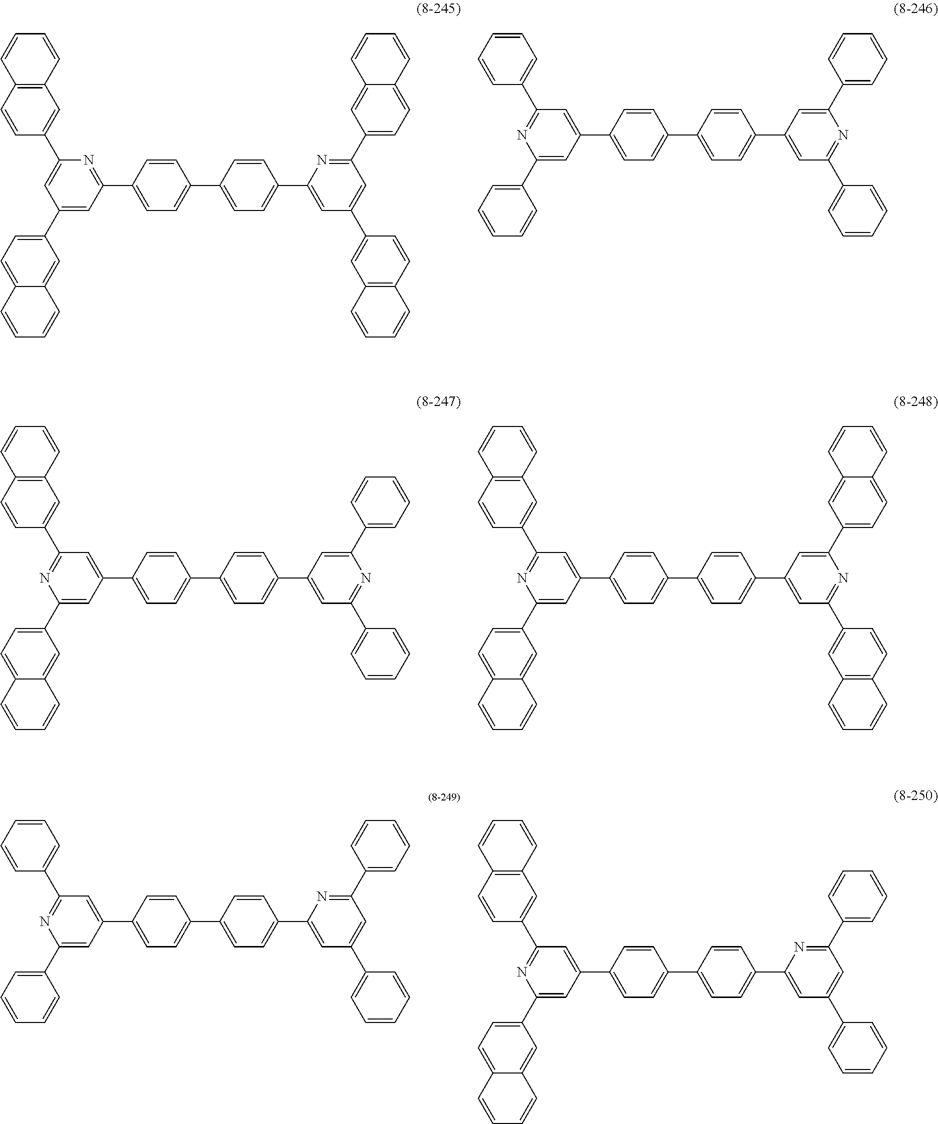

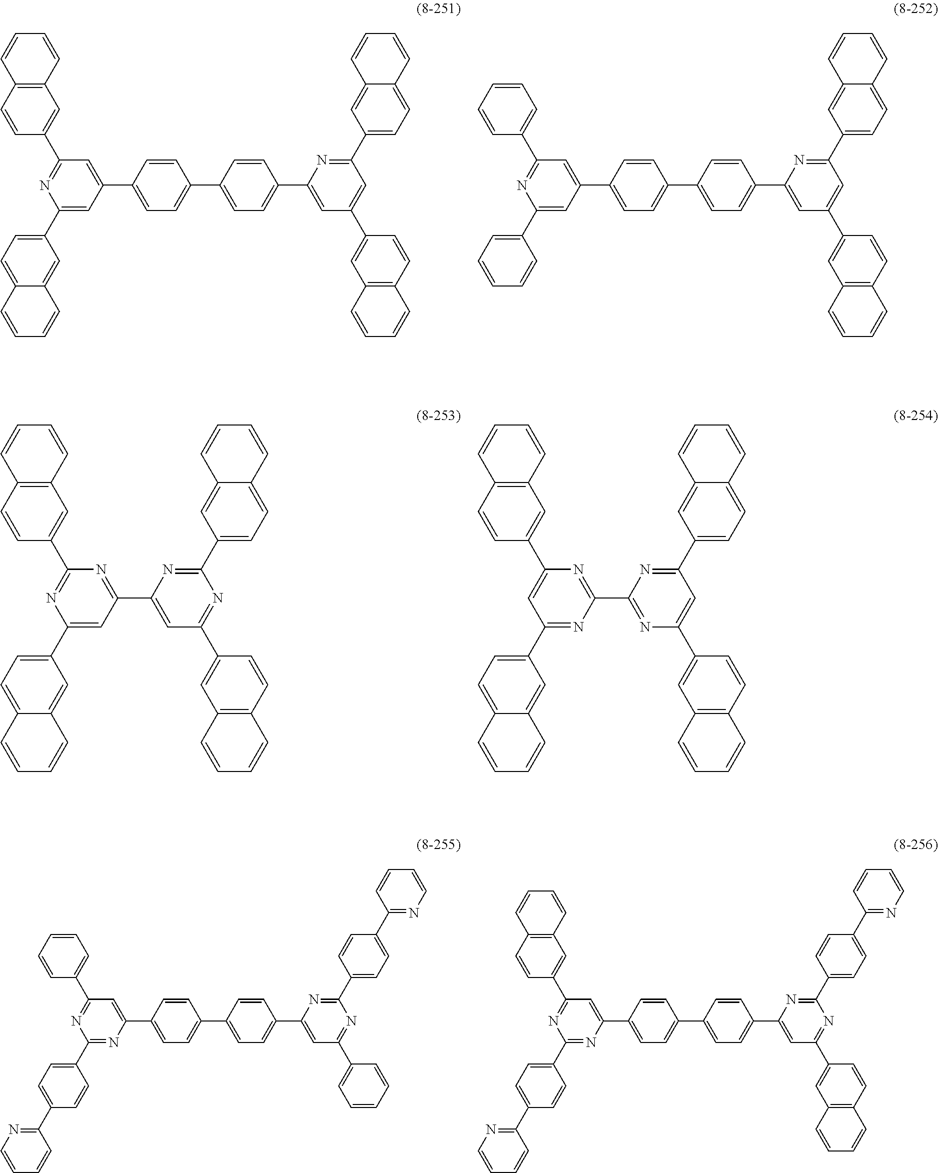

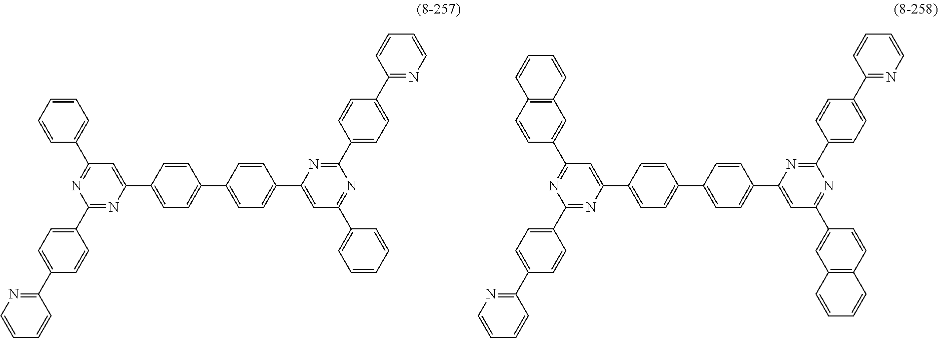

The compound for an organic photoelectric device may be used as a charge transporting material or a host material and thus, may help lower a driving voltage of an organic photoelectric device and may help improve luminous efficiency thereof.

In addition, when the compound for an organic photoelectric device is used as a host material, the compound for an organic photoelectric device may be mixed or blended with a suitable low molecular weight host material or a polymer host materials. In addition, the compound for an organic photoelectric device may be mixed with a binder resin, e.g., polyvinylcarbazole, polycarbonate, polyester, polyarylate, polystyrene, an acrylic polymer, a methacrylic polymer, polybutyral, polyvinylacetal, a diallylphthalate polymer, a phenol resin, an epoxy resin, a silicone resin, a polysulfone resin, a urea resin, or the like.

For example, the low molecular weight host material may include a compound represented by one of the following Chemical Formulae 9 to 12, and the polymer host material may include a polymer with a conjugated double bond, e.g., a fluorene-based polymer, a polyphenylenevinylene-based polymer, a polyparaphenylene-based polymer, or the like. However, the low molecular weight host material and polymer host material are not limited to the aforementioned examples.

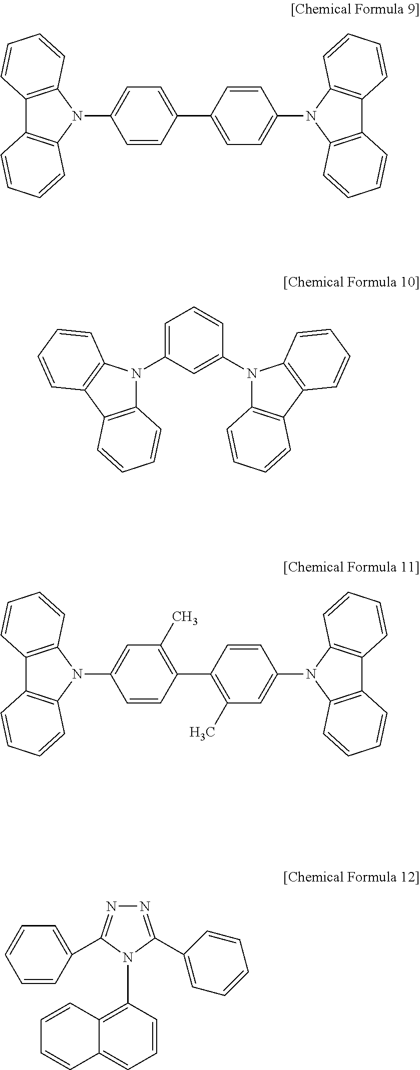

In an implementation, when the compound for an organic photoelectric device is used as a host material, the compound for an organic photoelectric device may be used singularly or mixed with a dopant. The dopant is a compound that may emit a light and may be referred to as a guest, because it may be mixed in a small amount with a host. For example, the dopant may be doped on or in a host material and may emit light. The dopant may include a metal complex or the like, which emit light by multiplet excitation, e.g., more than triplet excitation. The dopant may include any suitable red (R), green (G), blue (B), and/or white (W) fluorescent or phosphorescent dopants, e.g., red, green, blue, and/or white phosphorescent dopants. In addition, a dopant having high luminous efficiency, is not well-agglomerated, and is uniformly distributed in a host material may be used.

Examples of the phosphorescent dopant may include an organic metal compound including Ir, Pt, Os, Ti, Zr, Hf, Eu, Tb, Tm, Fe, Co, Ni, Ru, Rh, Pd, or a combination thereof. In an implementation, the red phosphorescent dopant may include platinum-octaethylporphyrina complex (PtOEP), Ir(btp)₂(acac) (bis(2-(2′-benzothienyl)-pyridinato-N,C3′)iridium(acetylacetonate)), Ir(Piq)₂(acac), Ir(Piq)₃, RD61 made by UDC Co., or the like. The green phosphorescent dopant may include Ir(PPy)₂(acac), Ir(PPy)₃, GD48 made by UDC Co., or the like. The blue phosphorescent dopant may include (4,6-F₂PPy)₂Irpic, FIrpic(Ir bis[4,6-di-fluorophenyl)-pyridinato-N,C2′]picolinate), or the like. Herein, Piq refers to 1-phenylisoquinoline, acac refers to acetylacetonate, and PPy refers to 2-phenylpyridine.

Another embodiment provides an organic photoelectric device including an anode, a cathode, and at least one or more organic thin layers between the anode and the cathode. The organic thin layer may include a compound for an organic photoelectric device according to an embodiment. Herein, the organic photoelectric device may refer to an organic light emitting diode, an organic solar cell, an organic transistor, an organic photo conductor drum, an organic memory device, and the like. In the organic solar cell, the compound for an organic photoelectric device according to an embodiment may be included in an electrode or an electrode buffer layer and thus, may help increase quantum efficiency. In the organic transistor, the compound for an organic photoelectric device according to an embodiment may be used as an electrode material in a gate electrode, a source-drain electrode, or the like.

The organic thin layer including a compound for an organic photoelectric device may include, e.g., an emission layer, a hole transport layer (HTL), a hole injection layer (HIL), a hole blocking layer, an electron transport layer (ETL), an electron injection layer (EIL), an electron blocking layer, and/or a combination thereof. In an implementation, when the organic thin layer including the compound for an organic photoelectric device is an emission layer, the compound for an organic photoelectric device may be used as a phosphorescent host or a fluorescent host. In another implementation, when the organic thin layer including the compound for an organic photoelectric device is an emission layer, the compound for an organic photoelectric device may be used as a fluorescent blue dopant.

Hereinafter, a detailed described relating to the organic photoelectric device will be provided.

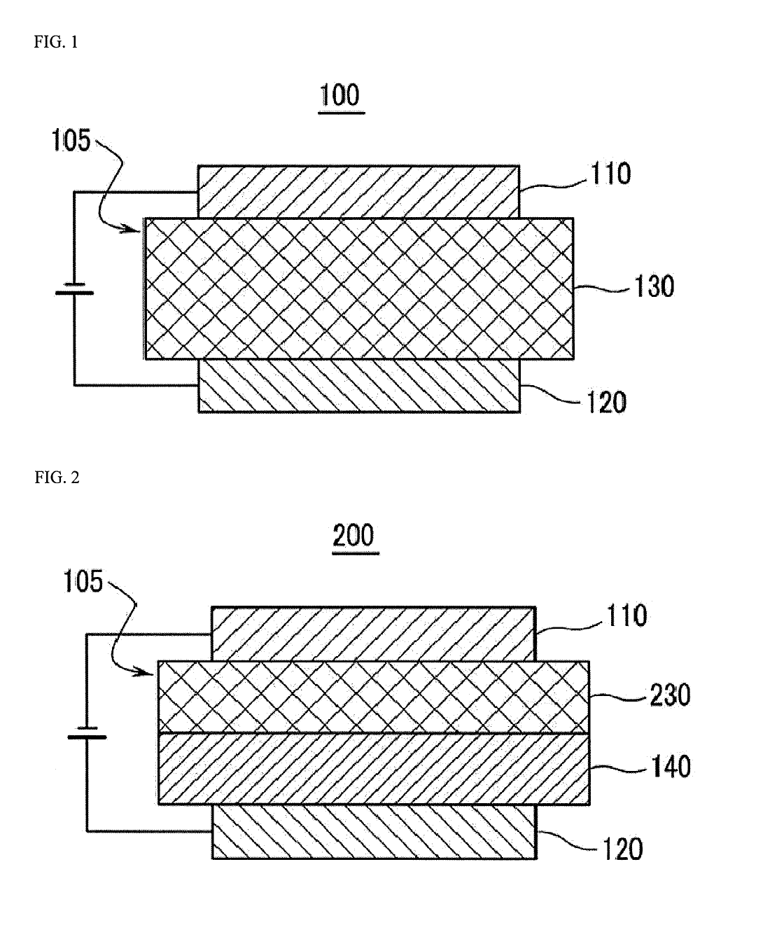

FIGS. 1 to 5 illustrate cross-sectional views showing an organic photoelectric device including the compound for an organic photoelectric device.

Referring to FIGS. 1 to 5, organic photoelectric devices 100, 200, 300, 400, and 500 may include at least one organic thin layer 105 interposed between an anode 120 and a cathode 110.

A substrate has no particular limit but may include any suitable substrate for an organic photoelectric device. For example, the substrate may include a glass substrate (with excellent transparency, surface flatness, easy management, and water impermeability), a transparent plastic substrate, or the like.

The anode 120 may include an anode material laving a large work function to facilitate smooth injection of holes into the organic thin layer 105. The anode material may include a metal, e.g., nickel, platinum, vanadium, chromium, copper, zinc, gold, and the like, or alloys thereof, a metal oxide, e.g., zinc oxide, indium oxide, indium tin oxide (ITO), and indium zinc oxide (IZO), and the like, a combined metal and oxide, e.g., ZnO/Al, SnO₂/Sb, and the like. However, the anode material is not limited to the above materials. In an implementation, the anode 120 may be an ITO transparent electrode.

The cathode 110 may include a cathode material having a small work function to facilitate smooth injection of electrons into the organic thin layer 105. The cathode material may include a metal, e.g., magnesium, calcium, sodium, potassium, titanium, indium, yttrium, lithium, gadolinium, aluminum, silver, tin, lead, cesium, barium, and the like, or alloys thereof, or a multi-layered material, e.g., LiF/Al, LiO₂/Al, LiF/Ca, LiF/Al, LiQ ((8-hydroxyquinolinolato)-lithium)/Al, BaF₂/Ca, and the like. However, the cathode material is not limited to the above materials. In an implementation, the cathode 110 may be an aluminum metal electrode.

FIG. 1 illustrates an organic photoelectric device 100 that includes only an emission layer 130 as the organic thin layer 105.

FIG. 2 illustrates a two-layered organic photoelectric device 200 that includes an emission layer 230 (including an electron transport layer (ETL)) and a hole transport layer (HTL) 140 as the organic thin layer 105. For example, the organic thin layer 105 may include two layers of an emission layer 230 and a hole transport layer (HTL) 140. The emission layer 230 may also function as an electron transport layer (ETL), and the hole transport layer (HTL) 140 may have an excellent binding property with a transparent electrode (such as ITO) and excellent hole transporting properties.

The hole transport layer (HTL) 140 may include any suitable hole transport material, e.g., PEDOT:PSS of poly(3,4-ethylenedioxy-thiophene) (PEDOT) doped with a poly(styrenesulfonate) (PSS) layer, N,N′-bis(3-methylphenyl)-N,N-diphenyl-[1,1′-biphenyl]-4,4′-diamine (TPD), N,N′-di(1-naphthyl)-N,N′-diphenylbenzidine (NPB), or the like, as well as a compound for an organic photoelectric device according to an embodiment. However, the hole transport material is not limited to the aforementioned materials.

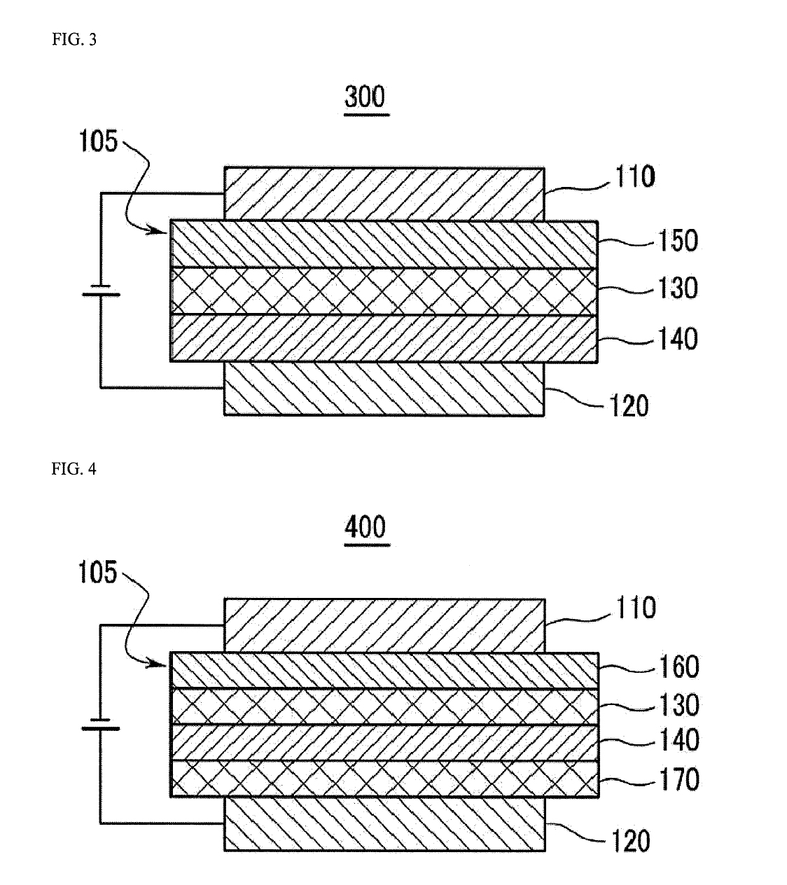

FIG. 3 illustrates a three-layered organic photoelectric device 300 that includes an electron transport layer (ETL) 150, an emission layer 130, and a hole transport layer (HTL) 140 as the organic thin layer 105. For example, the organic thin layer 105 may include an independently-installed emission layer 130 and separately-stacked layers (the electron transport layer (ETL) 150 and the hole transport layer (HTL) 140) having excellent electron transporting properties and excellent hole transporting properties, respectively.

The electron transport layer (ETL) 150 may include any suitable electron transport material, e.g., a 1,3,4-oxadiazole derivative such as aluminumtris (8-hydroxyquinoline) (Alq₃), 2-(4-biphenyl-5-phenyl-1,3,4-oxadiazole (PBD), and the like; a quinoxaline derivative such as 1,3,4-tris[(3-phenyl-6-trifluoromethyl)quinoxaline-2-yl]benzene (TPQ); a triazole derivative, and the like, as well as the compound for an organic photoelectric device according to an embodiment. However, electron transport material is not limited to the aforementioned materials.

FIG. 4 illustrates a four-layered organic photoelectric device 400 that includes an electron injection layer (EIL) 160, an emission layer 130, a hole transport layer (HTL) 140, and a hole injection layer (HIL) 170 as the organic thin layer 105. The hole injection layer (HIL) 170 may help improve binding properties with the anode 120, e.g., formed of ITO.

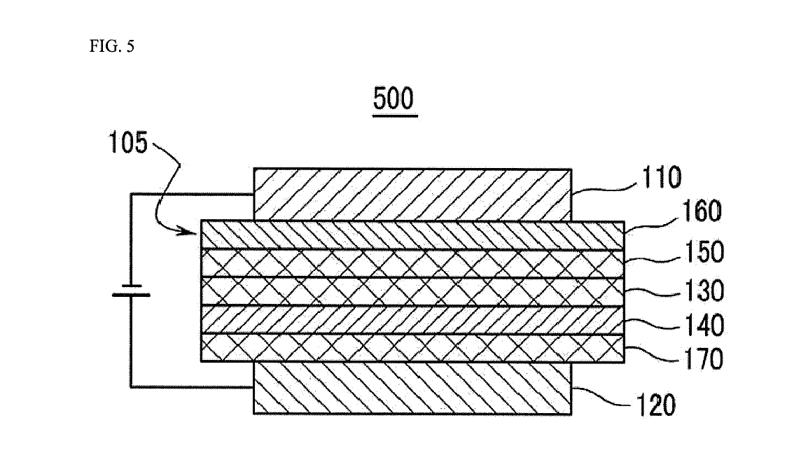

FIG. 5 illustrates a five-layered organic photoelectric device 500 that includes an electron injection layer (EIL) 160, an electron transport layer (ETL) 150, an emission layer 130, a hole transport layer (HTL) 140, and a hole injection layer (HIL) 170 as the organic thin layer 105. The electron injection layer (EIL) 160 may help effectively realize a low voltage.

The emission layers 130 and 230 may have a thickness of about 5 to about 1,000 nm. The hole transport layer (HTL) 140 and the electron transport layer (ETL) 150 may each have a thickness of about 10 to about 10,000 Å. However, the thickness ranges are not limited to the aforementioned range.

In FIGS. 1 to 5, the organic thin layer 105 (including one or more of the electron transport layer (ETL) 150, electron injection layer (EIL) 160, emission layers 130 and 230, hole transport layer (HTL) 140, hole injection layer (HIL) 170, or a combination thereof) may include the compound for an organic photoelectric device according to an embodiment. In an implementation, the compound for an organic photoelectric device may be included in the electron transport layer (ETL) 150 and/or the electron injection layer (EIL) 160. When the compound for an organic photoelectric device is used for or included in the electron transport layer (ETL), an organic photoelectric device having a simpler structure may be provided, because an additional hole blocking layer may not be required, e.g., a hole blocking layer may be omitted.

When the compound for an organic photoelectric device is included in the emission layers 130 and 230, the compound for an organic photoelectric device may be used as a phosphorescent host, and the emission layers 130 and 230 may further include a dopant. The dopant may be a red, green, blue, and/or white phosphorescent dopant.

The organic photoelectric device may be fabricated by forming an anode on a substrate; forming an organic thin layer (using a dry coating method such as vacuum deposition (evaporation), sputtering, plasma plating, ion plating, or a wet coating method such as spin coating, dipping, flow coating, and the like); and providing a cathode thereon.

Another embodiment provides a display device including the organic photoelectric device according to an embodiment.

The following Examples and Comparative Examples are provided in order to set forth particular details of one or more embodiments. However, it will be understood that the embodiments are not limited to the particular details described. Further, the Comparative Examples are set forth to highlight certain characteristics of certain embodiments, and are not to be construed as either limiting the scope of the invention as exemplified in the Examples or as necessarily being outside the scope of the invention in every respect.

Synthesis of Compound for an Organic Photoelectric Device

### Example 1

A compound for an organic photoelectric device was synthesized according to the following Reaction Scheme 1.

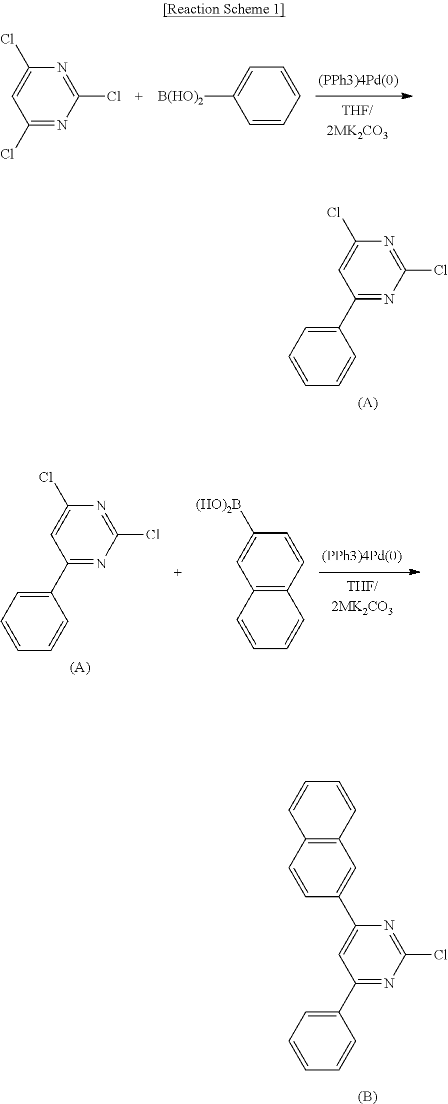

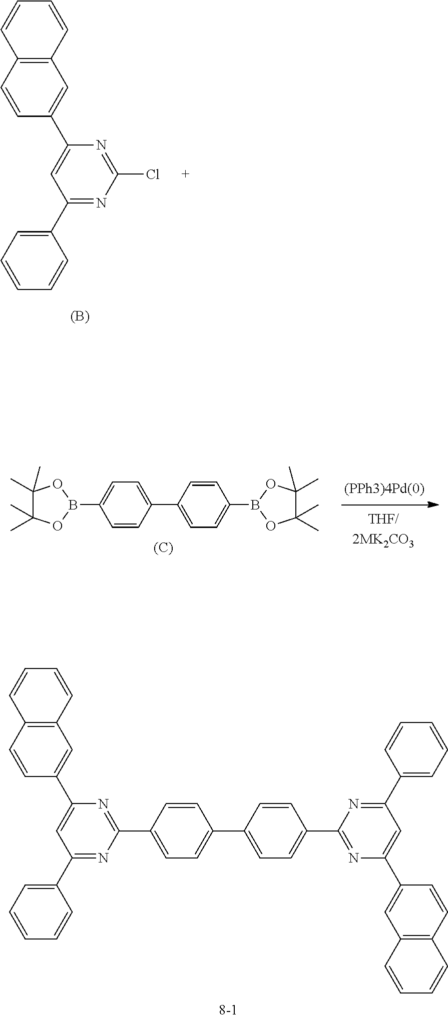

First Step: Synthesis of Intermediate Product A

99.7 g (543.7 mmol) of 2,4,6-trichloropyrimidine, 51 g (418 mmol) of phenylboronic acid, and 14 g (12.1 mmol) of tetrakis(triphenylphosphine)palladium were mixed with 600 mL of tetrahydrofuran (THF) in a 1 L round-bottomed flask (equipped with a thermometer, a reflux condenser, and an agitator) under a nitrogen atmosphere. The mixed solution was mixed with 200 mL of 2 M potassium carbonate (K₂CO₃), and the mixture was agitated at 70° C. for 12 hours.

The resulting mixture was cooled down to room temperature. When the reaction was complete, the reactant was extracted with methylene chloride and washed with water. Then, anhydrous magnesium sulfate was used to remove moisture from the reactant, and the reactant was filtered to remove an organic solvent. The final residue was purified through silica gel chromatography using a mixed solvent of methylene chloride and hexane mixed in a volume ratio of 3:2 at room temperature and recrystallized with hexane, obtaining 79 g of an intermediate product A (yield: 84%).

Second Step: Synthesis of Intermediate Product (B)

40.0 g (177.7 mmol) of the intermediate product A, 30.5 g (177.7 mmol) of 2-naphthaleneboronic acid, and 6 g (5.2 mmol) of tetrakis(triphenylphosphine)palladium were mixed with 500 mL of tetrahydrofuran in a 1 L round-bottomed flask (equipped with a thermometer, a reflux condenser, and an agitator) under a nitrogen atmosphere. 200 mL of 2 M potassium carbonate (K₂CO₃) was added thereto. The mixture was agitated at 70° C. for 12 hours.

The resulting mixture was cooled down to room temperature. When the reaction was complete, the reactant was extracted with methylene chloride and washed with water. Then, anhydrous magnesium sulfate was used to remove moisture from the reactant, and the resulting reactant was filtered to remove an organic solvent. The final residue was purified through silica gel chromatography using a mixed solvent of methylene chloride and hexane mixed in a volume ratio of 4:1 at room temperature and recrystallized with hexane, obtaining 43.2 g of a white intermediate product (B) (yield: 76.7%).

Third Step: Synthesis of Compound for Organic Photoelectric Device

35.0 g (110 mmol) of the intermediate product (B) from the second step, 19.5 g (48 mmol) of a compound (C), and 3.4 g (2.94 mmol) of tetrakis(triphenylphosphine)palladium were mixed with 600 mL of tetrahydrofuran in a 2 L round-bottomed flask (equipped with a thermometer, a reflux condenser, and an agitator) under a nitrogen atmosphere. 200 mL of 2 M potassium carbonate (K₂CO₃) was added thereto. The mixture was agitated at 80° C. for 12 hours, precipitating a white solid.

The reactant was cooled down to room temperature. When the reaction was complete, a potassium carbonate solution was removed from the reactant, and the resulting reactant was filtered, obtaining the white solid. The white solid was washed three times with tetrahydrofuran, three times with water, and three times with methanol and then, dried, obtaining 31 g of a compound for an organic photoelectric device (Compound 8-1) (yield: 90.3%).

The compound for an organic photoelectric device was analyzed regarding elements thereof. The result is provided as follows:

Calculated: C, 87.37; H, 4.79; N, 7.84.

Found: C, 87.36; H, 4.80; N, 7.84.

### Example 2

A compound for an organic photoelectric device was synthesized according to the following Reaction Scheme 2.

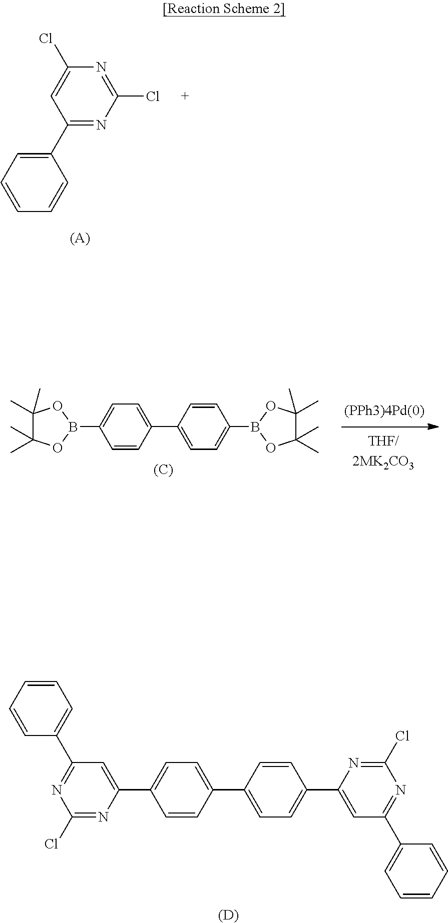

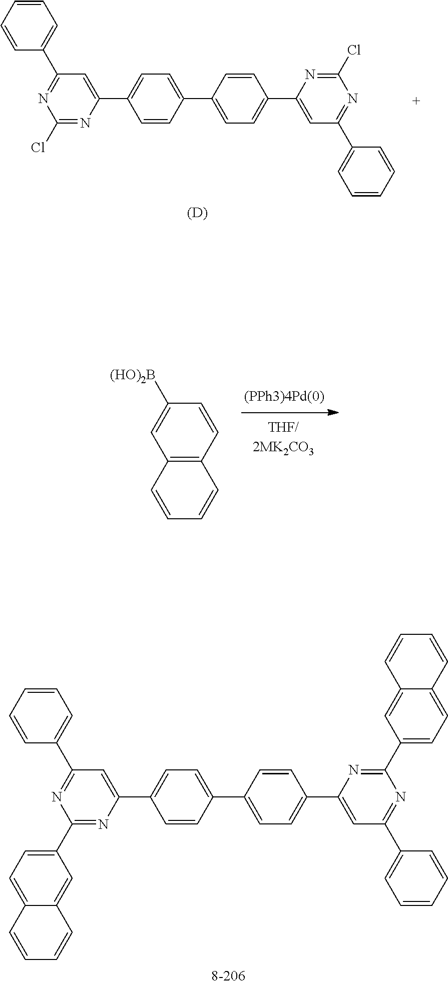

First Step: Synthesis of Intermediate Product (D)

17.0 g (75.8 mmol) of intermediate product (A) (synthesized in the first step of Example 1), 14 g (34.4 mmol) of compound (C), and 2 g (1.7 mmol) of tetrakis(triphenylphosphine)palladium were mixed with 300 mL of tetrahydrofuran in a 500 mL round-bottomed flask (equipped with a thermometer, a reflux-condenser, and an agitator) under a nitrogen atmosphere. 100 mL of 2 M potassium carbonate (K₂CO₃) was added thereto. The mixture was agitated at 70° C. for 12 hours.

The resulting reactant was cooled down to room temperature. When the reaction was complete, the reactant was extracted with methylene chloride and washed with water. Then, anhydrous magnesium sulfate was used to remove moisture from reactant, and an organic solvent was removed from the resulting reactant. The final residue was purified through silica gel chromatography using a methylene chloride solvent, obtaining 6.9 g of an intermediate product (D) (yield: 37%).

Second Step: Synthesis of Compound for Organic Photoelectric Device

6.8 g (12.7 mmol) of the intermediate product (D) synthesized in the first step, 5.5 g (31.9 mmol) of 2-naphthaleneboronic acid, and 0.88 g (0.76 mmol) of tetrakis(triphenylphosphine)palladium were mixed with 300 mL of tetrahydrofuran in a 2 L round-bottomed flask (equipped with a thermometer, a reflux condenser, and an agitator) under a nitrogen atmosphere. 100 mL of 2 M potassium carbonate (K₂CO₃) was added thereto. The mixture was agitated at 80° C. for 12, precipitating a white solid.

The reactant was cooled down to room temperature. When the reaction was complete, the reactant was filtered to remove a potassium carbonate solution, obtaining the white solid. The white solid was washed three times with tetrahydrofuran, three times with water, and three times with methanol and then, dried, obtaining 7 g of a compound for an organic photoelectric device (Compound 8-206) (yield: 76%).

The obtained compound for an organic photoelectric device was analyzed regarding elements thereof. The result is provided as follows:

Calculated: C, 87.37; H, 4.79; N, 7.84.

Found: C, 87.38; H, 4.78; N, 7.84.

### Example 3

A compound for an organic photoelectric device was synthesized according to the following Reaction Scheme 3.

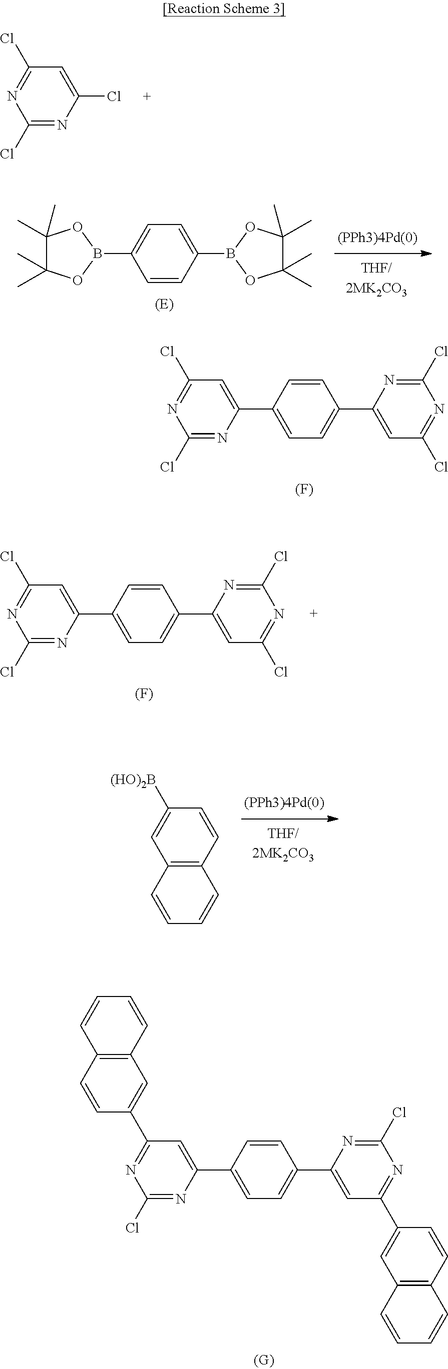

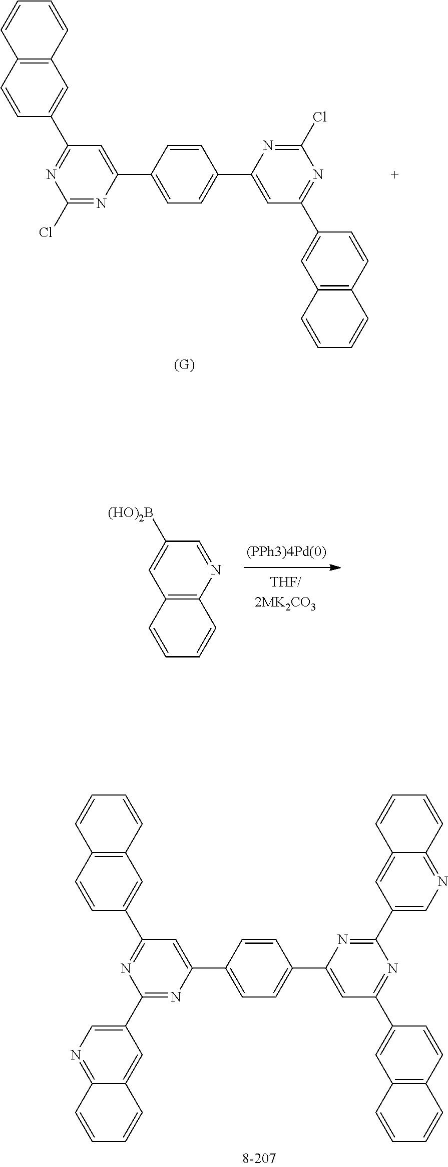

First Step: Synthesis of Intermediate Product (F)

55.5 g (303.3 mmol) of 2,4,6-trichloropyrimidine, 10 g (33.3 mmol) of a compound (E), and 2.3 g (1.9 mmol) of tetrakis(triphenylphosphine)palladium were mixed with 500 mL of tetrahydrofuran in a 1 L round-bottomed flask (equipped with a thermometer, a reflux-condenser, and an agitator) under a nitrogen atmosphere. 200 mL of 2 M potassium carbonate (K₂CO₃) was added thereto. The mixture was agitated at 70° C. for 12 hours.

The reactant was cooled down to room temperature. When the reaction was complete, the resulting reactant was filtered to remove a potassium carbonate solution, obtaining a white solid. The white solid was washed three times with tetrahydrofuran, three times with water, and three times with methanol and then, dried, obtaining 8 g of an intermediate product (F) (yield: 65%).

Second Step: Synthesis of Intermediate Product (G)

4.0 g (10.7 mmol) of the intermediate product (F) synthesized in the first step, 3.88 g (22.5 mmol) of 2-naphthaleneboronic acid and 1.2 g (1.0 mmol) of tetrakis(triphenylphosphine)palladium were mixed with 300 mL of tetrahydrofuran in a 500 mL round-bottomed flask (equipped with a thermometer, a reflux-condenser, and an agitator) under a nitrogen atmosphere. 100 mL of 2 M potassium carbonate (K₂CO₃) was added thereto. The mixture was agitated at 70° C. for 12 hours.

The reactant was cooled down to room temperature. When the reaction was complete, the reactant was filtered to remove a potassium carbonate solution, obtaining a white solid. The obtained white solid was washed three times with tetrahydrofuran, three times with water, and three times with methanol and then, dried, obtaining 4.3 g of an intermediate product (G) (yield: 72%).

Third Step: Synthesis of Compound for Organic Photoelectric Device

4.2 g (7.5 mmol) of the intermediate product (G) synthesized in the second step, 3.9 g (22.6 mmol) of quinoline-3-boronic acid, and 1 g (0.86 mmol) of tetrakis(triphenylphosphine)palladium were mixed with 300 mL of tetrahydrofuran in a 500 mL round-bottomed flask (equipped with a thermometer, a reflux-condenser, and an agitator) under a nitrogen atmosphere. 100 mL of 2 M potassium carbonate (K₂CO₃) was added thereto. The mixture was agitated at 80° C. for 12 hours, precipitating a white solid.

The reactant was cooled down to room temperature. When the reaction was complete, the reactant was filtered to remove a potassium carbonate solution to obtain the white solid. The filtered white solid was washed three times with tetrahydrofuran, three times with water, and three times with methanol and then, dried, obtaining 4.7 g of a compound for an organic photoelectric device (Compound 8-207) (yield: 84%).

The compound for an organic photoelectric device was analyzed regarding elements thereof. The result is provided as follows.

Calculated: C, 84.30; H, 4.35; N, 11.34.

Found: C, 84.32; H, 4.33; N, 11.34.

### Example 4

A compound for an organic photoelectric device was synthesized according to the following Reaction Scheme 4.

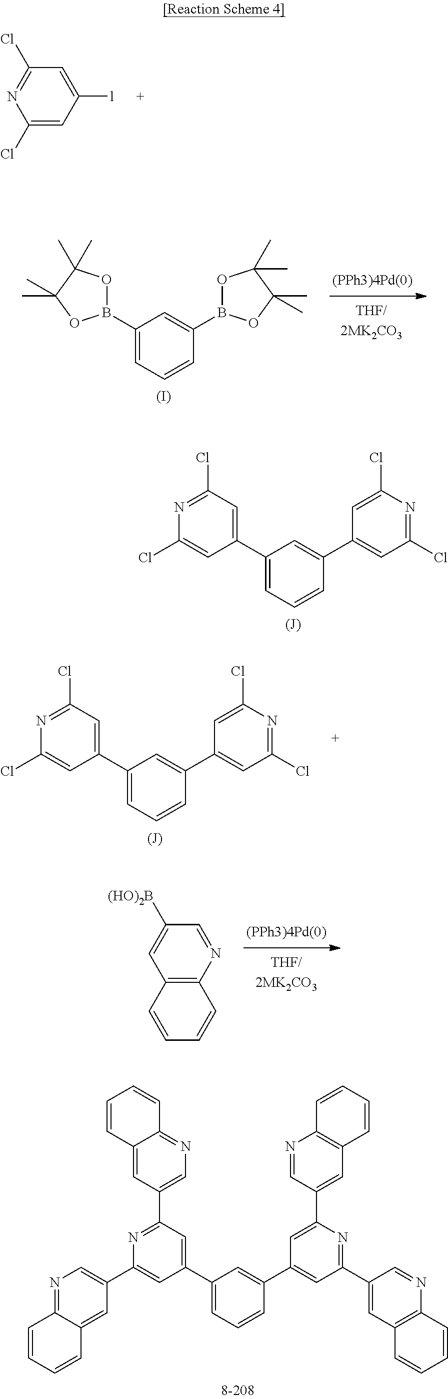

First Step: Synthesis of Intermediate Product (J)

8 g (24 mmol) of the compound (I), 14.6 g (53 mmol) of 4-iodine-2,6-dichloropyridine, and 2.8 g (2.4 mmol) of tetrakis(triphenylphosphine)palladium were mixed with 300 mL of tetrahydrofuran in a 500 mL round-bottomed flask (equipped with a thermometer, a reflux-condenser, and an agitator) under a nitrogen atmosphere. 100 mL of 2 M potassium carbonate (K₂CO₃) was added thereto. The mixture was agitated at 70° C. for 12 hours.

The reactant was cooled down to room temperature. When the reaction was complete, the reactant was filtered to remove a potassium carbonate solution, obtaining a white solid. The obtained white solid was washed three times with tetrahydrofuran, three times with water, and three times with methanol and then, dried, obtaining 5.4 g of an intermediate product (J) (yield: 60.8%).

Second Step: Synthesis of Compound for Organic Photoelectric Device

4 g (8.1 mmol) of the intermediate product (J) synthesized in the first step, 8.4 g (48.6 mmol) of quinoline-3-boronic acid and 1.2 g (1.0 mmol) of tetrakis(triphenylphosphine)palladium were mixed with 300 mL of tetrahydrofuran in a 2 L round-bottomed flask (equipped with a thermometer, a reflux-condenser, and an agitator) under a nitrogen atmosphere. 100 mL of 2 M potassium carbonate (K₂CO₃) was added thereto. The mixture was agitated at 80° C. 12 hours, precipitating a white solid.

The reactant was cooled down to room temperature. When the reaction was complete, the reactant was filtered to remove a potassium carbonate solution and obtain the white solid. The white solid was washed three times with tetrahydrofuran, three times with water, and three times with methanol and then, dried, obtaining 3.5 g of a compound for an organic photoelectric device (Compound 8-208) (yield: 59%).

The obtained compound for an organic photoelectric device was analyzed regarding elements thereof. The result is provided as follows.

Calculated: C, 85.76; H, 4.57; N, 9.67.

Found: C, 85.77; H, 4.56; N, 9.67.

### Example 5

A compound for an organic photoelectric device was synthesized according to the following Reaction Scheme 5.

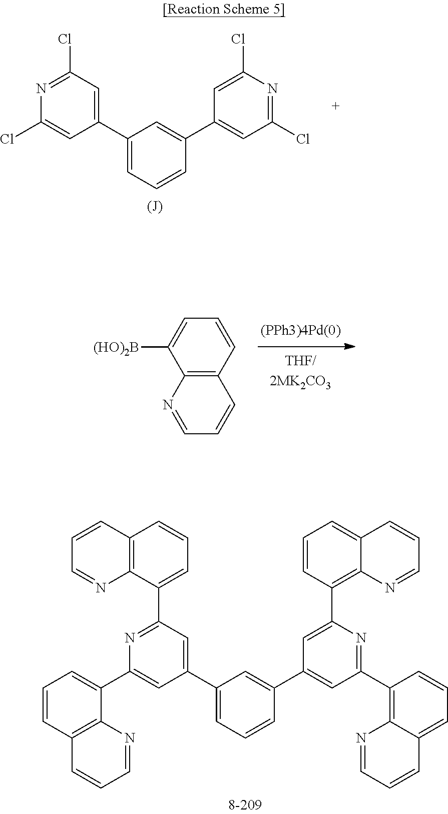

First Step: Synthesis of Compound for Organic Photoelectric Device

5.4 g (14.5 mmol) of the intermediate product (J) (synthesized in the first step of Example 4), 12.6 g (72.9 mmol) of quinoline-3-boronic acid, and 1.7 g (1.4 mmol) of tetrakis(triphenylphosphine)palladium were mixed with 300 mL of tetrahydrofuran in a 500 mL round-bottomed flask (equipped with a thermometer, a reflux-condenser, and an agitator) under a nitrogen atmosphere. 100 mL of 2 M potassium carbonate (K₂CO₃) was added thereto. The mixture was agitated at 80° C. for 12 hours.

The reactant was cooled down to room temperature. When the reaction was complete, a potassium carbonate solution and tetrahydrofuran were removed from the reactant under reduced pressure. Then, the reactant was extracted with methylene chloride and water, and the methylene chloride was removed under reduced pressure. The final residue was purified through silica gel chromatography using a mixed solvent of methylene chloride, ethyl acetate, and methanol mixed in a volume ratio of 4:2:0.1 (room temperature), obtaining 8.65 g of a compound for an organic photoelectric device (Compound 8-209) (yield: 80%).

The obtained compound for an organic photoelectric device was analyzed regarding elements thereof. The result is provided as follow.

Calculated: C, 85.76; H, 4.57; N, 9.67.

Found: C, 85.77; H, 4.56; N, 9.67.

### Example A-1

A compound for an organic photoelectric device was synthesized according to the following Reaction Scheme 6.

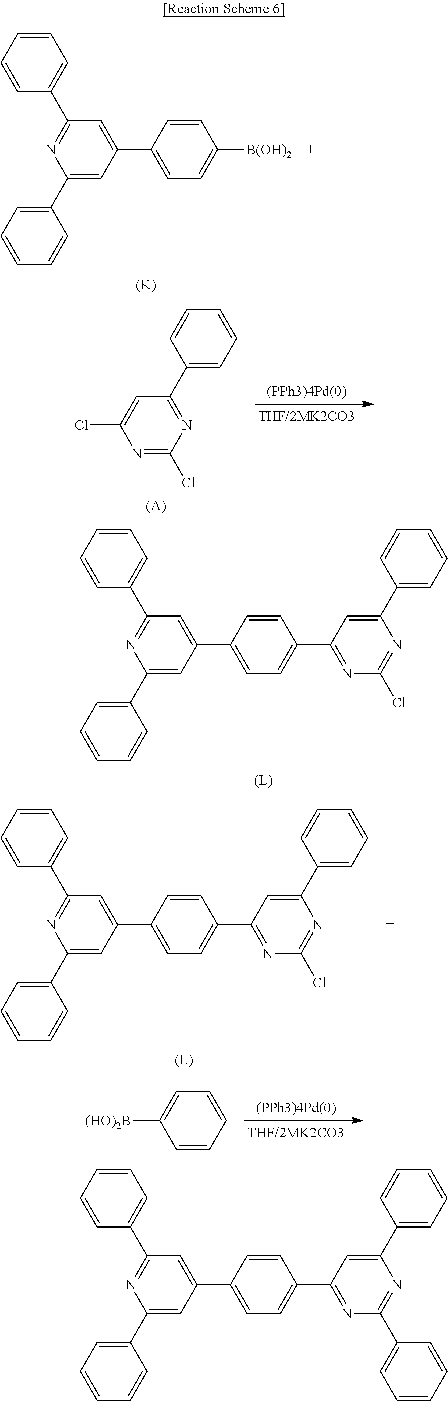

First Step: Synthesis of Intermediate Product (L)

10.0 g (28.4 mmol) of the compound (K), 7.04 g (31.3 mmol) of a compound (A), and 0.99 g (1.4 mmol) of tetrakis(triphenylphosphine)palladium were mixed with 300 mL of tetrahydrofuran in a 500 mL round-bottomed flask (equipped with a thermometer, a reflux-condenser, and an agitator) under a nitrogen atmosphere. 100 mL of 2 M potassium carbonate (K₂CO₃) was added thereto. The mixture was agitated at 80° C. for 12 hours.

The reactant was cooled down to room temperature. When the reaction was complete, a potassium carbonate solution and tetrahydrofuran were removed therefrom under reduced pressure. Then, the reactant was extracted with methylene chloride and water, and the methylene chloride was removed under reduced pressure. The final residue was recrystallized with toluene, obtaining 10.2 g of an intermediate compound (L) (yield: 72.2%).

The obtained compound for an organic photoelectric device was analyzed regarding elements thereof. The result is provided as follows.

Calculated: C, 79.91; H, 4.47; N, 8.47.

Found: C, 79.93; H, 4.45; N, 8.47.

Second Step: Synthesis of Compound for Organic Photoelectric Device

6 g (12.1 mmol) of the intermediate product (L) synthesized in the first step, 1.62 g (13.3 mmol) of phenylboronic acid, and 0.42 g (0.36 mmol) of tetrakis(triphenylphosphine)palladium were mixed with 500 mL of tetrahydrofuran in a 1 L round-bottomed flask (equipped with a thermometer, a reflux-condenser, and an agitator) under a nitrogen atmosphere. 200 mL of 2 M potassium carbonate (K₂CO₃) was added thereto. The mixture was agitated at 80° C. for 12 hours, precipitating a white solid.

The reactant was cooled down to room temperature. When the reaction was complete, the reactant was filtered to remove a potassium carbonate solution, obtaining the white solid. The white solid was washed three times with tetrahydrofuran, three times with water, and three times with methanol and then, dried, obtaining 5.5 g of a compound for an organic photoelectric device (yield: 84.6%).

The compound for an organic photoelectric device was analyzed regarding elements thereof. The result is provided as follows.

Calculated: C, 87.12; H, 5.06; N, 7.82.

Found: C, 87.10; H, 5.08; N, 7.82.

### Example A-2

A compound for an organic photoelectric device was synthesized according to the following Reaction Scheme 7.

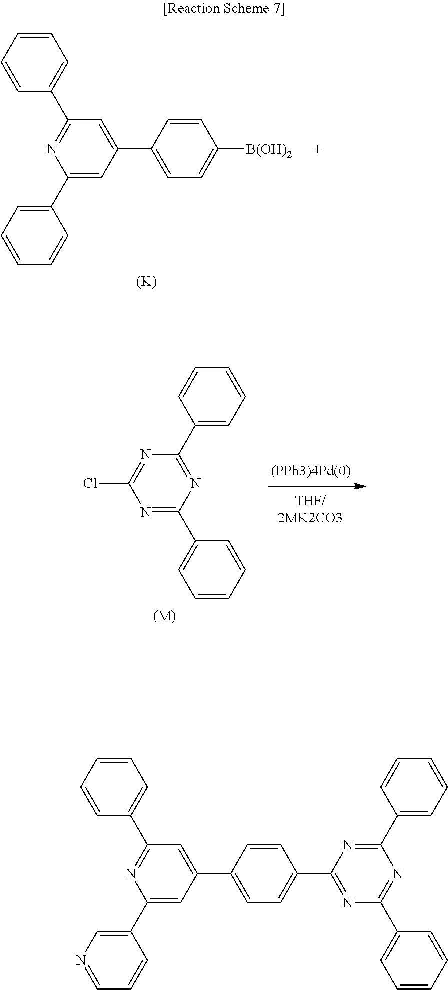

First Step: Synthesis of Compound for Organic Photoelectric Device

6 g (17.08 mmol) of a compound (K), 5.03 g (18.8 mmol) of a compound (M), and 0.6 g (0.51 mmol) of tetrakis(triphenylphosphine)palladium were mixed with 500 mL of tetrahydrofuran in a 1 L round-bottomed flask (equipped with a thermometer, a reflux-condenser, and an agitator) under a nitrogen atmosphere. 200 mL of 2 M potassium carbonate (K₂CO₃) was added thereto. The mixture was agitated at 80° C. for 12 hours, precipitating a white solid.

The reactant was cooled down to room temperature. When the reaction was complete, the reactant was filtered to remove a potassium carbonate solution, obtaining the white solid. The white solid washed was three times with tetrahydrofuran, three times with water, and three times with methanol, dried, and recrystallized in chlorobenzene, obtaining 7.5 g of a compound for an organic photoelectric device (yield: 81.5%).

The compound for an organic photoelectric device was analyzed regarding elements thereof. The result is provided as follows.

Calculated: C, 84.73; H, 4.87; N, 10.4.

Found: C, 84.72; H, 4.88; N, 10.4.

### Example A-3

A compound for an organic photoelectric device was synthesized according to the following Reaction Scheme 8.

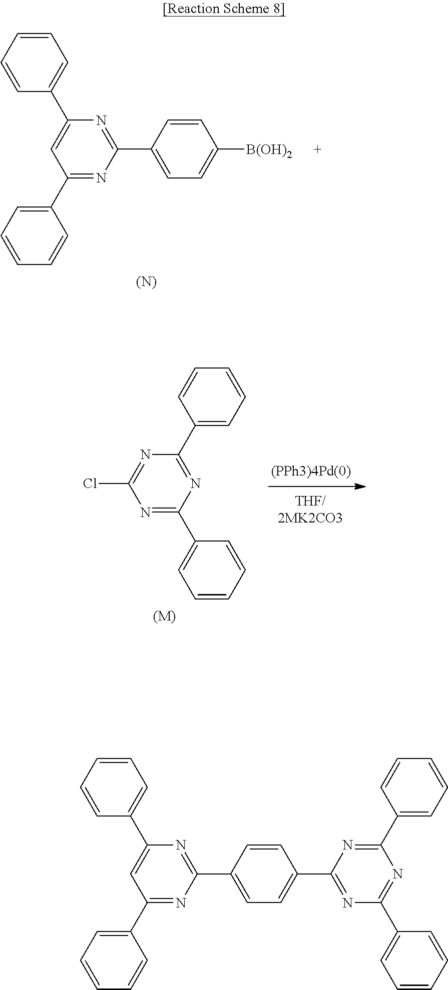

First Step: Synthesis of Compound for Organic Photoelectric Device

6 g (17.04 mmol) of a compound (N), 5.02 g (18.74 mmol) of a compound (M), and 0.6 g (0.51 mmol) of tetrakis(triphenylphosphine)palladium were mixed with 500 mL of tetrahydrofuran in a 1 L round-bottomed flask (equipped with a thermometer, a reflux-condenser, and an agitator) under a nitrogen atmosphere. 200 mL of 2M potassium carbonate (K₂CO₃) was added thereto. The mixture was agitated at 80° C. for 12 hours, precipitating a white solid.

The reactant was cooled down to room temperature. When the reaction was complete, the reaction was filtered to remove a potassium carbonate solution and obtain the white solid. The white solid was washed three times with tetrahydrofuran, three times with water, and three times with methanol, dried, and recrystallized in dichlorobenzene, obtaining 7.3 g of a compound for an organic photoelectric device (yield: 79.4%).

The obtained compound for an organic photoelectric device was analyzed regarding elements thereof. The result is provided as follows.

Calculated: C, 82.35; H, 4.67; N, 12.98.

Found: C, 82.33; H, 4.69; N, 12.98.

Fabrication of Organic Light Emitting Diode

### Example 6

As a positive electrode (anode), an ITO (120 nm) glass substrate with 15 Ω/cm² of sheet resistance was cut into a size of 50 mm×50 mm×0.7 mm, cleaned with ultrasonic wave in isopropyl alcohol and pure water for 5 minute respectively, and cleaned with UV ozone for 30 minutes.

On the substrate, NPB (58.5 nm) (HIL) was thermally vacuum-deposited with a vacuum degree of 650×10⁻⁷ Pa at deposition speed ranging from 0.1 to 0.3 nm/s to form a hole injection layer, and a hole transport layer (HTL) was formed thereon by depositing LG101 (LG Chem Ltd.) (5 nm) and NPB (58.5 nm).

Next, a 20 nm-thick emission layer was formed under the same thermal vacuum deposition condition by using 9,10-di(2-naphthyl)anthracene (ADN) as a host material, and 4,4′-bis(2,2-diphenylethen-1-yl)-diphenyl (DPVBI) as a dopant simultaneously deposited therewith. Herein, the dopant was deposited in an amount of 4 wt % (based on 100 wt % of an entire weight of the emission layer) by regulating its deposition speed.

On the emission layer, a 30 nm-thick electron transport layer (ETL) was formed by using a mixture of the compound synthesized according to Example 1 and LiQ (1:1 weight ratio) under the same thermal vacuum deposition condition.

On the electron transport layer (ETL), LiQ (0.5 nm) and Al (100 nm) were sequentially deposited to form a cathode under the same thermal vacuum deposition condition, fabricating an organic light emitting diode.

### Example 7

An organic light emitting diode was fabricated according to the same method as Example 6 except for using a mixture of the compound synthesized according to Example 2 and LiQ (1:1 weight ratio) instead of using the mixture of the compound synthesized according to Example 1 and LiQ (1:1 weight ratio).

### Comparative Example 1

An organic light emitting diode was fabricated according to the same method as Example 6 except for using a mixture of a compound represented by the following Chemical Formula 13 and LiQ (1:1 weight ratio) (instead of using the mixture of a compound synthesized according to Example 1 and LiQ (1:1 weight ratio)) to form an electron transport layer (ETL).

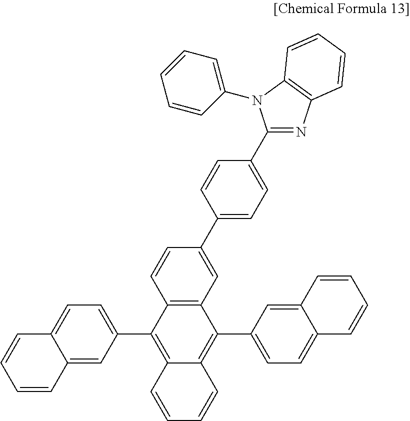

### Experimental Example 1

### Performance Evaluation of Organic Light Emitting Diode

The organic light emitting diodes according to Examples 6 and 7 and Comparative Example 1 were measured regarding current density change and luminance change depending on a voltage and luminous efficiency

The measurement was specifically performed, and the result is provided in Table 1, below.

(1) Current Density Change Depending on Voltage Change

The organic light emitting diode was measured regarding a current using a current-voltage meter (Keithley 2400) while its voltage was increased from 0 V to 14 V. Then, current density was obtained by dividing the current value by an area.

(2) Luminance Change Depending on Voltage Change

The organic light emitting diodes were measured regarding luminance by using a luminance meter (Minolta Cs-1000A) while its voltage was increased from 0 V to 14V.

(3) Luminous Efficiency

The luminance and current density measured from (1) and (2) and a voltage were used to calculate current efficiency (cd/A) and electric power efficiency (lm/W) at the same luminance (1000 cd/m²). The result is provided in the following Table 1.

(4) Color Coordinate

The organic light emitting diodes were measured regarding color coordinate by using a luminance meter (Minolta Cs-100A). The result is provided in the following Table 1.

TABLE 1

| | | At 1000 cd/m² | | | |
|---|---|---|---|---|---|
| | | Driving | Current | Electric power | Color |
| | Vₒₙ | voltage | efficiency | efficiency | coordinate |
| Devices | (V) | (V) | (cd/A) | (lm/W) | (x, y) |
| Example 6 | 2.7 | 3.9 | 9.75 | 7.69 | 0.15, 0.20 |
| Example 7 | 2.5 | 4.1 | 8.33 | 6.54 | 0.15, 0.20 |
| Comparative | 2.9 | 4.2 | 7.12 | 5.33 | 0.15, 0.20 |
| Example 1 | | | | | |

Referring to Table 1, when the organic light emitting diodes were evaluated regarding characteristics, the organic light emitting diode according to Examples 6 and 7 exhibited a low driving voltage and much improved device performance in terms of current efficiency and electric power efficiency, compared with the organic light emitting diode of Comparative Example 1. Accordingly, it may be seen that the compound synthesized according to the Examples lowered a driving voltage of the organic light emitting diodes and improved their luminance and efficiency.

By way of summation and review, when holes and electrons are recombined to produce light emitting excitons, three times as many triplet excitons may be produced, relative to singlet excitons. Therefore, fluorescence using only singlet excitons has a limit in terms of luminous efficiency, since singlet excitons is only 25% produced. However, phosphorescence can utilize 75% of the triplet exciton production ratio as well as 25% of the singlet exciton production ratio and thus, theoretically has internal quantum efficiency up to 100%. In other words, phosphorescence may have around four times higher luminous efficiency than fluorescence.

A dopant and a host material may be added in an emission layer in order to increase efficiency and stability of an organic light emitting diode. The host material may include 4,4-N,N-dicarbazolebiphenyl (CBP). CBP may be easily crystallized due to high structural symmetry and may cause a short cut off and/or a pixel defect due to low thermal stability, when a device including the CBP is tested regarding thermal resistance. In addition, host materials including CBP may have a greater hole transporting rate than an electron transporting rate. Thus, excitons may be ineffectively formed in the emission layer, thereby deteriorating luminous efficiency of a device.

Accordingly, the embodiments provide a compound for an organic photoelectric device having high electrical and thermal stability and being capable of transporting both holes and electrons, in order to realize a highly efficient and lifetime organic photoelectric device.

The embodiments provide a compound for an organic photoelectric device being capable of effectively transporting a hole and an electron.

The embodiments also provide an organic photoelectric device including the compound for an organic photoelectric device and having excellent efficiency and driving voltage characteristics.

The compound for an organic photoelectric device according to an embodiment may be applied to an organic thin layer for an organic photoelectric device and may provide an organic photoelectric device and a display device having high luminous efficiency at a low driving voltage and improved life-span.

Example embodiments have been disclosed herein, and although specific terms are employed, they are used and are to be interpreted in a generic and descriptive sense only and not for purpose of limitation. Accordingly, it will be understood by those of skill in the art that various changes in form and details may be made without departing from the spirit and scope of the present invention as set forth in the following claims.

What is claimed is:

1. A compound for an organic photoelectric device, the compound being represented by the following Chemical Formula 3a, 3b, or 3c: 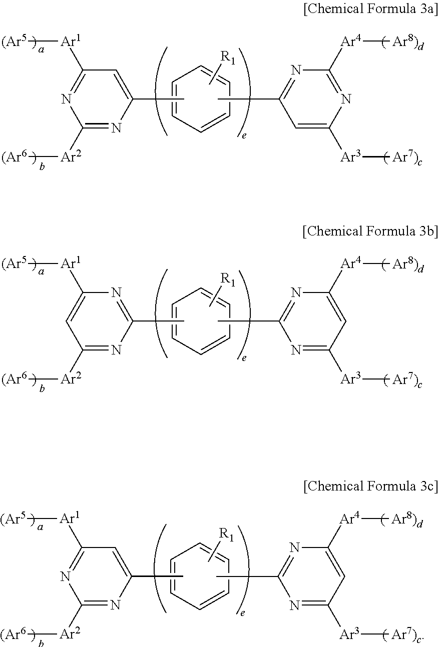
wherein, in Chemical Formulae 3a to 3c:
R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof,
Ar⁵ to Ar^{g} are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
a, b, c, and d are each independently integers ranging from 0 to 2, provided that when a, b, c, and d are 0, at least one selected from Ar¹ to Ar⁴ has 7 to 30 carbon atoms, and
e is 1 or 2, provided that when e is 2, two phenylene groups having the R1 substituents are in a para position relative to each other.

2. The compound as claimed in claim 1, wherein:
when any of R¹ and Ar¹ to Ar⁸ in the above Chemical Formulae 3a to 3c is an aryl group, the aryl group is a phenyl group, a biphenyl group, a terphenyl group, a stilbenzyl group, a naphthyl group, an anthracenyl group, a phenanthrenyl group, a pyrenyl group, a perylenyl group, or a combination thereof, and
when any of Ar¹ to Ar⁴ in the above Chemical Formulae 3a to 3c is an arylene group, the arylene group is a phenylene group, a biphenylene group, a terphenylene group, a stilbenzylene group, a naphthylene group, an anthracenylene group, a phenanthrenylene group, a pyrenylene group, a perylenylene group, or a combination thereof.

3. The compound as claimed in claim 1, wherein:
when any of R¹ and Ar¹ to Ar⁸ in the above Chemical Formulae 3a to 3c is a heteroaryl group, the heteroaryl group is a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and
when any of Ar¹ to Ar⁴ in the above Chemical Formulas 3a to 3c is a heteroarylene group, the heteroarylene group is a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

4. The compound as claimed in claim 1, wherein the compound for an organic photoelectric device is a charge transporting material or a host material.

5. A compound for an organic photoelectric device, the compound being represented by the following Chemical Formula 5a, 5b, 5c, or 5d: 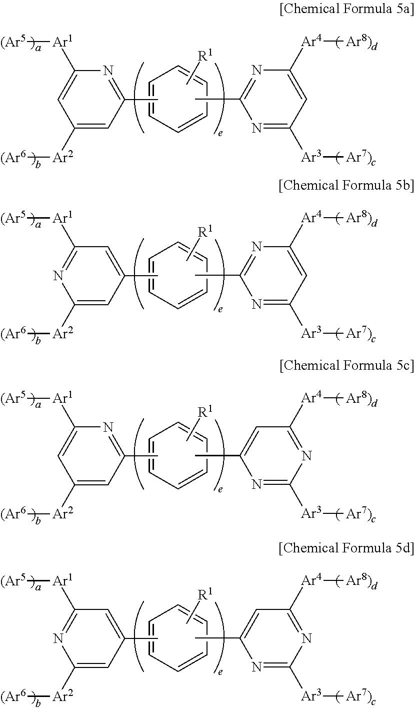
wherein, in Chemical Formulae 5a to 5d:
R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof,
Ar⁵ to Ar⁸ are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
a, b, c, and d are each independently integers ranging from 0 to 2, and
e is 1 or 2, provided that when e is 2, two phenylene groups having the R1 substituents are in a para position relative to each other.

6. The compound as claimed in claim 5, wherein:
when any of R¹ and Ar¹ to Ar⁸ in the above Chemical Formulae 5a to 5d is an aryl group, the aryl group is a phenyl group, a biphenyl group, a terphenyl group, a stilbenzyl group, a naphthyl group, an anthracenyl group, a phenanthrenyl group, a pyrenyl group, a perylenyl group, or a combination thereof, and
when any of Ar¹ to Ar⁴ in the above Chemical Formulae 5a to 5d is an arylene group, the arylene group is a phenylene group, a biphenylene group, a terphenylene group, a stilbenzylene group, a naphthylene group, an anthracenylene group, a phenanthrenylene group, a pyrenylene group, a perylenylene group, or a combination thereof.

7. The compound as claimed in claim 5, wherein:
when any of R¹ and Ar¹ to Ar⁸ in the above Chemical Formulae 5a to 5d is a heteroaryl group, the heteroaryl group is a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and
when any of Ar¹ to Ar⁴ in the above Chemical Formulae 5a to 5d is a heteroarylene group, the heteroarylene group is a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

8. The compound as claimed in claim 5, wherein the compound for an organic photoelectric device is a charge transporting material or a host material.

9. A compound for an organic photoelectric device, the compound being represented by the following Chemical Formula 6a or 6b: 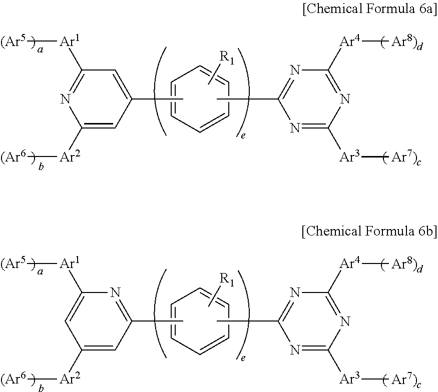
wherein, in Chemical Formulae 6a and 6b:
R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof,
Ar⁵ to Ar⁸ are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
a, b, c, and d are each independently integers ranging from 0 to 2, and
e is 1 or 2, provided that when e is 2, two phenylene groups having the R1 substituents are in a para position relative to each other.

10. The compound as claimed in claim 9, wherein:
when any of R¹ and Ar¹ to Ar⁸ in the above Chemical Formulae 6a and 6b is an aryl group, the aryl group is a phenyl group, a biphenyl group, a terphenyl group, a stilbenzyl group, a naphthyl group, an anthracenyl group, a phenanthrenyl group, a pyrenyl group, a perylenyl group, or a combination thereof, and
when any of Ar¹ to Ar⁴ in the above Chemical Formulae 6a and 6b is an arylene group, the arylene group is a phenylene group, a biphenylene group, a terphenylene group, a stilbenzylene group, a naphthylene group, an anthracenylene group, a phenanthrenylene group, a pyrenylene group, a perylenylene group, or a combination thereof.

11. The compound as claimed in claim 9, wherein:
when any of R¹ and Ar¹ to Ar⁸ in the above Chemical Formulae 6a and 6b is a heteroaryl group, the heteroaryl group is a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and
when any of Ar¹ to Ar⁴ in the above Chemical Formulae 6a and 6b is a heteroarylene group, the heteroarylene group is a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

12. A compound for an organic photoelectric device, the compound being represented by the following Chemical Formula 7a or 7b: 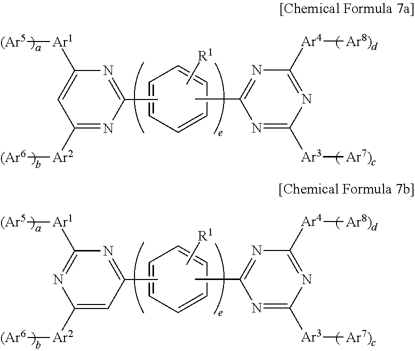
wherein, in Chemical Formulae 7a and 7b:
R¹ is hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
Ar¹ to Ar⁴ are each independently a substituted or unsubstituted C6 to C30 aryl group, a substituted or unsubstituted C6 to C30 arylene group, a substituted or unsubstituted C3 to C30 heteroaryl group, a substituted or unsubstituted C3 to C30 heteroarylene group, a substituted or unsubstituted C6 to C30 arylamine group, a substituted or unsubstituted carbazolyl group, a substituted or unsubstituted fluorenyl group, or a combination thereof,
Ar⁵ to Ar⁸ are each independently a substituted or unsubstituted C6 to C30 aryl group, a substituted or unsubstituted C3 to C30 heteroaryl group, a substituted or unsubstituted C6 to C30 arylamine group, a substituted or unsubstituted carbazolyl group, a substituted or unsubstituted fluorenyl group, or a combination thereof,
a, b, c, and d are each independently integers ranging from 0 to 2, and
e is 1 or 2, provided that when e is 2, two phenylene groups having the R1 substituents are in a para position relative to each other.

13. The compound as claimed in claim 12, wherein:
when any of R¹ and Ar¹ to Ar⁸ in the above Chemical Formulae 7a and 7b is an aryl group, the aryl group is a phenyl group, a biphenyl group, a terphenyl group, a stilbenzyl group, a naphthyl group, an anthracenyl group, a phenanthrenyl group, a pyrenyl group, a perylenyl group, or a combination thereof, and
when any of Ar¹ to Ar⁴ in the above Chemical Formulae 7a and 7b is an arylene group, the arylene group is a phenylene group, a biphenylene group, a terphenylene group, a stilbenzylene group, a naphthylene group, an anthracenylene group, a phenanthrenylene group, a pyrenylene group, a perylenylene group, or a combination thereof.

14. The compound as claimed in claim 12, wherein:
when any of R¹ and Ar¹ to Ar⁸ in the above Chemical Formulae 7a and 7b is a heteroaryl group, the heteroaryl group is a thiophenyl group, a furanyl group, a pyrrolyl group, an imidazolyl group, a thiazolyl group, an oxazolyl group, an oxadiazoly group, a triazolyl group, a pyridinyl group, a phenanthrolinyl group, a quinolinyl group, an isoquinolinyl group, an acridinyl group, an imidazopyridinyl group, an imidazopyrimidinyl group, a benzoquinolinyl group, a phenanthrolinyl group, or a combination thereof, and
when any of Ar¹ to Ar⁴ in the above Chemical Formulae 7a and 7b is a heteroarylene group, the heteroarylene group is a thiophenylene group, a furanylene group, a pyrrolylene group, an imidazolylene group, a thiazolylene group, an oxazolylene group, an oxadiazolyene group, a triazolylene group, a pyridinylene group, a pyradazinylene group, a quinolinylene group, an isoquinolinylene group, an acridinylene group, an imidazopyridinylene group, an imidazopyrimidinylene group, a benzoquinolinylene group, a phenanthrolinylene group, or a combination thereof.

15. A compound for an organic photoelectric device, the compound being represented by one of the following Compounds 8-1 to 8-258:

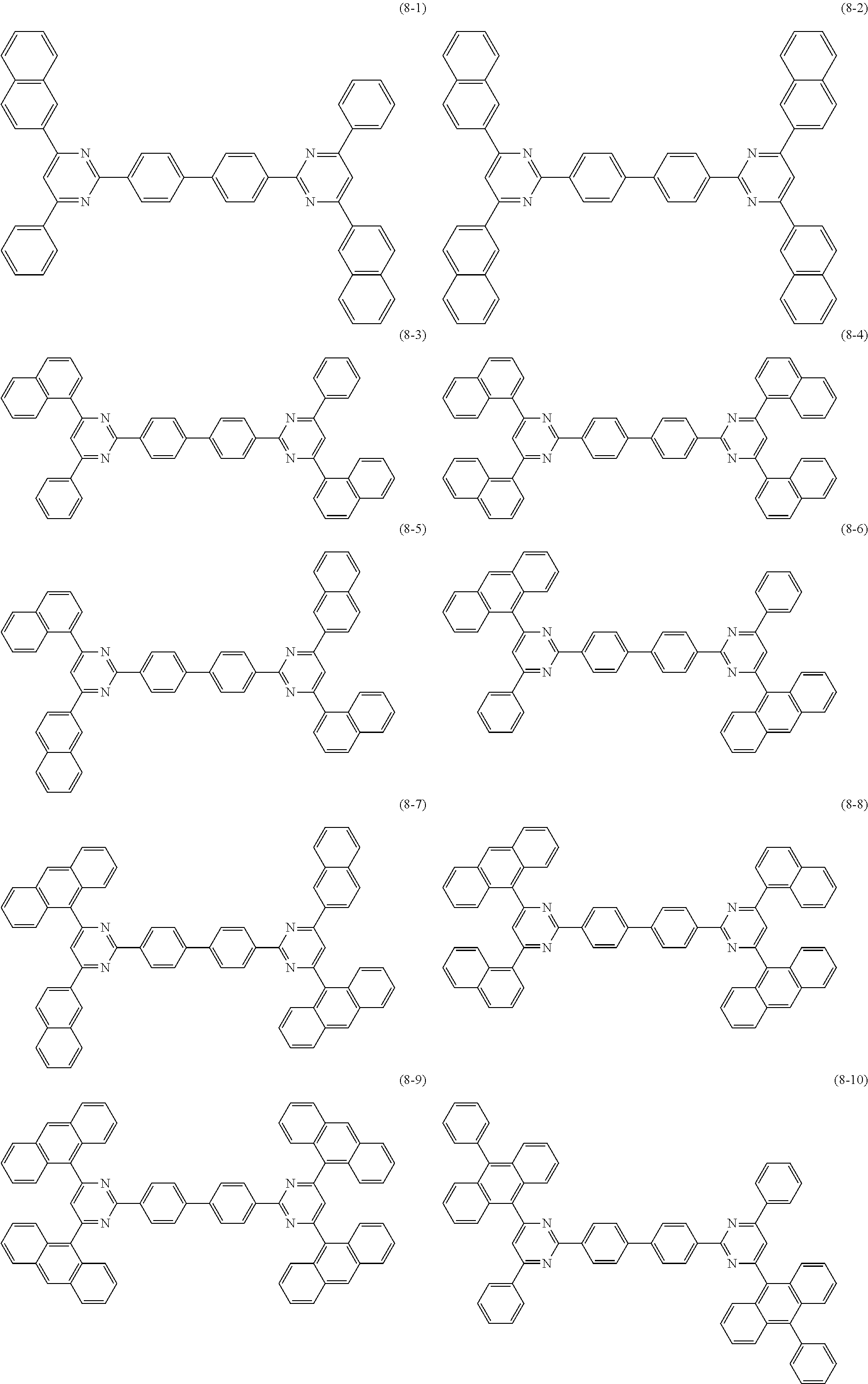

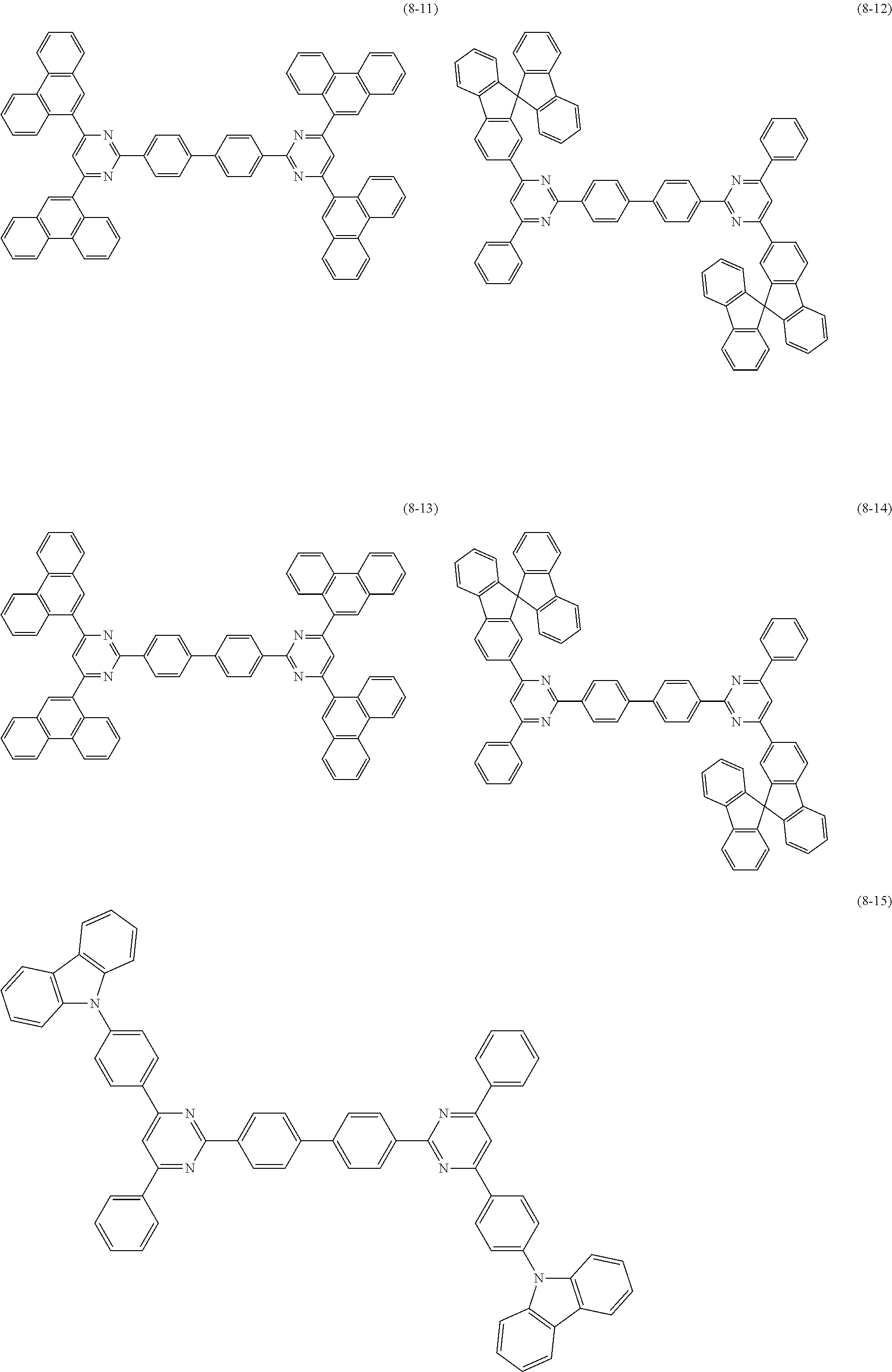

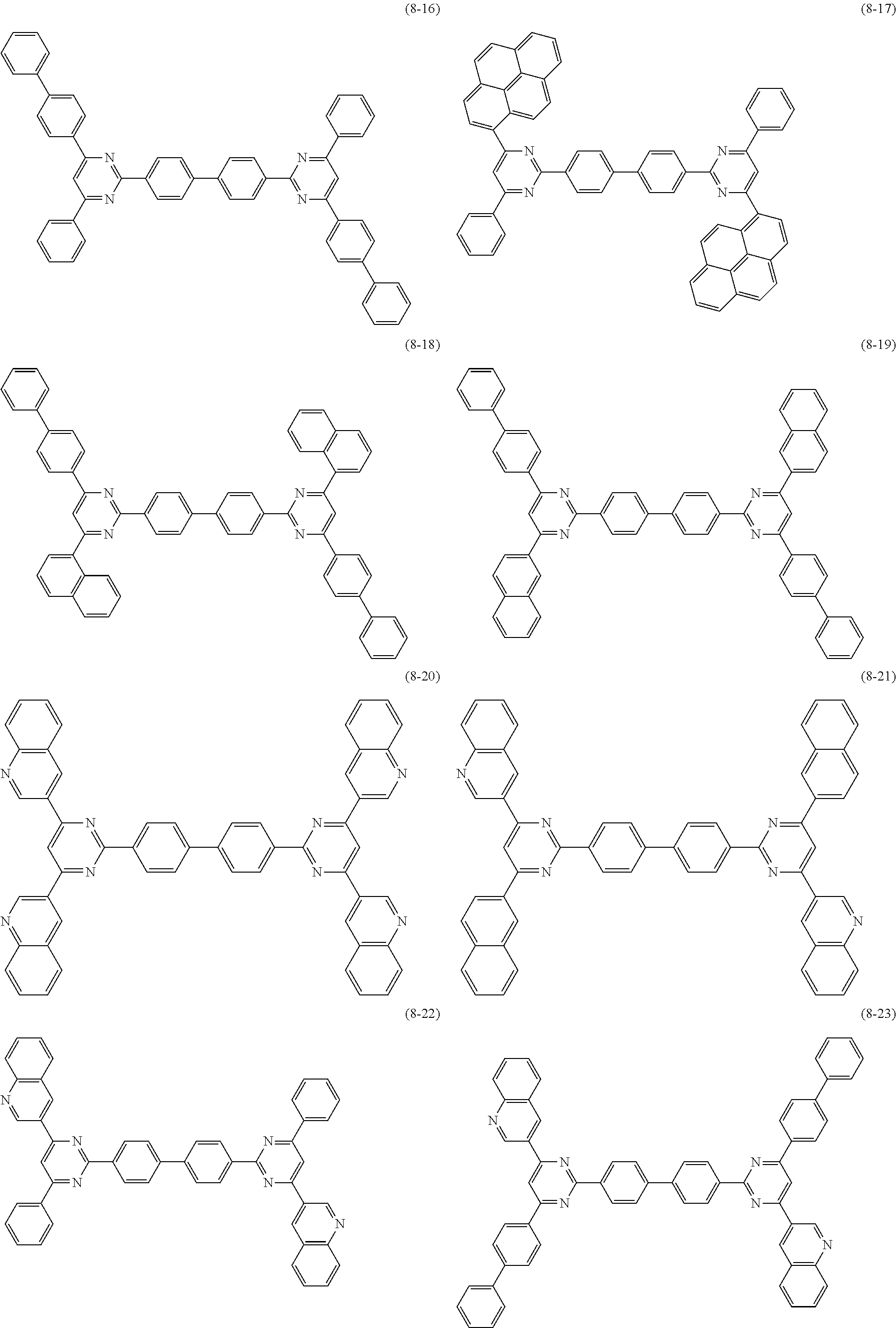

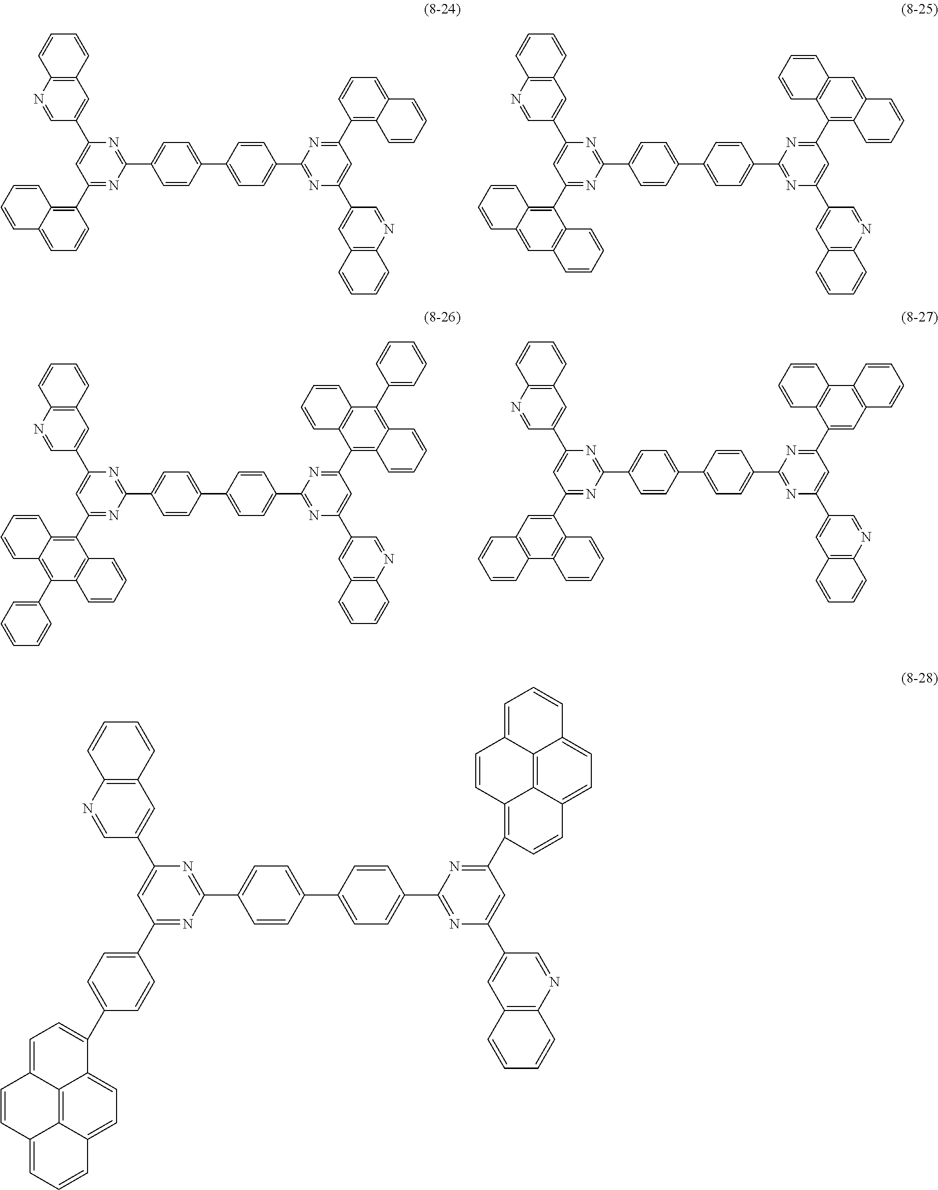

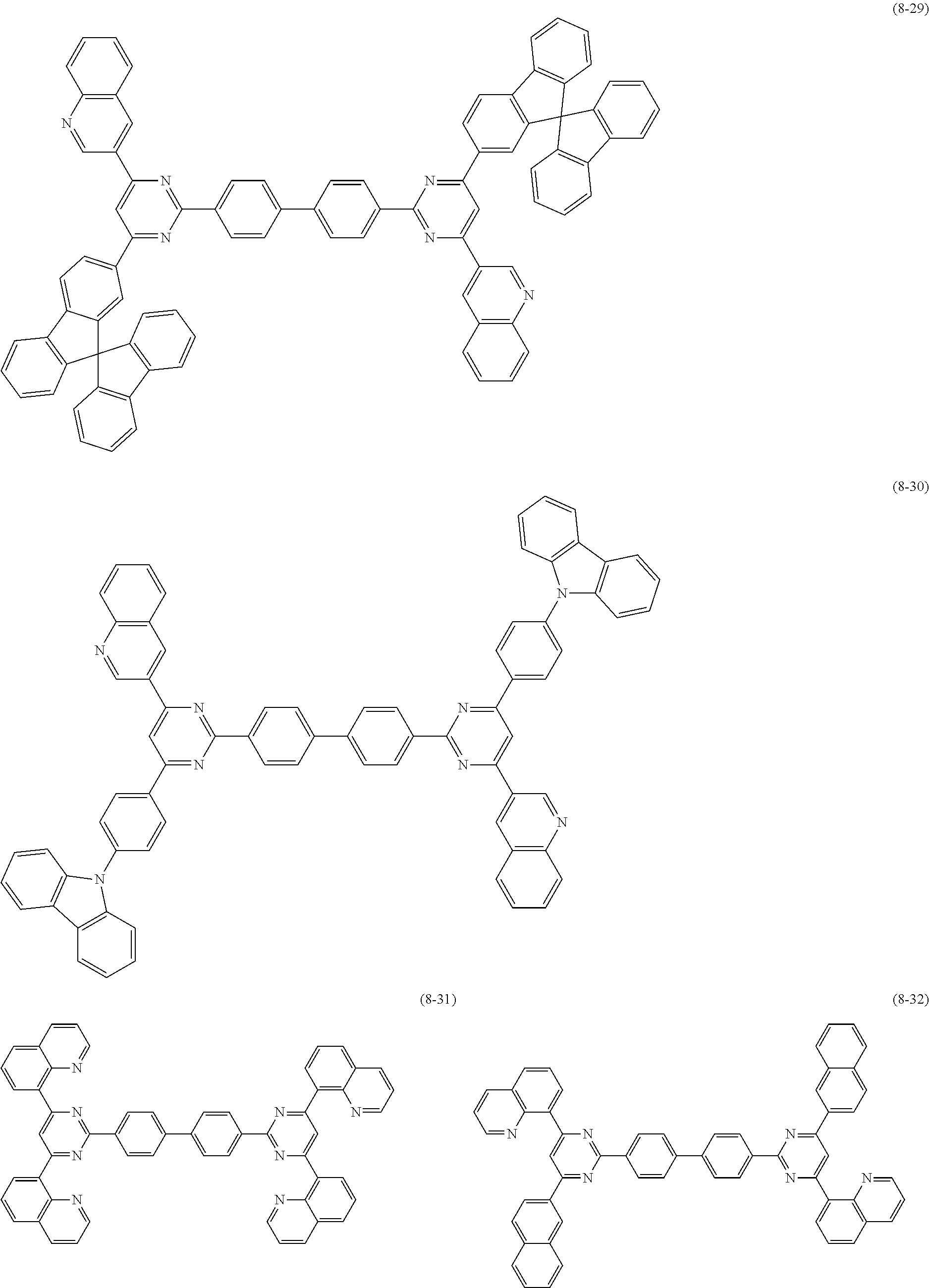

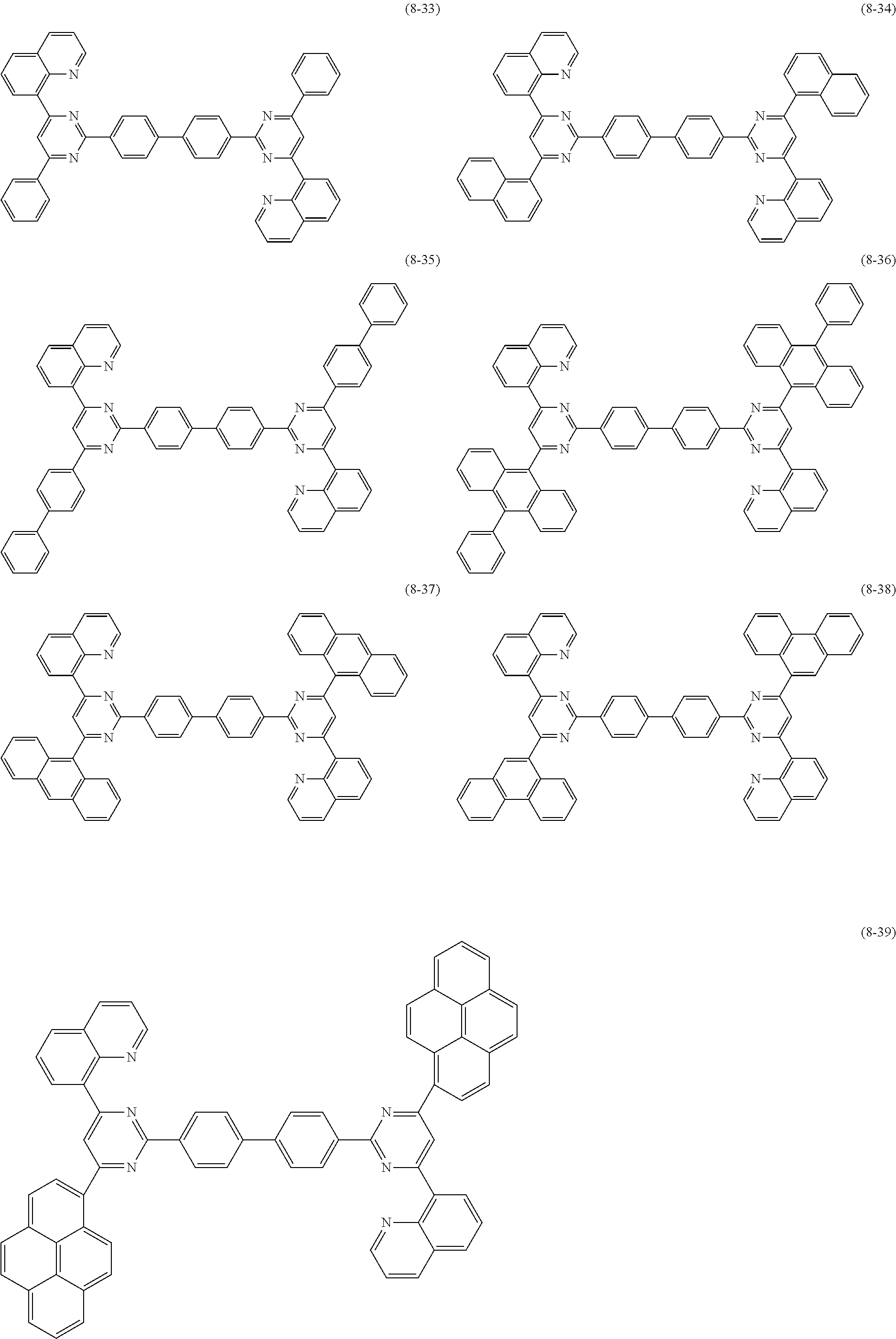

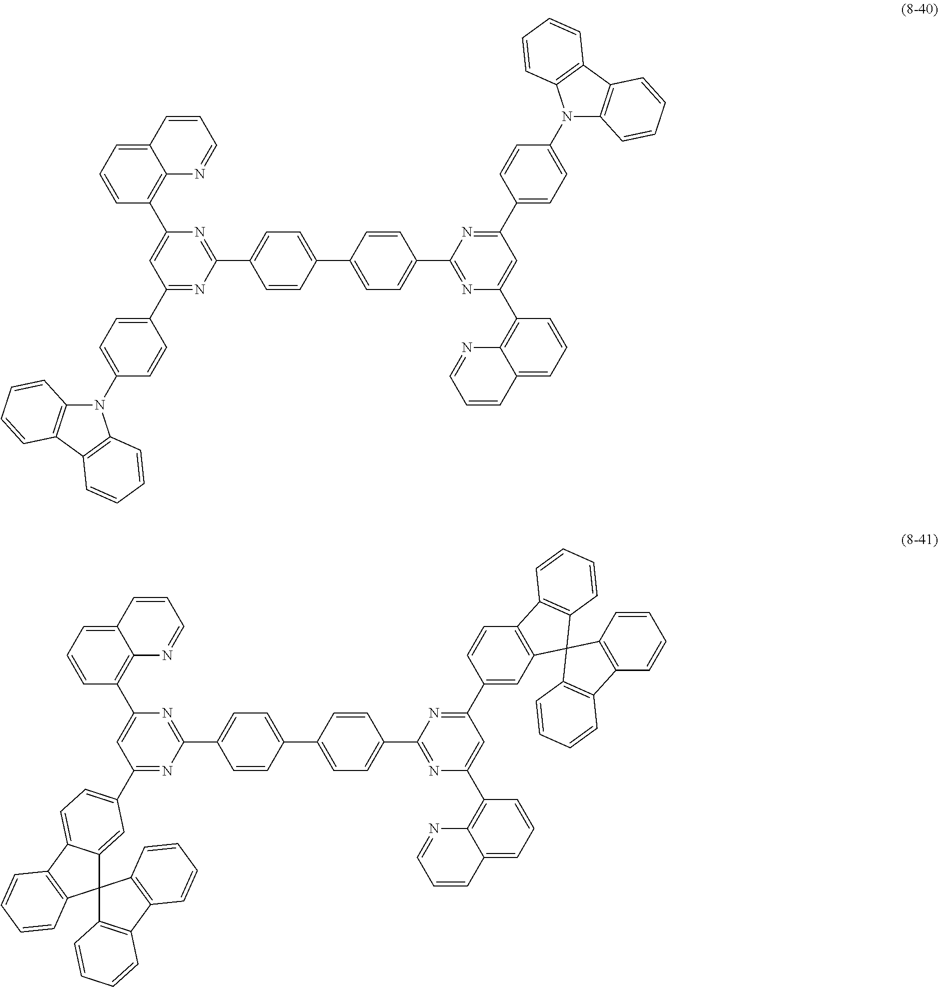

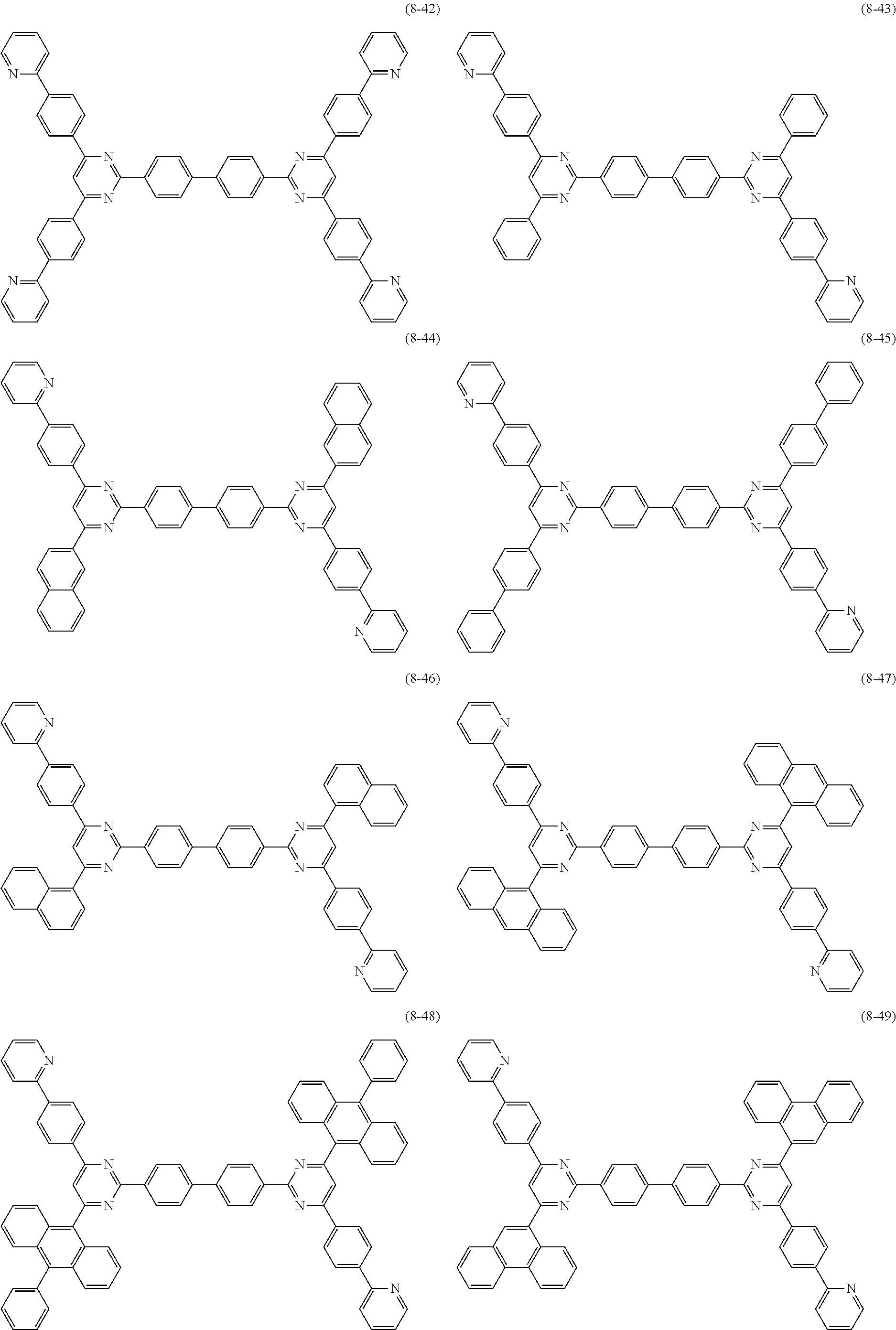

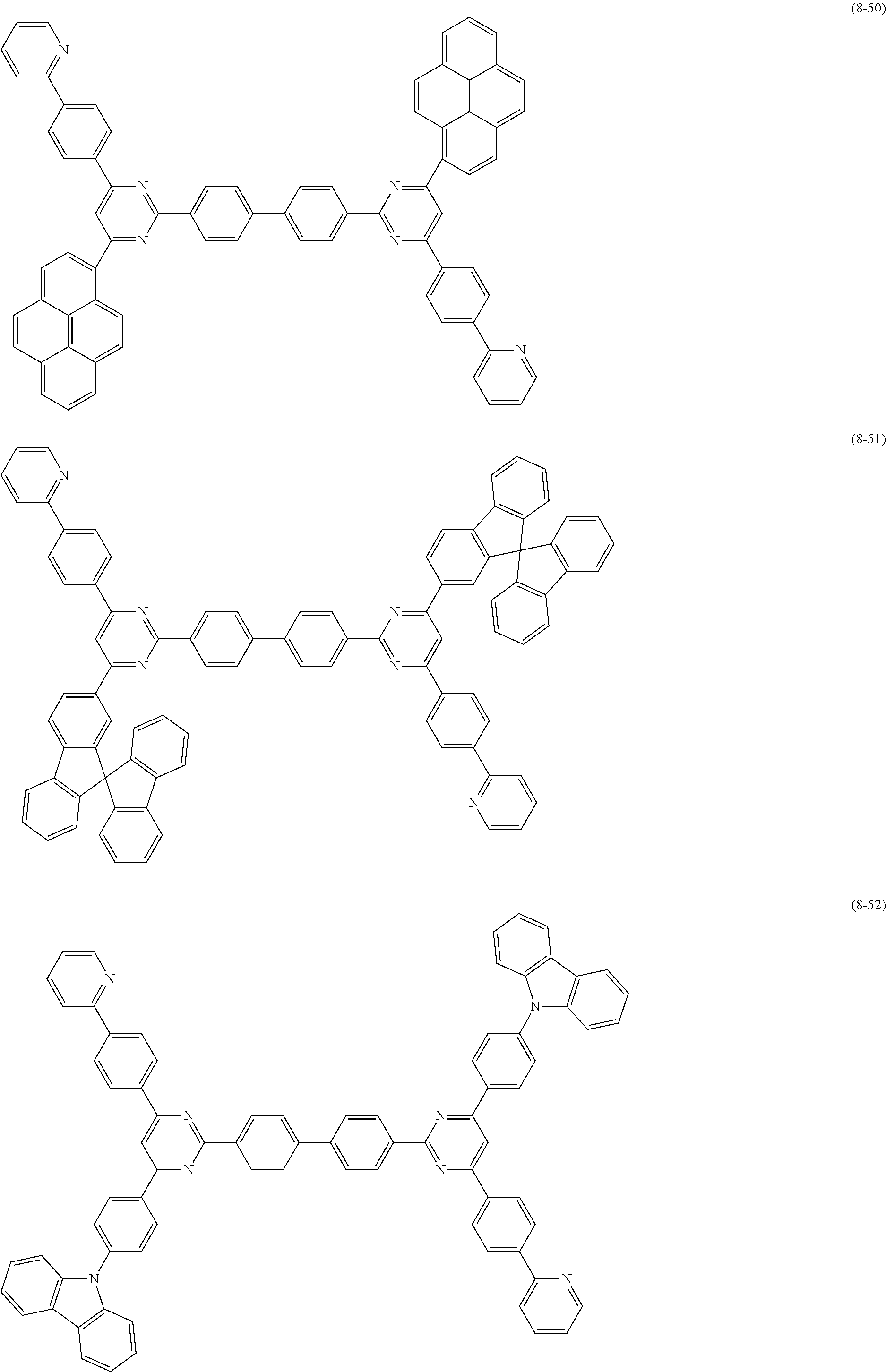

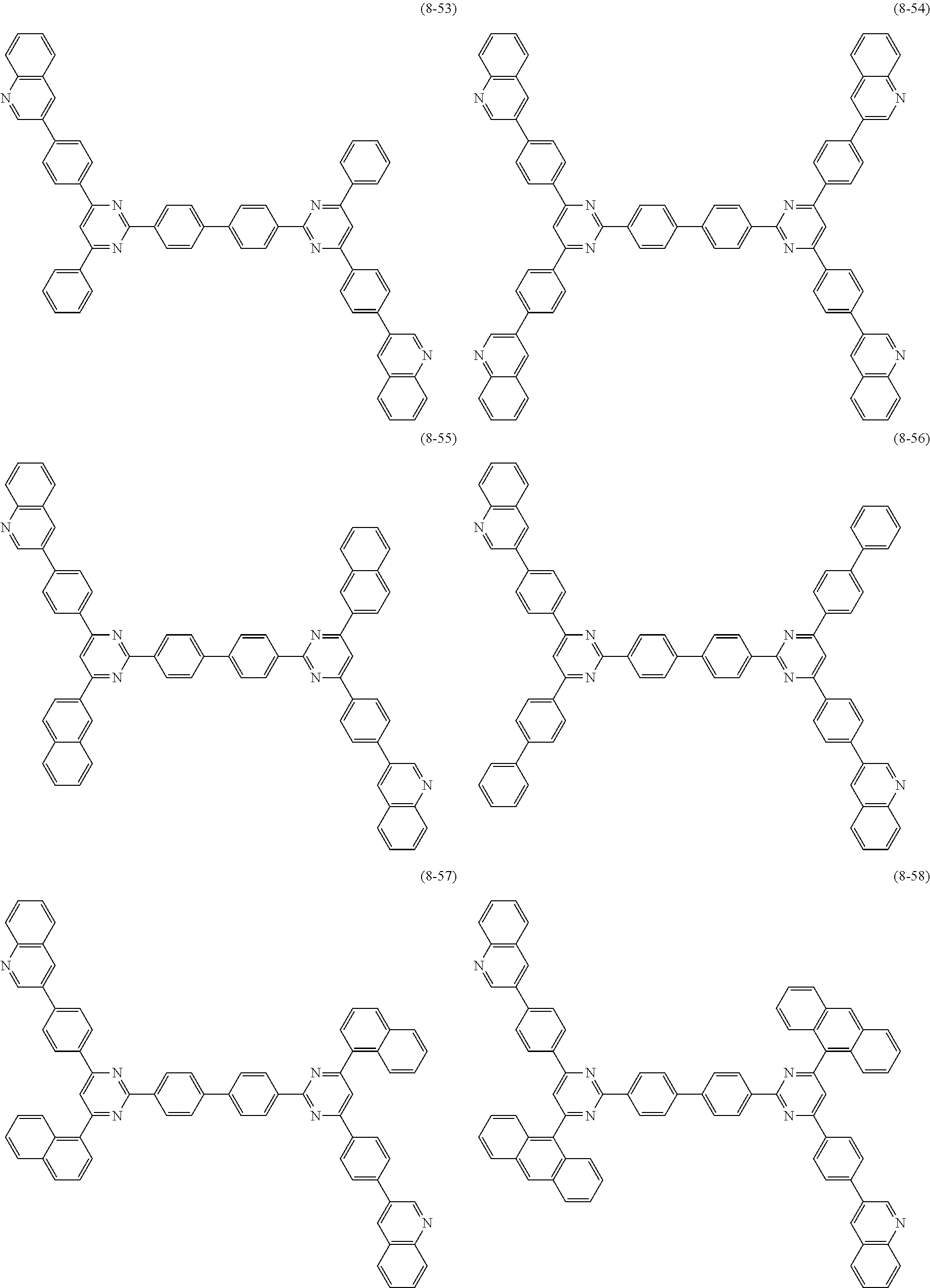

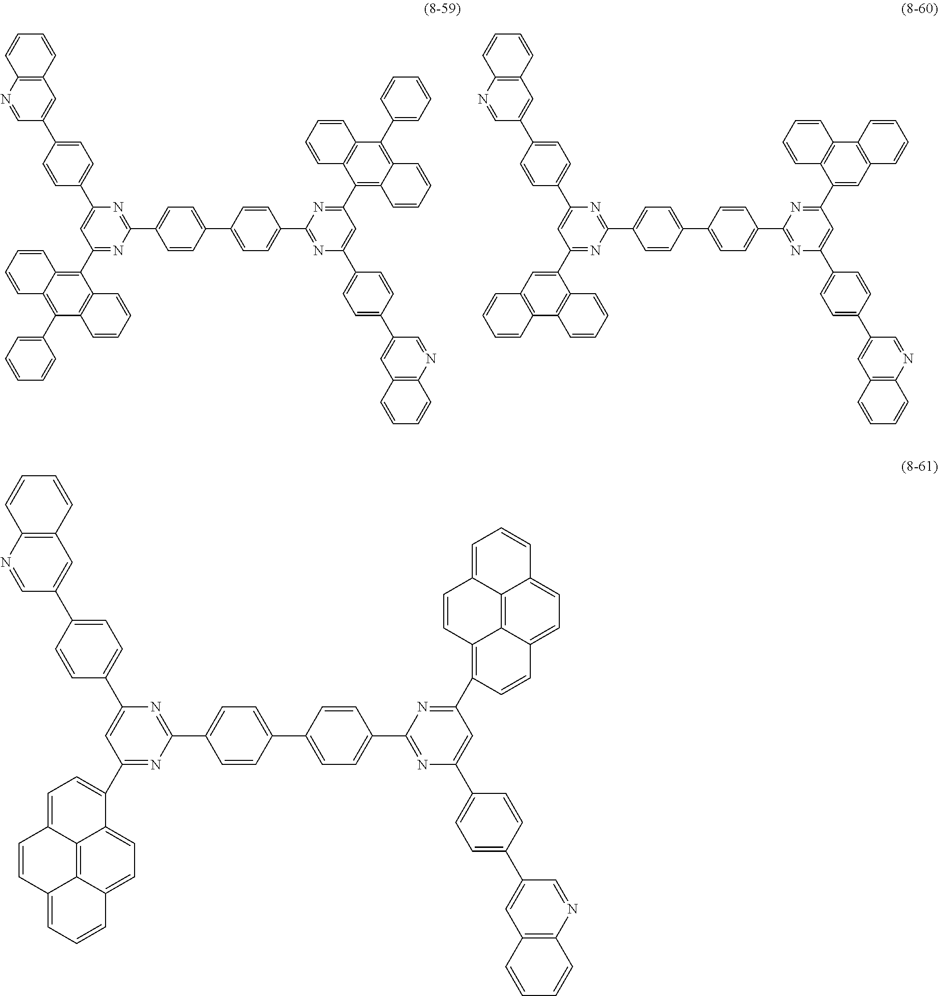

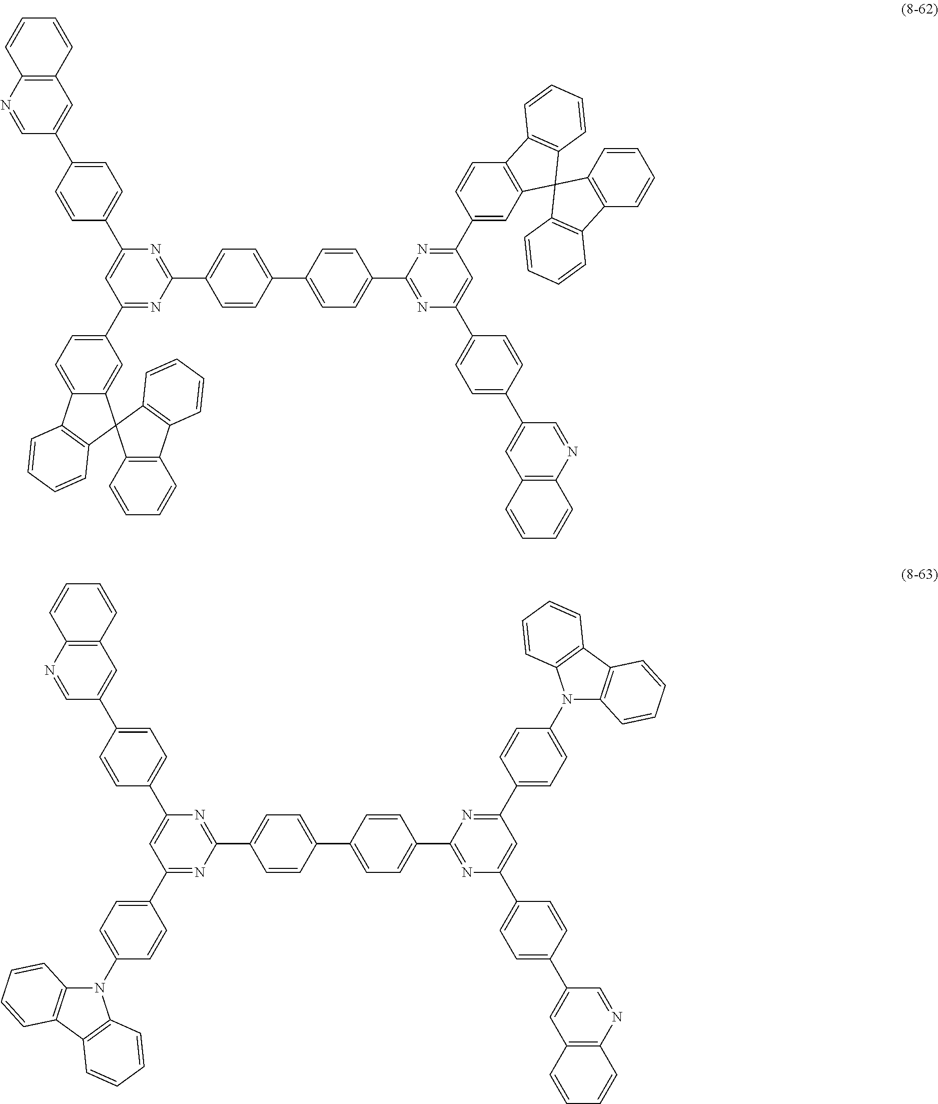

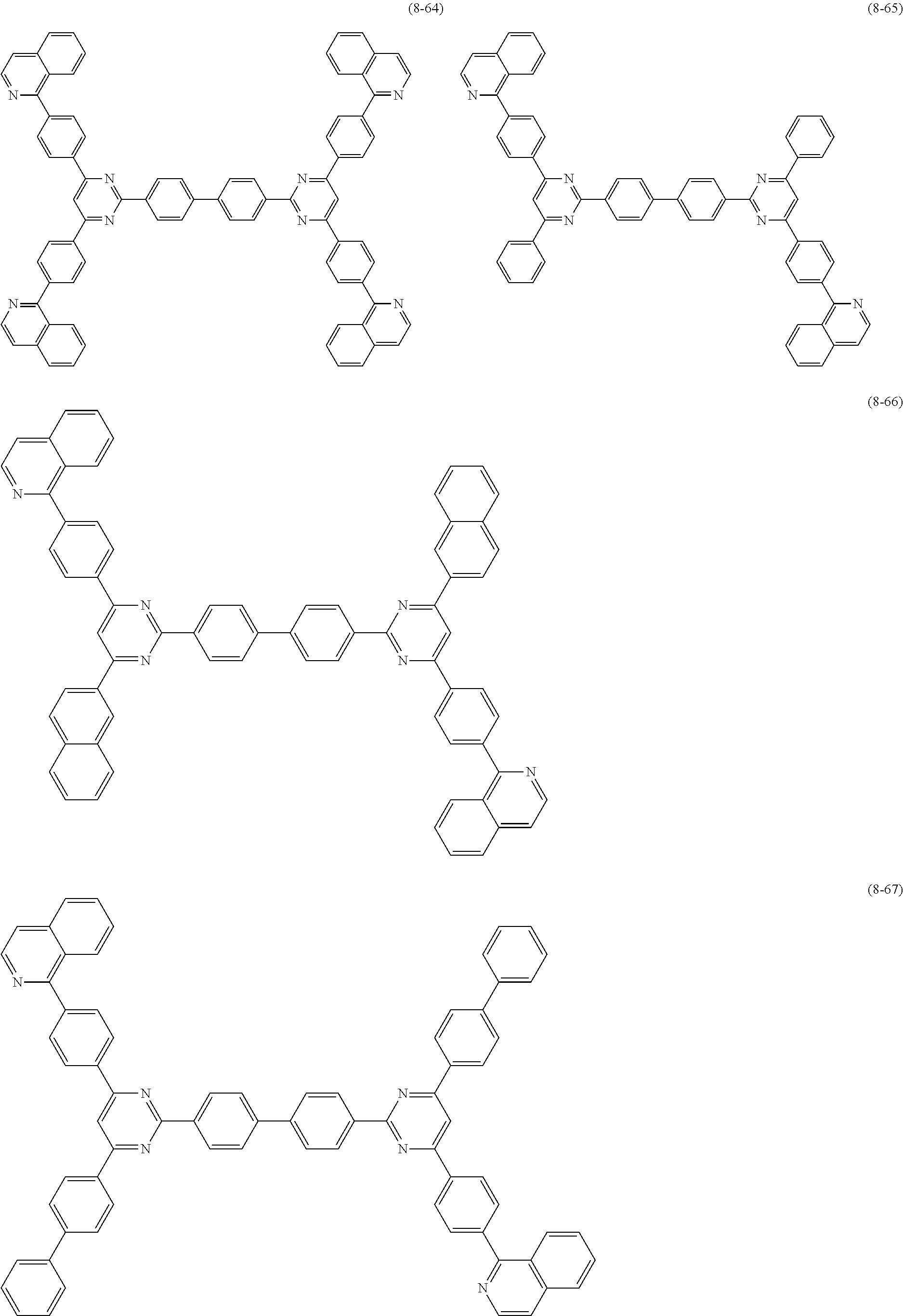

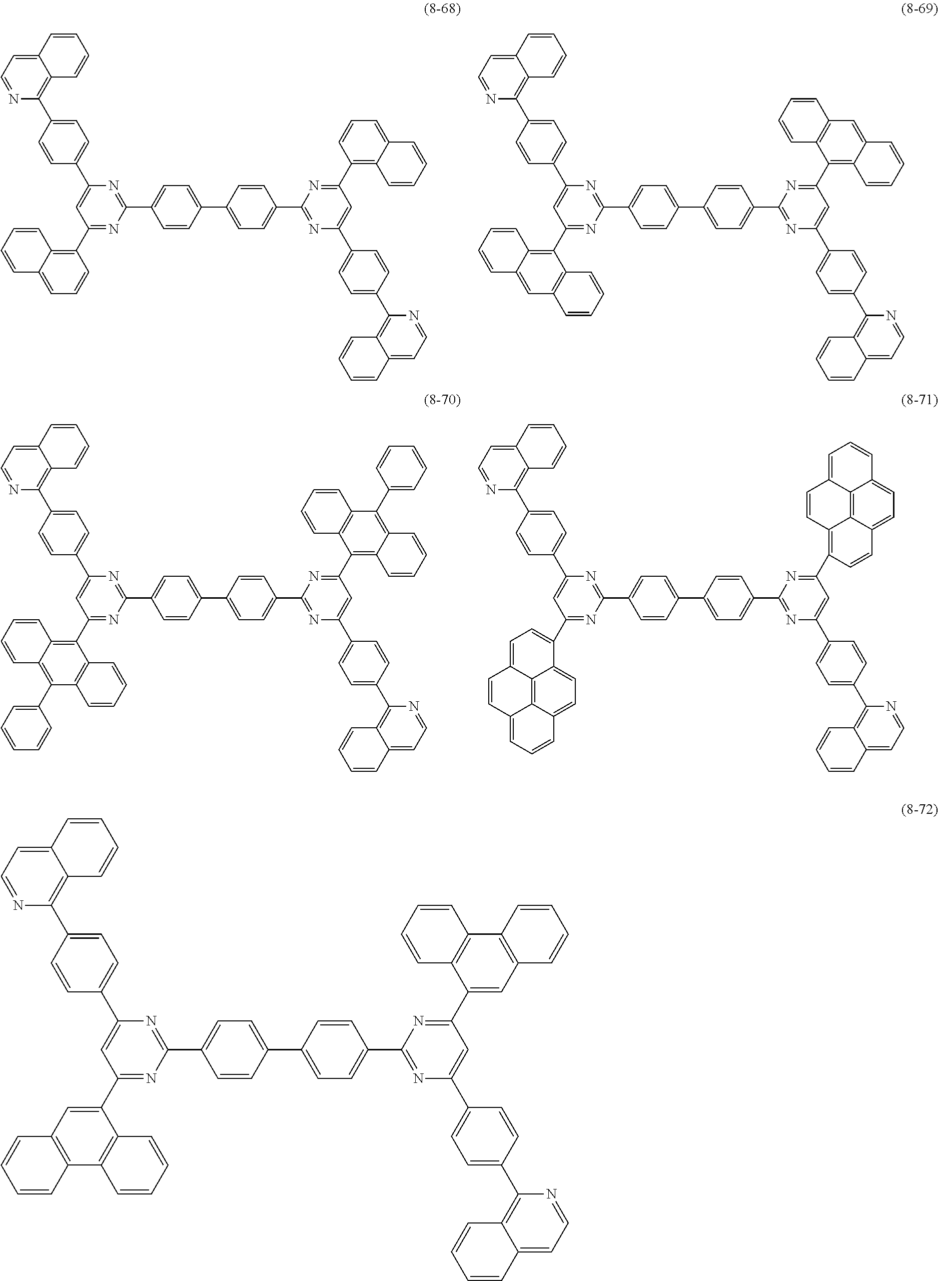

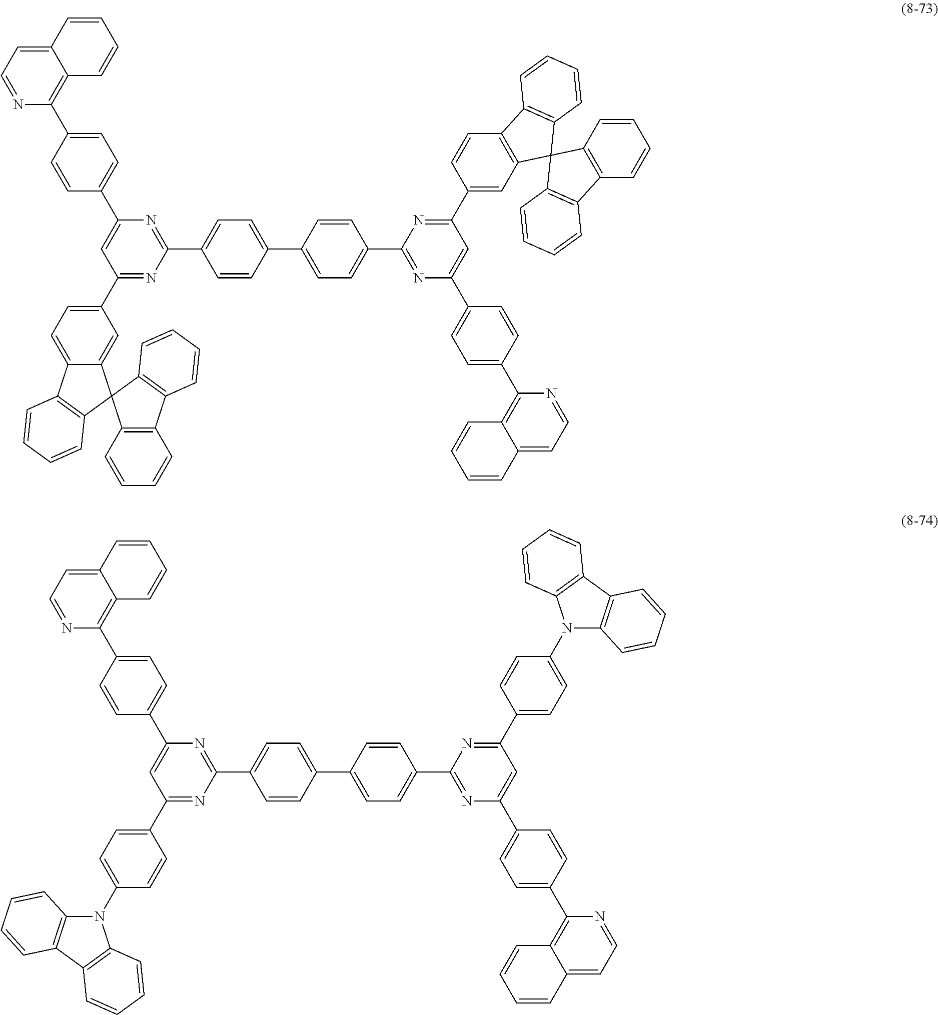

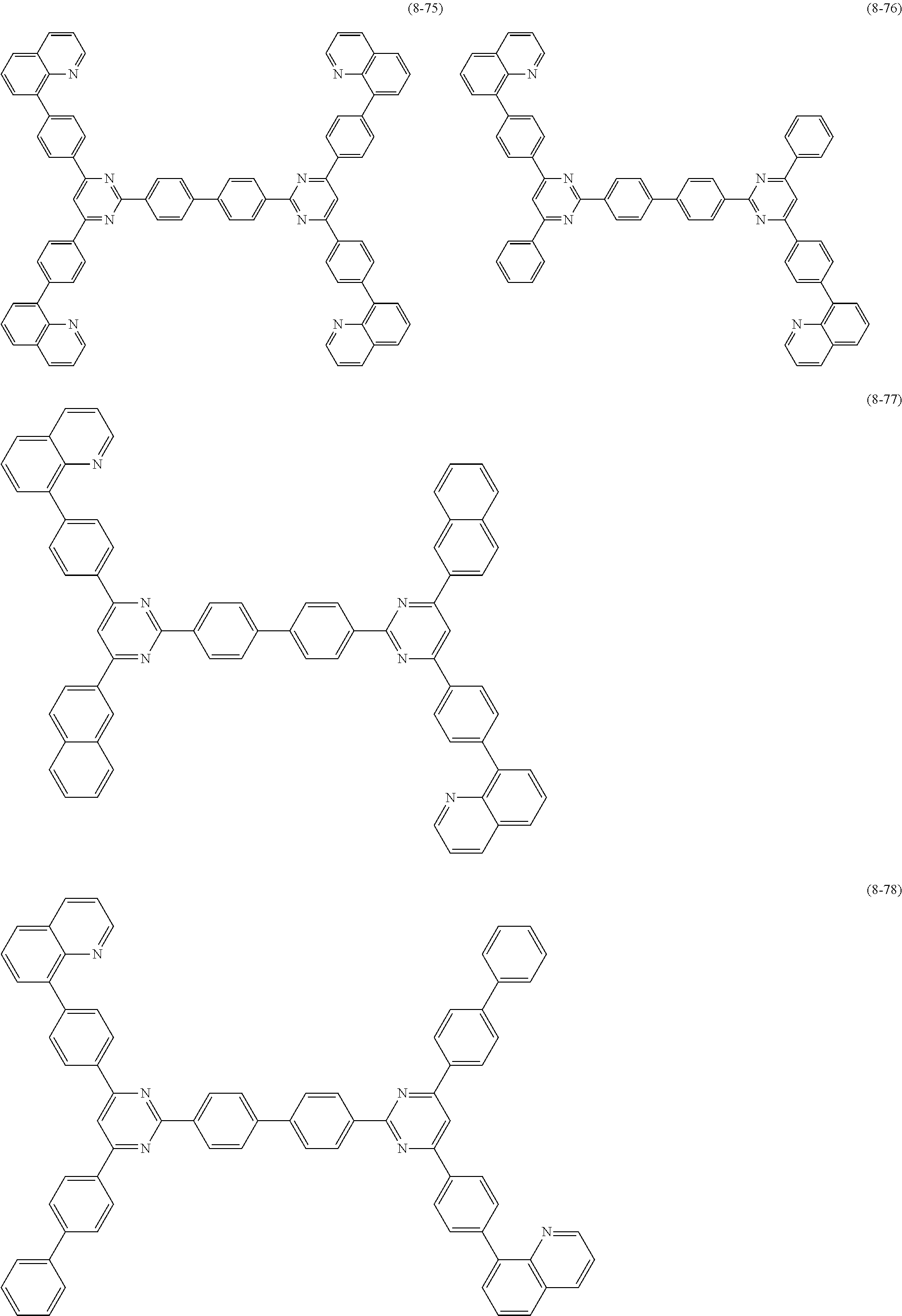

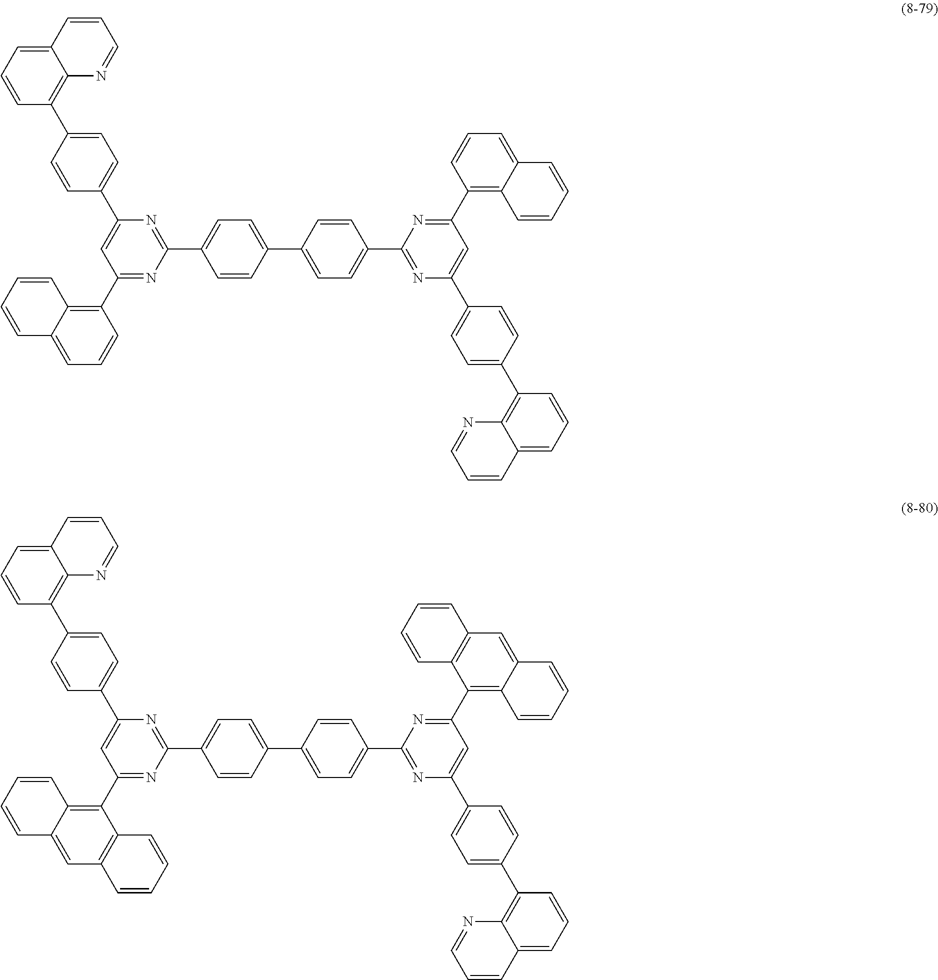

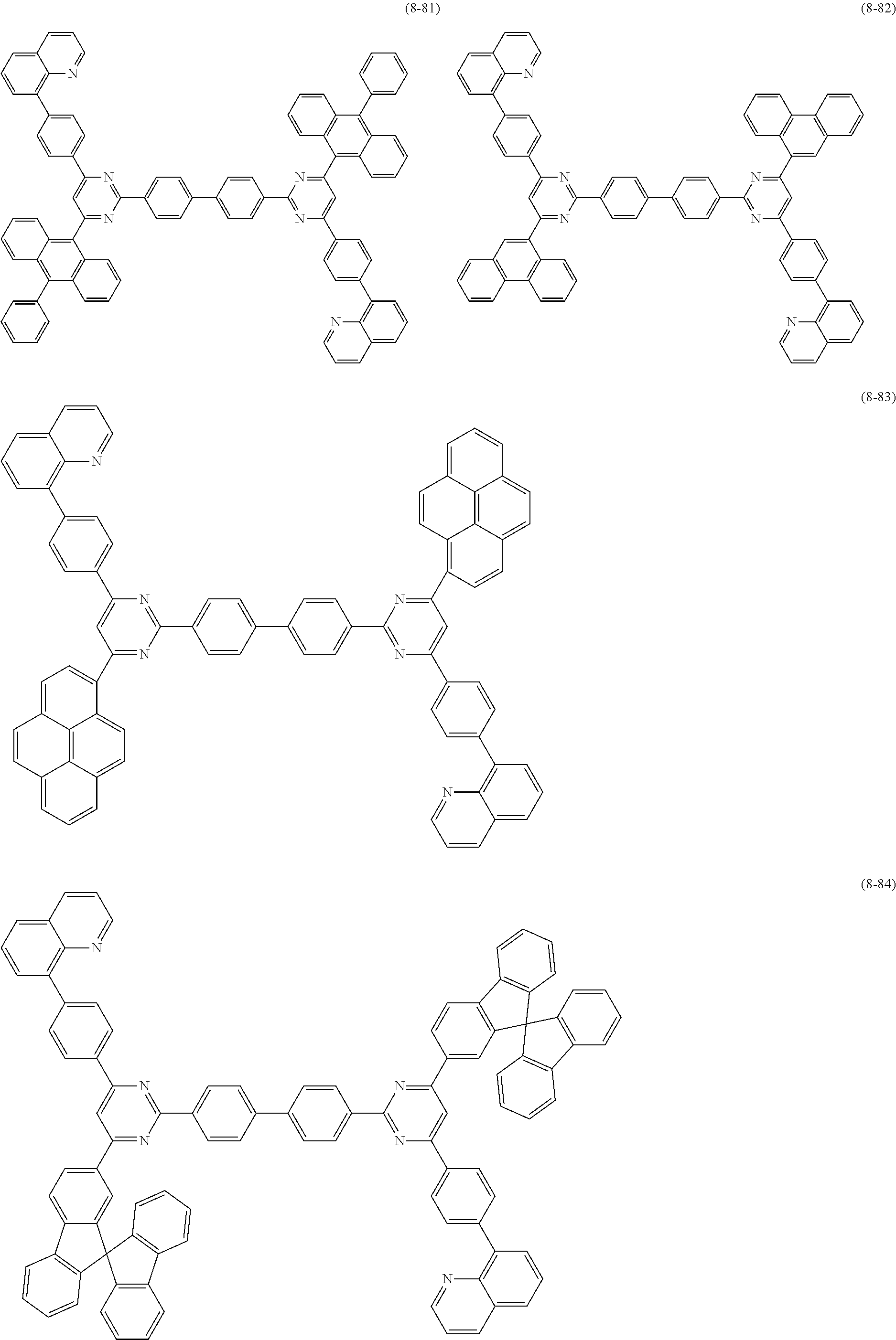

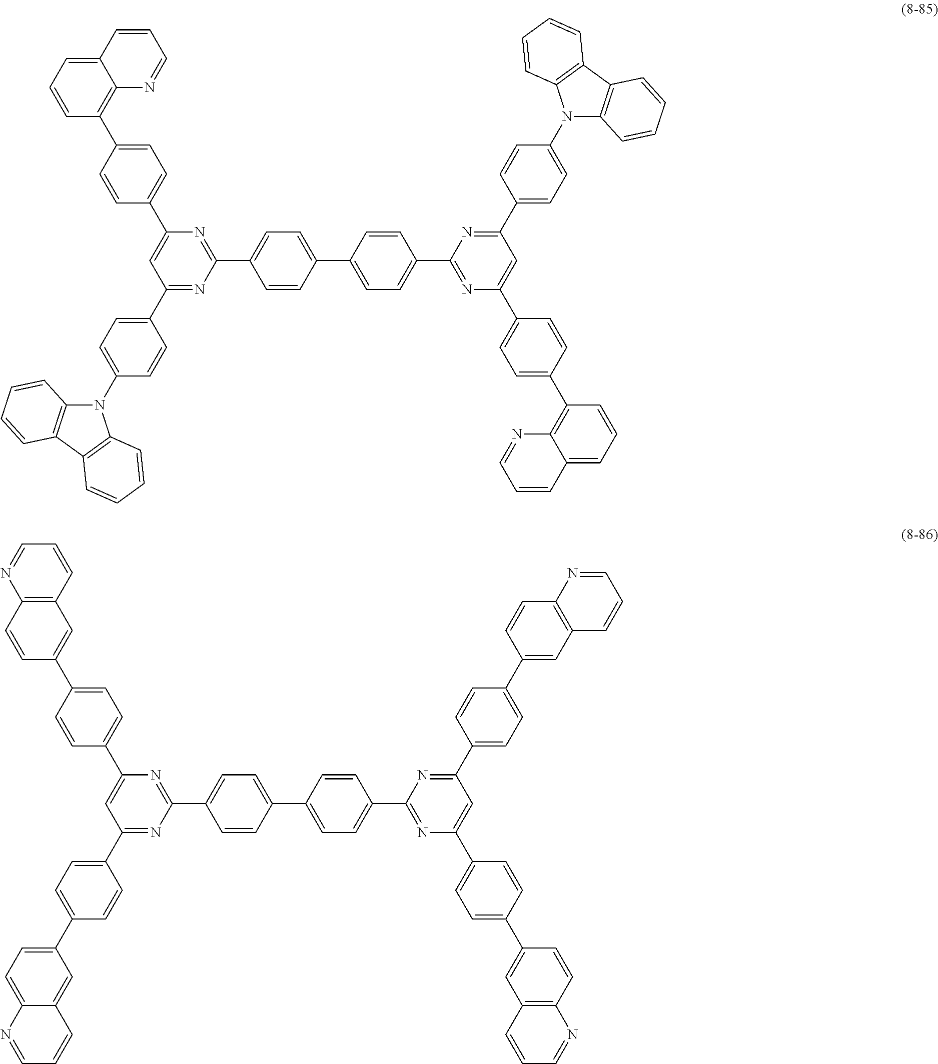

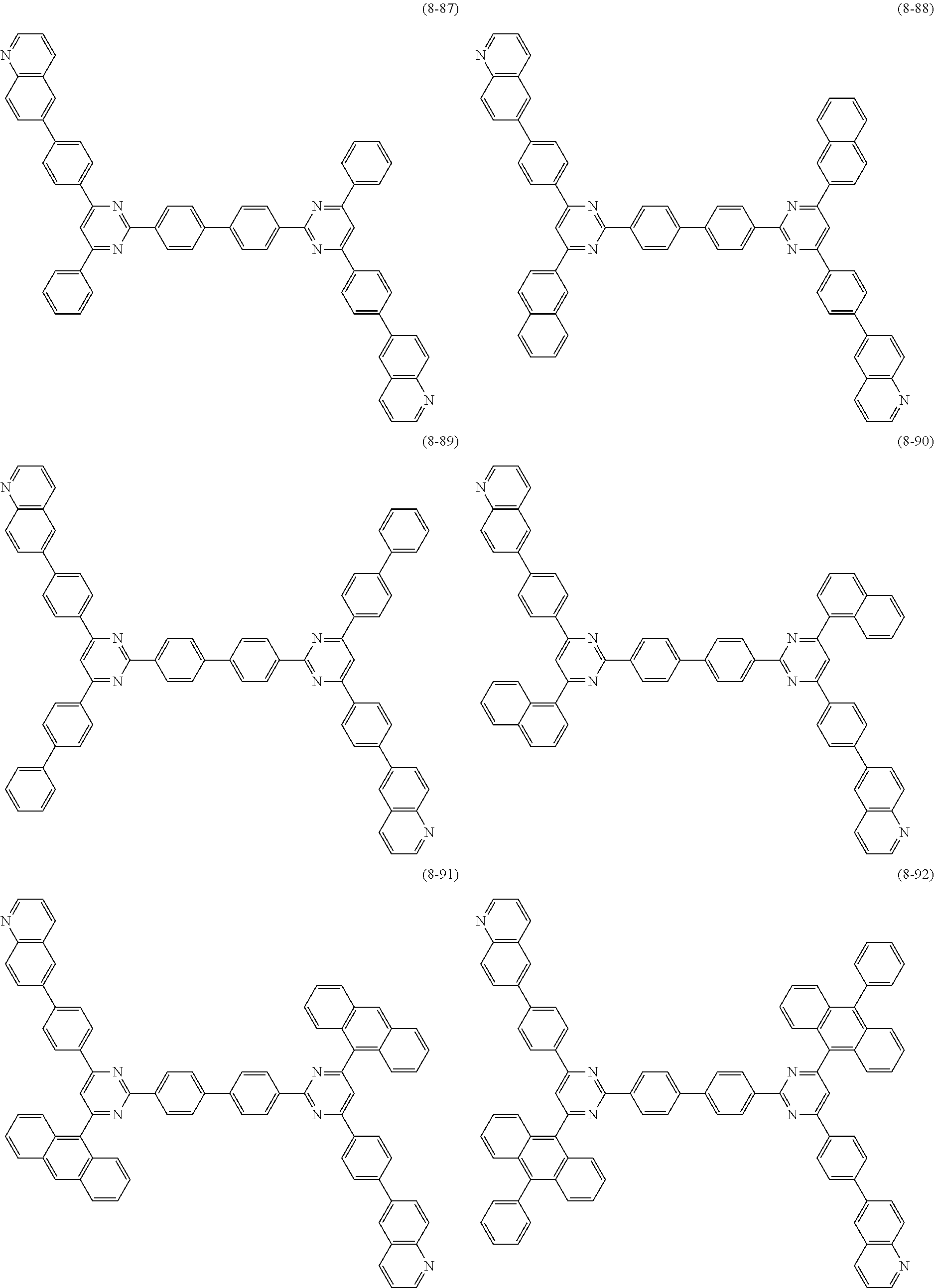

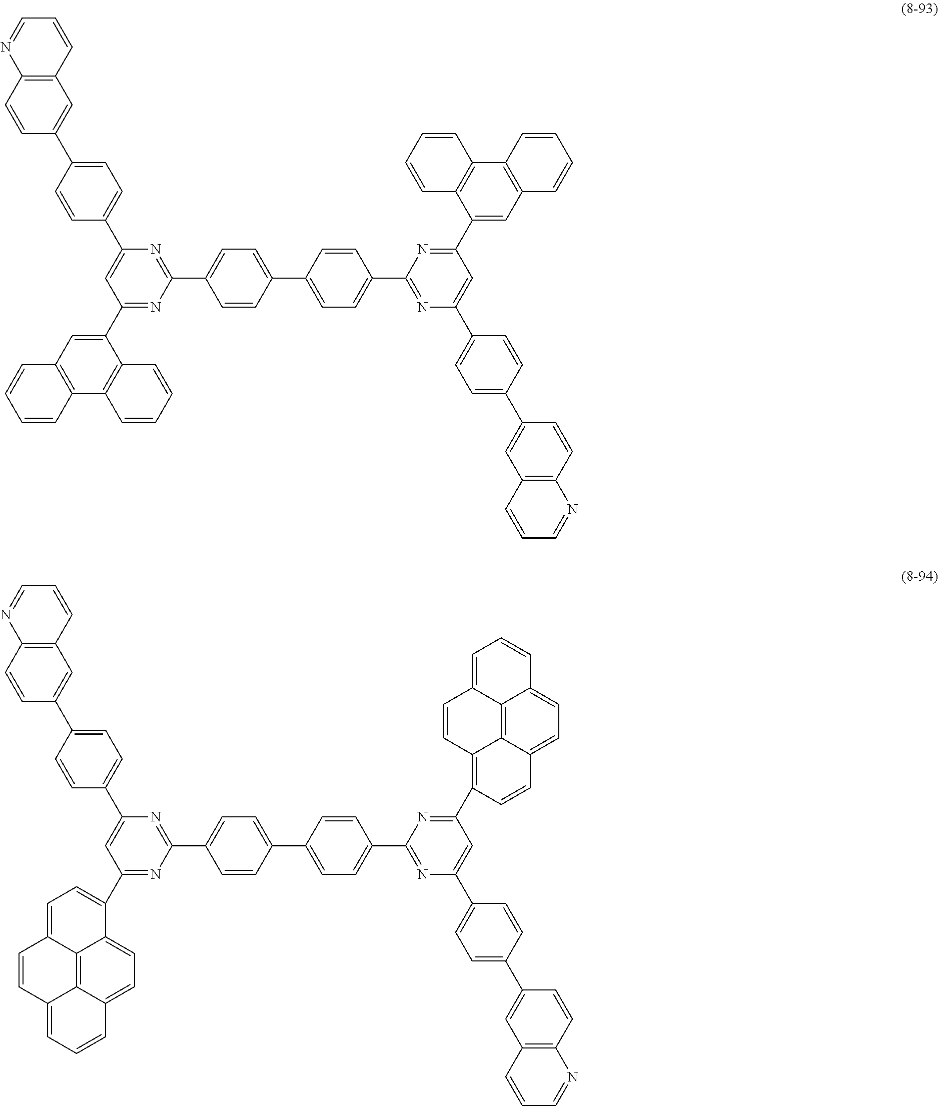

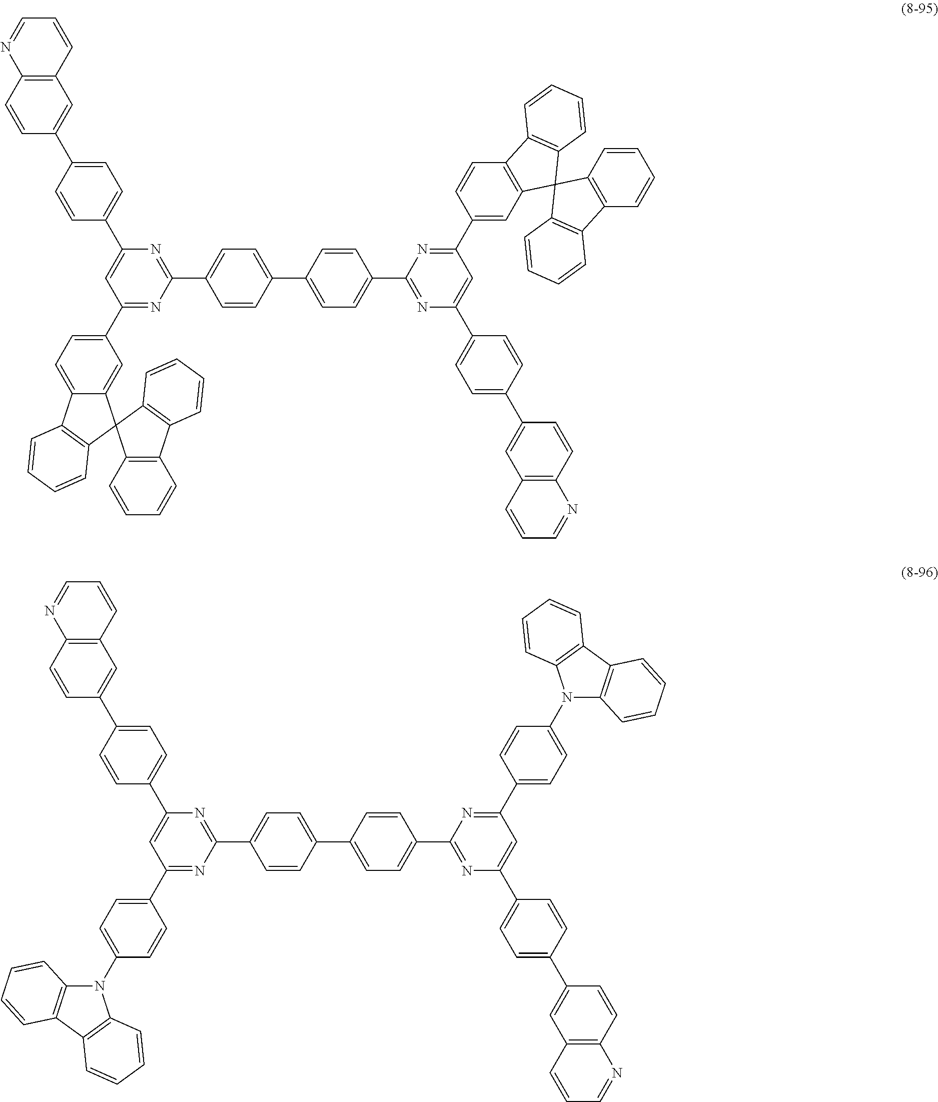

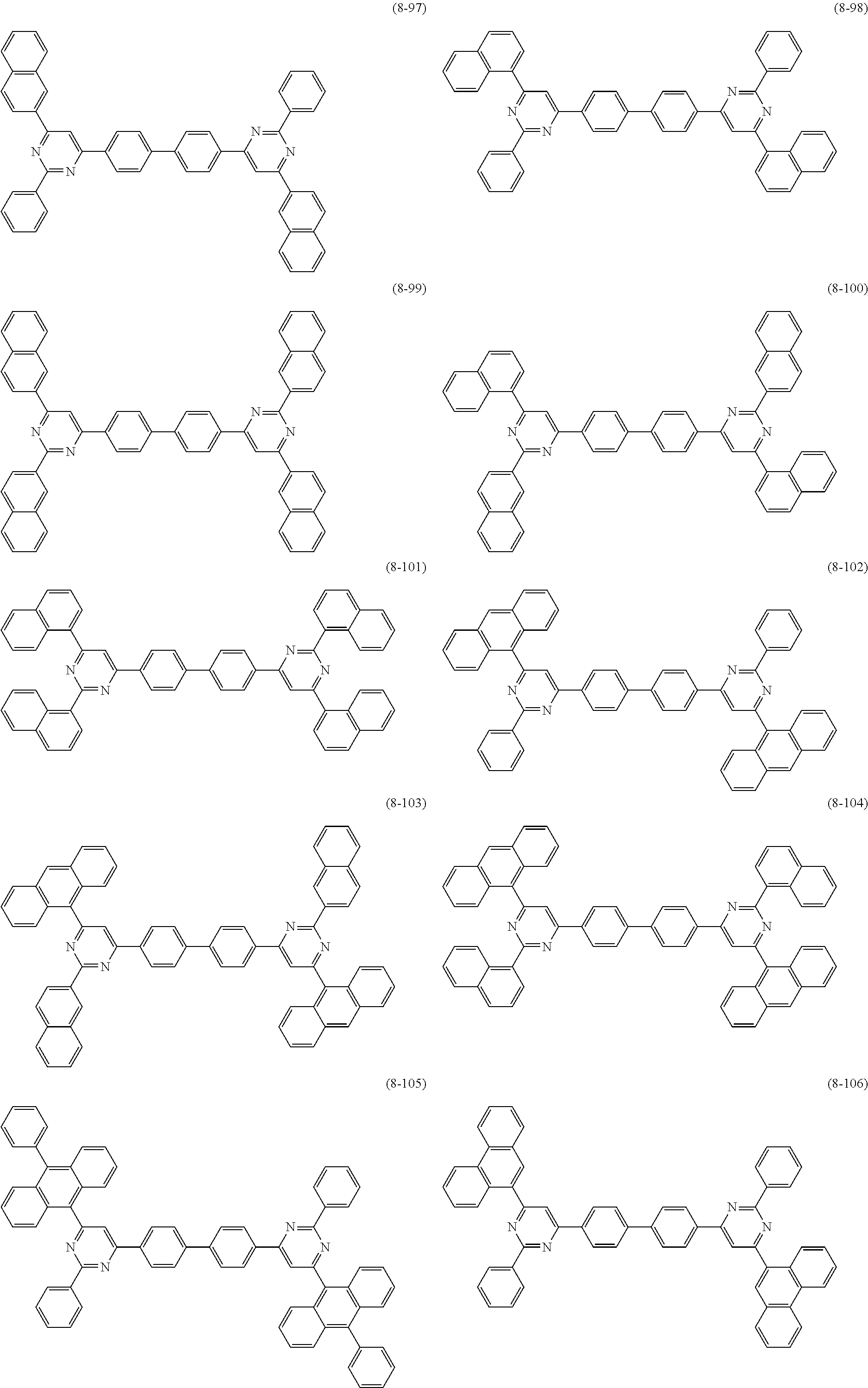

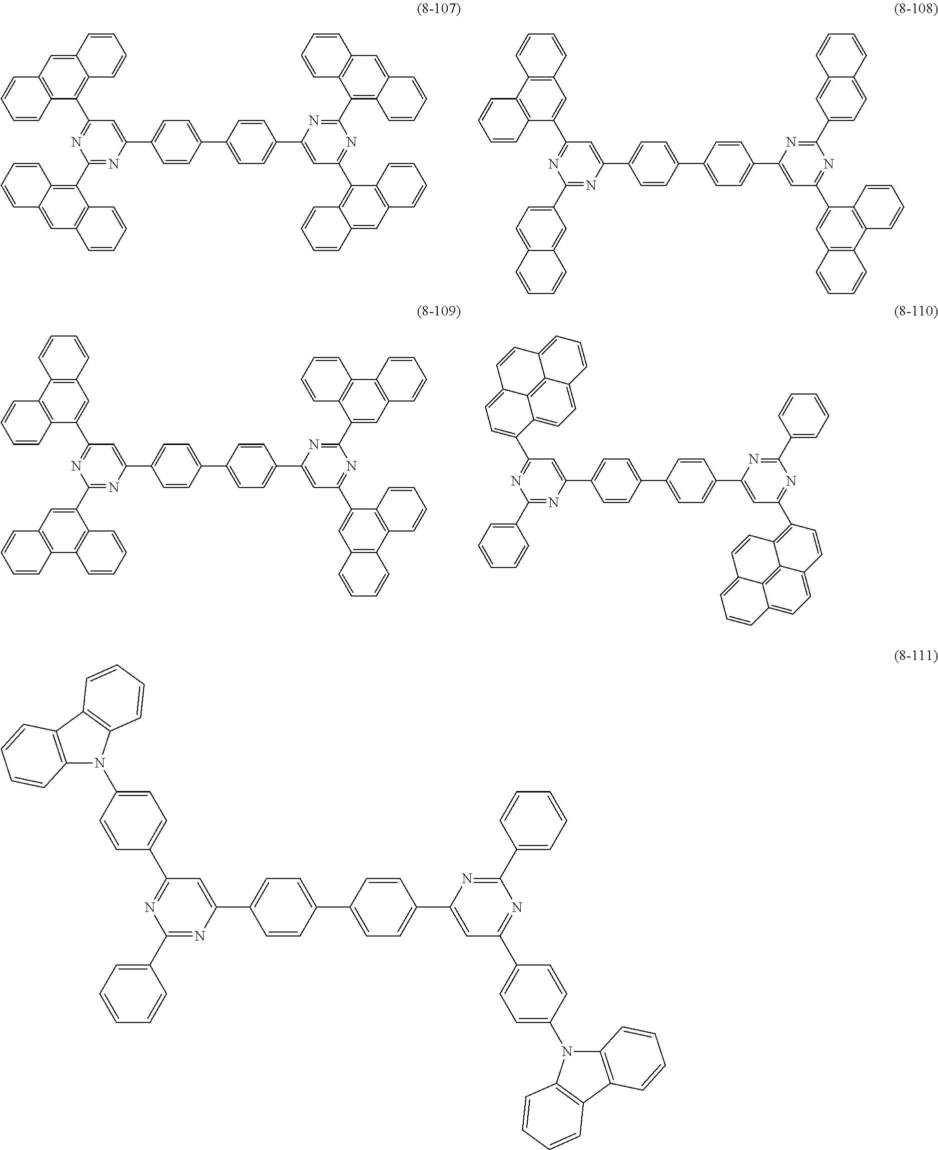

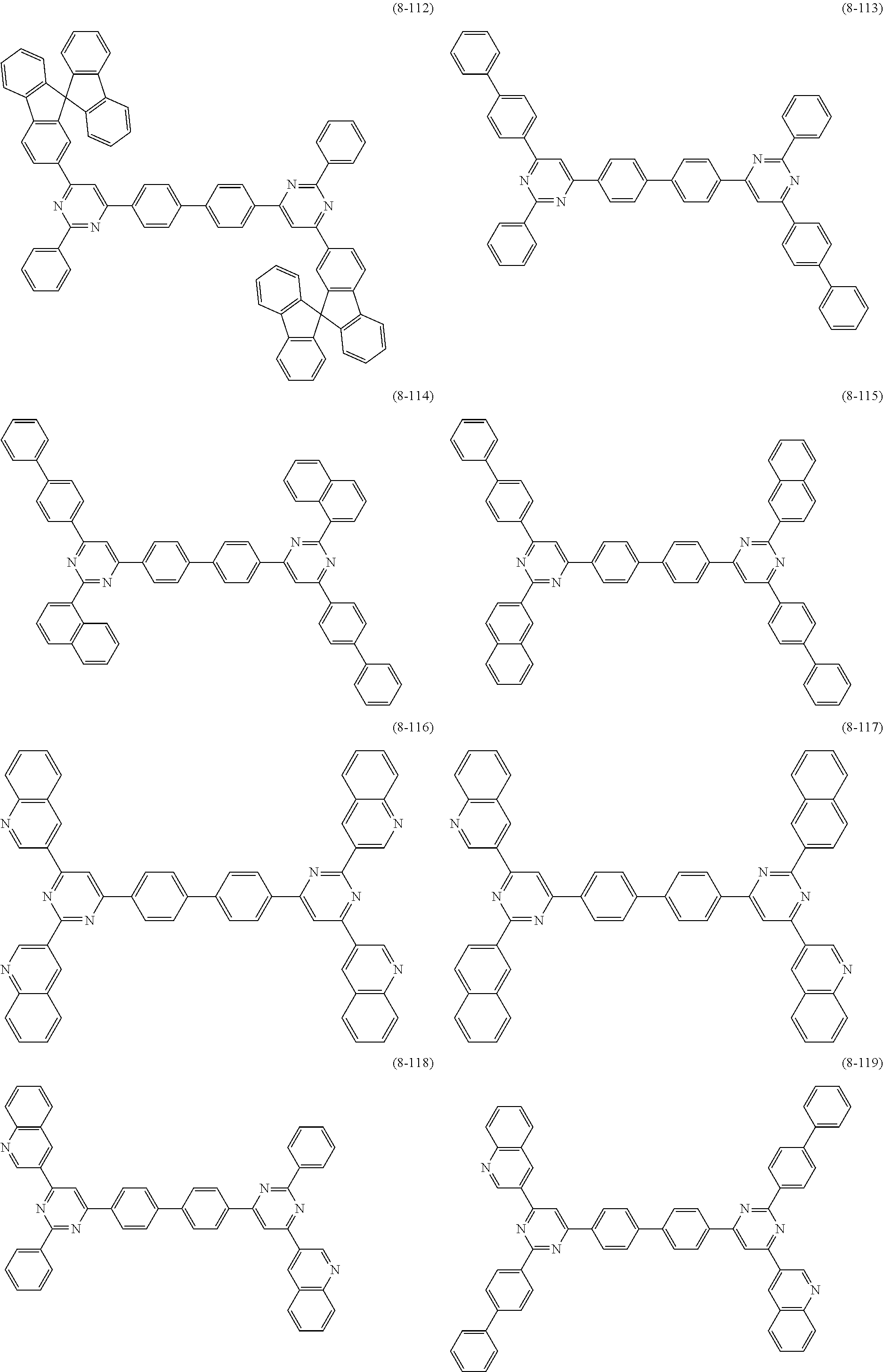

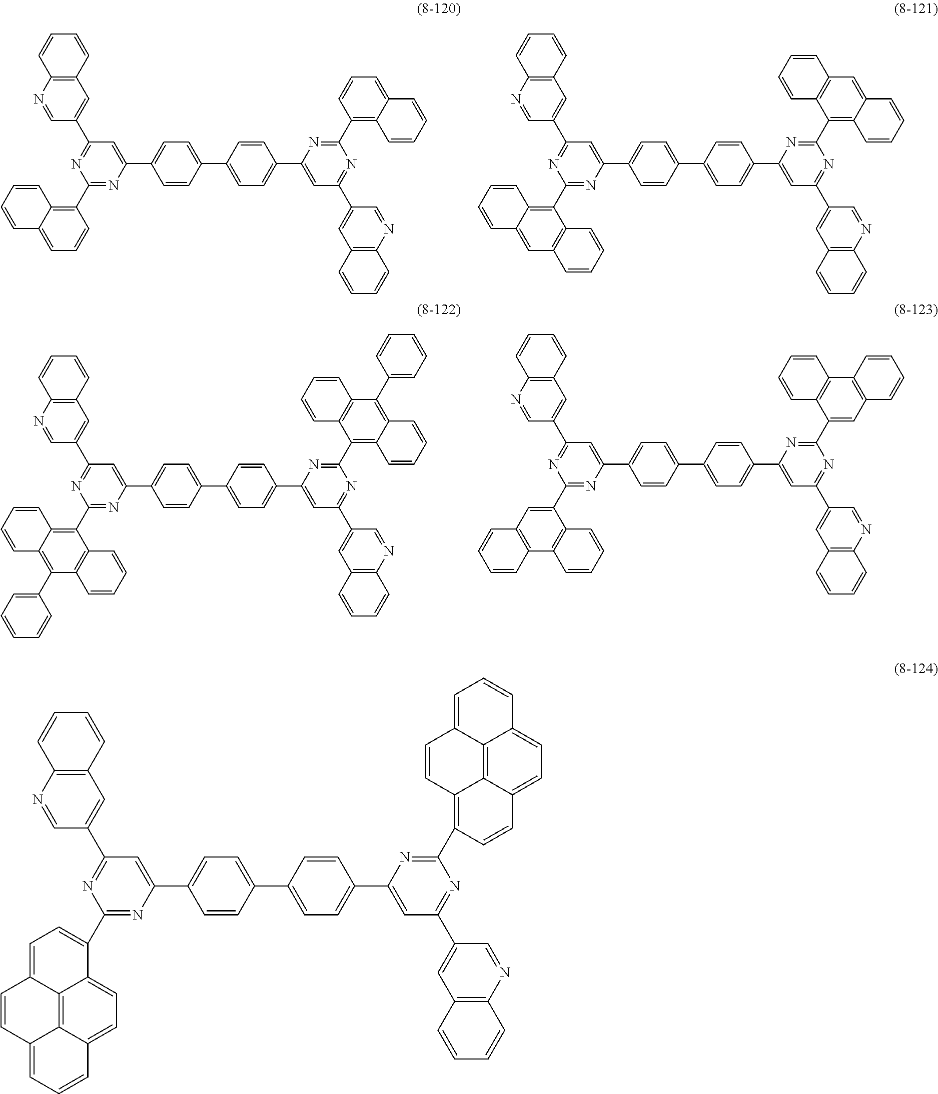

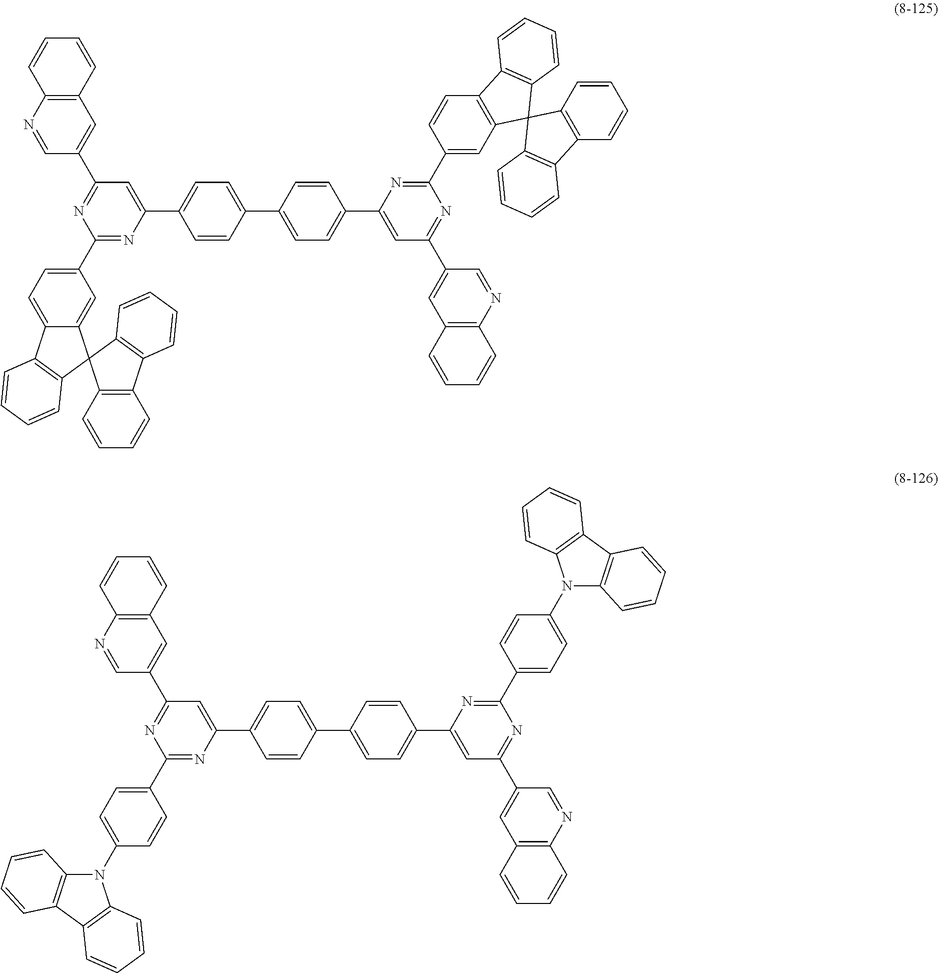

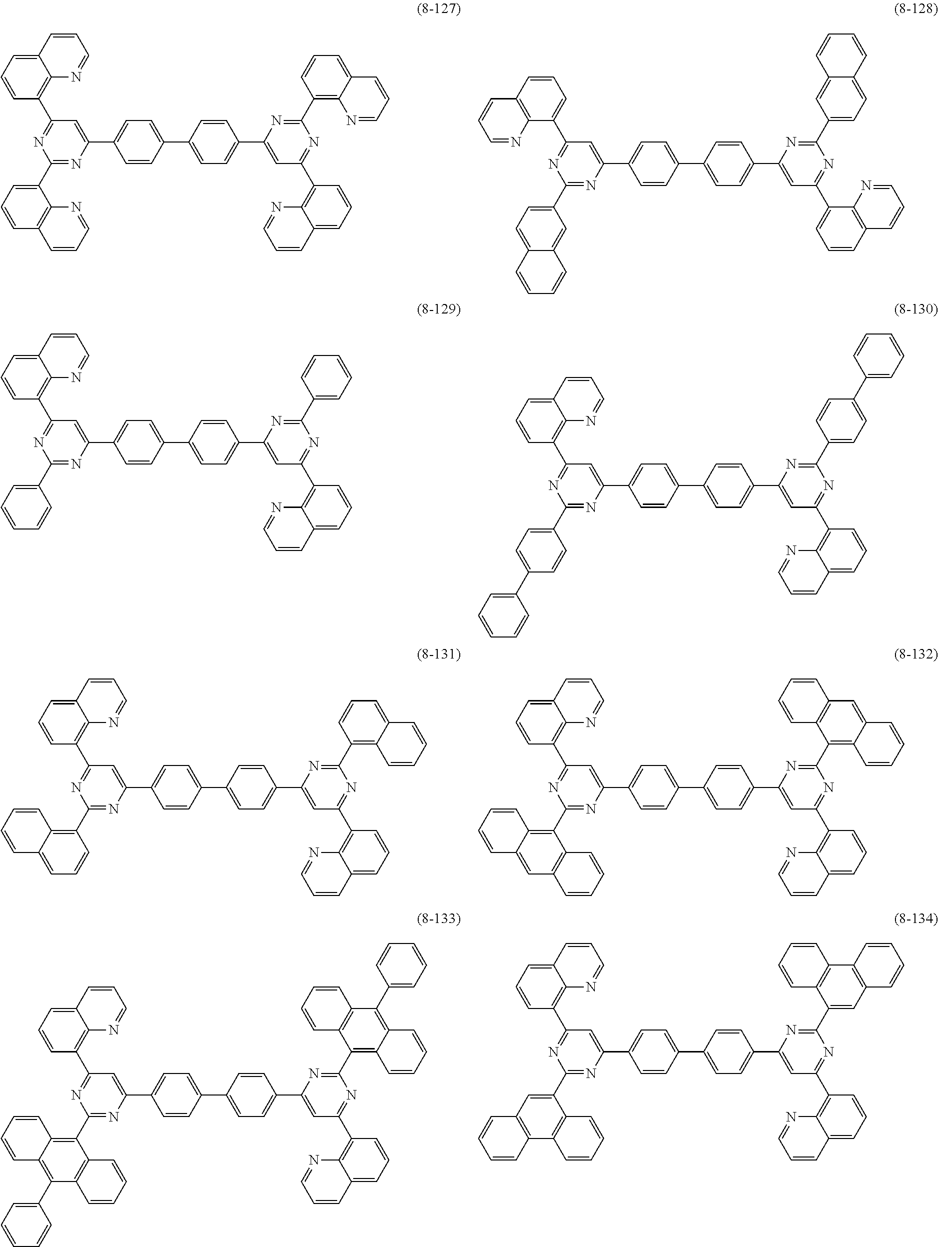

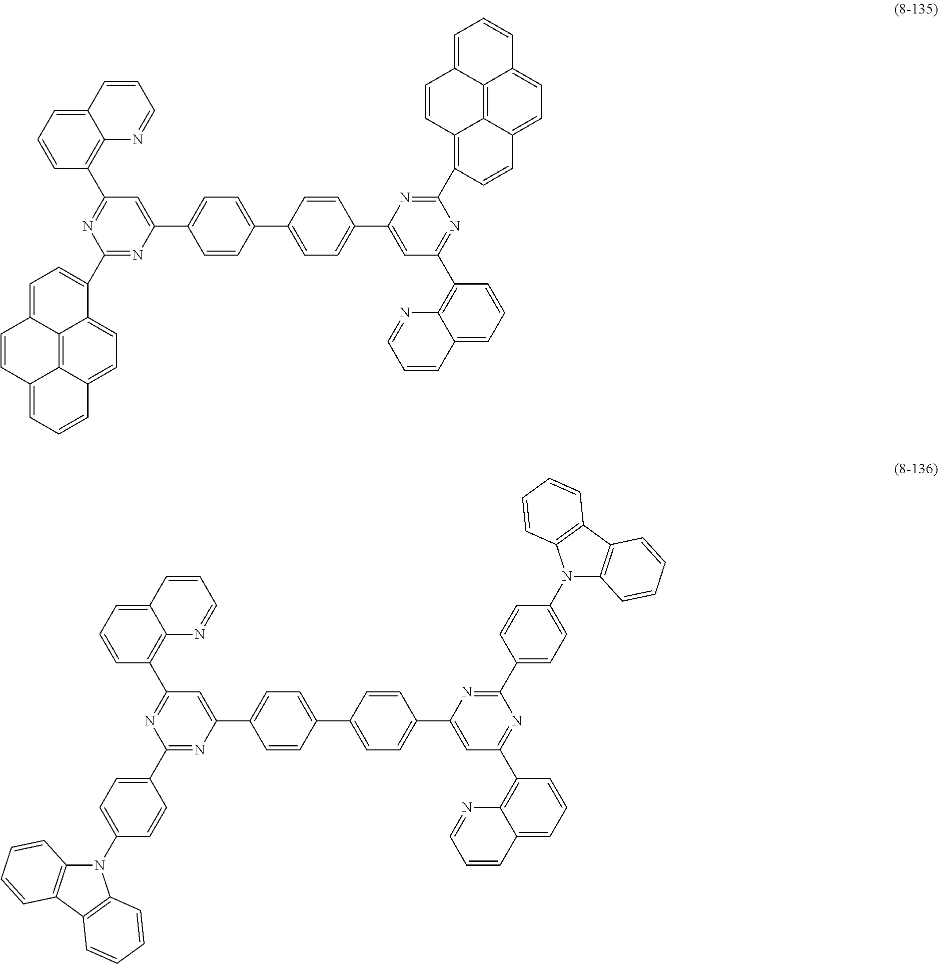

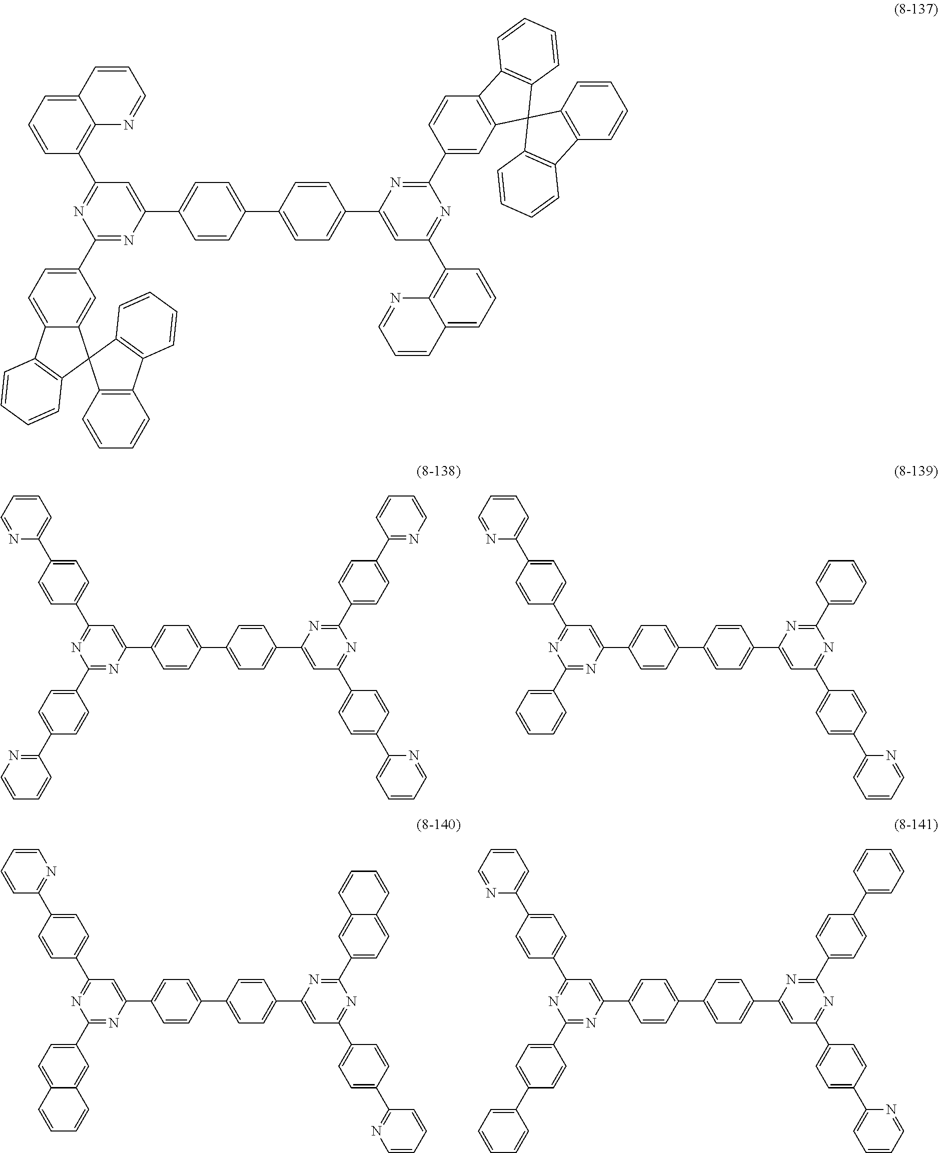

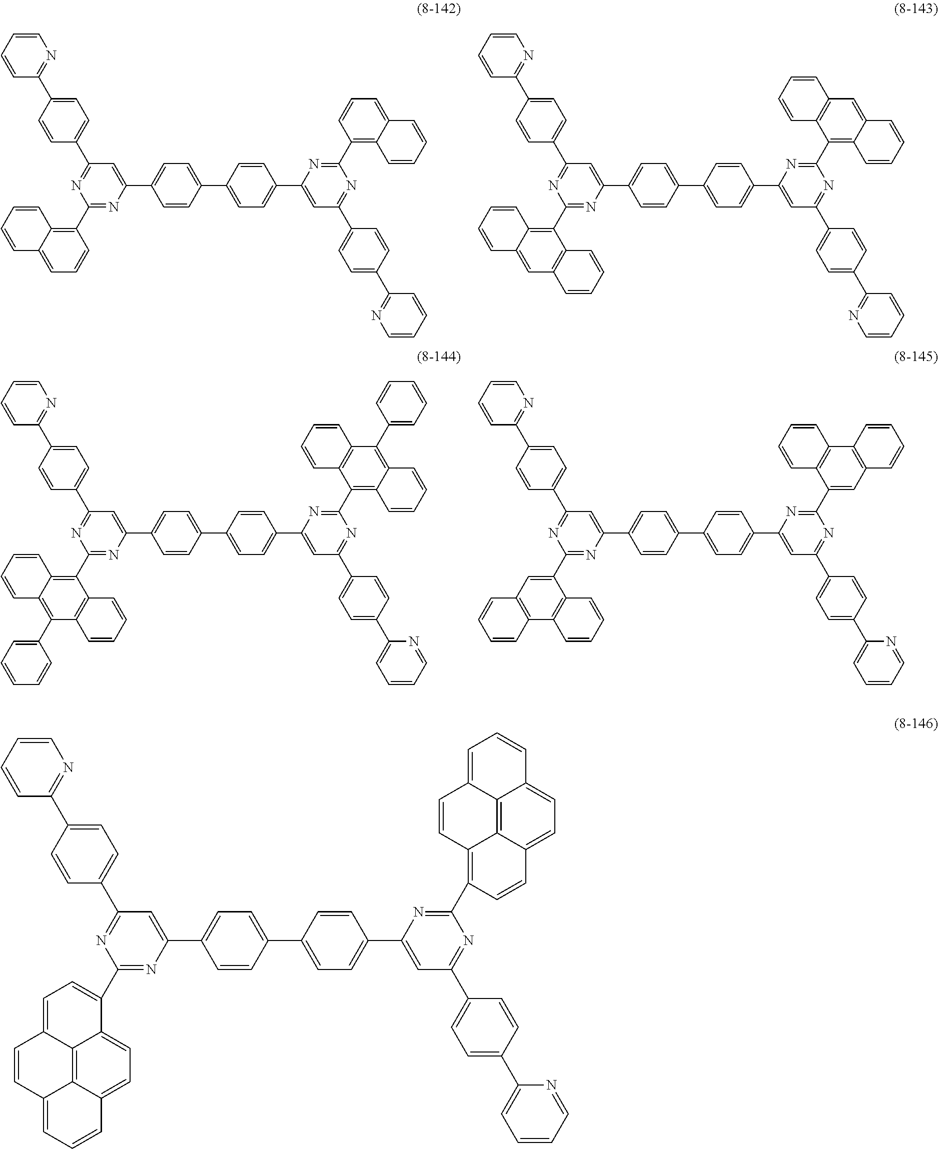

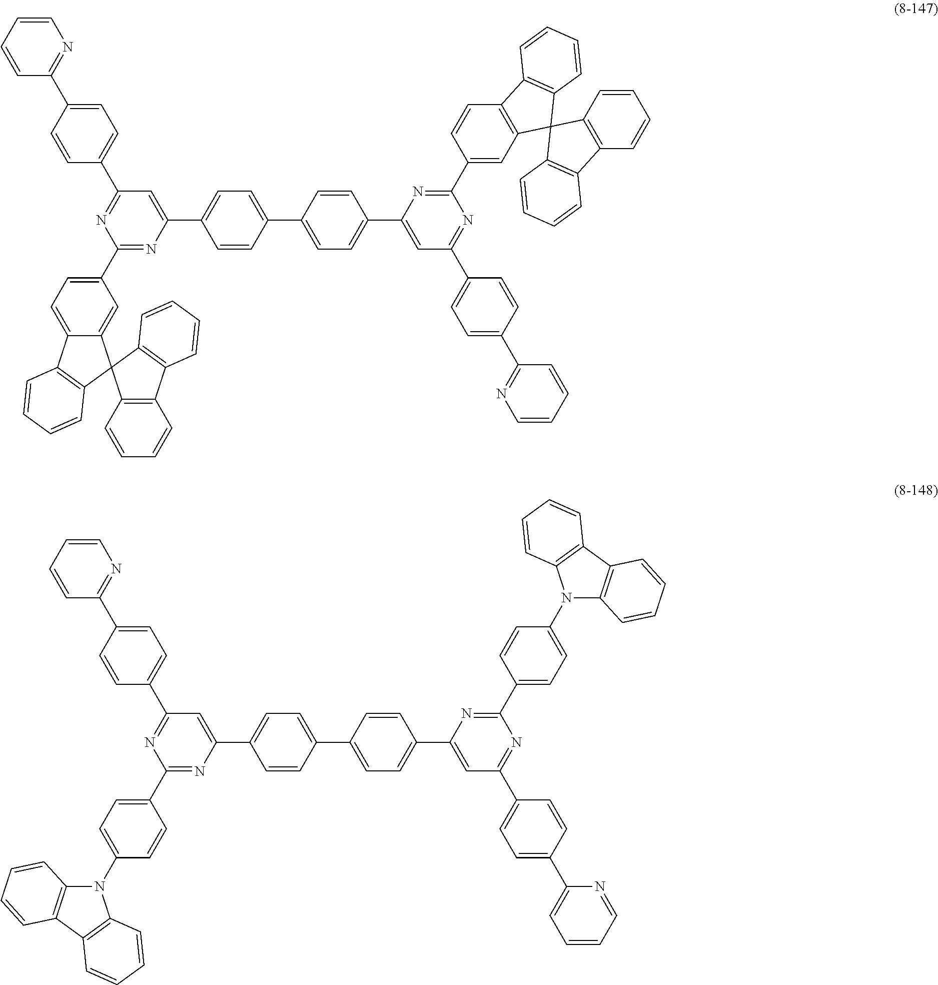

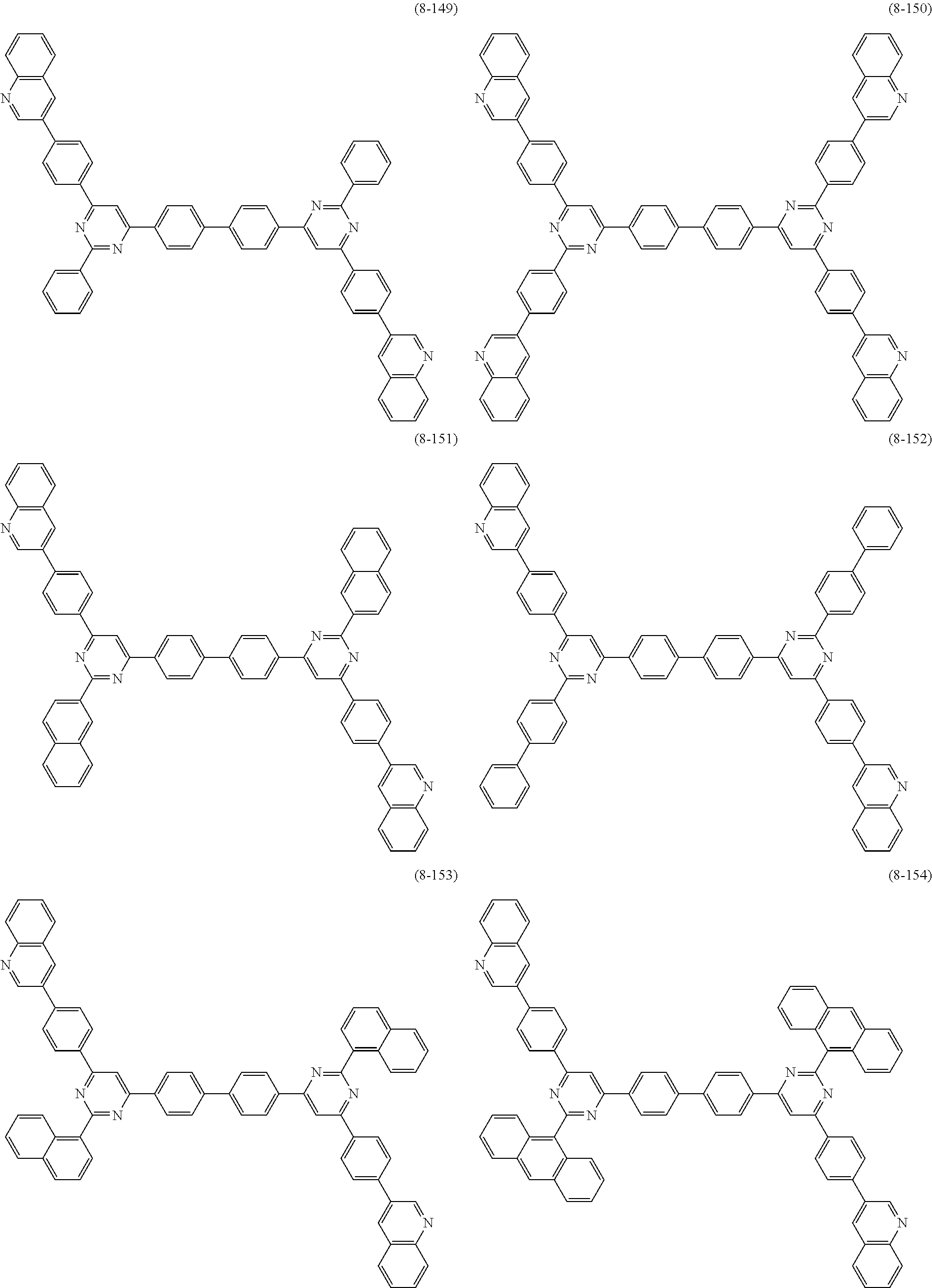

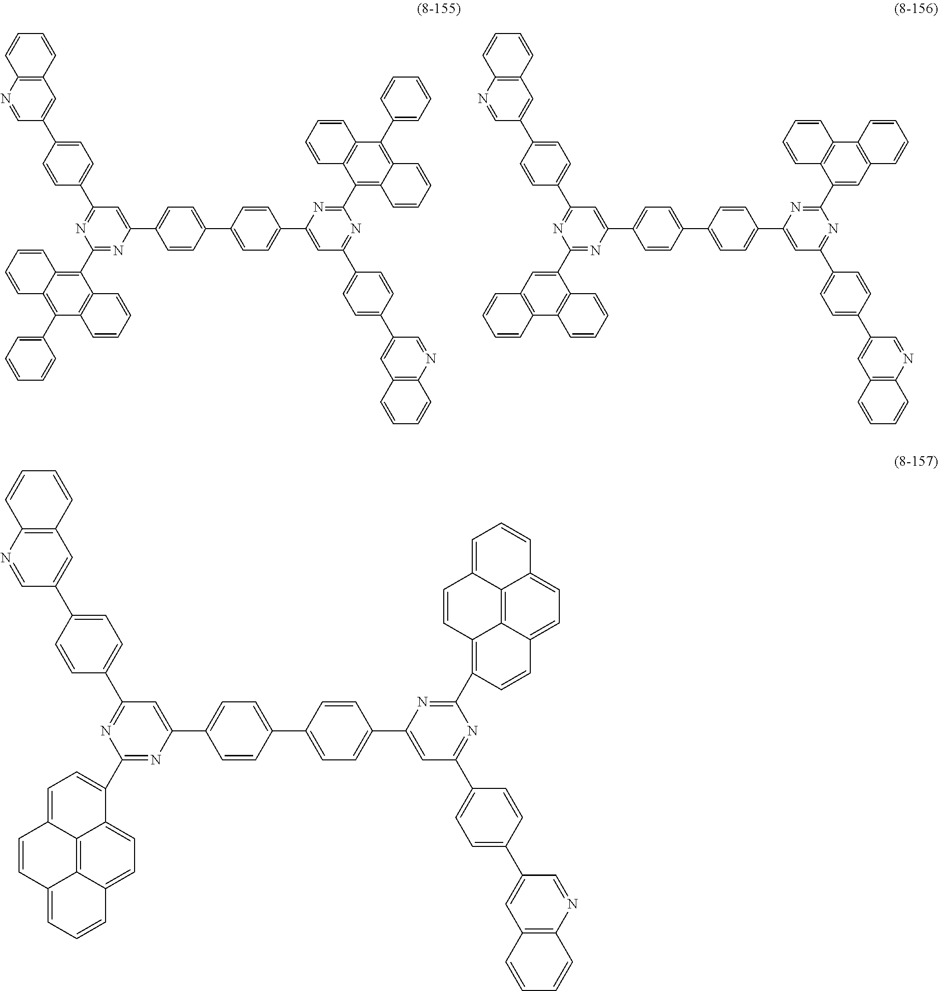

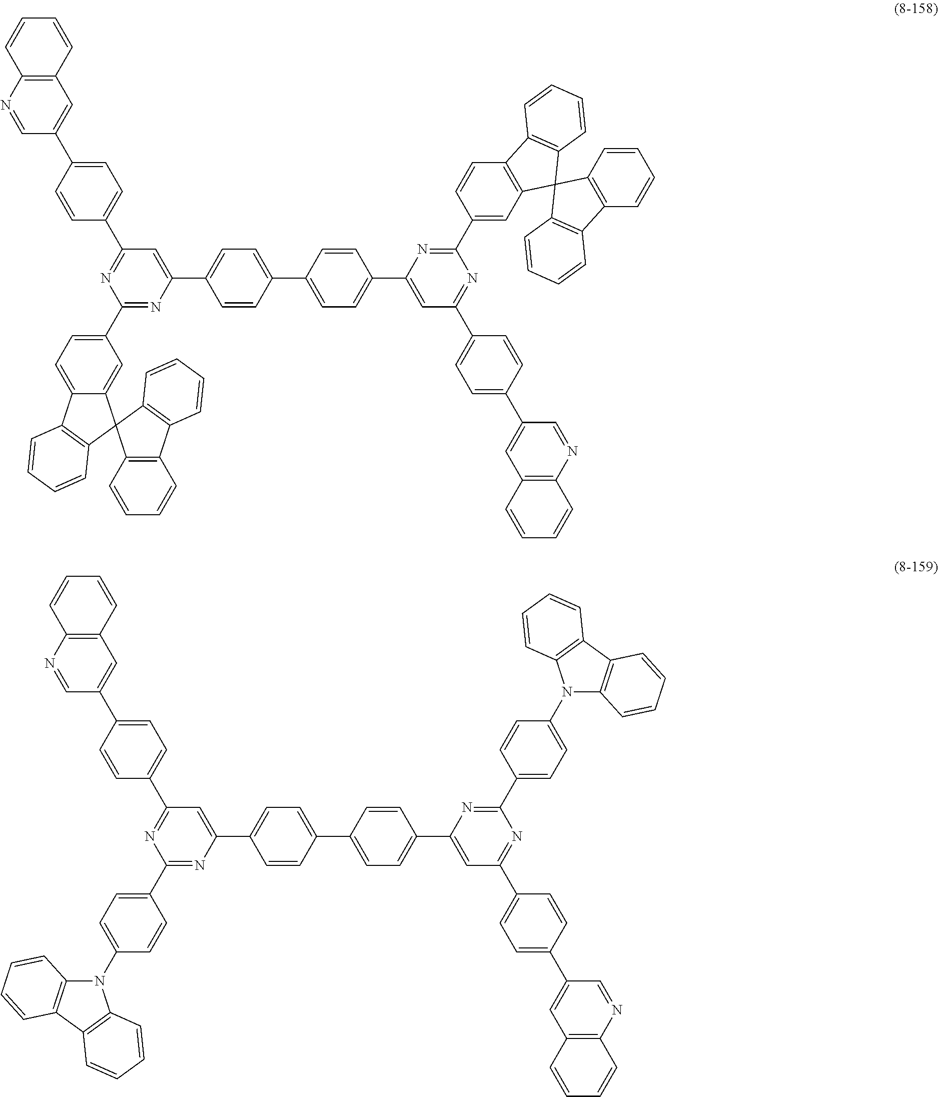

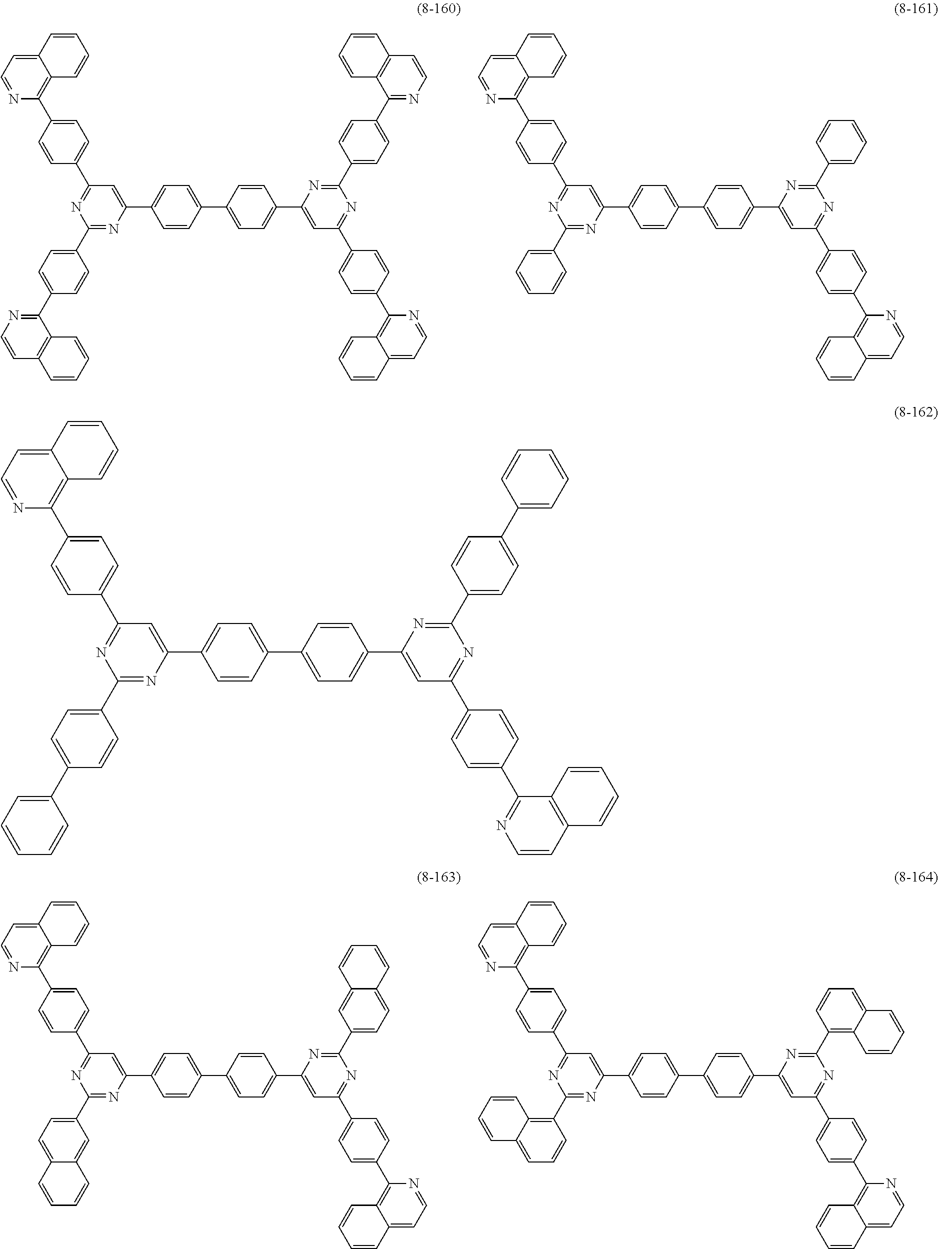

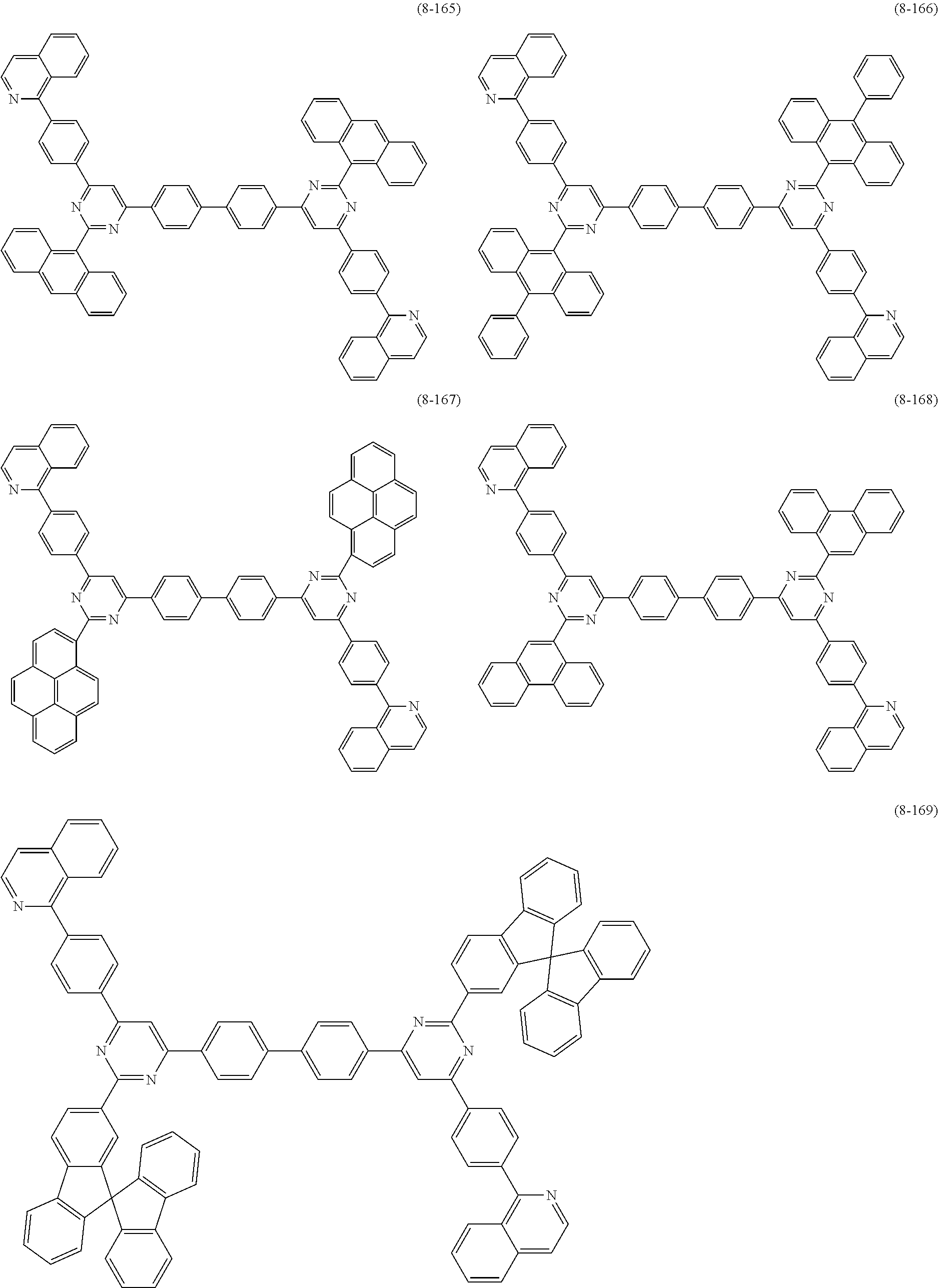

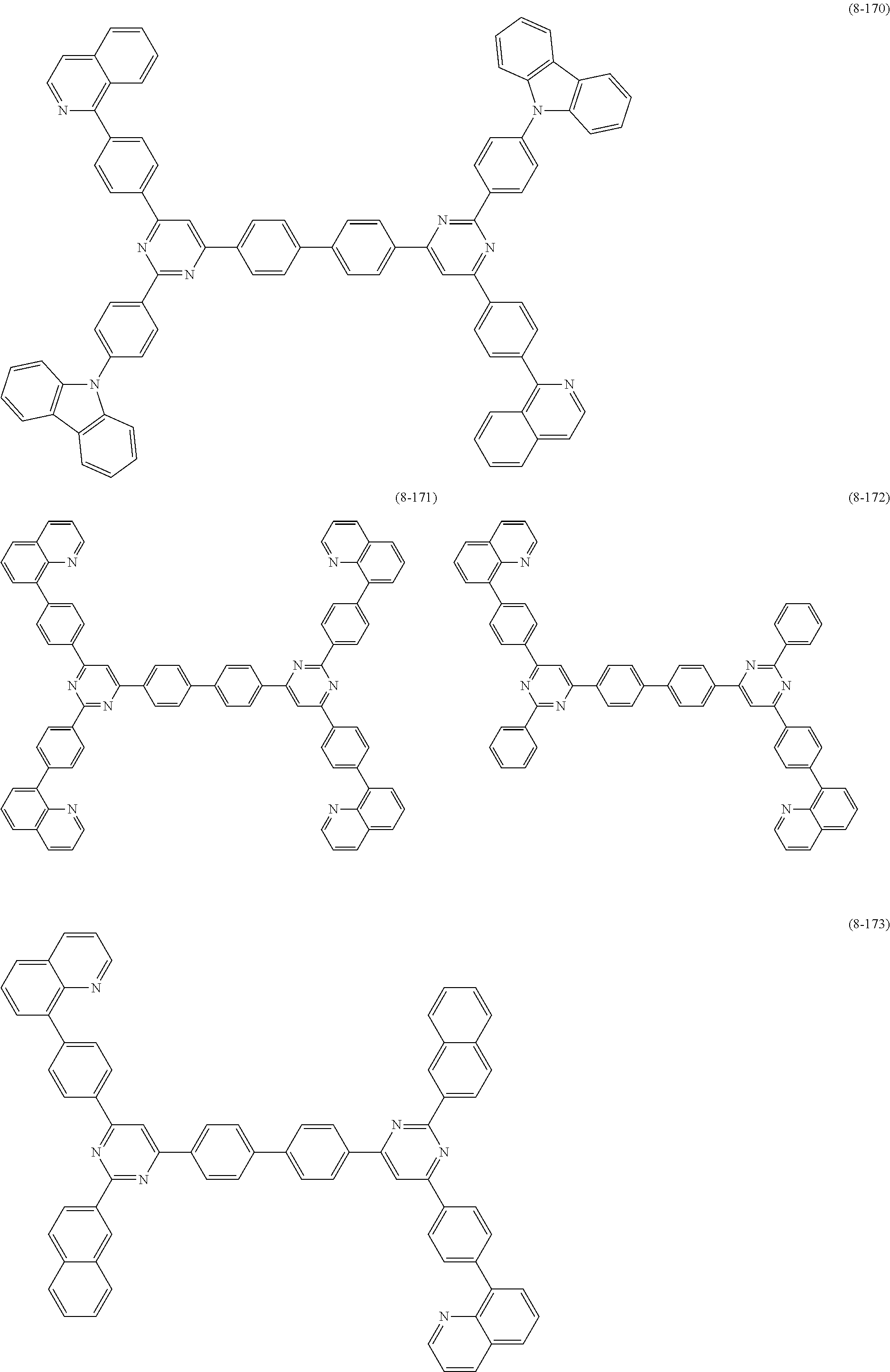

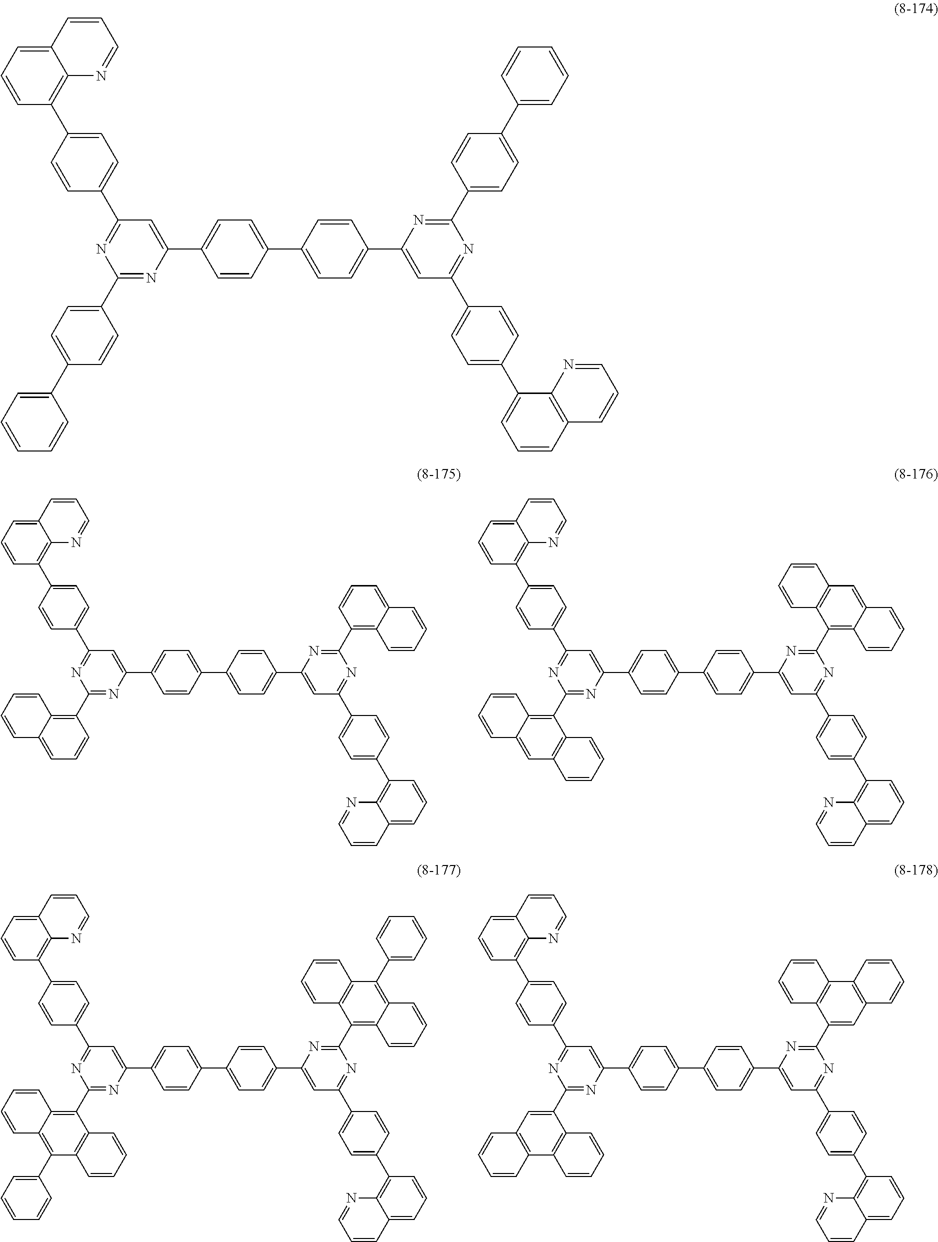

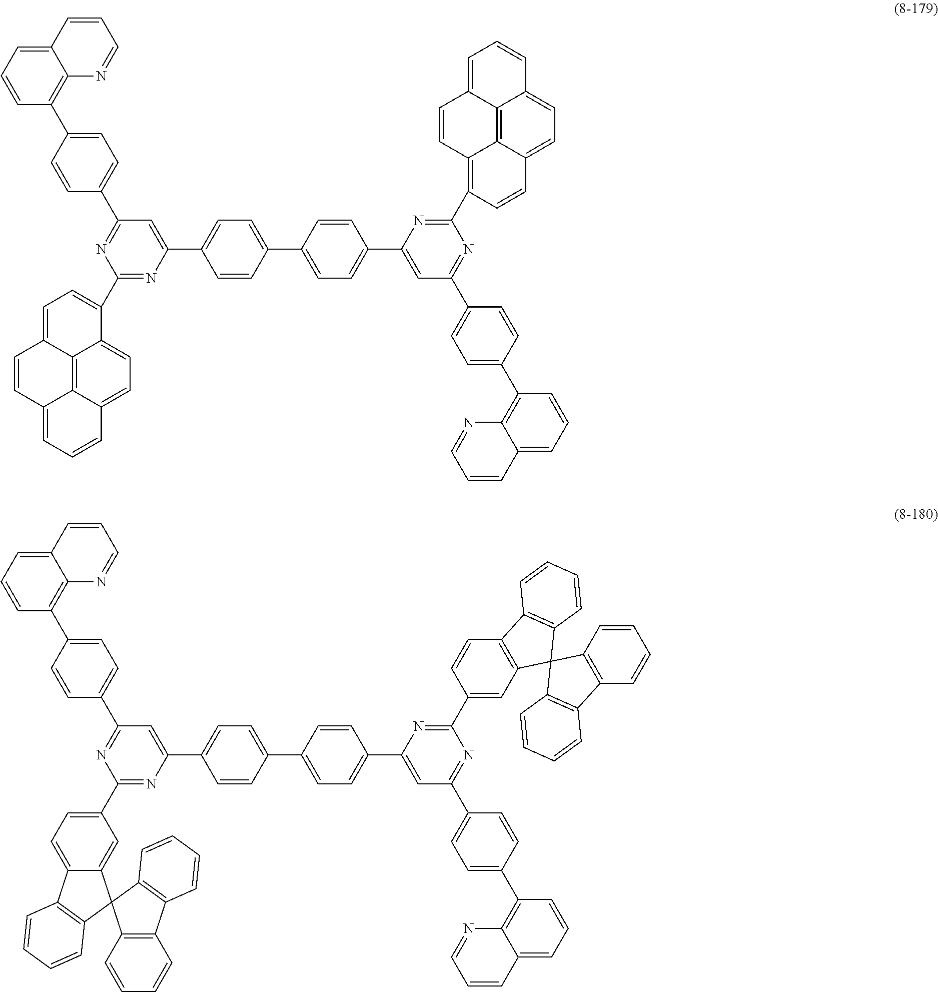

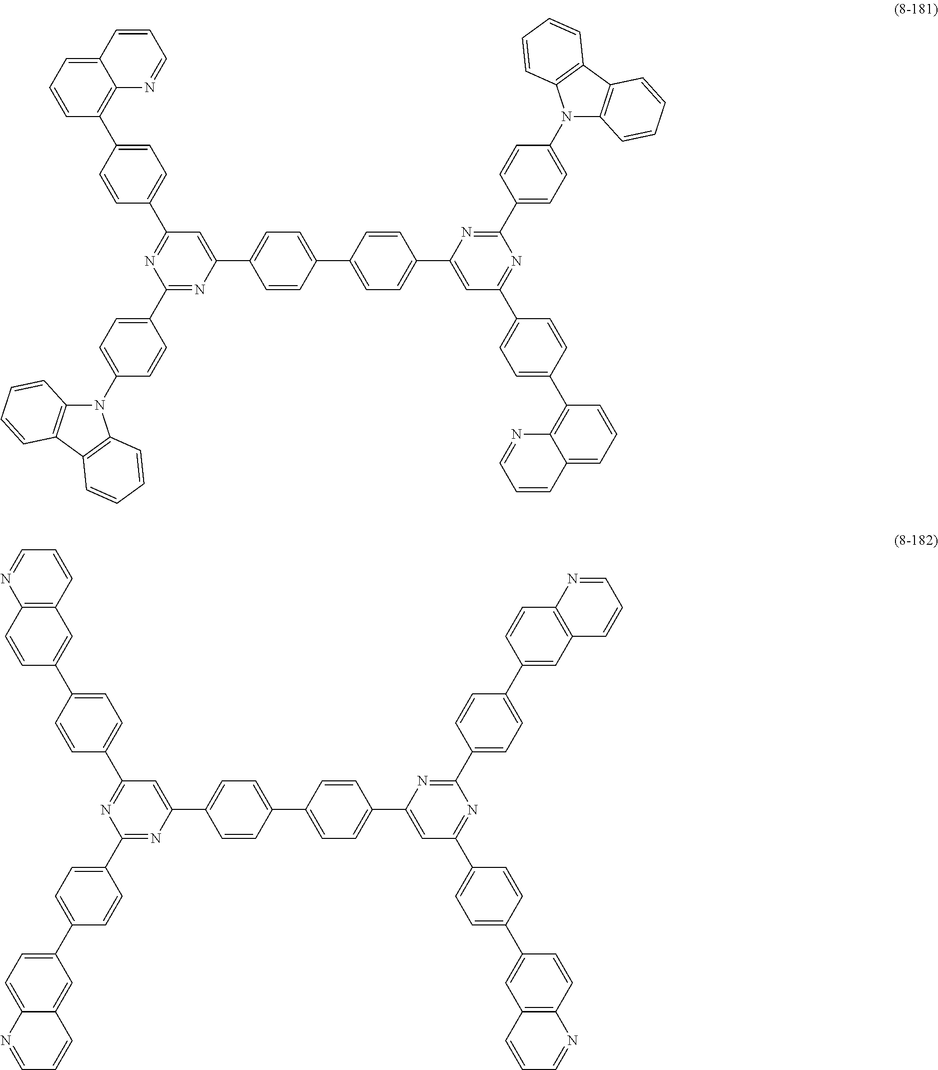

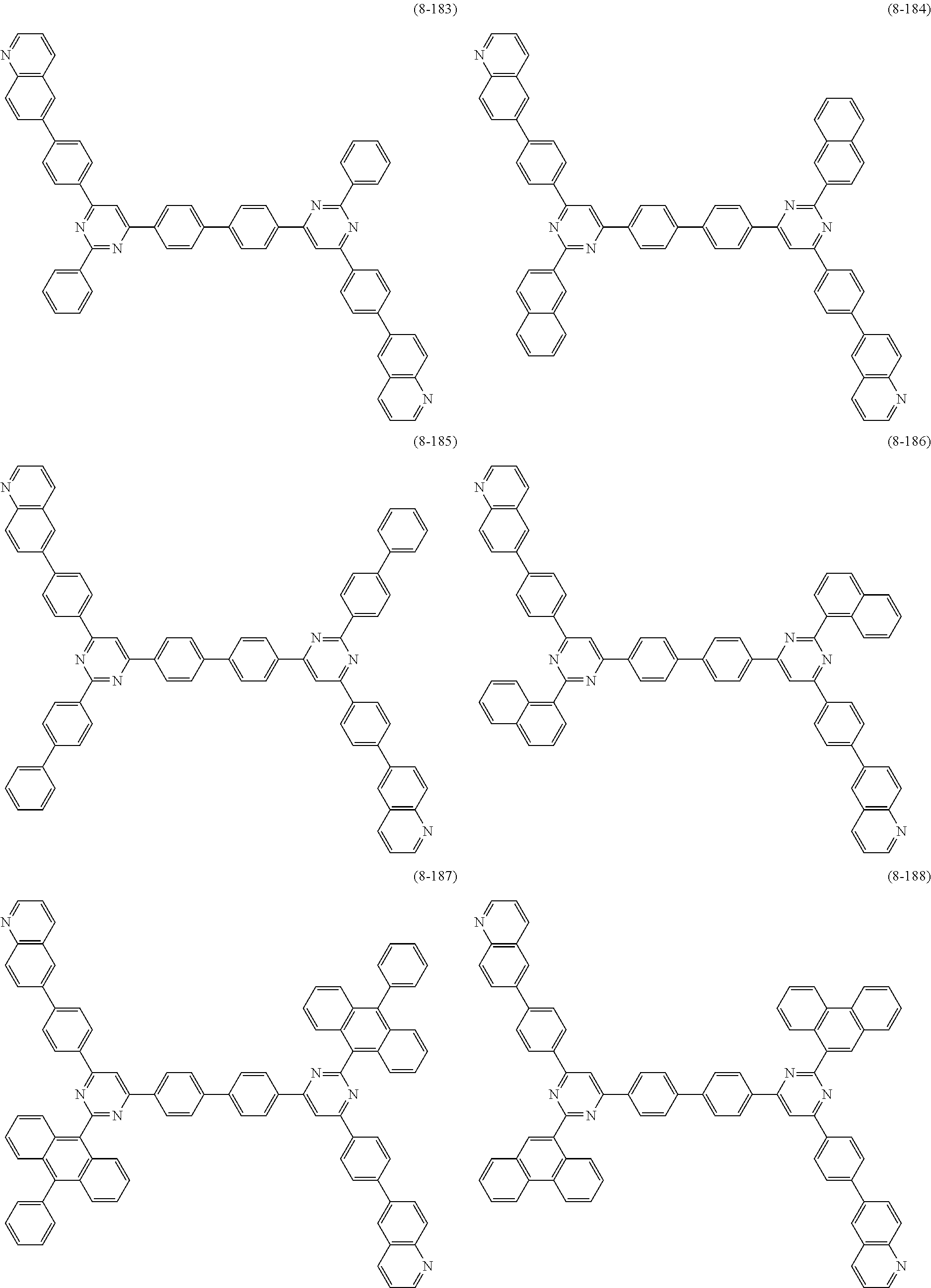

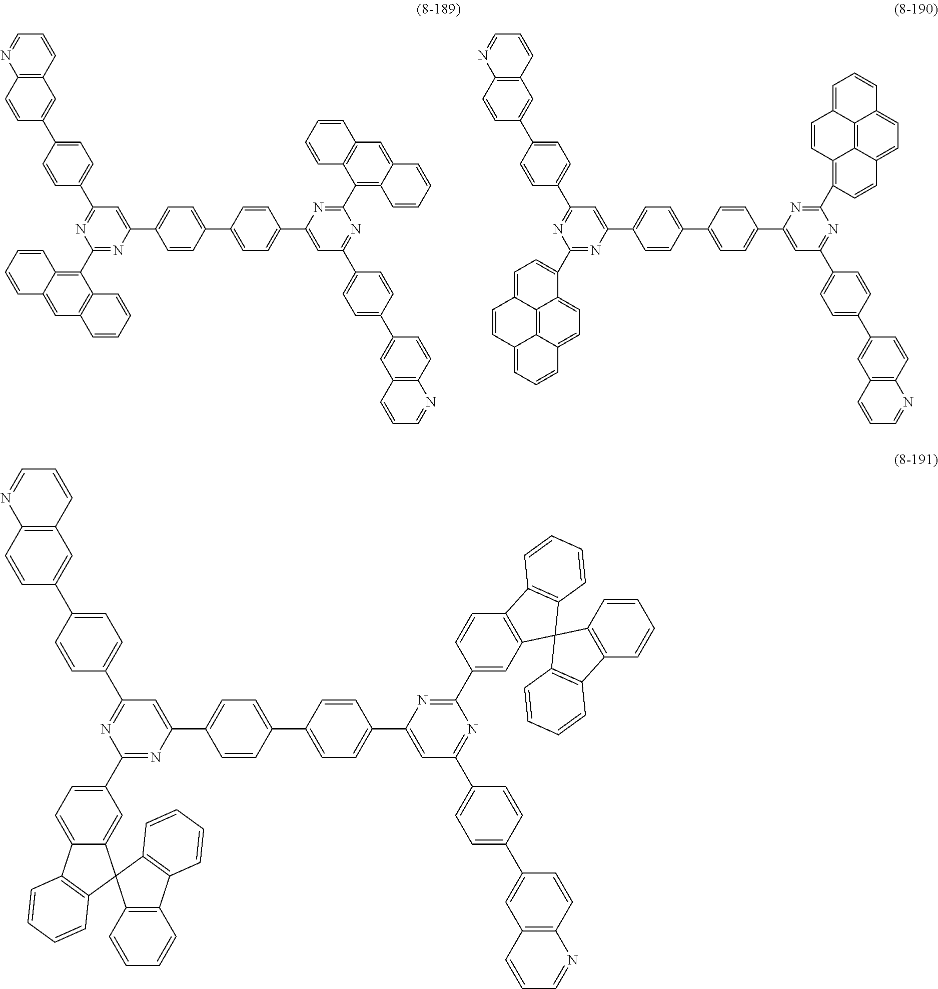

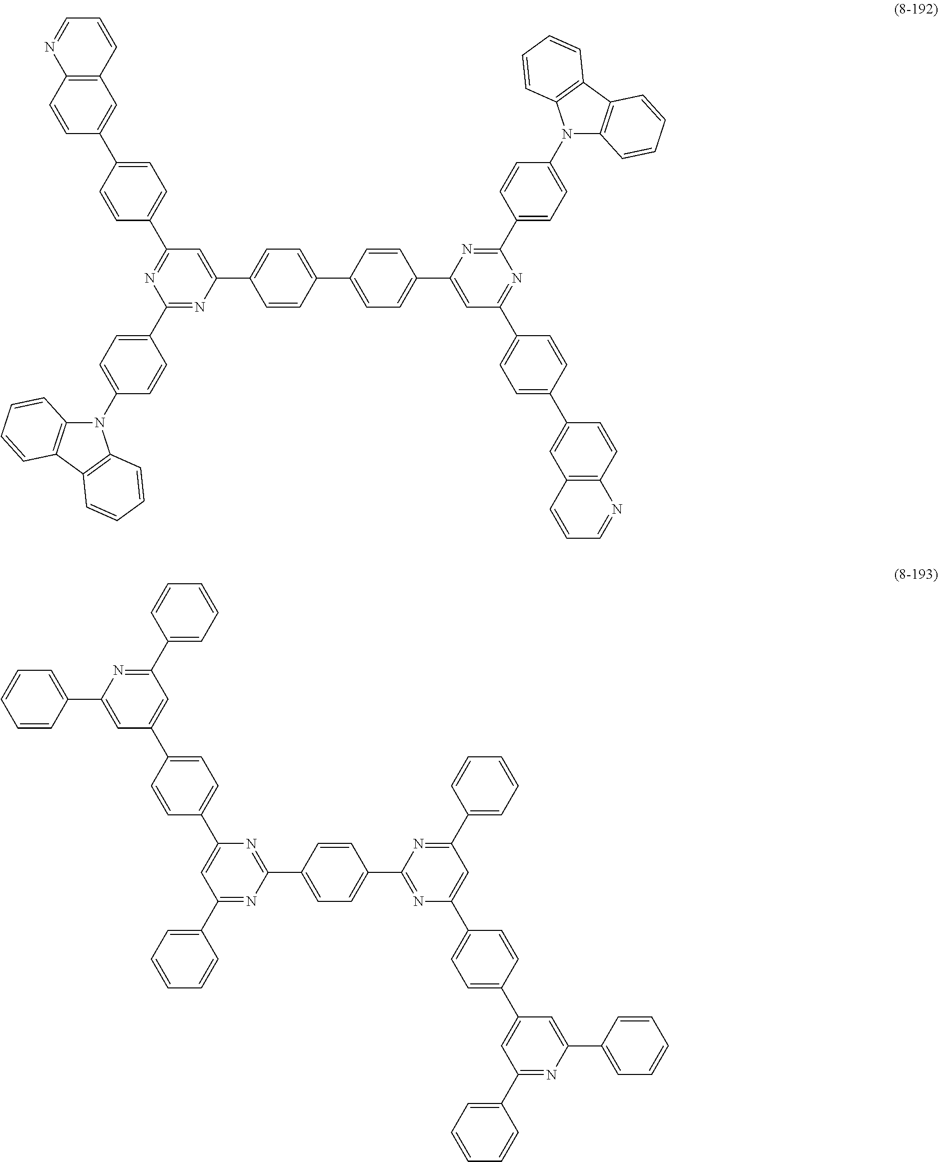

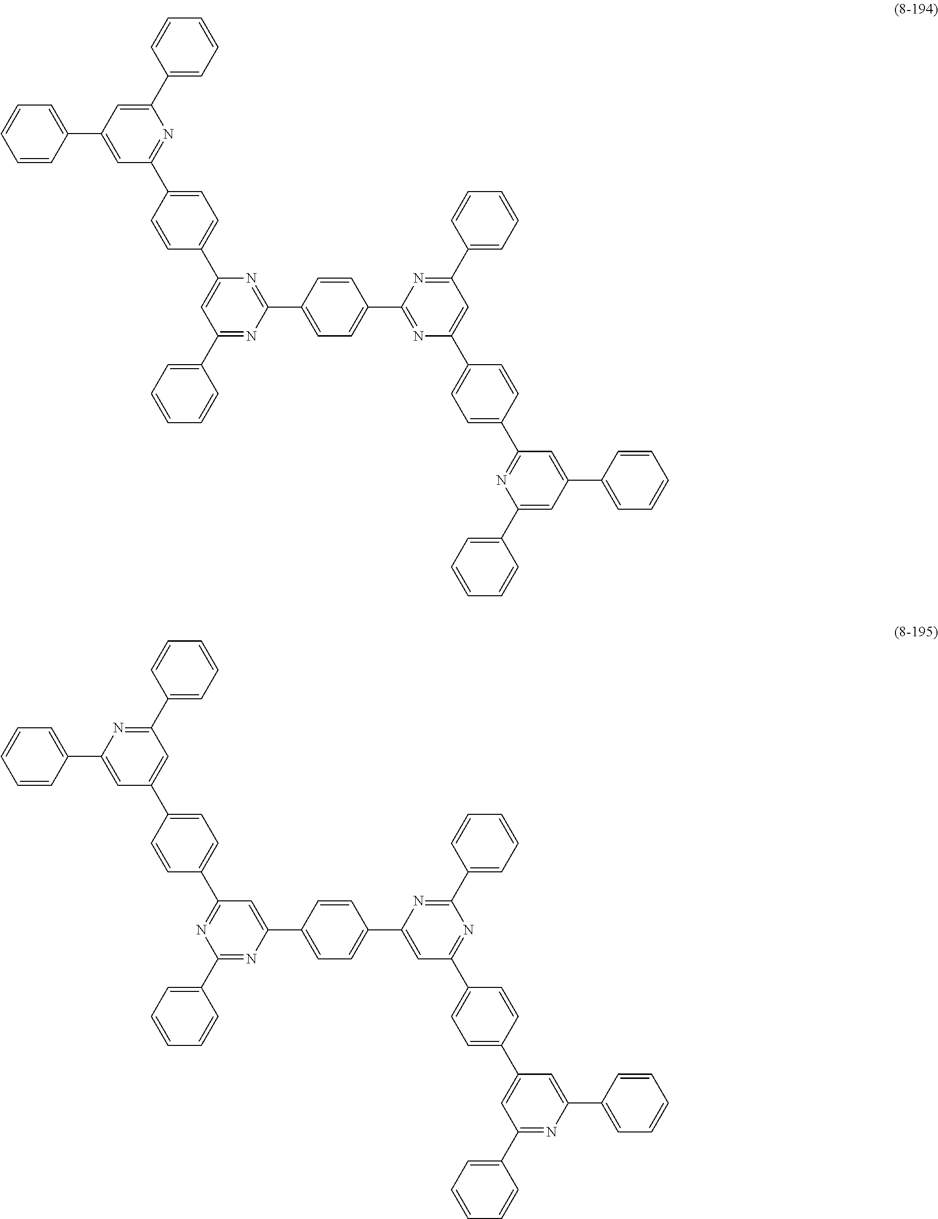

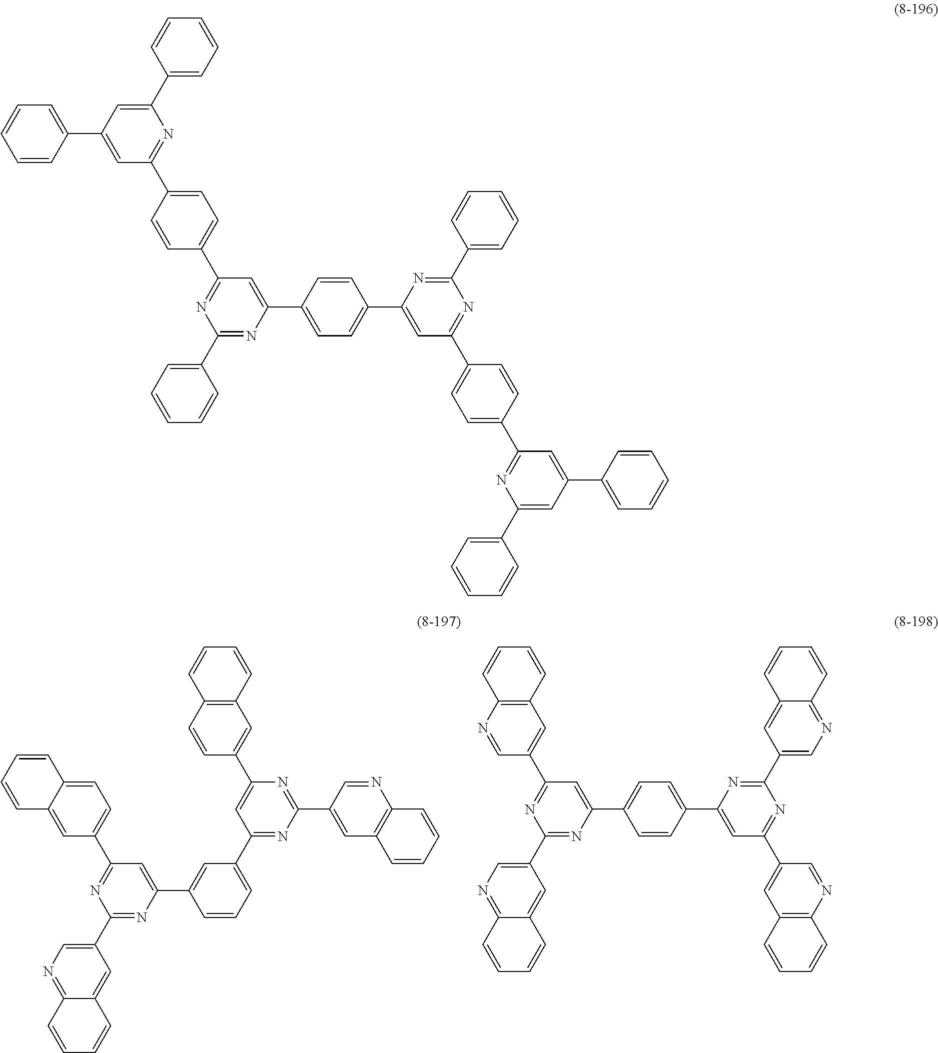

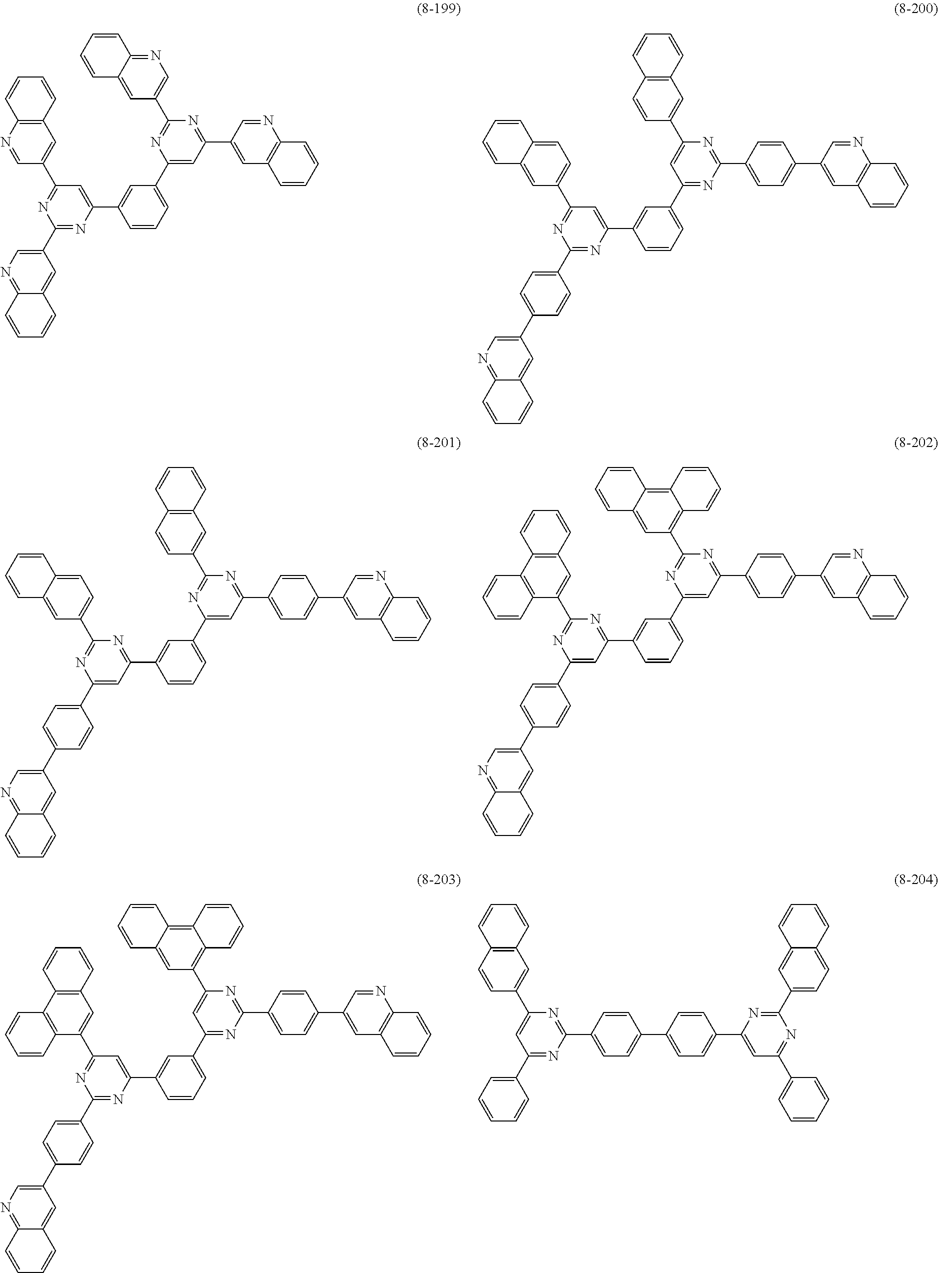

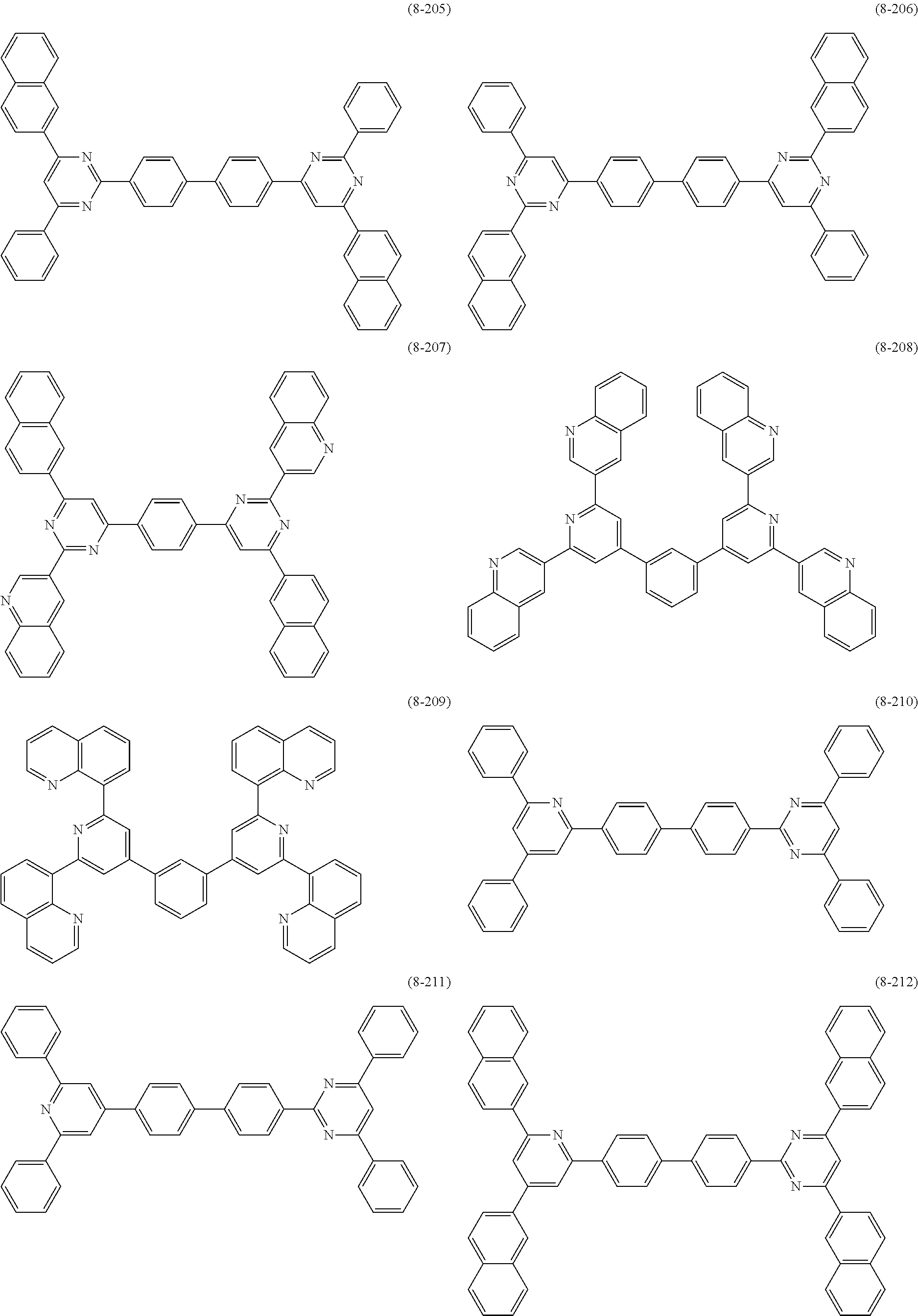

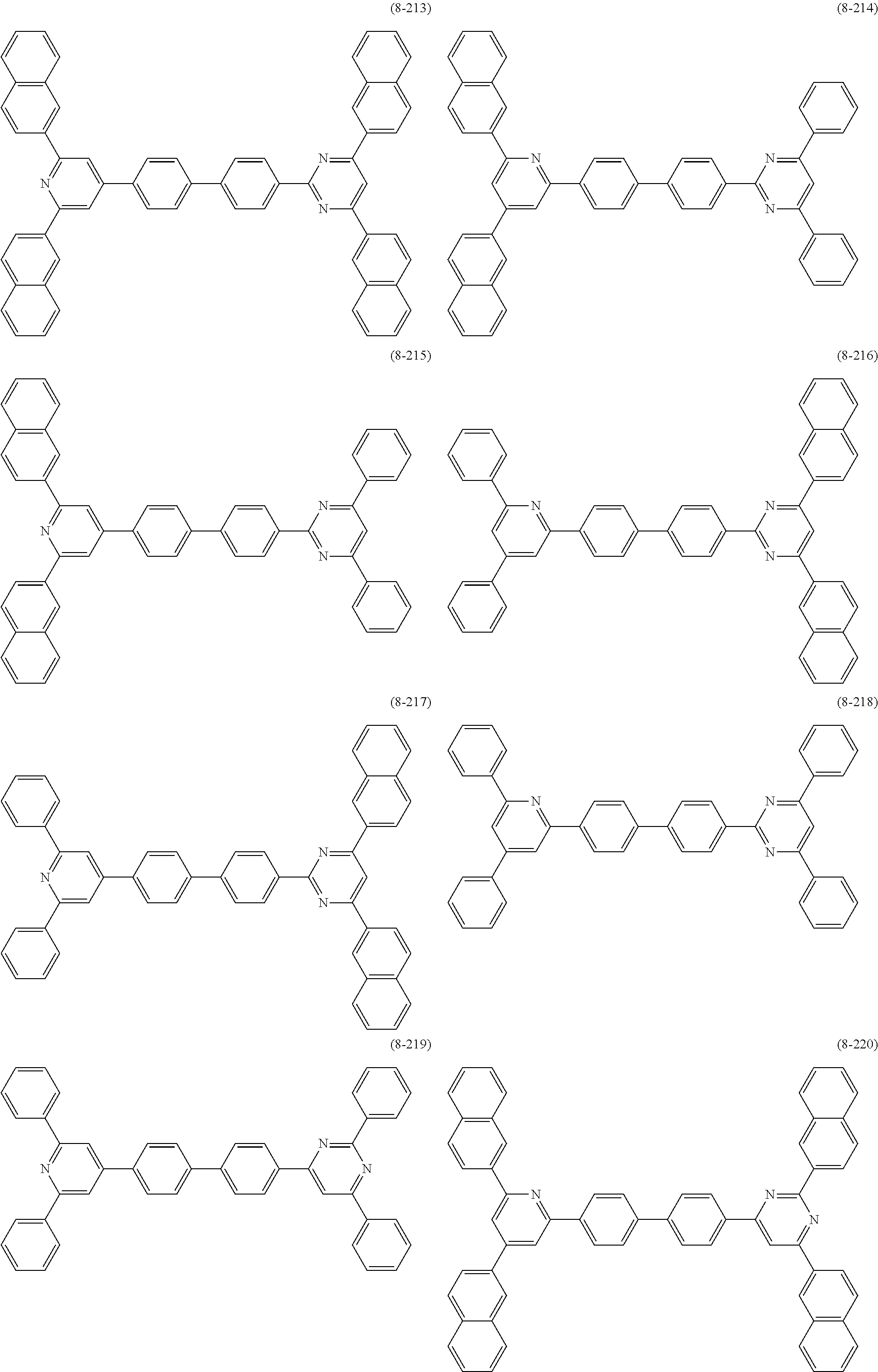

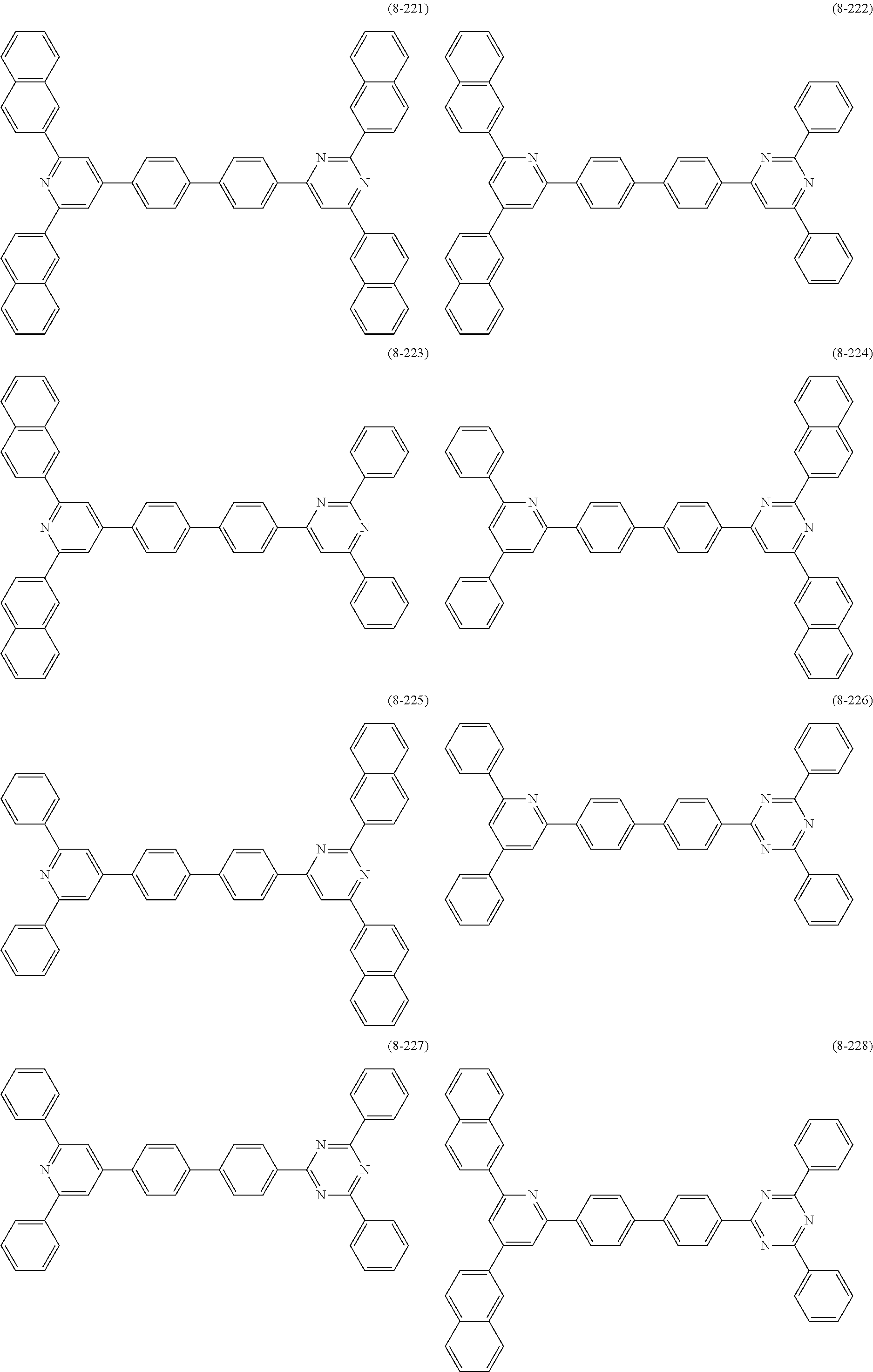

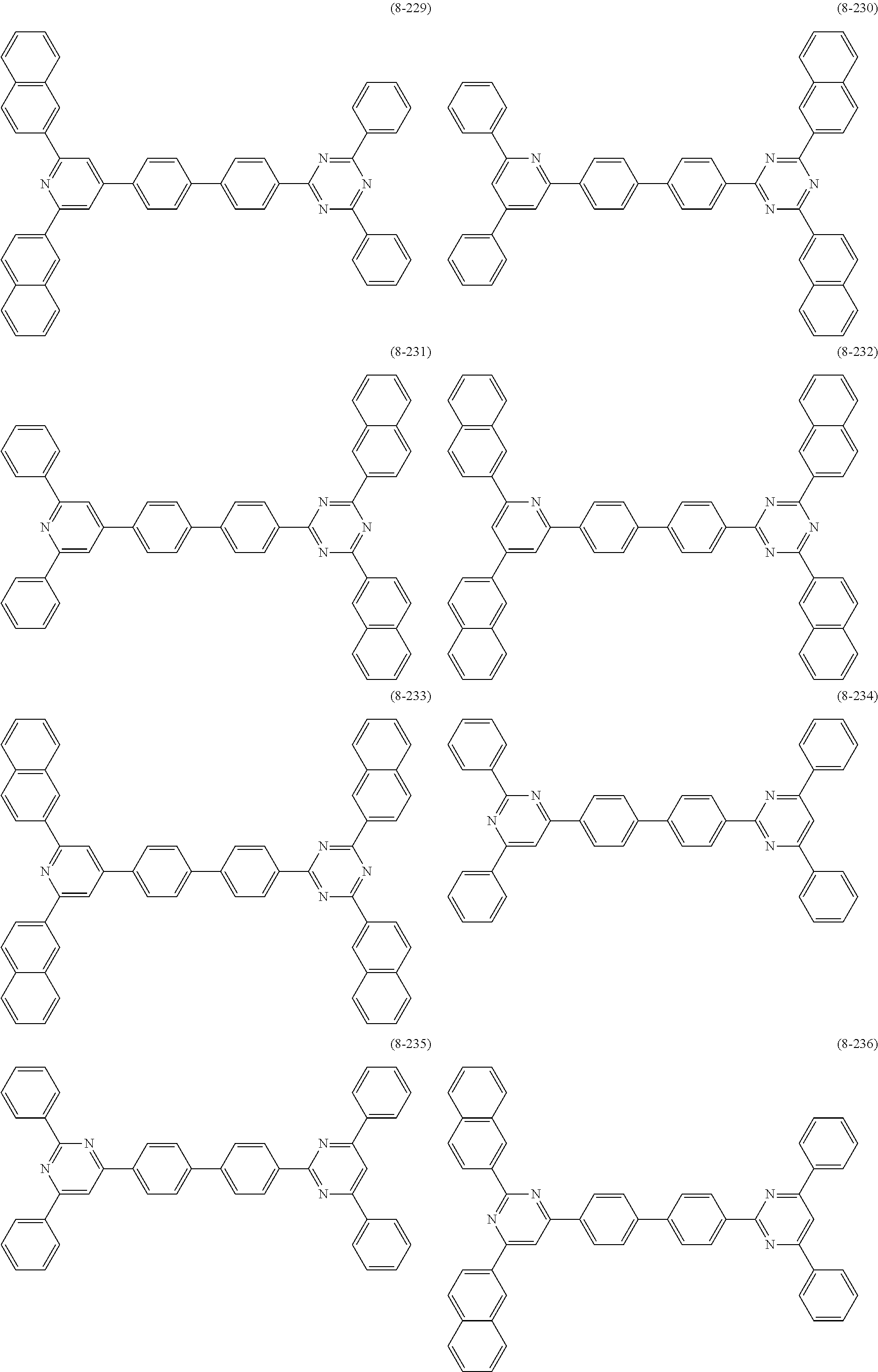

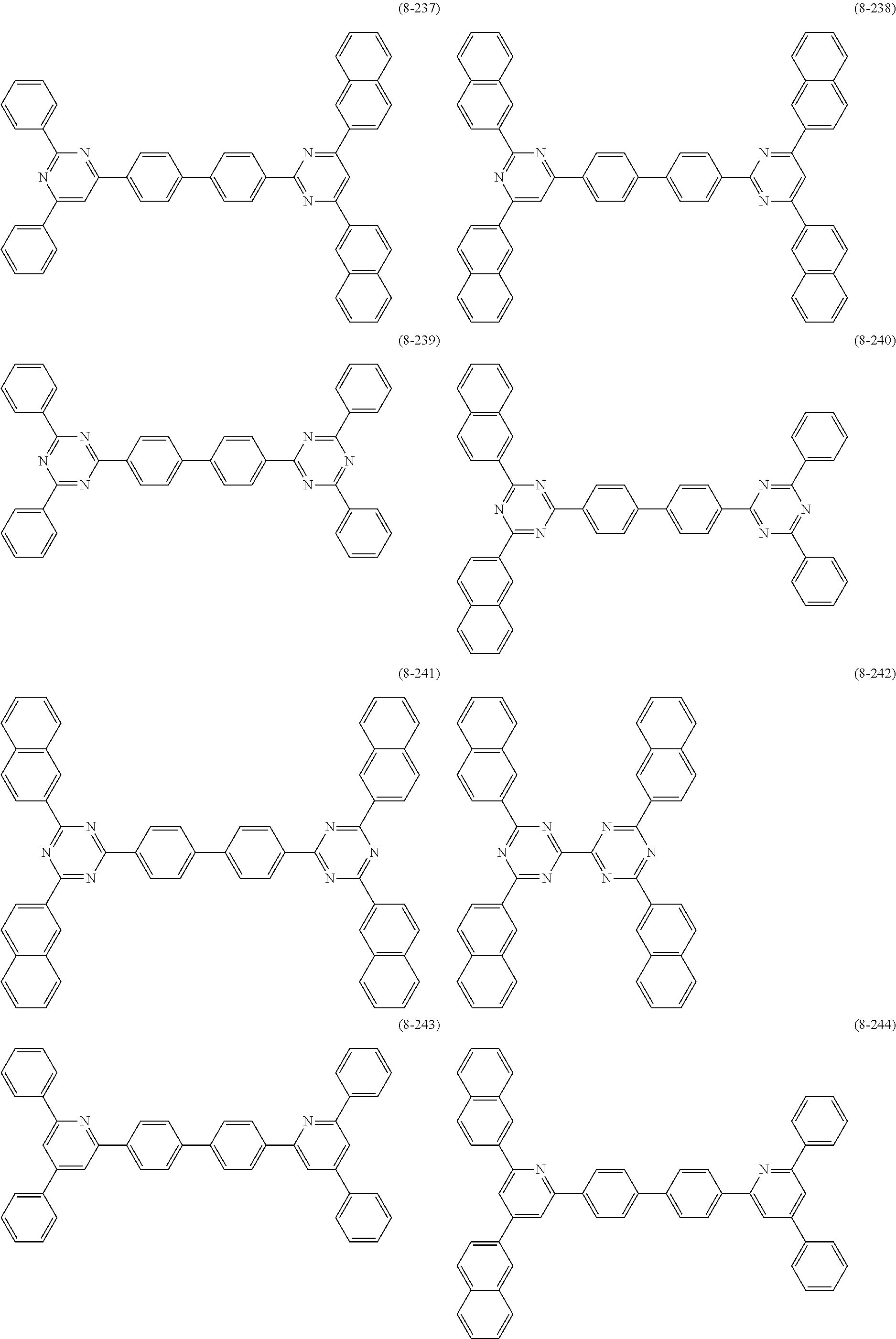

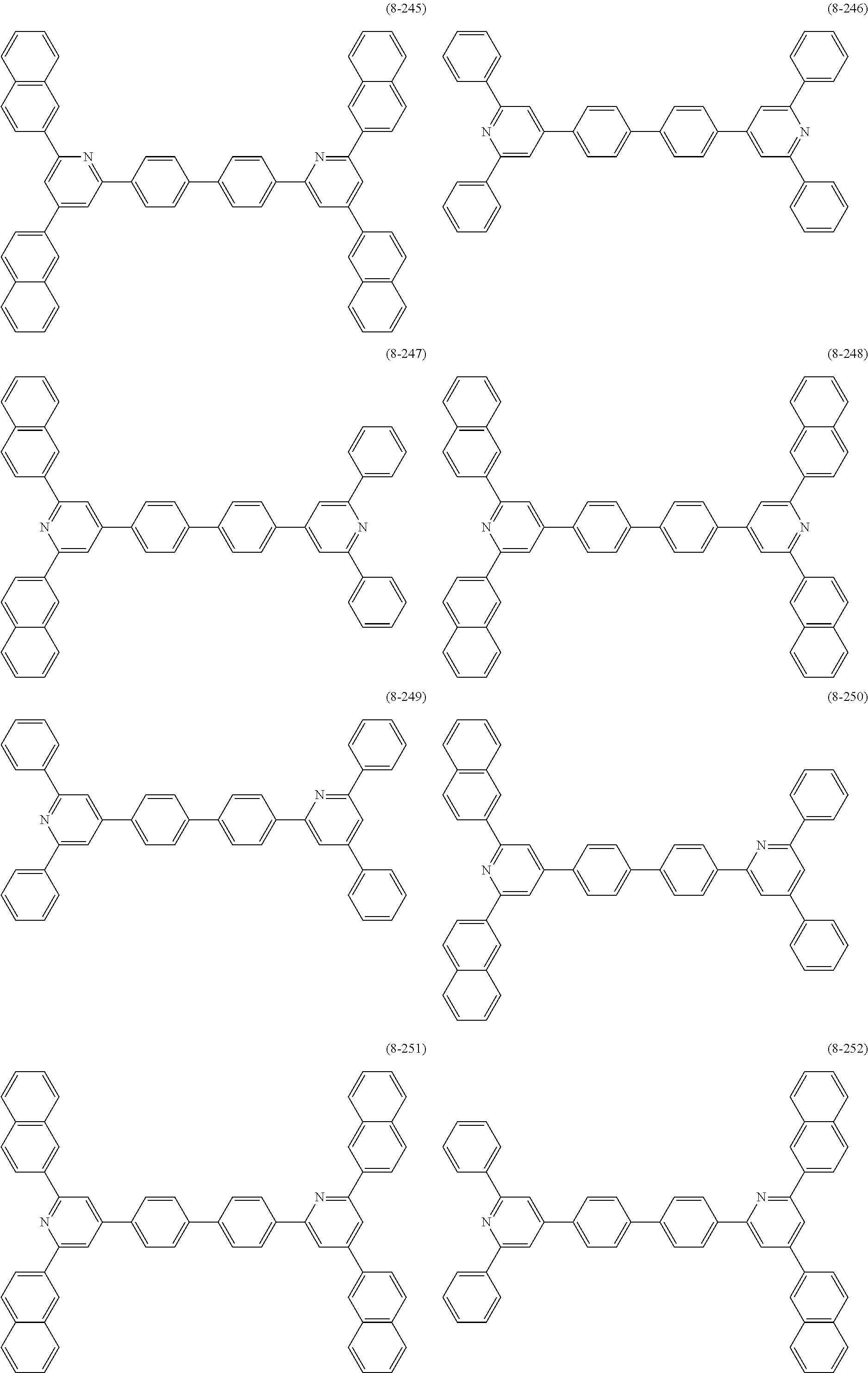

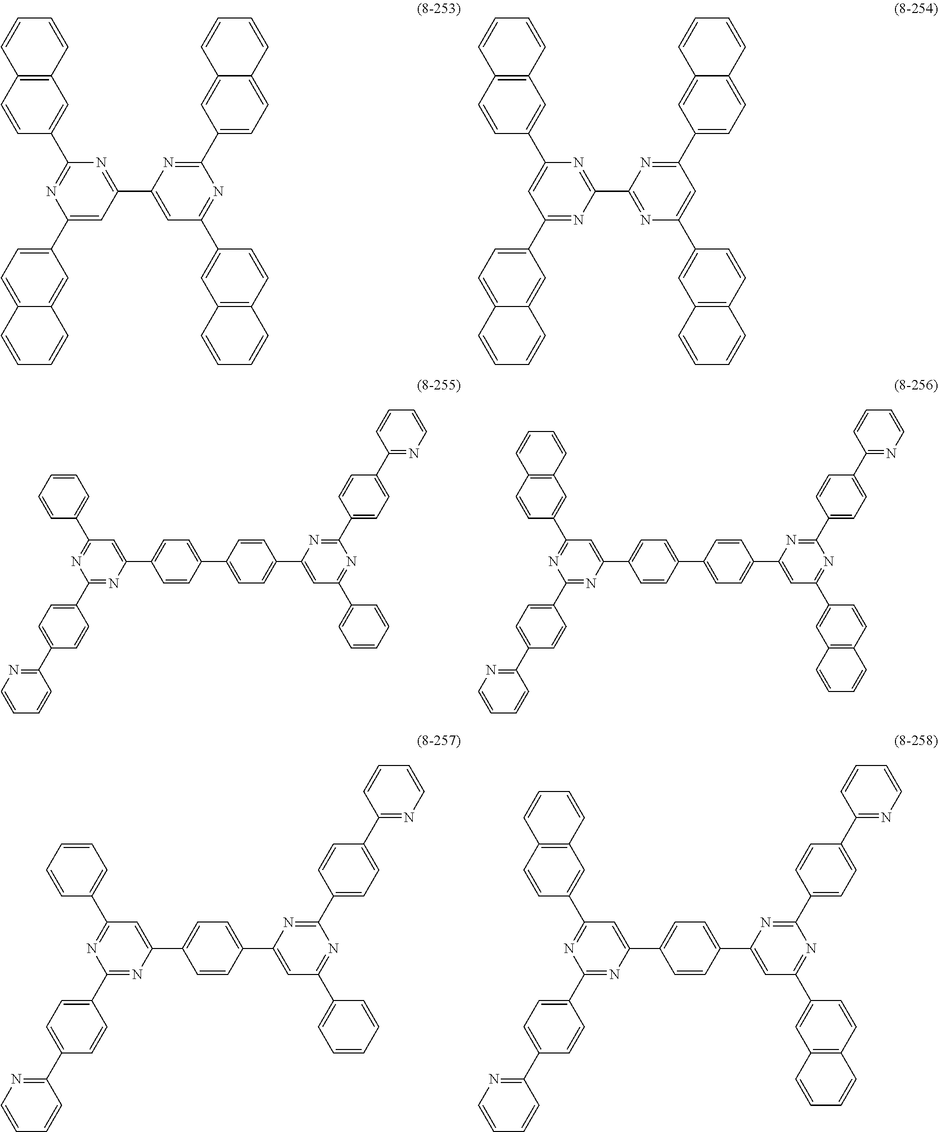

16. An organic photoelectric device, comprising:
an anode;
a cathode; and
at least one organic thin layer between the anode and the cathode, wherein the organic thin layer includes the compound for an organic photoelectric device as claimed in claim 1.

17. The organic photoelectric device as claimed in claim 16, wherein the organic thin layer including the compound for an organic photoelectric device includes an emission layer, a hole transport layer (HTL), a hole injection layer (HIL), a hole blocking layer, an electron transport layer (ETL), an electron injection layer (EIL), an electron blocking layer, or a combination thereof.

18. The organic photoelectric device as claimed in claim 16, wherein:
the organic thin layer including the compound for an organic photoelectric device is an emission layer, and
the compound for an organic photoelectric device is a phosphorescent host or a fluorescent host in the emission layer.

19. The organic photoelectric device as claimed in claim 16, wherein:
the organic thin layer including the compound for an organic photoelectric device is an emission layer, and
the compound for an organic photoelectric device is a fluorescent blue dopant in the emission layer.

20. A display device comprising the organic photoelectric device as claimed in claim 16.
